(19) United States
(12) Patent Application Publication
(10) Pub. No.: US 2024/0217983 A1
(43) Pub. Date: Jul. 4, 2024

(54) GPR119 AGONISTS

(71) Applicant: Kallyope, Inc., New York, NY (US)

(72) Inventor: Christopher MOYES, Westfield, NJ (US)

(21) Appl. No.: 18/285,812

(22) PCT Filed: Apr. 5, 2022
(86) PCT No.: PCT/US2022/023481

§ 371 Date: Oct. 5, 2023

Related U.S. Application Data
(60) Provisional application No. 63/171,342, filed on Apr. 6, 2021.

(51) Int. Cl. C07D 487/10; C07D 401/04; C07D 401/14; C07D 471/10; C07D 471/18; C07D 487/04; C07D 498/04; C07D 498/10

(52) U.S. Cl. CPC C07D 487/10; C07D 401/04; C07D 401/14; C07D 471/10; C07D 471/18; C07D 487/04; C07D 498/04; C07D 498/10

(57) ABSTRACT

This disclosure is directed, at least in part, to GPR119 agonists useful for the treatment of conditions or disorders involving the gut-brain axis. In some embodiments, the GPR119 agonists are gut-restricted compounds. In some embodiments, the condition or disorder is a metabolic disorder, such as diabetes, obesity, nonalcoholic steatohepatitis (NASH), or a nutritional disorder such as short bowel syndrome.

### CROSS REFERENCE TO RELATED APPLICATIONS

This application claims the benefit of U.S. Provisional Application No. 63/171,342 filed on Apr. 6, 2021, which is incorporated herein by reference in its entirety.

### BRIEF SUMMARY OF THE INVENTION

Disclosed herein, in certain embodiments, are G protein-coupled receptor 119 (GPR119) agonists useful for the treatment of conditions or disorders involving the gut-brain axis. In some embodiments, the GPR119 agonists are gut-restricted or selectively modulate GPR119 located in the gut. In some embodiments, the condition is selected from the group consisting of: central nervous system (CNS) disorders including mood disorders, anxiety, depression, affective disorders, schizophrenia, malaise, cognition disorders, addiction, autism, epilepsy, neurodegenerative disorders, Alzheimer's disease, and Parkinson's disease, Lewy Body dementia, episodic cluster headache, migraine, pain; metabolic conditions including diabetes and its complications such as chronic kidney disease/diabetic nephropathy, diabetic retinopathy, diabetic neuropathy, and cardiovascular disease, metabolic syndrome, obesity, dyslipidemia, and nonalcoholic steatohepatitis (NASH); eating and nutritional disorders including hyperphagia, cachexia, anorexia nervosa, short bowel syndrome, intestinal failure, intestinal insufficiency and other eating disorders; inflammatory disorders and autoimmune diseases such as inflammatory bowel disease, ulcerative colitis, Crohn's disease, psoriasis, celiac disease, and enteritis, including chemotherapy-induced enteritis or radiation-induced enteritis; necrotizing enterocolitis; diseases/disorders of gastrointestinal barrier dysfunction including environmental enteric dysfunction, spontaneous bacterial peritonitis; functional gastrointestinal disorders such as irritable bowel syndrome, functional dyspepsia, functional abdominal bloating/distension, functional diarrhea, functional constipation, and opioid-induced constipation; gastroparesis; nausea and vomiting; disorders related to microbiome dysbiosis, and other conditions involving the gut-brain axis.

Disclosed herein, in certain embodiments, is a compound of Formula (I):

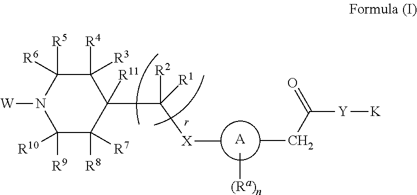

or a pharmaceutically acceptable salt, solvate, stereoisomer, or prodrug thereof, wherein:
Y is

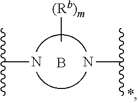

where Ring B is a heterocycloalkyl; wherein Ring B comprises 2 N atoms and 0 or 1 O or S atoms, and * represents the attachment point to K;
or Y is

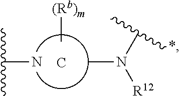

where Ring C is a bicyclic heterocycloalkyl; wherein Ring C comprises 1 or 2 N atoms and 0 or 1 O or S atoms, R¹² is hydrogen or C₁₋₄ alkyl, and * represents the attachment point to K;
each R^{b} is independently fluoro, C₁₋₆ alkyl, or C₁₋₆ fluoroalkyl;
K is C₁₋₈ alkyl, C₂₋₈ alkenyl, C₂₋₈ alkynyl, —C(═O)—C₁₋₈ alkyl, —C(═O)—C₂₋₃ alkenyl, —C(═O)—C₂₋₈ alkynyl, —[(CH₂)ₛ—Z]ₜ—R¹³, —[(CHR^{d})ₛ—Z]ₜ—R¹³, or —[(C(R^{d})₂)ₛ—Z]ₜ—R¹³; wherein each alkyl, alkenyl, or alkynyl is substituted by 1-6 R^{c} groups;
   each Z is independently —CH₂O—, —CH₂NH—, —CH₂NR^{d}—, —CH₂N⁺(R^{d})₂—, —NH—C(═O)—NH—, —C(═O)NH—, —CH₂S(═O)₂—, or —CH₂S(═O)—;
   each s is independently 1-6;
   each t is independently 1-6;
R¹³ is hydrogen, C₁₋₈ alkyl, C₂₋₈ alkenyl, or C₂₋₈ alkynyl, wherein the alkyl, alkenyl, or alkynyl is unsubstituted or substituted by 1-6 R^{c} groups;
each R^{c} is independently —OH, —CH₂OH, —CH₂CH₂OH, —NH₂, —CH₂NH₂, —NH(R^{d}), —CH₂NH(R^{d}), —N(R^{d})₂, —CH₂N(R^{d})₂, —N(R^{d})₃⁺, —C(═O)OH, —CH₂C(═O)OH, —CH₂CH₂C(═O)OH, —S(═O)₂OH, —S(═O)OH, —S(═O)₂NH₂, —P(═O)(OH)₂, —P(═O)(OH)(R^{d}), —P(═O)(OH)(H), P(═O)(OH)(OR^{d}), —B(OH)₂, —B(OR^{d})(OH), —NHCONHS(═O)₂(R^{d}), —N(R^{d})CONHS(═O)₂(R^{d}), —NHCON(R^{d})S(═O)₂(R^{d}), —C(═O)NHS(═O)₂(R^{d}), —S(═O)₂NHC(═O)R^{d}, —NHC(═NH)NH₂, —NHC(═NH)NHR^{d}, —NHC(═NH)N(R^{d})₂, —N(R^{d})C(═NH)NH₂, —N(R^{d})C(═NH)NH(R^{d}), —N(R^{d})C(═NH)N(R^{d})₂, —NHC(═N(R^{d}))NH₂, —NHC(═N(R^{d}))NHR^{d}, —NHC(═N(R^{d}))N(R^{d})₂, —N(R^{d})C(═N(R^{d}))NH₂, —N(R^{d})C(═N(R^{d}))NHR^{d}, —N(R^{d})C(═N(R^{d}))N(R^{d})₂, —NHC(═NH)NHC(═NH)NH₂, —N(R^{d})C(═NH)NHC(═NH)NH₂,

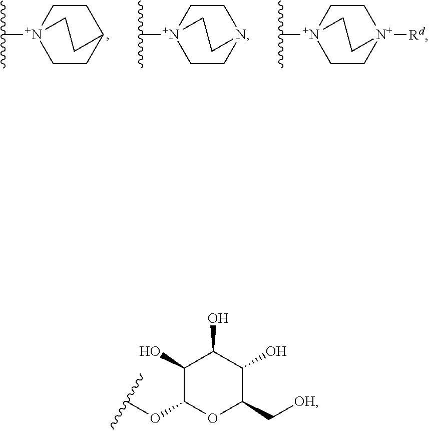

or a 4- to 6-membered heterocycle which is unsubstituted or substituted with 1, 2, 3, or 4 substituents selected from the group consisting of C₁₋₆ alkyl, —O—(C₁₋₆ alkyl), —OH, ═O and ═S;
each R^{d} is independently C₁₋₆ alkyl, C₁₋₆ fluoroalkyl, or C₃₋₆ cycloalkyl;
Ring A is phenyl, 5-membered monocyclic heteroaryl, or 6-membered monocyclic heteroaryl;
each R^{a} is independently halogen, —CN, C₁₋₆ alkyl, C₁₋₆ fluoroalkyl, or C₃₋₆ cycloalkyl;
X is —O—, —NR¹⁴—, #—CH₂O—, #—CH₂NR¹⁴—, #—C(═O)O—, #—C(═O)NR¹⁴—, #—CH₂C(═O)O—, #—CH₂C(═O)NR¹⁴—, #—OC(═O)—, #—NR¹⁴C(═O)—, #—CH₂OC(═O)—, or #—CH₂NR¹⁴C(═O)—, where # represents the attachment point to Ring A;
R¹⁴ is hydrogen or C₁₋₄ alkyl;
each R¹ is independently hydrogen, fluorine, —OH, C₁₋₆ alkyl, or C₁₋₆ alkoxy;
or two R¹ are taken together with the intervening atoms to which they are attached to form a C₃₋₆ cycloalkyl;
each R² is independently hydrogen, fluorine, or C₁₋₆ alkyl;
R³, R⁴, R⁵, R⁶, R⁷, R⁸, R⁹, and R¹⁰ are each independently hydrogen, C₁₋₆ alkyl, C₁₋₆ alkoxy, or C₁₋₆ fluoroalkyl;
or R³ and R⁷ or R³ and R⁹ or R⁵ and R⁹ are taken together with the intervening atoms to which they are attached to form a ring;
R¹¹ is hydrogen, C₁₋₆ alkyl, or C₁₋₆ fluoroalkyl;
or R¹¹ and one R¹ are taken together with the intervening atoms to which they are attached to form a C₃₋₆ cycloalkyl;
W is phenyl, 5-membered monocyclic heteroaryl, or 6-membered monocyclic heteroaryl, wherein the phenyl or heteroaryl is unsubstituted or substituted with 1, 2, or 3 substituents selected from R^{e};
each R^{e} is independently halogen, —OH, —CN, —C(O)OH, —C(O)O(C₁₋₆ alkyl), C₁₋₆ alkyl, C₁₋₆ alkoxy, C₃₋₆ cycloalkyl, phenyl, or 5- to 6-membered heteroaryl; wherein each alkyl, alkoxy, and cycloalkyl is unsubstituted or substituted with 1, 2, or 3 substituents selected from the group consisting of halogen, —OH, C₁₋₆ alkyl, and CL-6 alkoxy;
or W is —C(═O)O—R¹⁵;
R¹⁵ is C₁₋₆ alkyl, C₃₋₆ cycloalkyl, or 3- to 6-membered heterocycloalkyl, wherein the alkyl, cycloalkyl, and heterocycloalkyl are unsubstituted or substituted by 1-3 substituents independently selected from the group consisting of fluorine, —OH, C₁₋₆ alkyl, C₁₋₆ fluoroalkyl, or C₃₋₆ cycloalkyl;
m is 0, 1, 2, 3, or 4;
n is 0, 1, 2, 3, or 4; and
r is 1, 2, 3, 4, 5 or 6.

In some embodiments, disclosed herein is a compound of Formula (Ia), or a pharmaceutically acceptable salt, or solvate thereof:

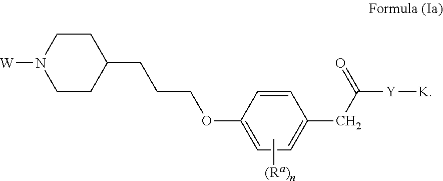

In some embodiments, disclosed herein is a compound of Formula (Ib), or a pharmaceutically acceptable salt, or solvate thereof:

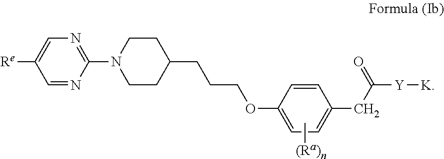

In some embodiments, disclosed herein is a compound of Formula (II), or a pharmaceutically acceptable salt, or solvate thereof:

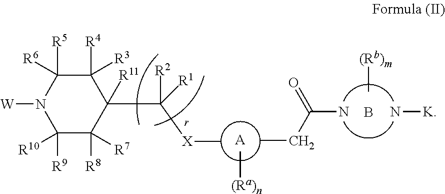

In some embodiments, disclosed herein is a compound of Formula (IIa), or a pharmaceutically acceptable salt, or solvate thereof:

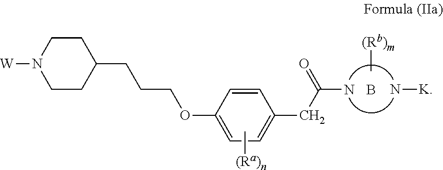

In some embodiments, disclosed herein is a compound of Formula (IIb), or a pharmaceutically acceptable salt, or solvate thereof:

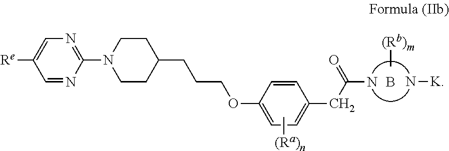

In some embodiments, disclosed herein is a compound of Formula (III), or a pharmaceutically acceptable salt, or solvate thereof:

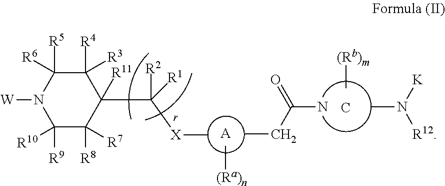

In some embodiments, disclosed herein is a compound of Formula (IIIa), or a pharmaceutically acceptable salt, or solvate thereof:

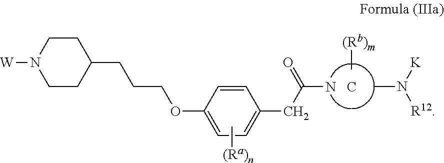

In some embodiments, disclosed herein is a compound of Formula (IIIb), or a pharmaceutically acceptable salt, or solvate thereof:

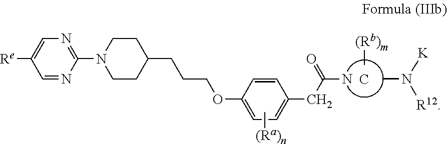

In some embodiments,
X is —O—, #—CH₂O—, #—C(═O)O—, or #—CH₂C(═O)O—, where # represents the attachment point to Ring A;
each R¹ is independently hydrogen, fluorine, —OH, C₁₋₆ alkyl, or C₁₋₆ alkoxy;
or two R¹ on adjacent carbon atoms are taken together with the intervening atoms to which they are attached to form a cyclopropyl;
each R² is independently hydrogen, fluorine, or C₁₋₆ alkyl;
R¹¹ is hydrogen or C₁₋₆ alkyl;
R³, R⁴, R⁵, R⁶, R⁷, R⁸, R⁹, and R¹⁰ are each independently hydrogen or C₁₋₆ alkyl;
Ring A is phenyl or pyridinyl;
each R^{a} is independently halogen or C₁₋₆ alkyl;
n is 1, 2, or 3;
W is 6-membered monocyclic heteroaryl, wherein the heteroaryl is unsubstituted or substituted with 1 or 2 substituents selected from R^{e};
   each R^{e} is independently halogen, —C(O)OH, —C(O)O(C₁₋₆ alkyl), C₁₋₆ alkyl, C₁₋₆ alkoxy, or C₃₋₆ cycloalkyl; wherein each alkyl, alkoxy, and cycloalkyl is unsubstituted or substituted with 1, 2, or 3 substituents selected from the group consisting of halogen, —OH, C₁₋₆ alkyl, and C₁₋₆ alkoxy;
or W is —C(═O)O—R¹⁵;
   R¹⁵ is C₁₋₆ alkyl, C₃₋₆ cycloalkyl, or 3- to 6-membered heterocycloalkyl, wherein the alkyl, cycloalkyl, or heterocycloalkyl is unsubstituted or substituted by 1-3 substituents independently selected from the group consisting of C₁₋₆ alkyl and C₁₋₆ fluoroalkyl;
Y is

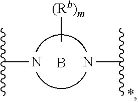

where Ring B is a monocyclic heterocycloalkyl or a bicyclic heterocycloalkyl; wherein Ring B comprises 2 N atoms and 0 or 1 O or S atoms, and * represents the attachment point to K;
or Y is

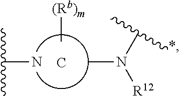

where Ring C is a fused bicyclic heterocycloalkyl, bridged bicyclic heterocycloalkyl, or spirocyclic bicyclic heterocycloalkyl; wherein Ring C comprises 1 or 2 N atoms and 0 or 1 O or S atoms; R¹² is hydrogen or C₁₋₄ alkyl; and * represents the attachment point to K;
each R^{b} is independently fluoro or C₁₋₄ alkyl;
m is 0, 1, or 2;
K is C₁₋₈ alkyl, —C(═O)—C₁₋₈ alkyl, —[(CH₂)ₛ—Z]ₜ—R³, —[(CHR^{d})ₛ—Z]ₜ—R¹³, or —[(C(R^{d})₂)ₛ—Z]ₜ—R³;
   wherein the alkyl is substituted by 1-6 R^{c} group;
each Z is independently —CH₂O—, —CH₂NH—, —CH₂NR^{d}—, or —CH₂N⁺(R^{d})₂;
s is 1-3;
t is 1-3;
R¹³ is hydrogen or a C₁₋₈ alkyl that is unsubstituted or substituted by 1-6 R^{c} groups;
each R^{c} is independently —OH, —CH₂OH, —NH₂, —N(R^{d})₃⁺, —C(═O)OH, —S(═O)₂OH, —S(═O)₂NH₂, —P(═O)(OH)₂, —P(═O)(OH)(R^{d}), —P(═O)(OH)(OR^{d}),

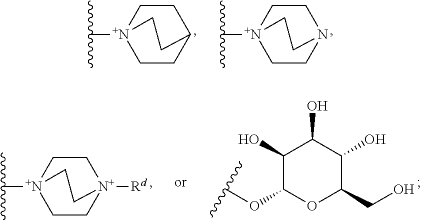

and
each R^{d} is independently C₁₋₆ alkyl.

In some embodiments,
X is —O—;
each R¹ is hydrogen;
each R² is hydrogen;
R¹¹ is hydrogen; and
R³, R⁴, R⁵, R⁶, R⁷, R⁸, R⁹, and R¹⁰ are each hydrogen;
Ring A is phenyl;
each R^{a} is independently halogen;
n is 1 or 2;
Y is

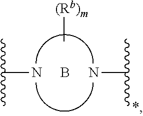

where Ring B is a monocyclic 4- to 8-membered heterocycloalkyl, 7- to 12-membered fused bicyclic heterocycloalkyl, 7- to 12-membered bridged bicyclic heterocycloalkyl, or 7- to 12-membered spirocyclic bicyclic heterocycloalkyl; wherein Ring B comprises 2 N atoms and 0 or 1 O or S atoms, and * represents the attachment point to K;
or Y is

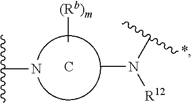

where Ring C is a 7- to 12-membered spirocyclic bicyclic heterocycloalkyl; wherein Ring C comprises 1 or 2 N atoms and 0 or 1 O or S atoms; R¹² is hydrogen or C₁₋₂ alkyl; and * represents the attachment point to K;
K is C₁₋₈ alkyl or —C(═O)—C₁₋₈ alkyl; wherein the alkyl is substituted by 1-6 R^{c} groups;
each R^{c} is independently —OH, —CH₂OH, —N(R^{d})₃⁺, —C(═O)OH, or —S(═O)₂OH; and
each R^{d} is independently C₁₋₆ alkyl.

In some embodiments, disclosed herein is a compound of Formula (IV):

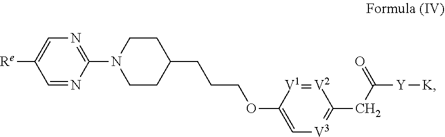

or a pharmaceutically acceptable salt, solvate, stereoisomer, or prodrug thereof, wherein:
V¹ is CH, CF, or N;
V² is CH, CF, or N; and
V³ is CH, CF, or N.

In some embodiments, disclosed herein is a compound of Formula (V):

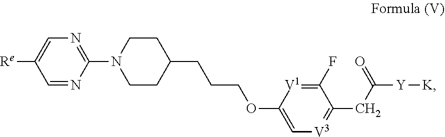

or a pharmaceutically acceptable salt, solvate, stereoisomer, or prodrug thereof, wherein:
V¹ is CH or CF; and
V³ is CH, CF, or N.

Disclosed herein, in certain embodiments, are pharmaceutical compositions comprising a compound disclosed herein, or a pharmaceutically acceptable salt, solvate, stereoisomer, or prodrug thereof, and at least one pharmaceutically acceptable excipient.

Disclosed herein, in certain embodiments, are methods of treating a condition or disorder involving the gut-brain axis in a subject in need thereof, the method comprising administering to the subject a therapeutically effective amount of a compound disclosed herein, or a pharmaceutically acceptable salt, solvate, stereoisomer, or prodrug thereof. In some embodiments, the condition or disorder is associated with GPR119 activity. In some embodiments, the condition or disorder is a metabolic disorder. In some embodiments, the condition or disorder is type 2 diabetes, hyperglycemia, metabolic syndrome, obesity, hypercholesterolemia, nonalcoholic steatohepatitis, or hypertension. In some embodiments, the condition or disorder is a nutritional disorder. In some embodiments, the condition or disorder is short bowel syndrome, intestinal failure, or intestinal insufficiency. In some embodiments, the condition or disorder is chemotherapy-induced enteritis or radiation-induced enteritis. In some embodiments, the compound disclosed herein is gut-restricted. In some embodiments, the compound disclosed herein has low systemic exposure.

In some embodiments, the methods disclosed herein further comprise administering one or more additional therapeutic agents to the subject. In some embodiments, the one or more additional therapeutic agents are selected from the group consisting of a TGR5 agonist, a GPR40 agonist, an SSTR5 antagonist, an SSTR5 inverse agonist, a CCK1 agonist, a PDE4 inhibitor, a DPP-4 inhibitor, a GLP-1 receptor agonist, metformin, or a combination thereof. In some embodiments, the TGR5 agonist, GPR40 agonist, SSTR5 antagonist, SSTR5 inverse agonist, or CCK1 agonist is gut-restricted.

Also disclosed herein, in certain embodiments, is the use of a compound disclosed herein, or a pharmaceutically acceptable salt, solvate, stereoisomer, or prodrug thereof, for the preparation of a medicament for the treatment of a condition or disorder involving the gut-brain axis in a subject in need thereof.

Also disclosed herein, in certain embodiments, are methods of treating a condition or disorder involving the gut-brain axis in a subject in need thereof, the method comprising administering to the subject a therapeutically effective amount of a gut-restricted GPR119 modulator.

Also disclosed herein, in certain embodiments, is the use of a gut-restricted GPR119 modulator for the preparation of a medicament for the treatment of a condition or disorder involving the gut-brain axis in a subject in need thereof.

### DETAILED DESCRIPTION OF THE INVENTION

This disclosure is directed, at least in part, to GPR119 agonists useful for the treatment of conditions or disorders involving the gut-brain axis. In some embodiments, the GPR119 agonists are gut-restricted compounds.

### Gut-Brain Axis

The gut-brain axis refers to the bidirectional biochemical signaling that connects the gastrointestinal tract (GI tract) with the central nervous system (CNS) through the peripheral nervous system (PNS) and endocrine, immune, and metabolic pathways.

In some instances, the gut-brain axis comprises the GI tract; the PNS including the dorsal root ganglia (DRG) and the sympathetic and parasympathetic arms of the autonomic nervous system including the enteric nervous system and the vagus nerve; the CNS; and the neuroendocrine and neuroimmune systems including the hypothalamic-pituitary-adrenal axis (HPA axis). The gut-brain axis is important for maintaining homeostasis of the body and is regulated and modulates physiology through the central and peripheral nervous systems and endocrine, immune, and metabolic pathways.

The gut-brain axis modulates several important aspects of physiology and behavior. Modulation by the gut-brain axis occurs via hormonal and neural circuits. Key components of these hormonal and neural circuits of the gut-brain axis include highly specialized, secretory intestinal cells that release hormones (enteroendocrine cells or EECs), the autonomic nervous system (including the vagus nerve and enteric nervous system), and the central nervous system. These systems work together in a highly coordinated fashion to modulate physiology and behavior.

Defects in the gut-brain axis are linked to a number of diseases, including those of high unmet need. Diseases and conditions affected by the gut-brain axis, include central nervous system (CNS) disorders including mood disorders, anxiety, depression, affective disorders, schizophrenia, malaise, cognition disorders, addiction, autism, epilepsy, neurodegenerative disorders, Alzheimer's disease, and Parkinson's disease, Lewy Body dementia, episodic cluster headache, migraine, pain; metabolic conditions including diabetes and its complications such as chronic kidney disease/diabetic nephropathy, diabetic retinopathy, diabetic neuropathy, and cardiovascular disease, metabolic syndrome, obesity, dyslipidemia, and nonalcoholic steatohepatitis (NASH); eating and nutritional disorders including hyperphagia, cachexia, anorexia nervosa, short bowel syndrome, intestinal failure, intestinal insufficiency and other eating disorders; inflammatory disorders and autoimmune diseases such as inflammatory bowel disease, ulcerative colitis, Crohn's disease, psoriasis, celiac disease, and enteritis, including chemotherapy-induced enteritis or radiation-induced enteritis; necrotizing enterocolitis; diseases/disorders of gastrointestinal barrier dysfunction including environmental enteric dysfunction, spontaneous bacterial peritonitis; functional gastrointestinal disorders such as irritable bowel syndrome, functional dyspepsia, functional abdominal bloating/distension, functional diarrhea, functional constipation, and opioid-induced constipation; gastroparesis; nausea and vomiting; disorders related to microbiome dysbiosis, and other conditions involving the gut-brain axis.

### GPR119 in the Gut-Brain Axis

In some instances, GPR119 is expressed in the pancreas and in enteroendocrine cells of the gastrointestinal tract. In some instances, GPR119 is expressed in enteroendocrine cells. GPR119 is activated by oleoylethanolamide (OEA) and other oleic acid derivatives and N-acylethanolamides. GPR119 agonists may be useful in the treatment of metabolic diseases such as diabetes and obesity, and other diseases involving the gut-brain axis.

In some instances, modulators of GPR119, for example, GPR119 agonists, induce the production of intracellular cAMP. In some instances, modulators of GPR119, for example, GPR119 agonists, induce the secretion of GLP-1, GLP-2, GIP, PYY, CCK, or other hormones.

In some instances, modulators of GPR119, for example, GPR119 agonists, induce the secretion of GLP-1 or PYY. In some instances, modulators of GPR119, for example, GPR119 agonists, induce the secretion of GLP-1. In some instances, modulators of GPR119, for example, GPR119 agonists, induce the secretion of PYY.

Described herein is a method of treating a condition or disorder involving the gut-brain axis in an individual in need thereof, the method comprising administering to the individual a GPR119 receptor modulator. In some embodiments, the GPR119 receptor modulator is a GPR119 agonist. In some embodiments, the GPR119 modulator is a gut-restricted GPR119 modulator.

In some embodiments, the condition or disorder involving the gut-brain axis is selected from the group consisting of: central nervous system (CNS) disorders including mood disorders, anxiety, depression, affective disorders, schizophrenia, malaise, cognition disorders, addiction, autism, epilepsy, neurodegenerative disorders, Alzheimer's disease, and Parkinson's disease, Lewy Body dementia, episodic cluster headache, migraine, pain; metabolic conditions including diabetes and its complications such as chronic kidney disease/diabetic nephropathy, diabetic retinopathy, diabetic neuropathy, and cardiovascular disease, metabolic syndrome, obesity, dyslipidemia, and nonalcoholic steatohepatitis (NASH); eating and nutritional disorders including hyperphagia, cachexia, anorexia nervosa, short bowel syndrome, intestinal failure, intestinal insufficiency and other eating disorders; inflammatory disorders and autoimmune diseases such as inflammatory bowel disease, ulcerative colitis, Crohn's disease, psoriasis, celiac disease, and enteritis, including chemotherapy-induced enteritis or radiation-induced enteritis; necrotizing enterocolitis; diseases/disorders of gastrointestinal barrier dysfunction including environmental enteric dysfunction, spontaneous bacterial peritonitis; functional gastrointestinal disorders such as irritable bowel syndrome, functional dyspepsia, functional abdominal bloating/distension, functional diarrhea, functional constipation, and opioid-induced constipation; gastroparesis; nausea and vomiting; disorders related to microbiome dysbiosis, other conditions involving the gut-brain axis. In some embodiments, the condition is a metabolic disorder. In some embodiments, the metabolic disorder is type 2 diabetes, hyperglycemia, metabolic syndrome, obesity, hypercholesterolemia, nonalcoholic steatohepatitis, or hypertension. In some embodiments, the metabolic disorder is diabetes. In other embodiments, the metabolic disorder is obesity. In other embodiments, the metabolic disorder is nonalcoholic steatohepatitis. In some embodiments, the condition involving the gut-brain axis is a nutritional disorder. In some embodiments, the nutritional disorder is short bowel syndrome, intestinal failure, or intestinal insufficiency. In some embodiments, the nutritional disorder is short bowel syndrome. In some embodiments, the condition involving the gut-brain axis is enteritis. In some embodiments, the condition involving the gut-brain axis is chemotherapy-induced enteritis or radiation-induced enteritis.

### Gut-Restricted Modulators

Differentiation of undesirable systemic effects of a GPR119 agonist from beneficial, gut-driven effects would be critical for the development of a GPR119 agonist for the treatment of disease. For example, activation of GPR119 in alpha cells of pancreatic islets by systemic GPR119 agonists can lead to secretion of glucagon, causing undesired metabolic effects, e.g., increased plasma glucose levels. Furthermore, systemic GPR119 agonists are typically hydrophobic ligands that suffer from undesirable off-target activity, such as hERG channel and/or CYP enzyme inhibition.

In contrast, some embodiments provided herein describe a GPR119 modulator that is non-systemic. In some embodiments, the GPR119 modulator described herein is substantially non-systemic. In some embodiments, the GPR119 modulator described herein has low bioavailability. In some embodiments, the GPR119 modulator described herein is bound to a kinetophore and is non-systemic. In some embodiments, the GPR119 modulator described herein is bound to a kinetophore and is substantially non-systemic. In some embodiments, the GPR119 modulator described herein is bound to a kinetophore and has lower bioavailability than a corresponding compound without a kinetophore.

In some embodiments, the GPR119 agonist is gut-restricted. In some embodiments, the GPR119 agonist is substantially non-permeable or substantially non-bioavailable in the blood stream. In some embodiments, the GPR119 agonist activates GPR119 activity in the gut and is substantially non-systemic. In some embodiments, the GPR119 agonist has low systemic exposure. In some embodiments, the gut-restricted GPR119 agonists described herein provide fewer undesired side effects than systemic GPR119 agonists.

In some embodiments, a gut-restricted GPR119 agonist has low oral bioavailability. In some embodiments, a gut-restricted GPR119 agonist has <20% oral bioavailability, <10% oral bioavailability, <8% oral bioavailability, <5% oral bioavailability, <3% oral bioavailability, or <2% oral bioavailability.

In some embodiments, the unbound plasma levels of a gut-restricted GPR119 agonist are lower than the EC₅₀ value of the GPR119 agonist against GPR119. In some embodiments, the unbound plasma levels of a gut-restricted GPR119 agonist are significantly lower than the EC₅₀ value of the gut-restricted GPR119 agonist against GPR119. In some embodiments, the unbound plasma levels of the GPR119 agonist are 2-fold, 10-fold, 20-fold, 30-fold, 40-fold, 50-fold, or 100-fold lower than the EC₅₀ value of the gut-restricted GPR119 agonist against GPR119.

In some embodiments, a gut-restricted GPR119 agonist has low systemic exposure. In some embodiments, the systemic exposure of a gut-restricted GPR119 agonist is, for example, less than 500, less than 200, less than 100, less than 50, less than 20, less than 10, or less than 5 nM, bound or unbound, in blood serum. In some embodiments, the systemic exposure of a gut-restricted GPR119 agonist is, for example, less than 500, less than 200, less than 100, less than 50, less than 20, less than 10, or less than 5 ng/mL, bound or unbound, in blood serum.

In some embodiments, a gut-restricted GPR119 agonist has low permeability. In some embodiments, a gut-restricted GPR119 agonist has low intestinal permeability. In some embodiments, the permeability of a gut-restricted GPR119 agonist is, for example, less than 5.0×10⁻⁶ cm/s, less than 2.0×10⁻⁶ cm/s, less than 1.5×10⁻⁶ cm/s, less than 1.0×10⁻⁶ cm/s, less than 0.75×10⁻⁶ cm/s, less than 0.50×10⁻⁶ cm/s, less than 0.25×10⁻⁶ cm/s, less than 0.10×10⁻⁶ cm/s, or less than 0.05×10⁻⁶ cm/s.

In some embodiments, a gut-restricted GPR119 agonist has low absorption. In some embodiments, the absorption of a gut-restricted GPR119 agonist is less than less than 20%, or less than 10%, less than 5%, or less than 1%.

In some embodiments, a gut-restricted GPR119 agonist has high plasma clearance. In some embodiments, a gut-restricted GPR119 agonist is undetectable in plasma in less than 8 hours, less than 6 hours, less than 4 hours, less than 3 hours, less than 120 min, less than 90 min, less than 60 min, less than 45 min, less than 30 min, or less than 15 min.

In some embodiments, a gut-restricted GPR119 agonist is rapidly metabolized upon administration. In some embodiments, a gut-restricted GPR119 agonist has a short half-life. In some embodiments, the half-life of a gut-restricted GPR119 agonist (e.g., in plasma) is less than less than 8 hours, less than 6 hours, less than 4 hours, less than 3 hours, less than 120 min, less than 90 min, less than 60 min, less than 45 min, less than 30 min, or less than 15 min. In some embodiments, the metabolites of a gut-restricted GPR119 agonist have rapid clearance (e.g., systemic clearance). In some embodiments, the metabolites of a gut-restricted GPR119 agonist are undetectable (e.g., in plasma) in less than 8 hours, less than 6 hours, less than 4 hours, less than 3 hours, less than 120 min, less than 90 min, less than 60 min, less than 45 min, less than 30 min, or less than 15 min. In some embodiments, the metabolites of a gut-restricted GPR119 agonist have low bioactivity. In some embodiments, the EC₅₀ value of the metabolites of a gut-restricted GPR119 agonist is 10-fold, 20-fold, 30-fold, 40-fold, 50-fold, 100-fold, 500-fold, or 1000-fold higher than the EC₅₀ value of the gut-restricted GPR119 agonist against GPR119. In some embodiments, the metabolites of a gut-restricted GPR119 agonist have rapid clearance and low bioactivity.

In some embodiments of the methods described herein, the GPR119 modulator is gut-restricted. In some embodiments, the GPR119 modulator is a gut-restricted GPR119 agonist. In some embodiments, the GPR119 agonist is covalently bonded to a kinetophore. In some embodiments, the GPR119 agonist is covalently bonded to a kinetophore through a linker.

In some instances, known GPR119 agonists are systemic. In some instances, known systemic GPR119 agonists are not bonded to a kinetophore as described herein. In some instances, known GPR119 agonists have high oral bioavailability. In some embodiments, the GPR119 modulator described herein is bound to a kinetophore and is non-systemic. In some embodiments, the GPR119 modulator described herein is bound to a kinetophore and is substantially non-systemic. In some embodiments, the GPR119 modulator described herein is bound to a kinetophore and has lower bioavailability than a corresponding compound without a kinetophore.

### Compounds

Disclosed herein, in certain embodiments, is a compound of Formula (I):

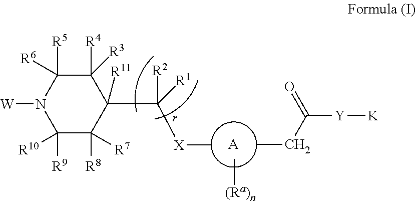

or a pharmaceutically acceptable salt, solvate, stereoisomer, or prodrug thereof, wherein:
Y is

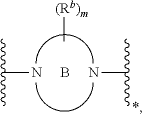

where Ring B is a heterocycloalkyl; wherein Ring B comprises 2 N atoms and 0 or 1 O or S atoms, and * represents the attachment point to K;
or Y is

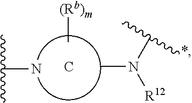

where Ring C is a bicyclic heterocycloalkyl; wherein Ring C comprises 1 or 2 N atoms and 0 or 1 O or S atoms, R¹² is hydrogen or C₁₋₄ alkyl, and * represents the attachment point to K;
each R^{b} is independently fluoro, C₁₋₆ alkyl, or C₁₋₆ fluoroalkyl;
K is C₁₋₈ alkyl, C₂₋₈ alkenyl, C₂₋₈ alkynyl, —C(═O)—C₁₋₈ alkyl, —C(═O)—C₂₋₈ alkenyl, —C(═O)—C₂₋₈ alkynyl, —[(CH₂)ₛ—Z]ₜ—R¹³, —[(CHR^{d})ₛ—Z]ₜ—R¹³, or —[(C(R^{d})₂)ₛ—Z]ₜ—R¹³; wherein each alkyl, alkenyl, or alkynyl is substituted by 1-6 R^{c} groups;
   each Z is independently —CH₂O—, —CH₂NH—, —CH₂NR^{d}—, —CH₂N⁺(R^{d})₂—, —NH—C(═O)—NH—, —C(═O)NH—, —CH₂S(═O)₂—, or —CH₂S(═O)—;
   each s is independently 1-6;
   each t is independently 1-6;
R¹³ is hydrogen, C₁₋₈ alkyl, C₂₋₈ alkenyl, or C₂₋₈ alkynyl, wherein the alkyl, alkenyl, or alkynyl is unsubstituted or substituted by 1-6 R^{c} groups;
each R^{c} is independently —OH, —CH₂OH, —CH₂CH₂OH, —NH₂, —CH₂NH₂, —NH(R^{d}), —CH₂NH(R^{d}), —N(R^{d})₂, —CH₂N(R^{d})₂, —N(R^{d})₃⁺, —C(═O)OH, —CH₂C(═O)OH, —CH₂CH₂C(═O)OH, —S(═O)₂OH, —S(═O)OH, —S(═O)₂NH₂, —P(═O)(OH)₂, —P(═O)(OH)(R^{d}), —P(═O)(OH)(H), P(═O)(OH)(OR^{d}), —B(OH)₂, —B(OR^{d})(OH), —NHCONHS(═O)₂(R^{d}), —N(R^{d})CONHS(═O)₂(R^{d}), —NHCON(R^{d})S(═O)₂(R^{d}), —C(═O)NHS(═O)₂(R^{d}), —S(═O)₂NHC(═O)R^{d}, —NHC(═NH)NH₂, —NHC(═NH)NHR^{d}, —NHC(═NH)N(R^{d})₂, —N(R^{d})C(═NH)NH₂, —N(R^{d})C(═NH)NH(R^{d}), —N(R^{d})C(═NH)N(R^{d})₂, —NHC(═N(R^{d}))NH₂, —NHC(═N(R^{d})NHR^{d}, —NHC(═N(R^{d}))N(R^{d})₂, —N(R^{d})C(═N(R^{d}))NH₂, —N(R^{d})C(═N(R^{d}))NR^{d}, —N(R^{d})C(═N(R^{d}))N(R^{d})₂, —NHC(═NH)NHC(═NH)NH₂, —N(R^{d})C(═NH)NHC(═NH)NH₂,

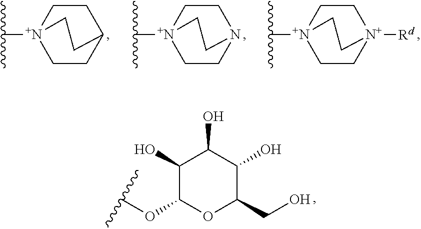

or a 4- to 6-membered heterocycle which is unsubstituted or substituted with 1, 2, 3, or 4 substituents selected from the group consisting of C₁₋₆ alkyl, —O—(C₁₋₆ alkyl), —OH, ═O and ═S;
each R^{d} is independently C₁₋₆ alkyl, C₁₋₆ fluoroalkyl, or C₃₋₆ cycloalkyl;
Ring A is phenyl, 5-membered monocyclic heteroaryl, or 6-membered monocyclic heteroaryl;
each R^{a} is independently halogen, —CN, C₁₋₆ alkyl, C₁₋₆ fluoroalkyl, or C₃₋₆ cycloalkyl;
X is —O—, —NR¹⁴—, #—CH₂O—, #—CH₂NR¹⁴—, #—C(═O)O—, #—C(═O)NR¹⁴—, #—CH₂C(═O)O—, #—CH₂C(═O)NR¹⁴—, #—OC(═O)—, #—NR¹⁴C(═O)—, #—CH₂OC(═O)—, or #—CH₂NR¹⁴C(═O)—, where # represents the attachment point to Ring A;
R¹⁴ is hydrogen or C₁₋₄ alkyl;
each R¹ is independently hydrogen, fluorine, —OH, C₁₋₆ alkyl, or C₁₋₆ alkoxy;
or two R¹ are taken together with the intervening atoms to which they are attached to form a C₃₋₆ cycloalkyl;
each R² is independently hydrogen, fluorine, or C₁₋₆ alkyl;
R³, R⁴, R⁵, R⁶, R⁷, R⁸, R⁹, and R¹⁰ are each independently hydrogen, C₁₋₆ alkyl, C₁₋₆ alkoxy, or C₁₋₆ fluoroalkyl;
or R³ and R⁷ or R³ and R⁹ or R⁵ and R⁹ are taken together with the intervening atoms to which they are attached to form a ring;
R¹¹ is hydrogen, C₁₋₆ alkyl, or C₁₋₆ fluoroalkyl;
or R¹¹ and one R are taken together with the intervening atoms to which they are attached to form a C₃₋₆ cycloalkyl;
W is phenyl, 5-membered monocyclic heteroaryl, or 6-membered monocyclic heteroaryl, wherein the phenyl or heteroaryl is unsubstituted or substituted with 1, 2, or 3 substituents selected from R^{e};
each R^{e} is independently halogen, —OH, —CN, —C(O)OH, —C(O)O(C₁₋₆ alkyl), C₁₋₆ alkyl, C₁₋₆ alkoxy, C₃₋₆ cycloalkyl, phenyl, or 5- to 6-membered heteroaryl; wherein each alkyl, alkoxy, and cycloalkyl is unsubstituted or substituted with 1, 2, or 3 substituents selected from the group consisting of halogen, —OH, C₁₋₆ alkyl, and C₁₋₆ alkoxy;
or W is —C(═O)O—R¹⁵;
R¹⁵ is C₁₋₆ alkyl, C₃₋₆ cycloalkyl, or 3- to 6-membered heterocycloalkyl, wherein the alkyl, cycloalkyl, and heterocycloalkyl are unsubstituted or substituted by 1-3 substituents independently selected from the group consisting of fluorine, —OH, C₁₋₆ alkyl, C₁₋₆ fluoroalkyl, or C₃₋₆ cycloalkyl;
m is 0, 1, 2, 3, or 4;
n is 0, 1, 2, 3, or 4; and
r is 1, 2, 3, 4, 5 or 6.

In some embodiments, disclosed herein is a compound of Formula (II):

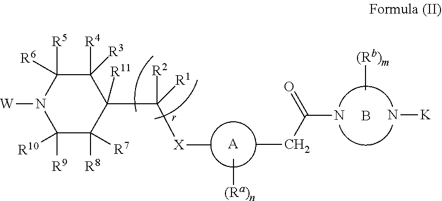

or a pharmaceutically acceptable salt, solvate, stereoisomer, or prodrug thereof, wherein:
Ring B is a heterocycloalkyl; wherein Ring B comprises 2 N atoms and 0 or 1 O or S atoms;
each R^{b} is independently fluoro, C₁₋₆ alkyl, or C₁₋₆ fluoroalkyl;
K is C₁₋₈ alkyl, C₂₋₈ alkenyl, C₂₋₈ alkynyl, —C(═O)—C₁₋₈ alkyl, —C(═O)—C₂₋₃ alkenyl, —C(═O)—C₂₋₈ alkynyl, —[(CH₂)ₛ—Z]ₜ—R³, —[(CHR^{d})ₛ—Z]ₜ—R¹³, or —[(C(R^{d})₂)ₛ—Z]ₜ—R¹³; wherein each alkyl, alkenyl, or alkynyl is substituted by 1-6 R^{c} groups;
   each Z is independently —CH₂O—, —CH₂NH—, —CH₂NR^{d}—, —CH₂N⁺(R^{d})₂—, —NH—C(═O)—NH—, —C(═O)NH—, —CH₂S(═O)₂—, or —CH₂S(═O)—;
   each s is independently 1-6;
   each t is independently 1-6;
R¹³ is hydrogen, C₁₋₈ alkyl, C₂₋₈ alkenyl, or C₂₋₈ alkynyl, wherein the alkyl, alkenyl, or alkynyl is unsubstituted or substituted by 1-6 R^{c} groups;
each R^{c} is independently —OH, —CH₂OH, —CH₂CH₂OH, —NH₂, —CH₂NH₂, —NH(R^{d}), —CH₂NH(R^{d}), —N(R^{d})₂, —CH₂N(R^{d})₂, —N(R^{d})₃⁺, —C(═O)OH, —CH₂C(═O)OH, —CH₂CH₂C(═O)OH, —S(═O)₂OH, —S(═O)OH, —S(═O)₂NH₂, —P(═O)(OH)₂, —P(═O)(OH)(R^{d}), —P(═O)(OH)(H), P(═O)(OH)(OR^{d}), —B(OH)₂, —B(OR^{d})(OH), —NHCONHS(═O)₂(R^{d}), —N(R^{d})CONHS(═O)₂(R^{d}), —NHCON(R^{d})S(═O)₂(R^{d}), —C(═O)NHS(═O)₂(R^{d}), —S(═O)₂NHC(═O)R^{d}, —NHC(═NH)NH₂, —NHC(═NH)NHR^{d}, —NHC(═NH)N(R^{d})₂, —N(R^{d})C(═NH)NH₂, —N(R^{d})C(═NH)NH(R^{d}), —N(R^{d})C(═NH)N(R^{d})₂, —NHC(═N(R^{d}))NH₂, —NHC(═N(R^{d}))NHR^{d}, —NHC(═N(R^{d}))N(R^{d})₂, —N(R^{d})C(═N(R^{d}))NH₂, —N(R^{d})C(═N(R^{d}))NHR^{d}, —N(R^{d})C(═N(R^{d}))N(R^{d})₂, —NHC(═NH)NHC(═NH)NH₂, —N(R^{d})C(═NH)NHC(═NH)NH₂,

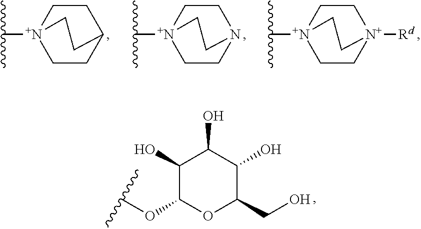

or a 4- to 6-membered heterocycle which is unsubstituted or substituted with 1, 2, 3, or 4 substituents selected from the group consisting of C₁₋₆ alkyl, —O—(C₁₋₆ alkyl), —OH, ═O and ═S;
each R^{d} is independently C₁₋₆ alkyl, C₁₋₆ fluoroalkyl, or C₃₋₆ cycloalkyl;
Ring A is phenyl, 5-membered monocyclic heteroaryl, or 6-membered monocyclic heteroaryl;
each R^{a} is independently halogen, —CN, C₁₋₆ alkyl, C₁₋₆ fluoroalkyl, or C₃₋₆ cycloalkyl;
X is —O—, —NR¹⁴—, #—CH₂O—, #—CH₂NR¹⁴—, #—C(═O)O—, #—C(═O)NR¹⁴—, #—CH₂C(═O)O—, #—CH₂C(═O)NR¹⁴—, #—OC(═O)—, #—NR¹⁴C(═O)—, #—CH₂OC(═O)—, or #—CH₂NR¹⁴C(═O)—, where # represents the attachment point to Ring A;
R¹⁴ is hydrogen or C₁₋₄ alkyl;
each R¹ is independently hydrogen, fluorine, —OH, C₁₋₆ alkyl, or C₁₋₆ alkoxy;
or two R¹ are taken together with the intervening atoms to which they are attached to form a C₃₋₆ cycloalkyl;
each R² is independently hydrogen, fluorine, or C₁₋₆ alkyl;
R³, R⁴, R⁵, R⁶, R⁷, R⁸, R⁹, and R¹⁰ are each independently hydrogen, C₁₋₆ alkyl, C₁₋₆ alkoxy, or C₁₋₆ fluoroalkyl;
or R³ and R⁷ or R³ and R⁹ or R⁵ and R⁹ are taken together with the intervening atoms to which they are attached to form a ring;
R¹¹ is hydrogen, C₁₋₆ alkyl, or C₁₋₆ fluoroalkyl;
or R¹¹ and one R¹ are taken together with the intervening atoms to which they are attached to form a C₃₋₆ cycloalkyl;
W is phenyl, 5-membered monocyclic heteroaryl, or 6-membered monocyclic heteroaryl, wherein the phenyl or heteroaryl is unsubstituted or substituted with 1, 2, or 3 substituents selected from R^{e};
each R^{e} is independently halogen, —OH, —CN, —C(O)OH, —C(O)O(C₁₋₆ alkyl), C₁₋₆ alkyl, C₁₋₆ alkoxy, C₃₋₆ cycloalkyl, phenyl, or 5- to 6-membered heteroaryl; wherein each alkyl, alkoxy, and cycloalkyl is unsubstituted or substituted with 1, 2, or 3 substituents selected from the group consisting of halogen, —OH, C₁₋₆ alkyl, and C₁₋₆ alkoxy;
or W is —C(═O)O—R¹⁵;
R¹⁵ is C₁₋₆ alkyl, C₃₋₆ cycloalkyl, or 3- to 6-membered heterocycloalkyl, wherein the alkyl, cycloalkyl, and heterocycloalkyl are unsubstituted or substituted by 1-3 substituents independently selected from the group consisting of fluorine, —OH, C₁₋₆ alkyl, C₁₋₆ fluoroalkyl, or C₃₋₆ cycloalkyl;
m is 0, 1, 2, 3, or 4;
n is 0, 1, 2, 3, or 4; and
r is 1, 2, 3, 4, 5 or 6.

In some embodiments, disclosed herein is a compound of Formula (III):

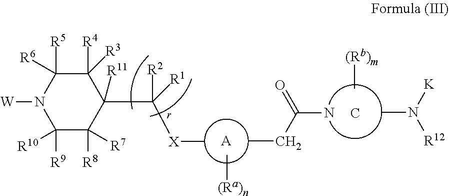

or a pharmaceutically acceptable salt, solvate, stereoisomer, or prodrug thereof, wherein:
Ring C is a bicyclic heterocycloalkyl; wherein Ring C comprises 1 or 2 N atoms and 0 or 1 O or S atoms;
R¹² is hydrogen or C₁₋₄ alkyl;
each R^{b} is independently fluoro, C₁₋₆ alkyl, or C₁₋₆ fluoroalkyl;
K is C₁₋₈ alkyl, C₂₋₈alkenyl, C₂₋₈alkynyl, —C(═O)—C₁₋₈ alkyl, —C(═O)—C₂₋₈ alkenyl, —C(═O)—C₂₋₈ alkynyl, —[(CH₂)ₛ—Z]ₜ—R³, —[(CHR^{d})ₛ—Z]ₜ—R³, or —[(C(R^{d})₂)ₛ—Z]ₜ—R¹³; wherein each alkyl, alkenyl, or alkynyl is substituted by 1-6 R^{c} groups;
   each Z is independently —CH₂O—, —CH₂NH—, —CH₂NR^{d}—, —CH₂N⁺(R^{d})₂—, —NH—C(═O)—NH—, —C(═O)NH—, —CH₂S(═O)₂—, or —CH₂S(═O)—;
   each s is independently 1-6;
   each t is independently 1-6;
R¹³ is hydrogen, C₁₋₈ alkyl, C₂₋₈ alkenyl, or C₂₋₈ alkynyl, wherein the alkyl, alkenyl, or alkynyl is unsubstituted or substituted by 1-6 R^{c} groups;
each R^{c} is independently —OH, —CH₂OH, —CH₂CH₂OH, —NH₂, —CH₂NH₂, —NH(R^{d}), —CH₂NH(R^{d}), —N(R^{d})₂, —CH₂N(R^{d})₂, —N(R^{d})₃⁺, —C(═O)OH, —CH₂C(═O)OH, —CH₂CH₂C(═O)OH, —S(═O)₂OH, —S(═O)OH, —S(═O)₂NH₂, —P(═O)(OH)₂, —P(═O)(OH)(R^{d}), —P(═O)(OH)(H), P(═O)(OH)(OR^{d}), —B(OH)₂, —B(OR^{d})(OH), —NHCONHS(═O)₂(R^{d}), —N(R^{d})CONHS(═O)₂(R^{d}), —NHCON(R^{d})S(═O)₂(R^{d}), —C(═O)NHS(═O)₂(R^{d}), —S(═O)₂NHC(═O)R^{d}, —NHC(═NH)NH₂, —NHC(═NH)NHR^{d}, —NHC(═NH)N(R^{d})₂, —N(R^{d})C(═NH)NH₂, —N(R^{d})C(═NH)NH(R^{d}), —N(R^{d})C(═NH)N(R^{d})₂, —NHC(═N(R^{d}))NH₂, —NHC(═N(R^{d}))NHR^{d}, —NHC(═N(R^{d}))N(R^{d})₂, —N(R^{d})C(═N(R^{d}))NH₂, —N(R^{d})C(═N(R^{d}))NHR^{d}, —N(R^{d})C(═N(R^{d}))N(R^{d})₂, —NHC(═NH)NHC(═NH)NH₂, —N(R^{d})C(═NH)NHC(═NH)NH₂,

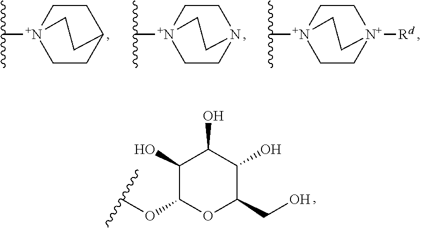

or a 4- to 6-membered heterocycle which is unsubstituted or substituted with 1, 2, 3, or 4 substituents selected from the group consisting of C₁₋₆ alkyl, —O—(C₁₋₆ alkyl), —OH, ═O and ═S;
each R^{d} is independently C₁₋₆ alkyl, C₁₋₆ fluoroalkyl, or C₃₋₆ cycloalkyl;
Ring A is phenyl, 5-membered monocyclic heteroaryl, or 6-membered monocyclic heteroaryl;
each R^{a} is independently halogen, —CN, C₁₋₆ alkyl, C₁₋₆ fluoroalkyl, or C₃₋₆ cycloalkyl;
X is —O—, —NR¹⁴—, #—CH₂O—, #—CH₂NR¹⁴—, #—C(═O)O—, #—C(═O)NR¹⁴—, #—CH₂C(═O)O—, #—CH₂C(═O)NR¹⁴—, #—OC(═O)—, #—NR¹⁴C(═O)—, #—CH₂OC(═O)—, or #—CH₂NR¹⁴C(═O)—, where # represents the attachment point to Ring A;
R¹⁴ is hydrogen or C₁₋₄ alkyl;
each R¹ is independently hydrogen, fluorine, —OH, C₁₋₆ alkyl, or C₁₋₆ alkoxy;
or two R¹ are taken together with the intervening atoms to which they are attached to form a C₃₋₆ cycloalkyl;
each R² is independently hydrogen, fluorine, or C₁₋₆ alkyl;
R³, R⁴, R⁵, R⁶, R⁷, R⁸, R⁹, and R¹⁰ are each independently hydrogen, C₁₋₆ alkyl, C₁₋₆ alkoxy, or C₁₋₆ fluoroalkyl;
or R³ and R⁷ or R³ and R⁹ or R⁵ and R⁹ are taken together with the intervening atoms to which they are attached to form a ring;
R¹¹ is hydrogen, C₁₋₆ alkyl, or C₁₋₆ fluoroalkyl;
or R¹¹ and one R¹ are taken together with the intervening atoms to which they are attached to form a C₃₋₆ cycloalkyl;
W is phenyl, 5-membered monocyclic heteroaryl, or 6-membered monocyclic heteroaryl, wherein the phenyl or heteroaryl is unsubstituted or substituted with 1, 2, or 3 substituents selected from R^{e};
each R^{e} is independently halogen, —OH, —CN, —C(O)OH, —C(O)O(C₁₋₆ alkyl), C₁₋₆ alkyl, C₁₋₆ alkoxy, C₃₋₆ cycloalkyl, phenyl, or 5- to 6-membered heteroaryl; wherein each alkyl, alkoxy, and cycloalkyl is unsubstituted or substituted with 1, 2, or 3 substituents selected from the group consisting of halogen, —OH, C₁₋₆ alkyl, and C₁₋₆ alkoxy;
or W is —C(═O)O—R¹⁵;
R¹⁵ is C₁₋₆ alkyl, C₃₋₆ cycloalkyl, or 3- to 6-membered heterocycloalkyl, wherein the alkyl, cycloalkyl, and heterocycloalkyl are unsubstituted or substituted by 1-3 substituents independently selected from the group consisting of fluorine, —OH, C₁₋₆ alkyl, C₁₋₆ fluoroalkyl, or C₃₋₆ cycloalkyl;
m is 0, 1, 2, 3, or 4;
n is 0, 1, 2, 3, or 4; and
r is 1, 2, 3, 4, 5 or 6.

In some embodiments, X is —O—, —NR¹⁴—, #—CH₂O—, #—CH₂NR¹⁴—, #—C(═O)O—, #—C(═O)NR¹⁴—, #—CH₂C(═O)O—, #—CH₂C(═O)NR¹⁴—, #—OC(═O)—, #—NR¹⁴C(═O)—, #—CH₂OC(═O)—, or #—CH₂NR¹⁴C(═O)—, where # represents the attachment point to Ring A. In some embodiments, X is —O—, —NR¹⁴—, #—CH₂O—, #—CH₂NR¹⁴—, #—C(═O)O—, #—C(═O)NR¹⁴—, #—CH₂C(═O)O—, #—CH₂C(═O)NR¹⁴—, #—OC(═O)—, #—CH₂OC(═O)—, or #—CH₂NR¹⁴C(═O)—, where # represents the attachment point to Ring A. In some embodiments, X is —O—, —NR¹⁴—, #—CH₂O—, or #—CH₂NR¹⁴—, where # represents the attachment point to Ring A. In some embodiments, X is —O— or —NR¹⁴—. In some embodiments, X is —O—, #—CH₂O—, #—C(═O)O—, or #—CH₂C(═O)O—, where # represents the attachment point to Ring A. In some embodiments, X is —O— or #—CH₂O—, where # represents the attachment point to Ring A. In some embodiments, X is —O—. In some embodiments, X is —NR¹⁴—.

In some embodiments, R¹⁴ is hydrogen, methyl, ethyl, n-propyl, i-propyl, n-butyl, i-butyl, s-butyl, or t-butyl. In some embodiments, R¹⁴ is hydrogen, methyl, or ethyl. In some embodiments, R¹⁴ is hydrogen or methyl. In some embodiments, R¹⁴ is hydrogen. In some embodiments, R¹⁴ is methyl.

In some embodiments, each R¹ is independently hydrogen, fluorine, C₁₋₆ alkyl, or C₁₋₆ alkoxy. In some embodiments, each R¹ is independently hydrogen, fluorine, or C₁₋₆ alkyl. In some embodiments, each R¹ is independently hydrogen or C₁₋₆ alkyl. In some embodiments, each R¹ is independently hydrogen, fluorine, or C₁₋₄ alkyl. In some embodiments, each R¹ is independently hydrogen or C₁₋₄ alkyl. In some embodiments, each R¹ is independently hydrogen, methyl, ethyl, n-propyl, i-propyl, n-butyl, i-butyl, s-butyl, or t-butyl. In some embodiments, each R¹ is hydrogen.

In some embodiments, each R² is independently hydrogen, fluorine, or C₁₋₄ alkyl. In some embodiments, each R² is independently hydrogen or C₁₋₄ alkyl. In some embodiments, each R² is independently hydrogen, methyl, ethyl, n-propyl, i-propyl, n-butyl, i-butyl, s-butyl, or t-butyl. In some embodiments, each R² is hydrogen.

In some embodiments, two R¹ are taken together with the intervening atoms to which they are attached to form a C₃₋₆ cycloalkyl. In some embodiments, two R¹ are taken together with the intervening atoms to which they are attached to form a C₃₋₄ cycloalkyl. In some embodiments, two R¹ are taken together with the intervening atoms to which they are attached to form a cyclopropyl, cyclobutyl, cyclopentyl, or cyclohexyl. In some embodiments, two R¹ are taken together with the intervening atoms to which they are attached to form a cyclopropyl or cyclobutyl. In some embodiments, two R¹ are taken together with the intervening atoms to which they are attached to form a cyclopropyl. In some embodiments, two R¹ are taken together with the intervening atoms to which they are attached to form a cyclobutyl.

In some embodiments, R³, R⁴, R⁵, R⁶, R⁷, R⁸, R⁹, and R¹⁰ are each independently hydrogen, C₁₋₆ alkyl, C₁₋₆ alkoxy, or C₁₋₆ fluoroalkyl. In some embodiments, R³, R⁴, R⁵, R⁶, R⁷, R⁸, R⁹, and R¹⁰ are each independently hydrogen, C₁₋₆ alkyl, or C₁₋₆ fluoroalkyl. In some embodiments, R³, R⁴, R⁵, R⁶, R⁷, R⁸, R⁹, and R¹⁰ are each independently hydrogen or C₁₋₆ alkyl. In some embodiments, R³, R⁴, R⁵, R⁶, R⁷, R⁸, R⁹, and R¹⁰ are each independently hydrogen, C₁₋₄ alkyl, or C₁₋₄ fluoroalkyl. In some embodiments, R³, R⁴, R⁵, R⁶, R⁷, R⁸, R⁹, and R¹⁰ are each independently hydrogen or C₁₋₄ alkyl. In some embodiments, R³, R⁴, R⁵, R⁶, R⁷, R⁸, R⁹, and R¹⁰ are each independently hydrogen, methyl, ethyl, n-propyl, i-propyl, n-butyl, i-butyl, s-butyl, t-butyl, —CF₃, CHF₂, or CH₂F. In some embodiments, R³, R⁴, R⁵, R⁶, R⁷, R⁸, R⁹, and R¹⁰ are each independently hydrogen, methyl, ethyl, n-propyl, i-propyl, n-butyl, i-butyl, s-butyl, or t-butyl. In some embodiments, R³, R⁴, R⁵, R⁶, R⁷, R⁸, R⁹, and R¹⁰ are each hydrogen.

In some embodiments, R³ and R⁷ or R³ and R⁹ or R⁵ and R⁹ are taken together with the intervening atoms to which they are attached to form a ring. In some embodiments, R³ and R⁷ or R³ and R⁹ or R⁵ and R⁹ are taken together with the intervening atoms to which they are attached to form a 4- to 6-membered ring. In some embodiments, R³ and R⁷ or R³ and R⁹ or R⁵ and R⁹ are taken together to form a bond, —CH₂—, or —CH₂CH₂—. In some embodiments, R³ and R⁷ or R³ and R⁹ or R⁵ and R⁹ are taken together to form a bond. In some embodiments, R³ and R⁷ are taken together with the intervening atoms to which they are attached to form a ring. In some embodiments, or R³ and R⁹ are taken together with the intervening atoms to which they are attached to form a ring. In some embodiments, R⁵ and R⁹ are taken together with the intervening atoms to which they are attached to form a ring. In some embodiments, R³ and R⁷ are taken together to form a bond. In some embodiments, or R³ and R⁹ are taken together to form a bond.

In some embodiments, R⁵ and R⁹ are taken together to form a bond.

In some embodiments, R¹¹ is hydrogen, C₁₋₆ alkyl, or C₁₋₆ fluoroalkyl. In some embodiments, R¹¹ is hydrogen or C₁₋₆ alkyl. In some embodiments, R¹¹ is hydrogen, C₁₋₄ alkyl, or C₁₋₄ fluoroalkyl. In some embodiments, R¹¹ is hydrogen or C₁₋₄ alkyl. In some embodiments, R¹¹ is hydrogen, methyl, ethyl, n-propyl, i-propyl, n-butyl, i-butyl, s-butyl, t-butyl, —CF₃, CHF₂, or CH₃F. In some embodiments, R¹¹ is hydrogen, methyl, ethyl, n-propyl, i-propyl, n-butyl, i-butyl, s-butyl, or t-butyl. In some embodiments, R¹¹ is hydrogen.

In some embodiments, R¹¹ and one R¹ are taken together with the intervening atoms to which they are attached to form a C₃₋₆ cycloalkyl. In some embodiments, R¹¹ and one R¹ are taken together with the intervening atoms to which they are attached to form a C₃₋₄ cycloalkyl.

In some embodiments, R¹¹ and one R¹ are taken together with the intervening atoms to which they are attached to form a cyclopropyl, cyclobutyl, cyclopentyl, or cyclohexyl. In some embodiments, R¹¹ and one R are taken together with the intervening atoms to which they are attached to form a cyclopropyl or cyclobutyl. In some embodiments, R¹¹ and one R¹ are taken together with the intervening atoms to which they are attached to form a cyclopropyl. In some embodiments, R¹¹ and one R are taken together with the intervening atoms to which they are attached to form a cyclobutyl.

In some embodiments, r is 1, 2, 3, 4, 5 or 6. In some embodiments, r is 3, 4, 5 or 6. In some embodiments, r is 3 or 4. In some embodiments, r is 1. In some embodiments, r is 2. In some embodiments, r is 3. In some embodiments, r is 4. In some embodiments, r is 5. In some embodiments, r is 6.

In some embodiments,

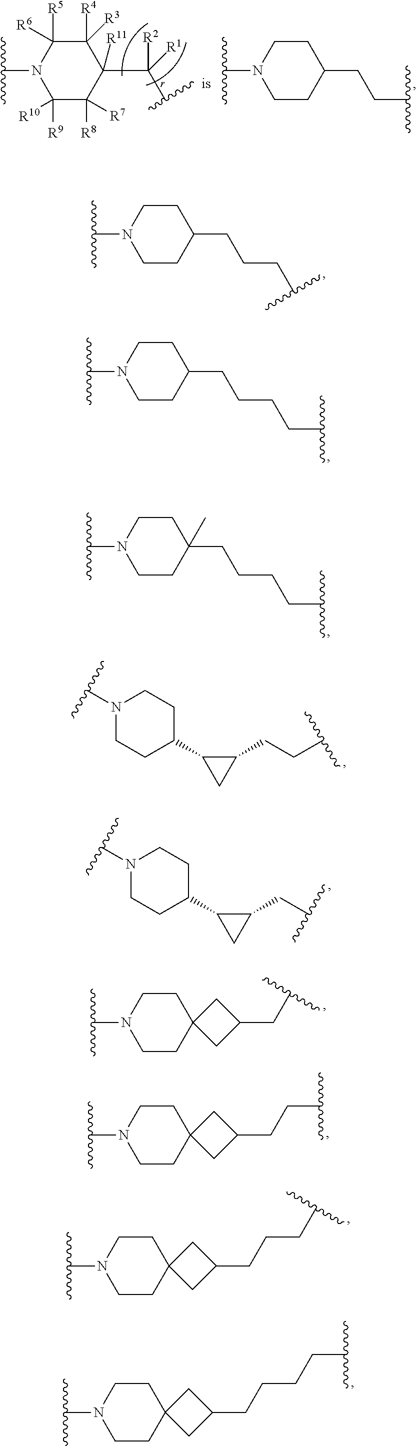

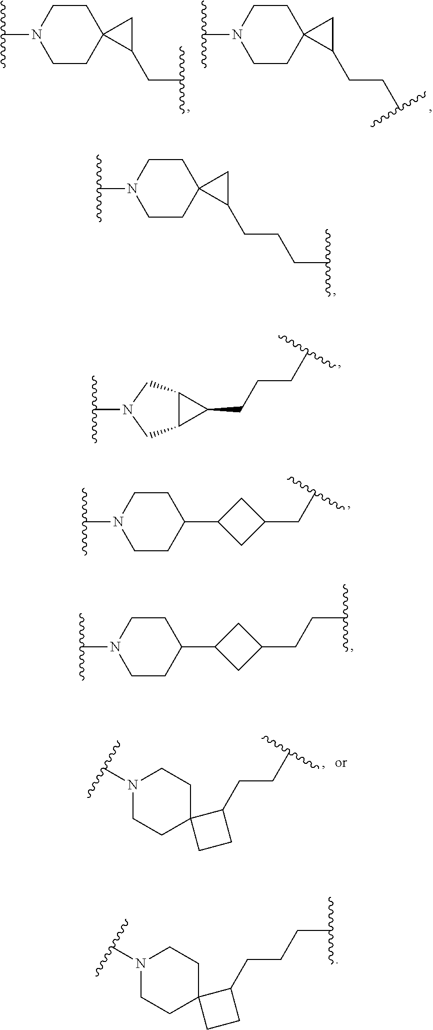

In some embodiments,

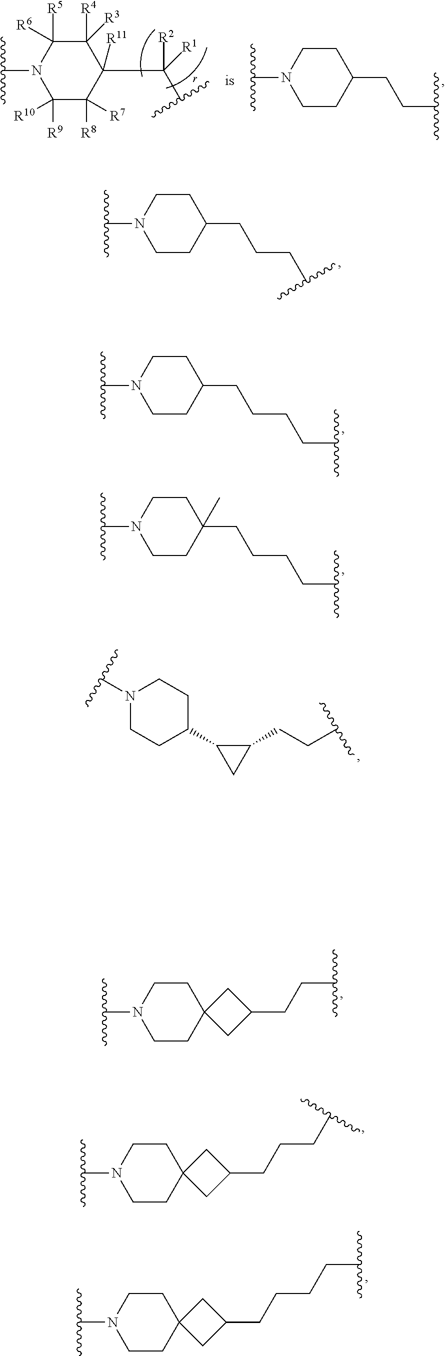

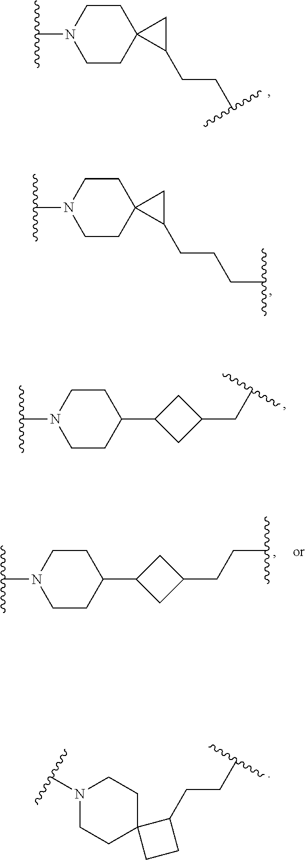 [SMILES: *CCC1CC12CCN(*)CC2 ; *CCCC1CC12CCN(*)CC2 ; *CC1CC(C2CCN(*)CC2)C1 ; *CCC1CC(C2CCN(*)CC2)C1 ; *CCC1CCC12CCN(*)CC2]

In some embodiments,

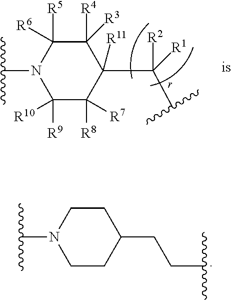

In some embodiments,

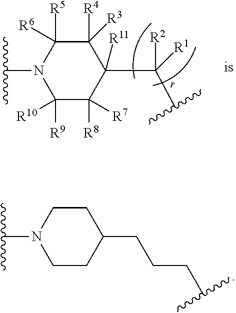

In some embodiments,

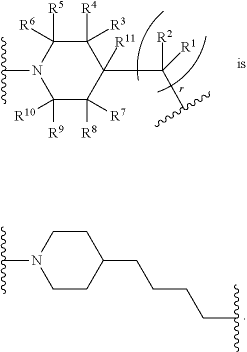

In some embodiments,

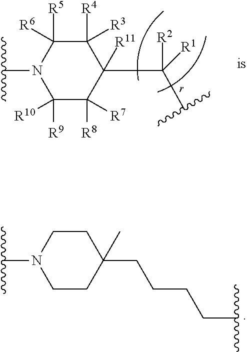

In some embodiments,

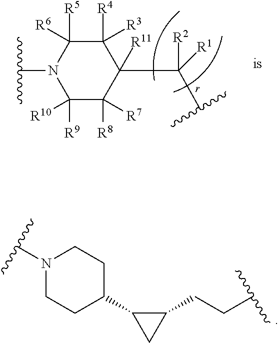

In some embodiments,

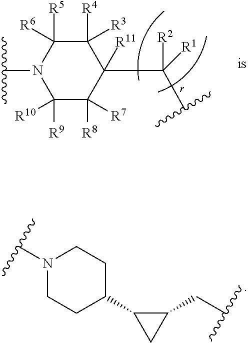

In some embodiments,

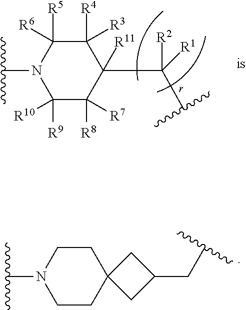

In some embodiments,

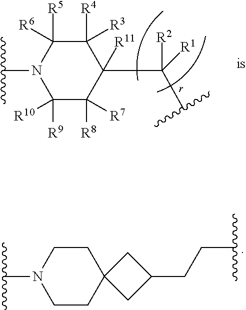

In some embodiments,

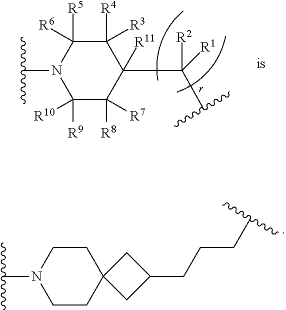

In some embodiments,

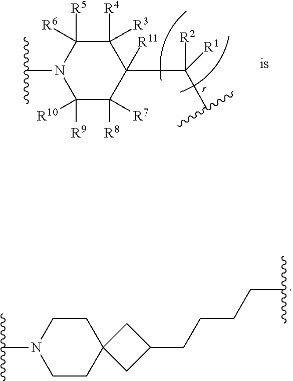

In some embodiments,

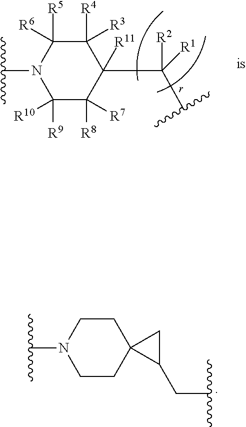

In some embodiments,

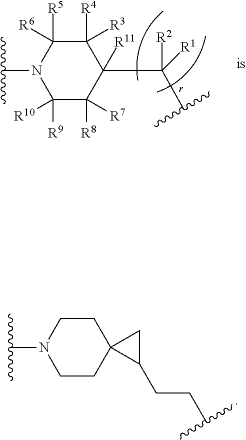

In some embodiments,

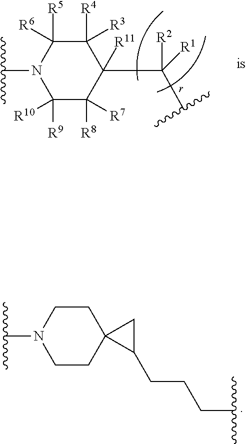

In some embodiments,

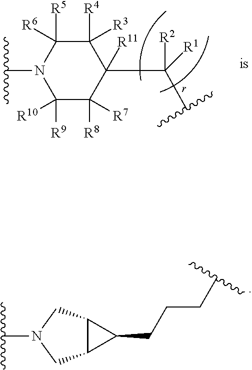

In some embodiments,

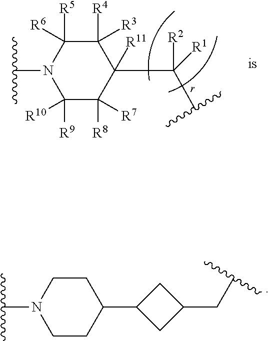

In some embodiments,

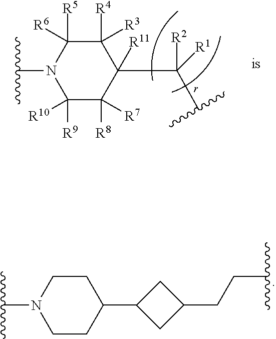

In some embodiments,

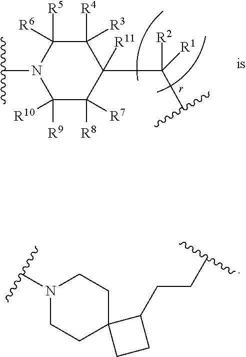

In some embodiments,

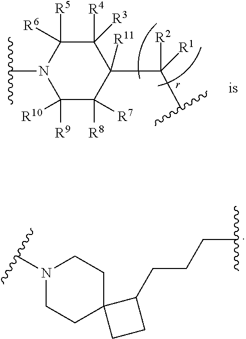

In some embodiments, X is —O—, #—CH₂O—, #—C(═O)O—, or #—CH₂C(═O)O—, where # represents the attachment point to Ring A; each R¹ is independently hydrogen, fluorine, —OH, C₁₋₆ alkyl, or C₁₋₆ alkoxy; or two R¹ on adjacent carbon atoms are taken together with the intervening atoms to which they are attached to form a cyclopropyl; each R² is independently hydrogen, fluorine, or C₁₋₆ alkyl; R¹¹ is hydrogen or C₁₋₈ alkyl; and R³, R⁴, R⁵, R⁶, R⁷, R⁸, R⁹, and R¹⁰ are each independently hydrogen or C₁₋₆ alkyl. In some embodiments, X is —O—; each R¹ is hydrogen; each R² is hydrogen; R¹¹ is hydrogen; and R³, R⁴, R⁵, R⁶, R⁷, R⁸, R⁹, and R¹⁰ are each hydrogen.

In some embodiments, Ring A is 5- or 6-membered monocyclic heteroaryl. In some embodiments, Ring A is 5-membered monocyclic heteroaryl. In some embodiments, Ring A is pyrrolyl, furanyl, thiophenyl, pyrazolyl, imidazolyl, oxazolyl, isoxazolyl, thiazolyl, isothiazolyl, triazolyl, oxadiazolyl, or thiadiazolyl. In some embodiments, Ring A is 6-membered monocyclic heteroaryl. In some embodiments, Ring A is pyridinyl, pyridazinyl, pyrimidinyl, pyrazinyl, or triazinyl. In some embodiments, Ring A is pyridinyl.

In some embodiments, Ring A is phenyl.

In some embodiments, Ring A is phenyl or 6-membered monocyclic heteroaryl. In some embodiments, Ring A is phenyl or pyridinyl.

In some embodiments, each R^{a} is independently halogen, —CN, C₁₋₆ alkyl, C₁₋₆ fluoroalkyl. In some embodiments, each R^{a} is independently halogen, C₁₋₆ alkyl, C₁₋₆ fluoroalkyl, or C₃₋₆ cycloalkyl. In some embodiments, each R^{a} is independently halogen, C₁₋₆ alkyl, or C₁₋₆ fluoroalkyl. In some embodiments, each R^{a} is independently halogen or C₁₋₆ alkyl.

In some embodiments, each R^{a} is independently halogen. In some embodiments, each R^{a} is independently —F, —Cl, —Br, C₁₋₄ alkyl, or C₁₋₄ fluoroalkyl. In some embodiments, each R^{a} is independently —F, —Cl, C₁₋₄ alkyl, or C₁₋₄ fluoroalkyl. In some embodiments, each R^{a} is —F.

In some embodiments, n is 0, 1, 2, 3, or 4. In some embodiments, n is 1, 2, 3, or 4. In some embodiments, n is 1, 2, or 3. In some embodiments, n is 1 or 2. In some embodiments, n is 0. In some embodiments, n is 1. In some embodiments, n is 2. In some embodiments, n is 3. In some embodiments, n is 4.

In some embodiments, Ring A is phenyl or pyridinyl; each R^{a} is independently halogen or C₁₋₆ alkyl; and n is 1, 2, or 3. In some embodiments, Ring A is phenyl; each R^{a} is independently halogen; and n is 1 or 2. In some embodiments, Ring A is phenyl; each R^{a} is independently —F; and n is 1. In some embodiments, Ring A is phenyl; each R^{a} is independently —F; and n is 2.

In some embodiments, disclosed herein is a compound of Formula (Ta), or a pharmaceutically acceptable salt, or solvate thereof:

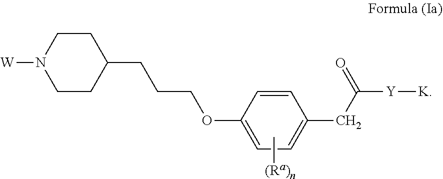

In some embodiments, disclosed herein is a compound of Formula (IIa), or a pharmaceutically acceptable salt, or solvate thereof:

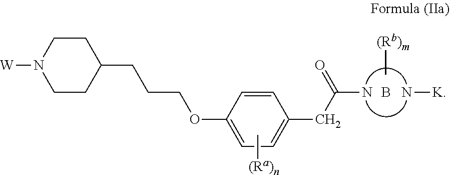

In some embodiments, disclosed herein is a compound of Formula (IIIa), or a pharmaceutically acceptable salt, or solvate thereof:

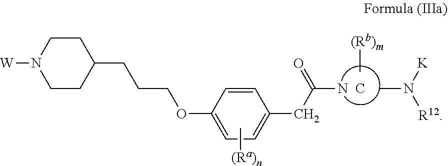

In some embodiments, W is phenyl, 5-membered monocyclic heteroaryl, or 6-membered monocyclic heteroaryl. In some embodiments, W is optionally substituted phenyl, optionally substituted 5-membered monocyclic heteroaryl, or optionally substituted 6-membered monocyclic heteroaryl. In some embodiments, W is phenyl, 5-membered monocyclic heteroaryl, or 6-membered monocyclic heteroaryl, wherein the phenyl or heteroaryl is unsubstituted or substituted with 1, 2, or 3 substituents selected from R^{c}. In some embodiments, W is phenyl, 5-membered monocyclic heteroaryl, or 6-membered monocyclic heteroaryl, wherein the phenyl or heteroaryl is unsubstituted or substituted with 1 or 2 substituents selected from R^{e}. In some embodiments, W is phenyl, 5-membered monocyclic heteroaryl, or 6-membered monocyclic heteroaryl, wherein the phenyl or heteroaryl is unsubstituted or substituted with 1 substituent selected from R^{e}.

In some embodiments, W is 5-membered monocyclic heteroaryl or 6-membered monocyclic heteroaryl. In some embodiments, W is 5-membered monocyclic heteroaryl or 6-membered monocyclic heteroaryl, wherein the heteroaryl is unsubstituted or substituted with 1, 2, or 3 substituents selected from R^{e}.

In some embodiments, W is 5-membered monocyclic heteroaryl. In some embodiments, W is 5-membered monocyclic heteroaryl. In some embodiments, W is 5-membered monocyclic heteroaryl which is unsubstituted or substituted with 1, 2, or 3 substituents selected from R^{e}. In some embodiments, W is pyrrolyl, furanyl, thiophenyl, pyrazolyl, imidazolyl, oxazolyl, isoxazolyl, thiazolyl, isothiazolyl, triazolyl, oxadiazolyl, or thiadiazolyl. In some embodiments, W is pyrrolyl, furanyl, thiophenyl, pyrazolyl, imidazolyl, oxazolyl, isoxazolyl, thiazolyl, isothiazolyl, triazolyl, oxadiazolyl, or thiadiazolyl which is unsubstituted or substituted with 1, 2, or 3 substituents selected from R^{e}.

In some embodiments, W is 6-membered monocyclic heteroaryl. In some embodiments, W is 6-membered monocyclic heteroaryl which is unsubstituted or substituted with 1, 2, or 3 substituents selected from R^{e}. In some embodiments, W is 6-membered monocyclic heteroaryl which is unsubstituted or substituted with 1 or 2 substituents selected from R^{e}. In some embodiments, W is pyridyl, pyridazinyl, pyrimidinyl, pyrazinyl, or triazinyl. In some embodiments, W is pyridyl, pyridazinyl, pyrimidinyl, pyrazinyl, or triazinyl which is unsubstituted or substituted with 1, 2, or 3 substituents selected from R^{e}.

In some embodiments, W is phenyl. In some embodiments, W is phenyl which is unsubstituted or substituted with 1, 2, or 3 substituents selected from R^{e}.

In some embodiments, W is phenyl or 6-membered monocyclic heteroaryl. In some embodiments, W is phenyl or pyrimidinyl. In some embodiments, W is phenyl or 6-membered monocyclic heteroaryl which is unsubstituted or substituted with 1, 2, or 3 substituents selected from R^{e}. In some embodiments, W is phenyl or pyrimidinyl which is unsubstituted or substituted with 1, 2, or 3 substituents selected from R^{e}.

In some embodiments, W is pyrimidinyl. In some embodiments, W is pyrimidinyl which is unsubstituted or substituted with 1, 2, or 3 substituents selected from R^{e}. In some embodiments, W is pyrimidinyl which is unsubstituted or substituted with 1 or 2 substituents selected from R^{e}. In some embodiments, W is pyrimidinyl which is unsubstituted or substituted with 1 substituent selected from R^{e}.

In some embodiments, W is unsubstituted or substituted with 1, 2, or 3 substituents selected from R^{e}. In some embodiments, W is unsubstituted or substituted with 1 or 2 substituents selected from R^{e}. In some embodiments, W is unsubstituted or substituted with 1 substituent selected from R^{e}. In some embodiments, W is unsubstituted. In some embodiments, W is substituted with 1 substituent selected from R^{e}.

In some embodiments, each R^{e} is independently halogen, —OH, —CN, —C(O)OH, —C(O)O(C₁₋₆ alkyl), C₁₋₆ alkyl, C₁₋₆ alkoxy, C₃₋₆ cycloalkyl, phenyl, or 5- to 6-membered heteroaryl. In some embodiments, each R^{e} is independently halogen, —OH, —CN, —C(O)OH, —C(O)O(C₁₋₆ alkyl), C₁₋₆ alkyl, C₁₋₆ alkoxy, C₃₋₆ cycloalkyl, phenyl, or 5- to 6-membered heteroaryl; wherein each alkyl, alkoxy, and cycloalkyl is unsubstituted or substituted with 1, 2, or 3 substituents selected from the group consisting of halogen, —OH, C₁₋₆ alkyl, and C₁₋₆ alkoxy.

In some embodiments, each R^{e} is independently halogen, —C(O)OH, —C(O)O(C₁₋₆ alkyl), C₁₋₆ alkyl, C₁₋₆ alkoxy, or C₃₋₆ cycloalkyl; wherein each alkyl, alkoxy, and cycloalkyl is unsubstituted or substituted with 1, 2, or 3 substituents selected from the group consisting of halogen, —OH, C₁₋₆ alkyl, and C₁₋₆ alkoxy. In some embodiments, each R^{e} is independently halogen, —C(O)O(C₁₋₆ alkyl), C₁₋₆ alkyl, or C₁₋₆ alkoxy; wherein each alkyl and alkoxy is unsubstituted or substituted with 1, 2, or 3 substituents selected from the group consisting of —OH and C₁₋₆ alkoxy. In some embodiments, each R^{c} is independently —F, —Cl, —Br, —C(O)O(C₁₋₄ alkyl), C₁₋₄ alkyl, or C₁₋₄ alkoxy; wherein each alkyl and alkoxy is unsubstituted or substituted with —OH or C₁₋₄ alkoxy.

In some embodiments, each R^{e} is independently —F, —Cl, —C(O)O(Me), —C(O)O(Et), methyl, ethyl, n-propyl, i-propyl, n-butyl, i-butyl, s-butyl, t-butyl, —OCH₃, —CH₂OCH₃, or —CH₂OH.

In some embodiments, W is 6-membered monocyclic heteroaryl, wherein the heteroaryl is unsubstituted or substituted with 1 or 2 substituents selected from R^{e}; and each R^{e} is independently halogen, —C(O)OH, —C(O)O(C₁₋₆ alkyl), C₁₋₆ alkyl, C₁₋₆ alkoxy, or C₃₋₆ cycloalkyl; wherein each alkyl, alkoxy, and cycloalkyl is unsubstituted or substituted with 1, 2, or 3 substituents selected from halogen, —OH, C₁₋₆ alkyl, and C₁₋₆ alkoxy. In some embodiments, W is 6-membered monocyclic heteroaryl, wherein the heteroaryl is unsubstituted or substituted with 1 or 2 substituents selected from R^{e}; and each R^{e} is independently halogen, —C(O)OH, —C(O)O(C₁₋₆ alkyl), C₁₋₆ alkyl, or C₁₋₆ alkoxy; wherein each alkyl is unsubstituted or substituted with 1 —OH or C₁₋₆ alkoxy substituent. In some embodiments, W is 6-membered monocyclic heteroaryl, wherein the heteroaryl is unsubstituted or substituted with 1 or 2 substituents R^{e}; and each R^{e} is independently —F, —Cl, —CH₃, —CH₂CH₃, —CH₂CH₂CH₃, —CH₂OH, —CH₂OCH₃, —OCH₃, —OCH₂CH₃, —C(O)OH, or —C(O)OCH₃.

In some embodiments, W is pyridinyl, wherein the pyridinyl is unsubstituted or substituted with 1 or 2 substituents selected from R^{e}; and each R^{e} is independently halogen, —C(O)OH, —C(O)O(C₁₋₆ alkyl), C₁₋₆ alkyl, C₁₋₆ alkoxy, or C₃₋₈cycloalkyl; wherein each alkyl, alkoxy, and cycloalkyl is unsubstituted or substituted with 1, 2, or 3 substituents selected from the group consisting of halogen, —OH, C₁₋₆ alkyl, and C₁₋₆ alkoxy. In some embodiments, W is pyridinyl, wherein the pyridinyl is unsubstituted or substituted with 1 or 2 substituents selected from R^{e}; and each R^{e} is independently —F, —Cl, —CH₃, —CH₂CH₃, —CH₂CH₂CH₃, —CH₂OH, —CH₂OCH₃, —OCH₃, —OCH₂CH₃, —C(O)OH, or —C(O)OCH₃.

In some embodiments, W is pyrimidinyl, wherein the pyrimidinyl is unsubstituted or substituted with 1 or 2 substituents selected from R^{e}; and each R^{e} is independently halogen, —C(O)OH, —C(O)O(C₁₋₆ alkyl), C₁₋₆ alkyl, C₁₋₆ alkoxy, or C₃₋₈cycloalkyl; wherein each alkyl, alkoxy, and cycloalkyl is unsubstituted or substituted with 1, 2, or 3 substituents selected from the group consisting of halogen, —OH, C₁₋₆ alkyl, and C₁₋₆ alkoxy. In some embodiments, W is pyrimidinyl, wherein the pyrimidinyl is unsubstituted or substituted with 1 or 2 substituents selected from R^{e}; and each R^{e} is independently —F, —Cl, —CH₃, —CH₂CH₃, —CH₂CH₂CH₃, —CH₂OH, —CH₂OCH₃, —OCH₃, —OCH₂CH₃, —C(O)OH, or —C(O)OCH₃.

In some embodiments, W is pyrazinyl, wherein the pyrazinyl is unsubstituted or substituted with 1 or 2 substituents selected from R^{e}; and each R^{e} is independently halogen, —C(O)OH, —C(O)O(C₁₋₆ alkyl), C₁₋₆ alkyl, C₁₋₆ alkoxy, or C₃₋₆ cycloalkyl; wherein each alkyl, alkoxy, and cycloalkyl is unsubstituted or substituted with 1, 2, or 3 substituents selected from the group consisting of halogen, —OH, C₁₋₆ alkyl, and C₁₋₆ alkoxy. In some embodiments, W is pyrazinyl, wherein the pyrazinyl is unsubstituted or substituted with 1 or 2 substituents selected from R^{e}; and each R^{e} is independently —F, —Cl, —CH₃, —CH₂CH₃, —CH₂CH₂CH₃, —CH₂OH, —CH₂OCH₃, —OCH₃, —OCH₂CH₃, —C(O)OH, or —C(O)OCH₃.

In some embodiments, W is pyridazinyl, wherein the pyridazinyl is unsubstituted or substituted with 1 or 2 substituents selected from R^{e}; and each R^{e} is independently halogen, —C(O)OH, —C(O)O(C₁₋₆ alkyl), C₁₋₆ alkyl, C₁₋₆ alkoxy, or C₃₋₆ cycloalkyl; wherein each alkyl, alkoxy, and cycloalkyl is unsubstituted or substituted with 1, 2, or 3 substituents selected from the group consisting of halogen, —OH, C₁₋₆ alkyl, and C₁₋₆ alkoxy. In some embodiments, W is pyridazinyl, wherein the pyridazinyl is unsubstituted or substituted with 1 or 2 substituents selected from R^{e}; and each R^{e} is independently —F, —Cl, —CH₃, —CH₂CH₃, —CH₂CH₂CH₃, —CH₂OH, —CH₂OCH₃, —OCH₃, —OCH₂CH₃, —C(O)OH, or —C(O)OCH₃.

In some embodiments, disclosed herein is a compound of Formula (Ib), or a pharmaceutically acceptable salt, or solvate thereof:

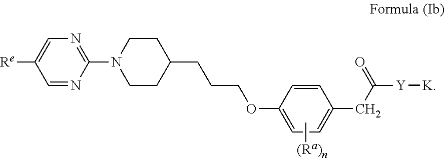

In some embodiments, disclosed herein is a compound of Formula (IIb), or a pharmaceutically acceptable salt, or solvate thereof:

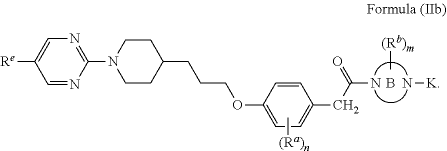

In some embodiments, disclosed herein is a compound of Formula (IIIb), or a pharmaceutically acceptable salt, or solvate thereof:

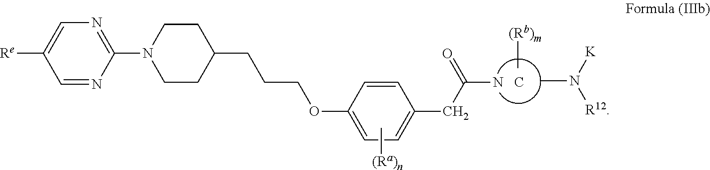

In some embodiments, W is —C(═O)O—R¹⁵. In some embodiments, W is —C(═O)O—R⁵; and R¹⁵ is C₁₋₆ alkyl, C₃₋₆ cycloalkyl, or 3- to 6-membered heterocycloalkyl. In some embodiments, W is —C(═O)O—R¹⁵; and R¹⁵ is C₁₋₆ alkyl, C₃₋₆ cycloalkyl, or 3- to 6-membered heterocycloalkyl, wherein the alkyl, cycloalkyl, or heterocycloalkyl is unsubstituted or substituted by 1-3 substituents independently selected from the group consisting of fluorine, —OH, C₁₋₆ alkyl, C₁₋₆ haloalkyl, and C₃₋₆ cycloalkyl. In some embodiments, W is —C(═O)O—R¹; and R¹⁵ is C₁₋₆ alkyl, C₃₋₆ cycloalkyl, or 3- to 6-membered heterocycloalkyl, wherein the alkyl, cycloalkyl, or heterocycloalkyl is unsubstituted or substituted by 1-3 substituents independently selected from the group consisting of C₁₋₆ alkyl and C₁₋₆ fluoroalkyl. In some embodiments, W is —C(═O)O—R¹⁵; and R¹⁵ is C₁₋₆ alkyl or 3- to 6-membered heterocycloalkyl, wherein the alkyl or heterocycloalkyl is unsubstituted or substituted by 1-3 substituents independently selected from the group consisting of C₁₋₆ alkyl and C₁₋₆ fluoroalkyl.

In some embodiments, Y is

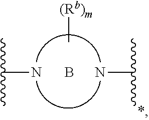

where Ring B is a heterocycloalkyl; wherein Ring B comprises 2 N atoms and 0 or 1 O or S atoms, and * represents the attachment point to K. In some embodiments, Ring B is a monocyclic heterocycloalkyl or a bicyclic heterocycloalkyl; wherein Ring B comprises 2 N atoms and 0 or 1 O or S atoms. In some embodiments, Ring B is a monocyclic heterocycloalkyl, fused bicyclic heterocycloalkyl, bridged bicyclic heterocycloalkyl, or spirocyclic bicyclic heterocycloalkyl; wherein Ring B comprises 2 N atoms and 0 or 1 O or S atoms.

In some embodiments, Ring B is a monocyclic heterocycloalkyl; wherein Ring B comprises 2 N atoms and 0 or 1 O or S atoms. In some embodiments, Ring B is a monocyclic 4- to 8-membered heterocycloalkyl; wherein Ring B comprises 2 N atoms and 0 or 1 O or S atoms. In some embodiments, Ring B is a monocyclic 4-membered heterocycloalkyl, 5-membered heterocycloalkyl, 6-membered heterocycloalkyl, 7-membered heterocycloalkyl, or 8-membered heterocycloalkyl; wherein Ring B comprises 2 N atoms and 0 or 1 O or S atoms. In some embodiments, Ring B is a monocyclic 4- to 8-membered heterocycloalkyl; wherein Ring B comprises 2 N atoms and 0 O or S atoms. In some embodiments, Ring B is a monocyclic 4-membered heterocycloalkyl, 5-membered heterocycloalkyl, 6-membered heterocycloalkyl, 7-membered heterocycloalkyl, or 8-membered heterocycloalkyl; wherein Ring B comprises 2 N atoms and 0 O or S atoms. In some embodiments, Ring B is a 1,3-diazetidinyl, imidazolidinyl, piperazinyl, 1,4-diazepanyl, 1,4-diazocanyl, or 1,5-diazocanyl. In some embodiments, Ring B is a piperazinyl or 1,4-diazepanyl. In some embodiments, Ring B is a piperazinyl.

In some embodiments, Ring B is a bicyclic heterocycloalkyl; wherein Ring B comprises 2 N atoms and 0 or 1 O or S atoms. In some embodiments, Ring B is a 7- to 12-membered bicyclic heterocycloalkyl; wherein Ring B comprises 2 N atoms and 0 or 1 O or S atoms. In some embodiments, Ring B is a fused bicyclic heterocycloalkyl, bridged bicyclic heterocycloalkyl, or spirocyclic bicyclic heterocycloalkyl; wherein Ring B comprises 2 N atoms and 0 or 1 O or S atoms. In some embodiments, Ring B is a 7- to 12-membered fused bicyclic heterocycloalkyl, 7- to 12-membered bridged bicyclic heterocycloalkyl, or 7- to 12-membered spirocyclic bicyclic heterocycloalkyl; wherein Ring B comprises 2 N atoms and 0 or 1 O or S atoms.

In some embodiments, Ring B is a fused bicyclic heterocycloalkyl; wherein Ring B comprises 2 N atoms and 0 or 1 O or S atoms. In some embodiments, Ring B is a 7- to 12-membered fused bicyclic heterocycloalkyl; wherein Ring B comprises 2 N atoms and 0 or 1 O or S atoms. In some embodiments, Ring B is a 7- to 12-membered fused bicyclic heterocycloalkyl that is a 3,4-fused heterocycloalkyl, a 3,5-fused heterocycloalkyl, a 3,6-fused heterocycloalkyl, a 4,4-fused heterocycloalkyl, a 4,5-fused heterocycloalkyl, a 4,6-fused heterocycloalkyl, a 5,5-fused heterocycloalkyl, a 5,6-fused heterocycloalkyl, or a 6,6-fused heterocycloalkyl; wherein Ring B comprises 2 N atoms and 0 or 1 O or S atoms.

In some embodiments, Ring B is a bridged bicyclic heterocycloalkyl; wherein Ring B comprises 2 N atoms and 0 or 1 O or S atoms. In some embodiments, Ring B is a 7- to 12-membered bridged bicyclic heterocycloalkyl; wherein Ring B comprises 2 N atoms and 0 or 1 O or S atoms. In some embodiments, Ring B is a 7- to 12-membered bridged bicyclic heterocycloalkyl that is a bicyclo[2.2.1]heterocycloalkyl, a bicyclo[3.1.1]heterocycloalkyl, a bicyclo[3.2.1]heterocycloalkyl, a bicyclo[2.2.2]heterocycloalkyl, a bicyclo[3.3.1]heterocycloalkyl, or a bicyclo[3.2.2]heterocycloalkyl; wherein Ring B comprises 2 N atoms and 0 or 1 O or S atoms.

In some embodiments, Ring B is a spirocyclic bicyclic heterocycloalkyl; wherein Ring B comprises 2 N atoms and 0 or 1 O or S atoms. In some embodiments, Ring B is a 7- to 12-membered spirocyclic bicyclic heterocycloalkyl; wherein Ring B comprises 2 N atoms and 0 or 1 O or S atoms. In some embodiments, Ring B is a 7- to 12-membered spirocyclic bicyclic heterocycloalkyl that is a 4,4-spiroheterocycloalkyl, a 4,5-spiroheterocycloalkyl, a 4,6-spiroheterocycloalkyl, a 5,5-spiroheterocycloalkyl, a 5,6-spiroheterocycloalkyl, or a 6,6-spiroheterocycloalkyl; wherein Ring B comprises 2 N atoms and 0 or 1 O or S atoms.

In some embodiments, Ring B is

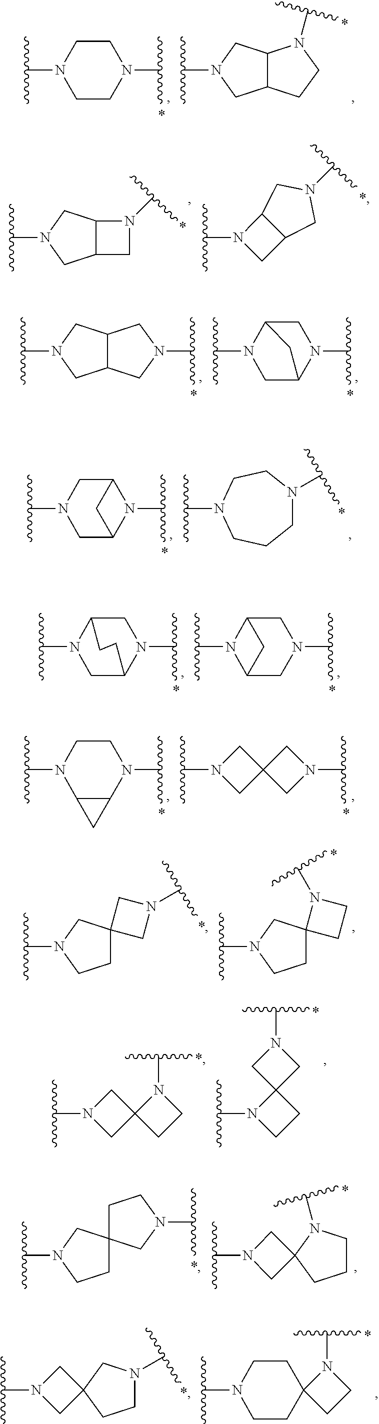

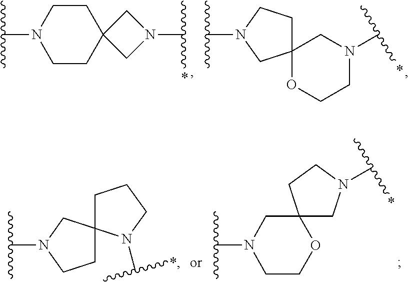

where * represents the attachment point to K.

In some embodiments,

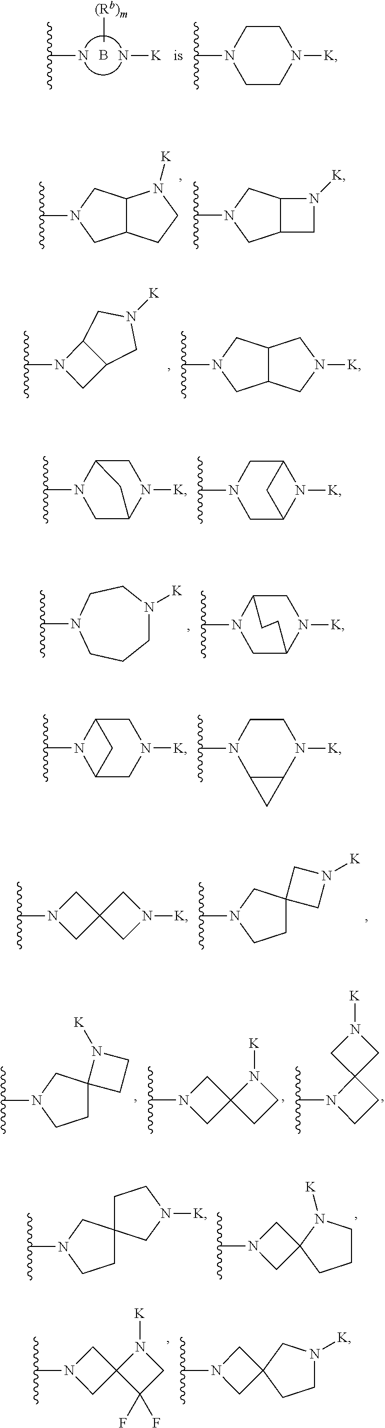

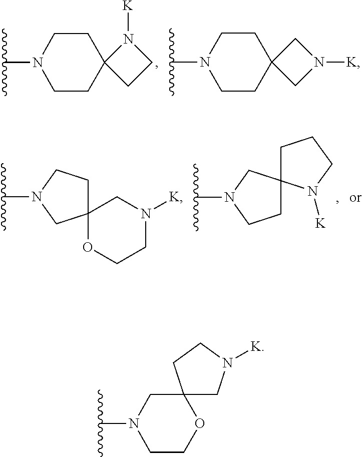

In some embodiments, Y is

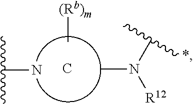

where Ring C is a bicyclic heterocycloalkyl; wherein Ring C comprises 1 or 2 N atoms and 0 or 1 O or S atoms, R¹² is hydrogen or C₁₋₄ alkyl, and * represents the attachment point to K.

In some embodiments, Ring C is a fused bicyclic heterocycloalkyl, bridged bicyclic heterocycloalkyl, or spirocyclic bicyclic heterocycloalkyl; wherein Ring C comprises 1 or 2 N atoms and 0 or 1 O or S atoms. In some embodiments, Ring C is a 7- to 12-membered fused bicyclic heterocycloalkyl, 7- to 12-membered bridged bicyclic heterocycloalkyl, or 7- to 12-membered spirocyclic bicyclic heterocycloalkyl; wherein Ring C comprises 1 or 2 N atoms and 0 or 1 O or S atoms.

In some embodiments, Ring C is a fused bicyclic heterocycloalkyl; wherein Ring C comprises 1 or 2 N atoms and 0 or 1 O or S atoms. In some embodiments, Ring C is a 7- to 12-membered fused bicyclic heterocycloalkyl; wherein Ring C comprises 1 or 2 N atoms and 0 or 1 O or S atoms. In some embodiments, Ring C is a 7- to 12-membered fused bicyclic heterocycloalkyl that is a 3,4-fused heterocycloalkyl, a 3,5-fused heterocycloalkyl, a 3,6-fused heterocycloalkyl, a 4,4-fused heterocycloalkyl, a 4,5-fused heterocycloalkyl, a 4,6-fused heterocycloalkyl, a 5,5-fused heterocycloalkyl, a 5,6-fused heterocycloalkyl, or a 6,6-fused heterocycloalkyl; wherein Ring C comprises 1 or 2 N atoms and 0 or 1 O or S atoms.

In some embodiments, Ring C is a bridged bicyclic heterocycloalkyl; wherein Ring C comprises 1 or 2 N atoms and 0 or 1 O or S atoms. In some embodiments, Ring C is a 7- to 12-membered bridged bicyclic heterocycloalkyl; wherein Ring C comprises 1 or 2 N atoms and 0 or 1 O or S atoms. In some embodiments, Ring C is a 7- to 12-membered bridged bicyclic heterocycloalkyl that is a bicyclo[2.2.1]heterocycloalkyl, a bicyclo[3.1.1]heterocycloalkyl, a bicyclo[3.2.1]heterocycloalkyl, a bicyclo[2.2.2]heterocycloalkyl, a bicyclo[3.3.1]heterocycloalkyl, or a bicyclo[3.2.2]heterocycloalkyl; wherein Ring C comprises 1 or 2 N atoms and 0 or 1 O or S atoms.

In some embodiments, Ring C is a spirocyclic bicyclic heterocycloalkyl; wherein Ring C comprises 1 or 2 N atoms and 0 or 1 O or S atoms. In some embodiments, Ring C is a 7- to 12-membered spirocyclic bicyclic heterocycloalkyl; wherein Ring C comprises 1 or 2 N atoms and 0 or 1 O or S atoms. In some embodiments, Ring C is a 7- to 12-membered spirocyclic bicyclic heterocycloalkyl that is a 4,4-spiroheterocycloalkyl, a 4,5-spiroheterocycloalkyl, a 4,6-spiroheterocycloalkyl, a 5,5-spiroheterocycloalkyl, a 5,6-spiroheterocycloalkyl, or a 6,6-spiroheterocycloalkyl; wherein Ring C comprises 1 or 2 N atoms and 0 or 1 O or S atoms.

In some embodiments, Ring C is a spirocyclic bicyclic heterocycloalkyl; wherein Ring C comprises 1 N atom and 0 or 1 O or S atoms. In some embodiments, Ring C is a 7- to 12-membered spirocyclic bicyclic heterocycloalkyl; wherein Ring C comprises 1 N atom and 0 or 1 O or S atoms. In some embodiments, Ring C is a 7- to 12-membered spirocyclic bicyclic heterocycloalkyl that is a 4,4-spiroheterocycloalkyl, a 4,5-spiroheterocycloalkyl, a 4,6-spiroheterocycloalkyl, a 5,5-spiroheterocycloalkyl, a 5,6-spiroheterocycloalkyl, or a 6,6-spiroheterocycloalkyl; wherein Ring C comprises 1 N atom and 0 or 1 O or S atoms.

In some embodiments, R¹² is hydrogen or C₁₋₄ alkyl. In some embodiments, R¹² is hydrogen or C₁₋₂ alkyl. In some embodiments, R¹² is hydrogen, methyl, ethyl, n-propyl, i-propyl, n-butyl, i-butyl, s-butyl, or t-butyl. In some embodiments, R¹² is hydrogen or methyl. In some embodiments, R¹² is hydrogen. In some embodiments, R¹² is methyl.

In some embodiments, Ring C is a fused bicyclic heterocycloalkyl, bridged bicyclic heterocycloalkyl, or spirocyclic bicyclic heterocycloalkyl; wherein Ring C comprises 1 or 2 N atoms and 0 or 1 O or S atoms; and R¹² is hydrogen or C₁₋₄ alkyl. In some embodiments, Ring C is a 7- to 12-membered fused bicyclic heterocycloalkyl, 7- to 12-membered bridged bicyclic heterocycloalkyl, or 7- to 12-membered spirocyclic bicyclic heterocycloalkyl; wherein Ring C comprises 1 or 2 N atoms and 0 or 1 O or S atoms; and R¹² is hydrogen or C₁₋₄ alkyl. In some embodiments, Ring C is a 7- to 12-membered spirocyclic bicyclic heterocycloalkyl; wherein Ring C comprises 1 N atom and 0 or 1 O or S atoms; and R¹² is hydrogen or C₁₋₂ alkyl.

In some embodiments, Ring C is a 7- to 12-membered spirocyclic bicyclic heterocycloalkyl that is a 3,4-spiroheterocycloalkyl, a 3,5-spiroheterocycloalkyl, a 3,6-spiroheterocycloalkyl, 4,4-spiroheterocycloalkyl, a 4,5-spiroheterocycloalkyl, a 4,6-spiroheterocycloalkyl, a 5,5-spiroheterocycloalkyl, a 5,6-spiroheterocycloalkyl, or a 6,6-spiroheterocycloalkyl; wherein Ring C comprises 1 or 2 N atoms and 0 or 1 O or S atoms; and R¹² is hydrogen or methyl.

In some embodiments, Ring C is

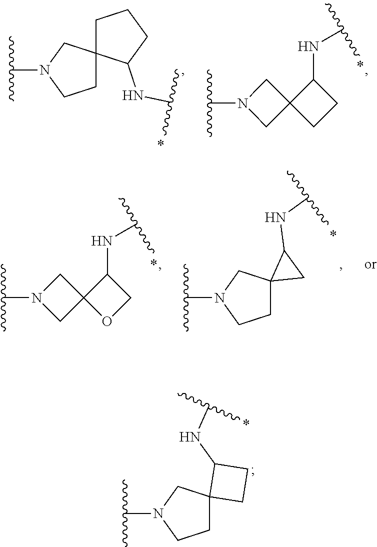

where * represents the attachment point to K.

In some embodiments,

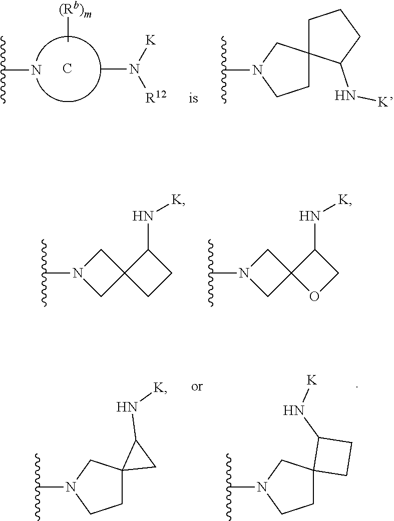

In some embodiments, each R^{b} is independently fluoro or C₁₋₄ alkyl.

In some embodiments, m is 0, 1, 2, 3, or 4. In some embodiments, m is 1, 2, 3, or 4.

In some embodiments, m is 0, 1, or 2. In some embodiments, m is 0. In some embodiments, m is 1. In some embodiments, m is 2. In some embodiments, m is 3. In some embodiments, m is 4.

In some embodiments, each R^{b} is independently fluoro or C₁₋₄ alkyl; and m is 0, 1, or 2.

In some embodiments, —Y—K is

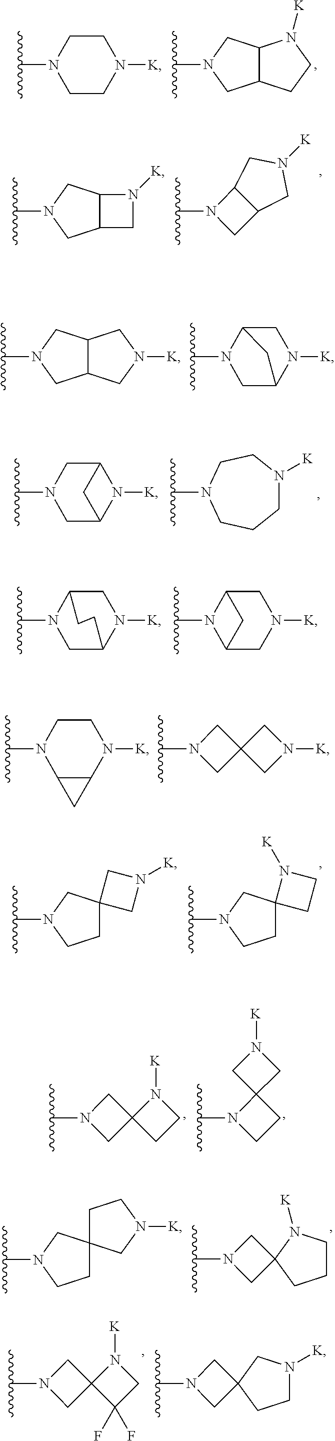

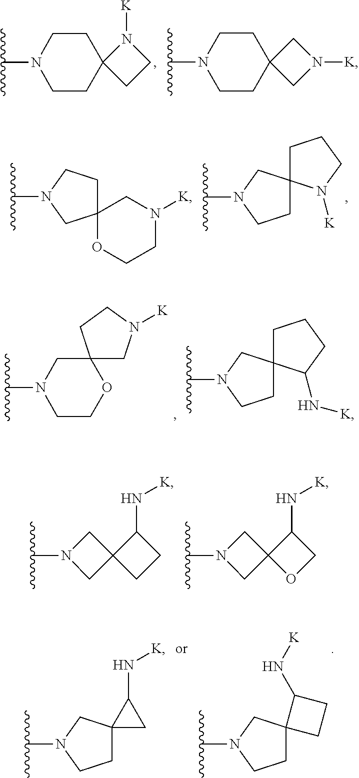

In some embodiments, —Y—K is

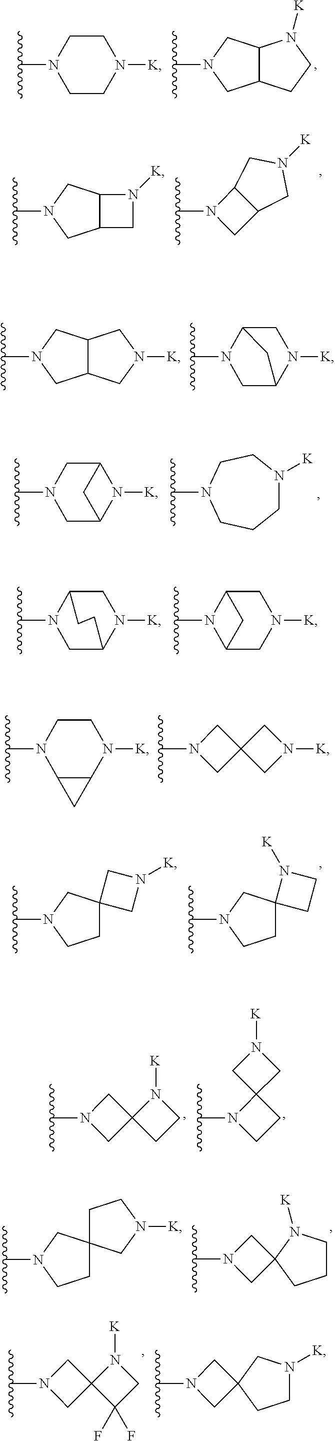

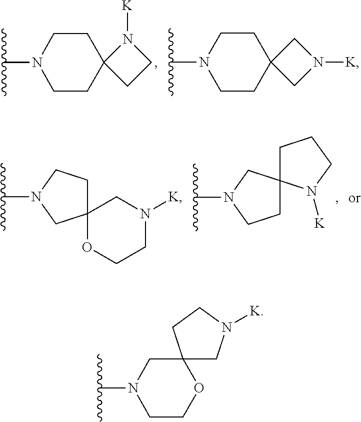

In some embodiments, —Y—K is

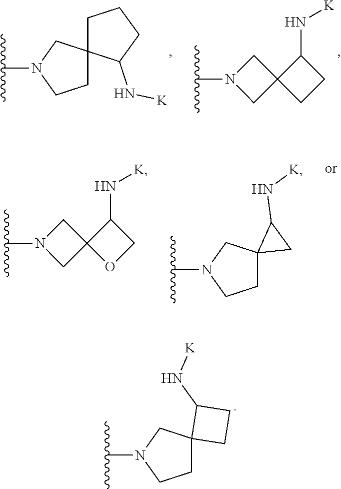

In some embodiments, K is Cis alkyl, —C(═O)—C₁₋₈ alkyl, —[(CH₂)ₛ—Z]ₜ—R¹, —[(CHR^{d})ₛ—Z]ₜ—R¹³, or —[(C(R^{d})₂)ₛ—Z]ₜ—R¹³. In some embodiments, K is C₁₋₈ alkyl, —C(═O)—C₁₋₈ alkyl, —[(CH₂)ₛ—Z]ₜ—R¹³, —[(CHR^{d})ₛ—Z]ₜ—R¹³, or —[(C(R^{d})₂)ₛ—Z]ₜ—R¹³; wherein the alkyl is substituted by 1-6 R^{e} group. In some embodiments, K is C₁₋₈ alkyl, —C(═O)—C₁₋₈ alkyl, —[(CH₂)ₛ—Z]ₜ—R¹³, —[(CHR^{d})ₛ—Z]ₜ—R¹³, or —[(C(R^{d})₂)ₛ—Z]ₜ—R¹³; wherein the alkyl is substituted by 1-6 R^{c} group; and each Z is independently —CH₂O—, —CH₂NH—, —CH₂NR^{d}—, or —CH₂N⁺(R^{d})₂—. In some embodiments, K is C₁₋₈ alkyl, —C(═O)—C₁₋₈ alkyl, —[(CH₂)ₛ—Z]ₜ—R¹³, —[(CHR^{d})ₛ—Z]ₜ—R¹³, or —[(C(R^{d})₂)ₛ—Z]ₜ—R¹³; wherein the alkyl is substituted by 1-6 R^{c} group; each Z is independently —CH₂O—, —CH₂NH—, —CH₂NR^{d}—, or —CH₂N⁺(R^{d})₂—; s is 1-3, t is 1-3; and R¹³ is hydrogen or a C₁₋₈ alkyl that is unsubstituted or substituted by 1-6 R^{c} groups.

In some embodiments, K is C₁₋₈ alkyl, C₂₋₈ alkenyl, C₂₋₈ alkynyl, —C(═O)—C₁₋₈ alkyl, —C(═O)—C₂₋₈alkenyl, or —C(═O)—C₂₋₈alkynyl; wherein each alkyl, alkenyl, or alkynyl is substituted by 1-6 R^{e} groups. In some embodiments, K is C₁₋₈ alkyl or —C(═O)—C₁₋₈ alkyl; wherein the alkyl is substituted by 1-6 R^{c} groups. In some embodiments, K is C₄₋₆ alkyl or —C(═O)—C₄₋₆ alkyl; wherein each alkyl, alkenyl, or alkynyl is substituted by 1-6 R^{c} groups.

In some embodiments, each R^{c} is independently —OH, —CH₂OH, —CH₂CH₂OH, —NH₂, —CH₂NH₂, —NH(R^{d}), —CH₂NH(R^{d}), —N(R^{d})₂, —CH₂N(R^{d})₂, —N(R^{d})₃⁺, —C(═O)OH, —CH₂C(═O)OH, —CH₂CH₂C(═O)OH, —S(═O)₂OH, —S(═O)OH, —S(═O)₂NH₂, —P(═O)(OH)₂, —P(═O)(OH)(R^{d}), —P(═O)(OH)(H), P(═O)(OH)(OR^{d}), —B(OH)₂, —B(OR^{d})(OH), —NHCONHS(═O)₂(R^{d}), —N(R^{d})CONHS(═O)₂(R^{d}), —NHCON(R^{d})S(═O)₂(R^{d}), —C(═O)NHS(═O)₂(R^{d}), —S(═O)₂NHC(═O)R^{d}, —NHC(═NH)NH₂, —NHC(═NH)NHR^{d}, —NHC(═NH)N(R^{d})₂, —N(R^{d})C(═NH)NH₂, —N(R^{d})C(═NH)NH(R^{d}), —N(R^{d})C(═NH)N(R^{d})₂, —NHC(═N(R^{d}))NH₂, —NHC(═N(R^{d}))NHR^{d}, —NHC(═N(R^{d}))N(R^{d})₂, —N(R^{d})C(═N(R^{d}))NH₂, —N(R^{d})C(═N(R^{d}))(NHR^{d}, —N(R^{d})C(═N(R^{d})) N(R^{d})₂, —NHC(═NH)NHC(═NH)NH₂, —N(R^{d})C(═NH)NHC(═NH)NH₂,

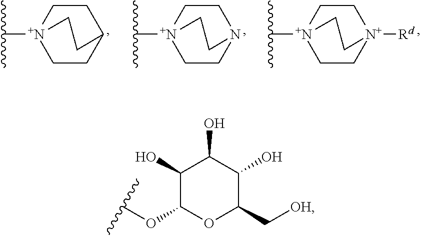

or a 4- to 6-membered heterocycle which is unsubstituted or substituted with 1, 2, 3, or 4 substituents selected from the group consisting of C₁₋₆ alkyl, —O—(C₁₋₆ alkyl), —OH, ═O and ═S. In some embodiments, each R⁴ is independently —OH, —CH₂OH, —CH₂CH₂OH, —NH₂, —CH₂NH₂, —NH(R^{d}), —CH₂NH(R^{d}), —N(R^{d})₂, —CH₂N(R^{d})₂, —N(R^{d})₃⁺, —C(═O)OH, —CH₂C(═O)OH, —CH₂CH₂C(═O)OH, —S(═O)₂OH, —S(═O)OH, —S(═O)₂NH₂, —P(═O)(OH)₂, —P(═O)(OH)(R^{d}), —P(═O)(OH)(H), —P(═O)(OH)(OR^{d}), —B(OH)₂, —B(OR^{d})(OH), —N(R^{d})CONHS(═O)₂(R^{d}), —C(═O)NHS(═O)₂(R^{d}), —NHC(═NH)NH₂, —N(R^{d})C(═NH)NH₂, —NHC(═NH)NHC(═NH)NH₂,

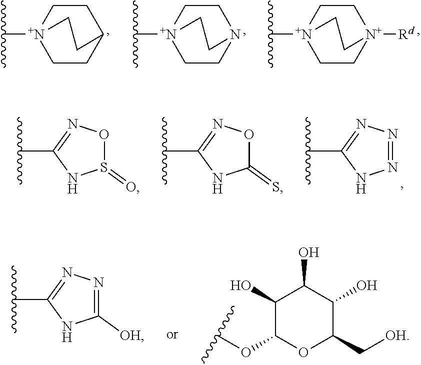

In some embodiments, each R^{c} is independently —OH, —CH₂OH, —NH₂, —N(R^{d})₃⁺, —C(═O)OH, —S(═O)₂OH, —S(═O)₂NH₂, —P(═O)(OH)₂, —P(═O)(OH)(R^{d}), —P(═O)(OH)(OR^{d}),

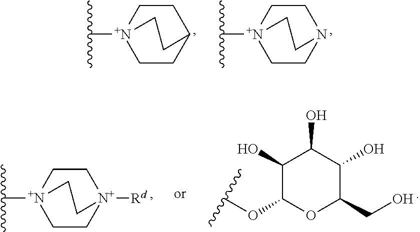

In some embodiments, each R^{c} is independently —OH, —NH₂, —N(R^{d})₃⁺, —C(═O)OH, —S(═O)₂OH, —S(═O)₂NH₂, —P(═O)(OH)₂, —P(═O)(OH)(R^{d}), —P(═O)(OH)(OR^{d}),

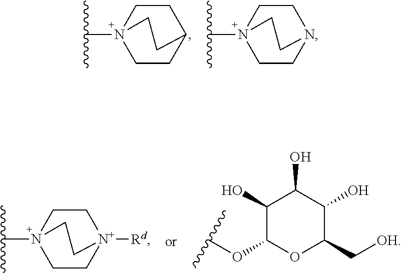

In some embodiments, each R^{c} is independently —OH, —CH₂OH, —NH₂, —N(R^{d})₃⁺, —C(═O)OH, or —S(═O)₂OH. In some embodiments, each R^{c} is independently —OH, —CH₂OH, —N(R^{d})₃⁺, —C(═O)OH, or —S(═O)₂OH. In some embodiments, each R^{c} is independently —OH, —N(R^{d})₃⁺, —C(═O)OH, or —S(═O)₂OH. In some embodiments, each R^{c} is independently —OH, —CH₂OH, or —S(═O)₂OH. In some embodiments, each R^{c} is —OH, —C(O)OH, or —S(═O)₂OH. In some embodiments, each R^{c} is —OH.

In some embodiments, each R^{c} is —C(O)OH. In some embodiments, each R^{c} is —S(═O)₂OH.

In some embodiments, each R^{d} is independently C₁₋₆ alkyl, C₁₋₆ fluoroalkyl, or C₃₋₆ cycloalkyl. In some embodiments, each R^{d} is independently C₁₋₆ alkyl or C₃₋₆ cycloalkyl. In some embodiments, each R^{d} is independently C₁₋₆ alkyl or C₁₋₆ fluoroalkyl. In some embodiments, each R^{d} is independently C₁₋₆ alkyl. In some embodiments, each R^{d} is independently C₁₋₄ alkyl. In some embodiments, each R^{d} is independently methyl, ethyl, n-propyl, i-propyl, n-butyl, i-butyl, s-butyl, or t-butyl. In some embodiments, each R^{d} is methyl.

In some embodiments, K is C₁₋₈ alkyl, —C(═O)—C₁₋₈ alkyl, —[(CH₂)ₛ—Z]ₜ—R¹, —[(CHR^{d})ₛ—Z]ₜ—R¹³, or —[(C(R^{d})₂)ₛ—Z]ₜ—R¹³; wherein the alkyl is substituted by 1-6 R^{c} group; each Z is independently —CH₂O—, —CH₂NH—, —CH₂NR^{d}—, or —CH₂N⁺(R^{d})₂—; s is 1-3; t is 1-3; R¹³ is hydrogen or a C₁₋₈ alkyl that is unsubstituted or substituted by 1-6 R^{c} groups; each R^{c} is independently —OH, —CH₂OH, —NH₂, —N(R^{d})₃⁺, —C(═O)OH, —S(═O)₂OH, —S(═O)₂NH₂, —P(═O)(OH)₂, —P(═O)(OH)(R^{d}), —P(═O)(OH)(OR^{d}),

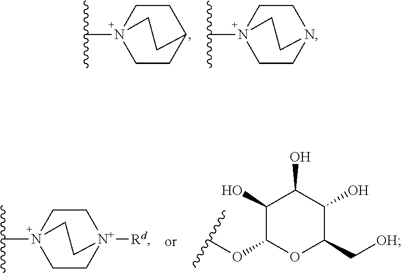

and each R^{d} is independently C₁₋₆ alkyl. In some embodiments, each R^{c} is independently —OH, —CH₂OH, —NH₂, —N(R^{d})₃⁺, —C(═O)OH, or —S(═O)₂OH. In some embodiments, K is C₁₋₈ alkyl or —C(═O)—C₁₋₈ alkyl; wherein the alkyl is substituted by 1-6 R^{c} groups; and each R^{c} is independently —OH, —CH₂OH, —N(R^{d})₃⁺, —C(═O)OH, or —S(═O)₂OH.

In some embodiments, K is C₄₋₆ alkyl or —C(═O)—C₄₋₆ alkyl; wherein the alkyl is substituted by 1-6 —OH or —S(═O)₂OH groups.

In some embodiments, K is

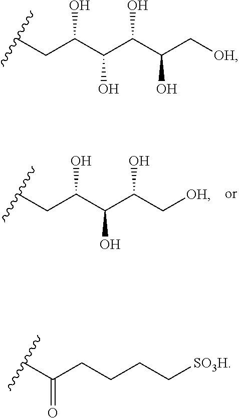 [SMILES: *C[C@H](O)[C@@H](O)[C@H](O)[C@H](O)CO ; *C[C@H](O)[C@H](O)[C@H](O)CO ; *C(=O)CCCCS(=O)(=O)O]

In some embodiments,
X is —O—, #—CH₂O—, #—C(═O)O—, or #—CH₂C(═O)O—, where # represents the attachment point to Ring A;
each R¹ is independently hydrogen, fluorine, —OH, C₁₋₆ alkyl, or C₁₋₆ alkoxy;
or two R¹ on adjacent carbon atoms are taken together with the intervening atoms to which they are attached to form a cyclopropyl;
each R² is independently hydrogen, fluorine, or C₁₋₆ alkyl;
R¹¹ is hydrogen or C₁₋₆ alkyl;
R³, R⁴, R⁵, R⁶, R⁷, R⁸, R⁹, and R¹⁰ are each independently hydrogen or C₁₋₆ alkyl;
Ring A is phenyl or pyridinyl;
each R^{a} is independently halogen or C₁₋₆ alkyl;
n is 1, 2, or 3;
W is 6-membered monocyclic heteroaryl, wherein the heteroaryl is unsubstituted or substituted with 1 or 2 substituents selected from R^{e};
   each R^{c} is independently halogen, —C(O)OH, —C(O)O(C₁₋₆ alkyl), C₁₋₆ alkyl, C₁₋₆ alkoxy, or C₃₋₆ cycloalkyl; wherein each alkyl, alkoxy, and cycloalkyl is unsubstituted or substituted with 1, 2, or 3 substituents selected from the group consisting of halogen, —OH, C₁₋₆ alkyl, and C₁₋₆ alkoxy;
or W is —C(═O)O—R¹⁵;
   R¹⁵ is C₁₋₆ alkyl, C₃₋₆ cycloalkyl, or 3- to 6-membered heterocycloalkyl, wherein the alkyl, cycloalkyl, or heterocycloalkyl is unsubstituted or substituted by 1-3 substituents independently selected from the group consisting of C₁₋₆ alkyl and C₁₋₆ fluoroalkyl;
Y is

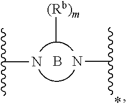

where Ring B is a monocyclic heterocycloalkyl or a bicyclic heterocycloalkyl; wherein Ring B comprises 2 N atoms and 0 or 1 O or S atoms, and * represents the attachment point to K;
or Y is

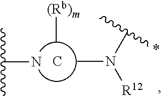

where Ring C is a fused bicyclic heterocycloalkyl, bridged bicyclic heterocycloalkyl, or spirocyclic bicyclic heterocycloalkyl; wherein Ring C comprises 1 or 2 N atoms and 0 or 1 O or S atoms; R¹² is hydrogen or C₁₋₄ alkyl; and * represents the attachment point to K;
each R^{b} is independently fluoro or C₁₋₄ alkyl;
m is 0, 1, or 2;
K is C₁₋₈ alkyl, —C(═O)—C₁₋₈ alkyl, —[(CH₂)ₛ—Z]ₜ—R¹³, —[(CHR^{d})ₛ—Z]ₜ—R¹³, or —[(C(R^{d})₂)ₛ—Z]ₜ—R¹; wherein the alkyl is substituted by 1-6 R^{c} group;
each Z is independently —CH₂O—, —CH₂NH—, —CH₂NR^{d}—, or —CH₂N⁺(R^{d})₂—;
s is 1-3;
t is 1-3;
R¹³ is hydrogen or a C₁₋₈ alkyl that is unsubstituted or substituted by 1-6 R^{c} groups;
each R^{c} is independently —OH, —CH₂OH, —NH₂, —N(R^{d})₃⁺, —C(═O)OH, —S(═O)₂OH, —S(═O)₂NH₂, —P(O)(OH)₂, —P(═O)(OH)(R^{d}), —P(═O)(OH)(OR^{d}),

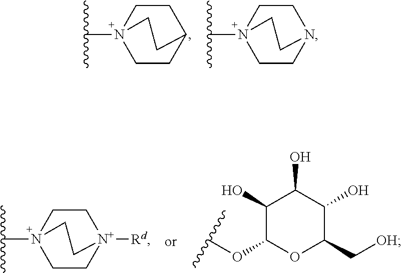

and
each R^{d} is independently C₁₋₆ alkyl.

In some embodiments,
X is —O—;
each R¹ is hydrogen;
each R² is hydrogen;
R¹¹ is hydrogen; and
R³, R⁴, R⁵, R⁶, R⁷, R⁸, R⁹, and R¹⁰ are each hydrogen;
Ring A is phenyl;
each R^{a} is independently halogen;
n is 1 or 2;
Y is

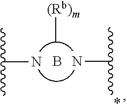

where Ring B is a monocyclic 4- to 8-membered heterocycloalkyl, 7- to 12-membered fused bicyclic heterocycloalkyl, 7- to 12-membered bridged bicyclic heterocycloalkyl, or 7- to 12-membered spirocyclic bicyclic heterocycloalkyl; wherein Ring B comprises 2 N atoms and 0 or 1 O or S atoms, and * represents the attachment point to K;
or Y is

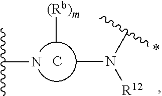

where Ring C is a 7- to 12-membered spirocyclic bicyclic heterocycloalkyl; wherein Ring C comprises 1 or 2 N atoms and 0 or 1 O or S atoms; R¹² is hydrogen or C₁₋₂ alkyl; and * represents the attachment point to K;
K is C₁₋₈ alkyl or —C(═O)—C₁₋₈ alkyl; wherein the alkyl is substituted by 1-6 R^{c} groups;
each R^{c} is independently —OH, —CH₂OH, —N(R^{d})₃⁺, —C(═O)OH, or —S(═O)₂OH; and
each R^{d} is independently C₁₋₆ alkyl.

In some embodiments, —Y—K is

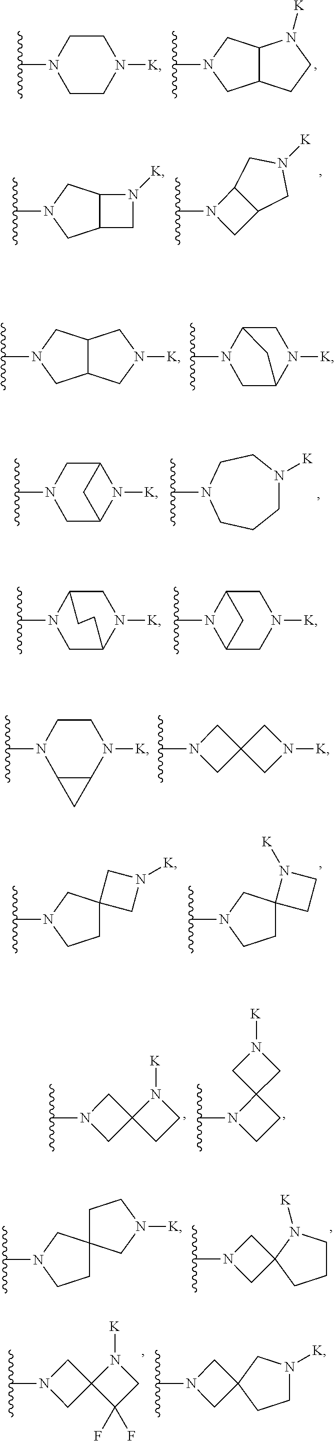

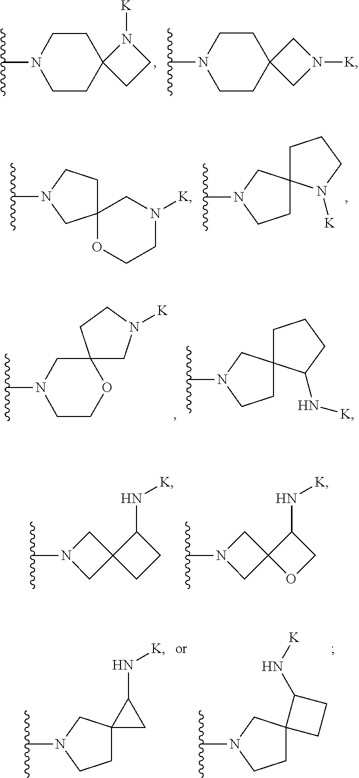

and K is C₄₋₆ alkyl or —C(═O)—C₄₋₆ alkyl; wherein the alkyl is substituted by 1-6 —OH or —S(═O)₂OH groups.

In some embodiments, disclosed herein is a compound of Formula (IV):

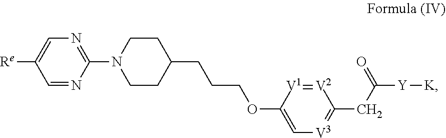

or a pharmaceutically acceptable salt, solvate, stereoisomer, or prodrug thereof, wherein V¹ is CH, CF, or N; V² is CH, CF, or N; V³ is CH, CF, or N; and the other substituents are as defined herein.

In some embodiments, V¹ is CH, CF, or N; V² is CH or CF; and V³ is CH, CF, or N.

In some embodiments, V¹ is CH or CF; V² is CH or CF; and V³ is CH, CF, or N.

In some embodiments, V¹ is CH or CF; V² is CF; and V³ is CH, CF, or N.

In some embodiments, disclosed herein is a compound of Formula (V):

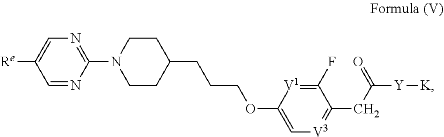

or a pharmaceutically acceptable salt, solvate, stereoisomer, or prodrug thereof, wherein V¹ is CH or CF; V³ is CH, CF, or N; and the other substituents are as defined herein.

In some embodiments, V¹ is CH. In some embodiments, V¹ is CF.

In some embodiments, V³ is CH or CF. In some embodiments, V³ is CH. In some embodiments, V³ is CF. In some embodiments, V³ is N.

In some embodiments,
Y is

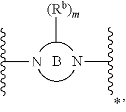

where Ring B is a monocyclic heterocycloalkyl or a bicyclic heterocycloalkyl; wherein Ring B comprises 2 N atoms and 0 or 1 O or S atoms, and * represents the attachment point to K;
or Y is

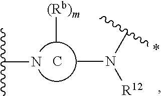

where Ring C is a fused bicyclic heterocycloalkyl, bridged bicyclic heterocycloalkyl, or spirocyclic bicyclic heterocycloalkyl; wherein Ring C comprises 1 or 2 N atoms and 0 or 1 O or S atoms; R¹² is hydrogen or C₁₋₄ alkyl; and * represents the attachment point to K;
each R^{b} is independently fluoro or C₁₋₄ alkyl;
m is 0, 1, or 2;
K is C₁₋₈ alkyl, —C(═O)—C₁₋₈alkyl, —[(CH₂)ₛ—Z]ₜ—R¹³, —[(CHR^{d})ₛ—Z]ₜ—R¹³, or —[(C(R^{d})₂)ₛ—Z]ₜ—R¹³; wherein the alkyl is substituted by 1-6 R^{c} group;
each Z is independently CH₂O—, —CH₂NH, CH₂NR^{d}, or —CH₂N⁺(R^{d})₂;
s is 1-3;
t is 1-3;
R¹³ is hydrogen or a C₁₋₈ alkyl that is unsubstituted or substituted by 1-6 R^{c} groups;
each R^{c} is independently —OH, —CH₂OH, —NH₂, —N(R^{d})₃⁺, —C(═O)OH, —S(═O)₂OH, —S(═O)₂NH₂, —P(═O)(OH)₂, —P(═O)(OH)(R^{d}), —P(═O)(OH)(OR^{d}),

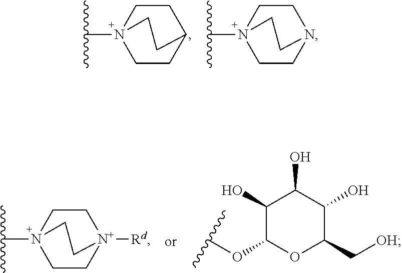

and
each R^{d} is independently C₁₋₆ alkyl.

In some embodiments,
Y is

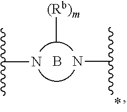

where Ring B is a monocyclic 4- to 8-membered heterocycloalkyl, 7- to 12-membered fused bicyclic heterocycloalkyl, 7- to 12-membered bridged bicyclic heterocycloalkyl, or 7- to 12-membered spirocyclic bicyclic heterocycloalkyl; wherein Ring B comprises 2 N atoms and 0 or 1 O or S atoms, and * represents the attachment point to K;
or Y is

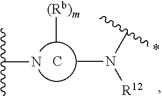

where Ring C is a 7- to 12-membered spirocyclic bicyclic heterocycloalkyl; wherein Ring C comprises 1 or 2 N atoms and 0 or 1 O or S atoms; R¹² is hydrogen or C₁₋₂ alkyl; and * represents the attachment point to K;
K is C₁₋₈ alkyl or —C(═O)—C₁₋₈ alkyl; wherein the alkyl is substituted by 1-6 R^{c} groups;
each R^{c} is independently —OH, —CH₂OH, —N(R^{d})₃⁺, —C(═O)OH, or —S(═O)₂OH; and
each R^{d} is independently C₁₋₆ alkyl.

In some embodiments, disclosed herein is a compound of Formula (IVa):

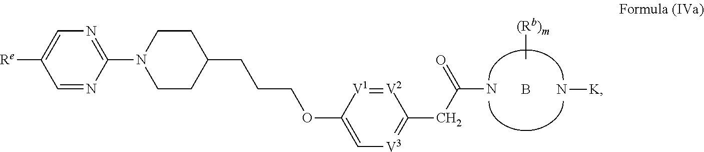

or a pharmaceutically acceptable salt, solvate, stereoisomer, or prodrug thereof, wherein V¹ is CH or CF; V³ is CH, CF, or N; and the other substituents are as defined herein.

In some embodiments, V¹ is CH, CF, or N; V² is CH or CF; and V³ is CH, CF, or N.

In some embodiments, V¹ is CH or CF; V² is CH or CF; and V³ is CH, CF, or N.

In some embodiments, V¹ is CH or CF; V² is CF; and V³ is CH, CF, or N.

In some embodiments, disclosed herein is a compound of Formula (Va):

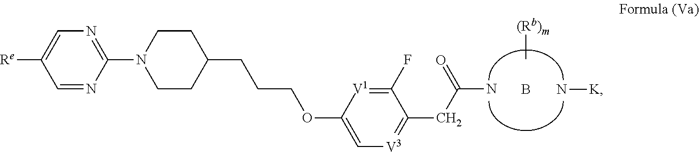

or a pharmaceutically acceptable salt, solvate, stereoisomer, or prodrug thereof, wherein V¹ is CH or CF; V³ is CH, CF, or N; and the other substituents are as defined herein.

In some embodiments,
Ring B is a monocyclic heterocycloalkyl or a bicyclic heterocycloalkyl; wherein Ring B comprises 2 N atoms and 0 or 1 O or S atoms, and * represents the attachment point to K;
each R^{b} is independently fluoro or C₁₋₄ alkyl;
m is 0, 1, or 2;
K is C₁₋₈ alkyl, —C(═O)—C₁₋₈ alkyl, —[(CH₂)ₛ—Z]ₜ—R¹³, —[(CHR^{d})ₛ—Z]ₜ—R¹³, or —[(C(R^{d})₂)ₛ—Z]ₜ—R¹³;
wherein the alkyl is substituted by 1-6 R^{c} group;
each Z is independently —CH₂O—, —CH₂NH—, —CH₂NR^{d}—, or —CH₂N⁺(R^{d})₂—;
s is 1-3;
t is 1-3;
R¹³ is hydrogen or a C₁₋₈ alkyl that is unsubstituted or substituted by 1-6 R^{c} groups;
each R^{c} is independently —OH, —CH₂OH, —NH₂, —N(R^{d})₃⁺, —C(═O)OH, —S(═O)₂OH, —S(═O)₂NH₂, —P(═O)(OH)₂, —P(═O)(OH)(R^{d}), —P(═O)(OH)(OR^{d}),

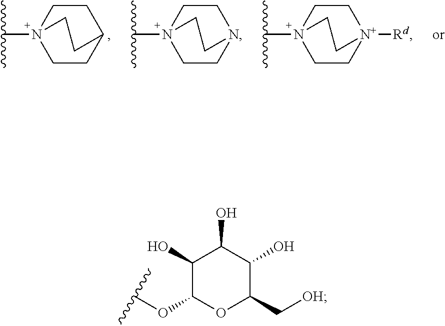

and
each R^{d} is independently C₁₋₆ alkyl.

In some embodiments,
Ring B is a monocyclic 4- to 8-membered heterocycloalkyl, 7- to 12-membered fused bicyclic heterocycloalkyl, 7- to 12-membered bridged bicyclic heterocycloalkyl, or 7- to 12-membered spirocyclic bicyclic heterocycloalkyl; wherein Ring B comprises 2 N atoms and 0 or 1 O or S atoms, and * represents the attachment point to K;
K is C₁₋₈ alkyl or —C(═O)—C₁₋₈ alkyl; wherein the alkyl is substituted by 1-6 R^{c} groups;
each R^{c} is independently —OH, —CH₂OH, —N(R^{d})₃⁺, —C(═O)OH, or —S(═O)₂OH; and
each R^{d} is independently C₁₋₆ alkyl.

In some embodiments, disclosed herein is a compound of Formula (IVb):

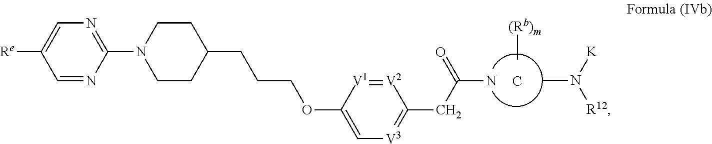

or a pharmaceutically acceptable salt, solvate, stereoisomer, or prodrug thereof, wherein V¹ is CH or CF; V³ is CH, CF, or N; and the other substituents are as defined herein.

In some embodiments, V¹ is CH or CF; V² is CH or CF; and V³ is CH, CF, or N.

In some embodiments, V¹ is CH or CF; V² is CF; and V³ is CH, CF, or N.

In some embodiments, disclosed herein is a compound of Formula (Vb):

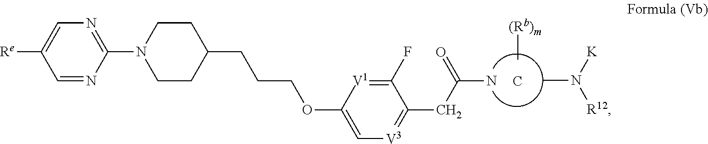

or a pharmaceutically acceptable salt, solvate, stereoisomer, or prodrug thereof, wherein V¹ is CH or CF; V³ is CH, CF, or N; and the other substituents are as defined herein.

In some embodiments,
Ring C is a fused bicyclic heterocycloalkyl, bridged bicyclic heterocycloalkyl, or spirocyclic bicyclic heterocycloalkyl; wherein Ring C comprises 1 or 2 N atoms and 0 or 1 O or S atoms; R¹² is hydrogen or C₁₋₄ alkyl; and * represents the attachment point to K;
each R^{b} is independently fluoro or C₁₋₄ alkyl;
m is 0, 1, or 2;
K is C₁₋₈ alkyl, —C(═O)—C₁₋₈ alkyl, —[(CH₂)ₛ—Z]ₜ—R¹³, —[(CHR^{d})ₛ—Z]ₜ—R¹³, or —[(C(R^{d})₂)ₛ—Z]ₜ—R¹; wherein the alkyl is substituted by 1-6 R^{c} group;
each Z is independently —CH₂O—, —CH₂NH—, —CH₂NR^{d}—, or —CH₂N⁺(R^{d})₂—;
s is 1-3;
t is 1-3;
R¹³ is hydrogen or a C₁₋₈ alkyl that is unsubstituted or substituted by 1-6 R^{c} groups;
each R^{c} is independently —OH, —CH₂OH, —NH₂, —N(R^{d})₃⁺, —C(═O)OH, —S(═O)₂₀H, —S(═O)₂NH₂, —P(O)(OH)₂, —P(═O)(OH)(R^{d}), —P(═O)(OH)(OR^{d}),

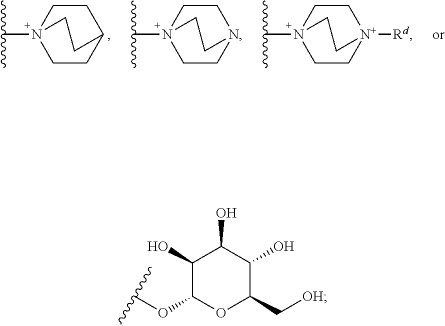

and
each R^{d} is independently C₁₋₆ alkyl.

In some embodiments,
Ring C is a 7- to 12-membered spirocyclic bicyclic heterocycloalkyl; wherein Ring C comprises 1 or 2 N atoms and 0 or 1 O or S atoms; R¹² is hydrogen or C₁₋₂ alkyl; and * represents the attachment point to K;
K is C₁₋₈ alkyl or —C(═O)—C₁₋₈ alkyl; wherein the alkyl is substituted by 1-6 R^{c} groups;
each R^{c} is independently —OH, —CH₂OH, —N(R^{d})₃⁺, —C(═O)OH, or —S(═O)₂OH; and
each R^{d} is independently C₁₋₆ alkyl.

In some embodiments, —Y—K is

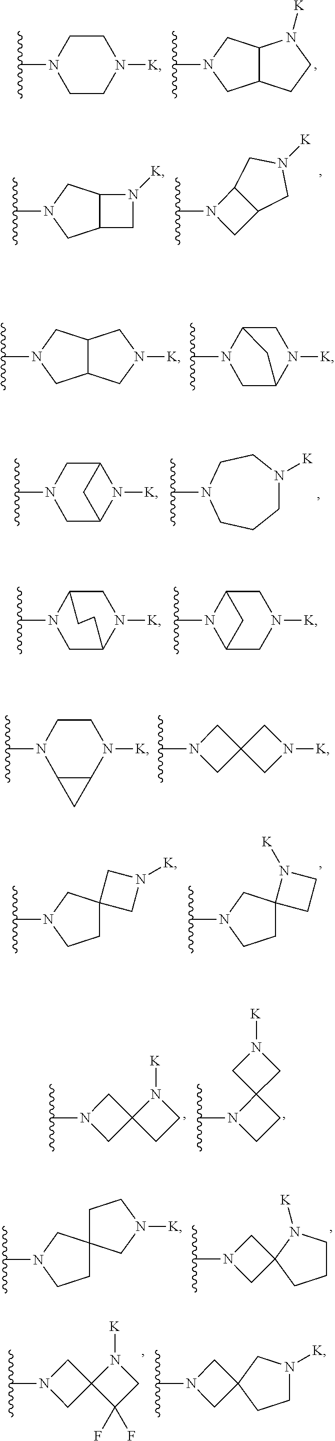

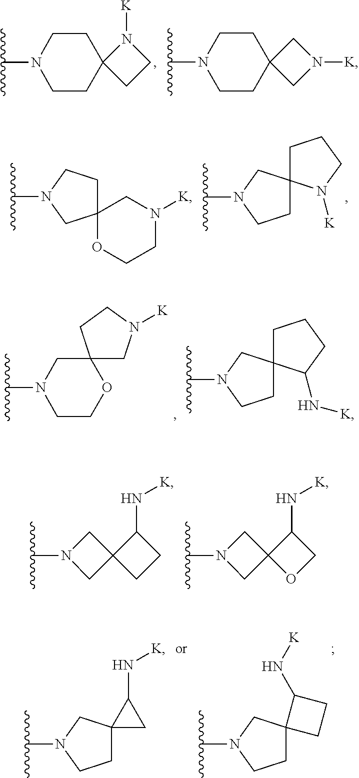

and K is C₄₋₆ alkyl or —C(═O)—C₄₋₆ alkyl; wherein the alkyl is substituted by 1-6 —OH or —S(═O)₂OH groups.

In some embodiments, K is

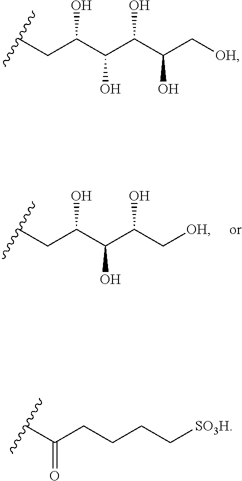 [SMILES: *C[C@H](O)[C@@H](O)[C@H](O)[C@H](O)CO ; *C[C@H](O)[C@H](O)[C@H](O)CO ; *C(=O)CCCCS(=O)(=O)O]

In some embodiments, the compound described herein has a structure provided in Table 1.

TABLE 1

| Ex. # | Structure | Name |
|---|---|---|
| 1 | 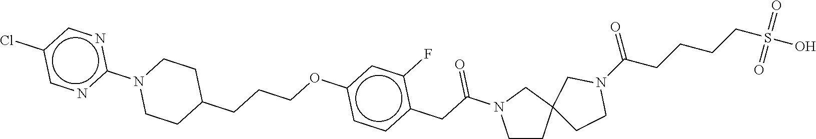 | 5-(7-(2-(4-(3-(1-(5-chloropyrimidin-2- yl)piperidin-4-yl)propoxy)-2- fluorophenyl)acetyl)-2,7-diazaspiro[4.4]nonan- 2-yl)-5-oxopentane-1-sulfonic acid |
| 2 | 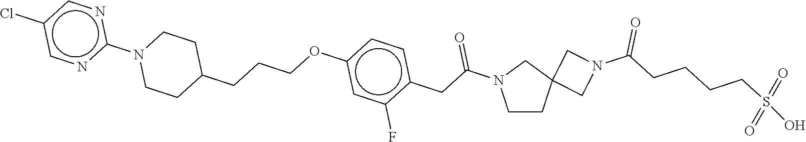 | 5-(6-(2-(4-(3-(1-(5-chloropyrimidin-2- yl)piperidin-4-yl)propoxy)-2- fluorophenyl)acetyl)-2,6-diazaspiro[3.4]octan-2- yl)-5-oxopentane-1-sulfonic acid |
| 3 | 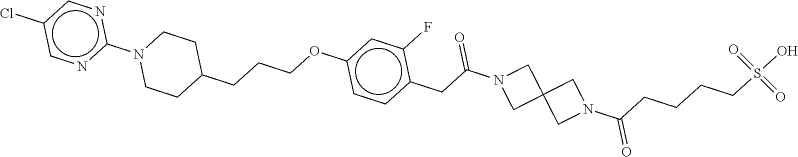 | 5-(6-(2-(4-(3-(1-(5-chloropyrimidin-2- yl)piperidin-4-yl)propoxy)-2- fluorophenyl)acetyl)-2,6-diazaspiro[3.3]heptan- 2-yl)-5-oxopentane-1-sulfonic acid |
| 4 | 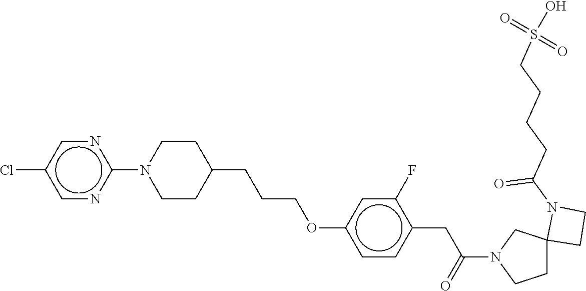 | 5-(6-(2-(4-(3-(1-(5-chloropyrimidin-2- yl)piperidin-4-yl)propoxy)-2- fluorophenyl)acetyl)-1,6-diazaspiro[3.4]octan-1- yl)-5-oxopentane-1-sulfonic acid |
| 5 | 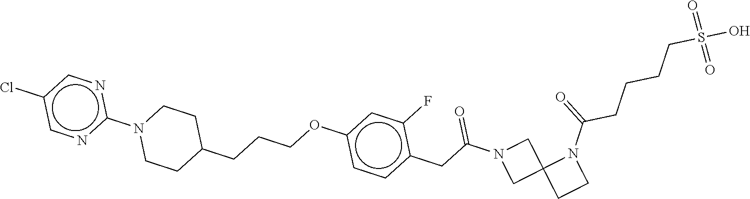 | 5-(6-(2-(4-(3-(1-(5-chloropyrimidin-2- yl)piperidin-4-yl)propoxy)-2- fluorophenyl)acetyl)-1,6-diazaspiro[3.3]heptan- 1-yl)-5-oxopentane-1-sulfonic acid |
| 6 | 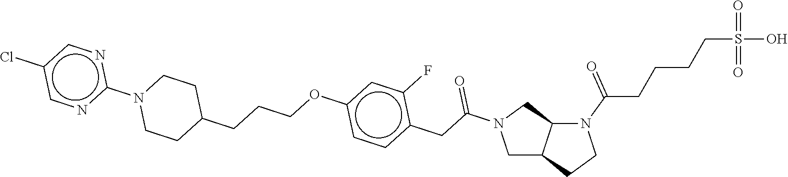 | 5-((3aS,6aS)-5-(2-(4-(3-(1-(5-chloropyrimidin-2- yl)piperidin-4-yl)propoxy)-2- fluorophenyl)acetyl)hexahydropyrrolo[3,4- b]pyrrol-1(2H)-yl)-5-oxopentane-1-sulfonic acid |
| 7 | 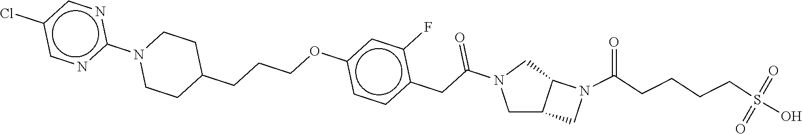 | 5-((1S,5R)-3-(2-(4-(3-(1-(5-chloropyrimidin-2- yl)piperidin-4-yl)propoxy)-2- fluorophenyl)acetyl)-3,6- diazabicyclo[3.2.0]heptan-6-yl)-5-oxopentane-1- sulfonic acid |
| 8 | 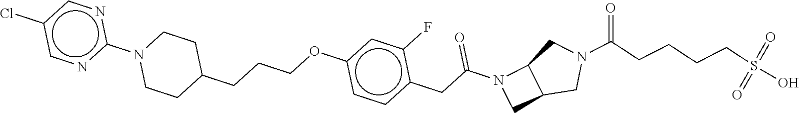 | 5-((1S,5R)-6-(2-(4-(3-(1-(5-chloropyrimidin-2- yl)piperidin-4-yl)propoxy)-2- fluorophenyl)acetyl)-3,6- diazabicyclo[3.2.0]heptan-3-yl)-5-oxopentane-1- sulfonic acid |
| 9 | 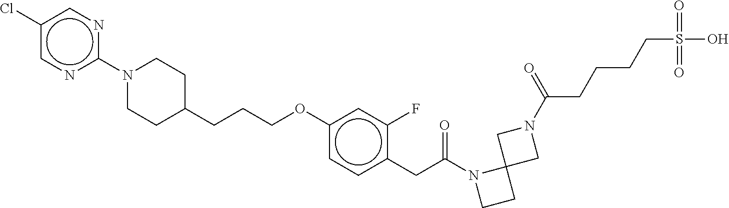 | 5-(1-(2-(4-(3-(1-(5-chloropyrimidin-2- yl)piperidin-4-yl)propoxy)-2- fluorophenyl)acetyl)-1,6-diazaspiro[3.3]heptan- 6-yl)-5-oxopentane-1-sulfonic acid |
| 10 | 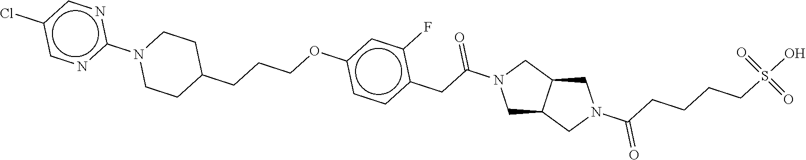 | 5-((3aR,6aS)-5-(2-(4-(3-(1-(5-chloropyrimidin- 2-yl)piperidin-4-yl)propoxy)-2- fluorophenyl)acetyl)hexahydropyrrolo[3,4- c]pyrrol-2(1H)-yl)-5-oxopentane-1-sulfonic acid |
| 11 | 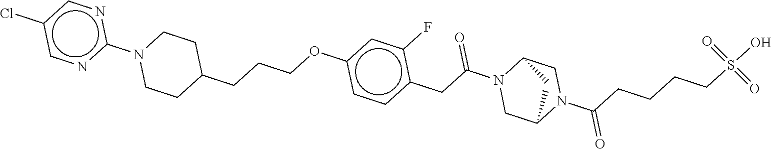 | 5-((1R,4R)-5-(2-(4-(3-(1-(5-chloropyrimidin-2- yl)piperidin-4-yl)propoxy)-2- fluorophenyl)acetyl)-2,5- diazabicyclo[2.2.1]heptan-2-yl)-5-oxopentane-1- sulfonic acid |
| 12 | 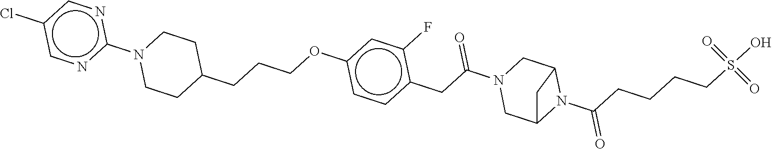 | 5-(3-(2-(4-(3-(1-(5-chloropyrimidin-2- yl)piperidin-4-yl)propoxy)-2- fluorophenyl)acetyl)-3,6- diazabicyclo[3.1.1]heptan-6-yl)-5-oxopentane-1- sulfonic acid |
| 13 | 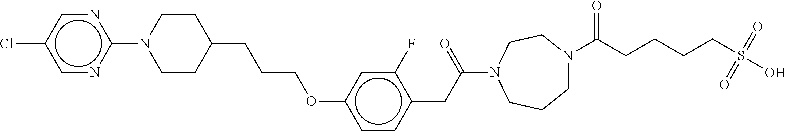 | 5-(4-(2-(4-(3-(1-(5-chloropyrimidin-2- yl)piperidin-4-yl)propoxy)-2- fluorophenyl)acetyl)-1,4-diazepan-1-yl)-5- oxopentane-1-sulfonic acid |
| 14 | 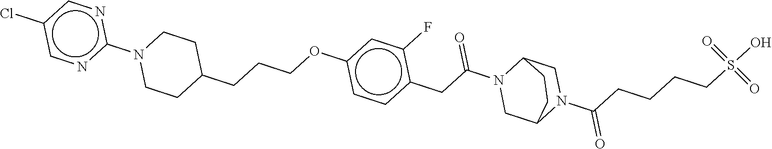 | 5-(5-(2-(4-(3-(1-(5-chloropyrimidin-2- yl)piperidin-4-yl)propoxy)-2- fluorophenyl)acetyl)-2,5- diazabicyclo[2.2.2]octan-2-yl)-5-oxopentane-1- sulfonic acid |
| 15 | 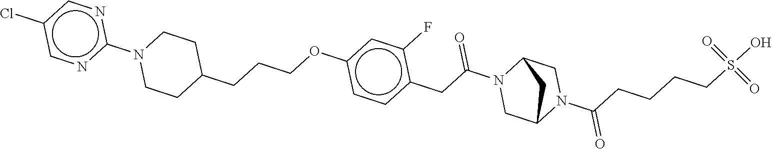 | 5-((1S,4S)-5-(2-(4-(3-(1-(5-chloropyrimidin-2- yl)piperidin-4-yl)propoxy)-2- fluorophenyl)acetyl)-2,5- diazabicyclo[2.2.1]heptan-2-yl)-5-oxopentane-1- sulfonic acid |
| 16 | 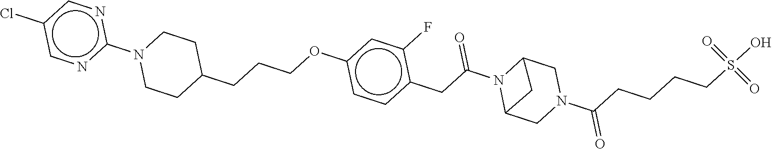 | 5-(6-(2-(4-(3-(1-(5-chloropyrimidin-2- yl)piperidin-4-yl)propoxy)-2- fluorophenyl)acetyl)-3,6- diazabicyclo[3.1.1]heptan-3-yl)-5-oxopentane-1- sulfonic acid |
| 17 | 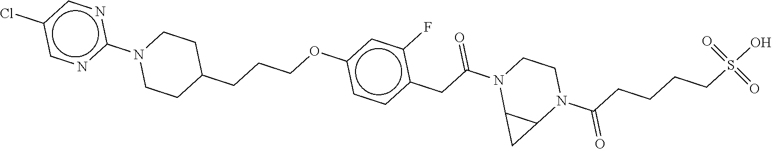 | 5-(5-(2-(4-(3-(1-(5-chloropyrimidin-2- yl)piperidin-4-yl)propoxy)-2- fluorophenyl)acetyl)-2,5- diazabicyclo[4.1.0]heptan-2-yl)-5-oxopentane-1- sulfonic acid |
| 18 | 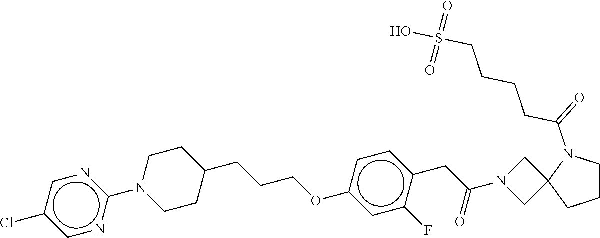 | 5-(2-(2-(4-(3-(1-(5-chloropyrimidin-2- yl)piperidin-4-yl)propoxy)-2- fluorophenyl)acetyl)-2,5-diazaspiro[3.4]octan-5- yl)-5-oxopentane-1-sulfonic acid |
| 19 | 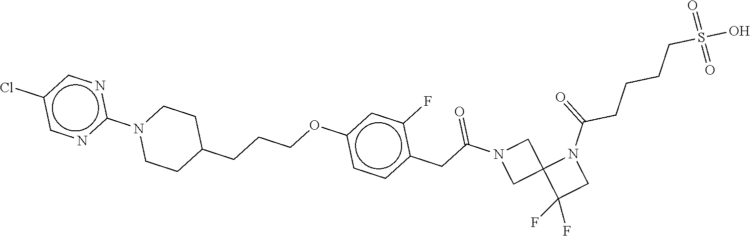 | 5-(6-(2-(4-(3-(1-(5-chloropyrimidin-2- yl)piperidin-4-yl)propoxy)-2- fluorophenyl)acetyl)-3,3-difluoro-1,6- diazaspiro[3.3]heptan-1-yl)-5-oxopentane-1- sulfonic acid |
| 20 | 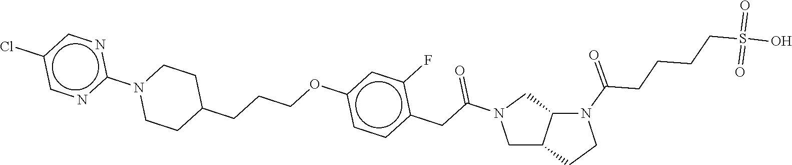 | 5-((3aR,6aR)-5-(2-(4-(3-(1-(5-chloropyrimidin- 2-yl)piperidin-4-yl)propoxy)-2- fluorophenyl)acetyl)hexahydropyrrolo[3,4- b]pyrrol-1(2H)-yl)-5-oxopentane-1-sulfonic acid |
| 21 | 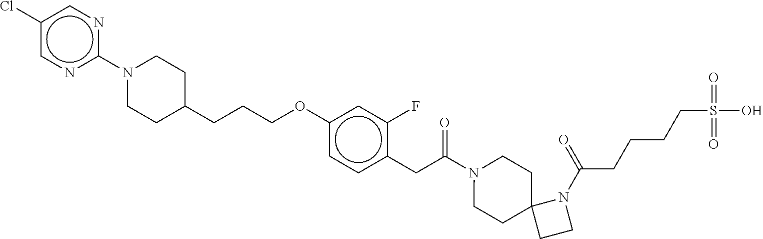 | 5-(7-(2-(4-(3-(1-(5-chloropyrimidin-2- yl)piperidin-4-yl)propoxy)-2- fluorophenyl)acetyl)-1,7-diazaspiro[3.5]nonan- 1-yl)-5-oxopentane-1-sulfonic acid |
| 22 | 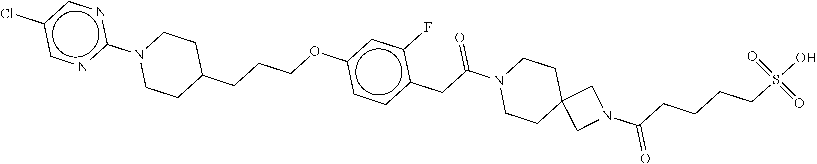 | 5-(7-(2-(4-(3-(1-(5-chloropyrimidin-2- yl)piperidin-4-yl)propoxy)-2- fluorophenyl)acetyl)-2,7-diazaspiro[3.5]nonan- 2-yl)-5-oxopentane-1-sulfonic acid |
| 23 | 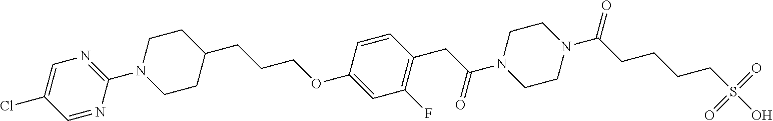 [SMILES: O=C(CCCCS(=O)(=O)O)N1CCN(C(=O)Cc2ccc(OCCCC3CCN(c4ncc(Cl)cn4)CC3)cc2F)CC1] | 5-(4-(2-(4-(3-(1-(5-chloropyrimidin-2- yl)piperidin-4-yl)propoxy)-2- fluorophenyl)acetyl)piperazin-1-yl)-5- oxopentane-1-sulfonic acid |
| 24 | 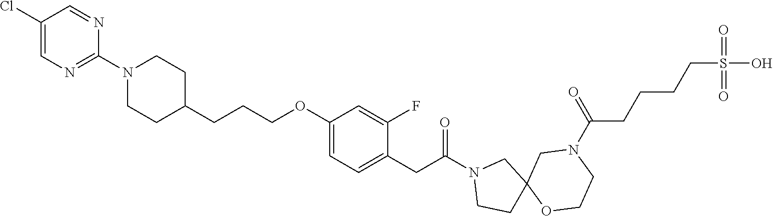 [SMILES: O=C(CCCCS(=O)(=O)O)N1CCOC2(CCN(C(=O)Cc3ccc(OCCCC4CCN(c5ncc(Cl)cn5)CC4)cc3F)C2)C1] | 5-(2-(2-(4-(3-(1-(5-chloropyrimidin-2- yl)piperidin-4-yl)propoxy)-2- fluorophenyl)acetyl)-6-oxa-2,9- diazaspiro[4.5]decan-9-yl)-5-oxopentane-1- sulfonic acid |
| 25 | 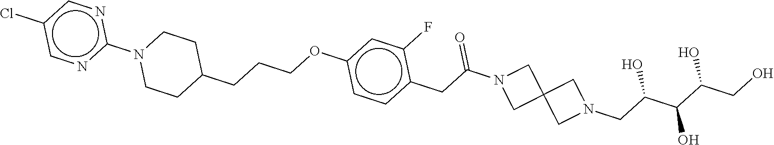 | 2-(4-(3-(1-(5-chloropyrimidin-2-yl)piperidin-4- yl)propoxy)-2-fluorophenyl)-1-(6-((2S,3S,4R)- 2,3,4,5-tetrahydroxypentyl)-2,6- diazaspiro[3.3]heptan-2-yl)ethan-1-one |
| 26 | 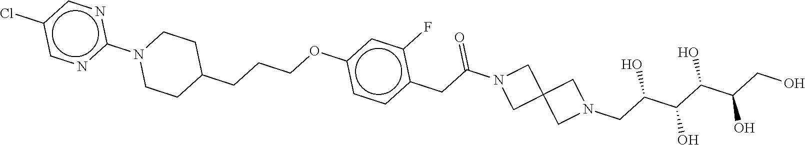 | 2-(4-(3-(1-(5-chloropyrimidin-2-yl)piperidin-4- yl)propoxy)-2-fluorophenyl)-1-(6- ((2S,3R,4R,5R)-2,3,4,5,6-pentahydroxyhexyl)- 2,6-diazaspiro[3.3]heptan-2-yl)ethan-1-one |
| 27 | 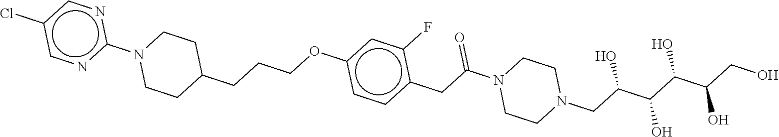 | 2-(4-(3-(1-(5-chloropyrimidin-2-yl)piperidin-4- yl)propoxy)-2-fluorophenyl)-1-(4- ((2S,3R,4R,5R)-2,3,4,5,6- pentahydroxyhexyl)piperazin-1-yl)ethan-1-one |
| 28 | 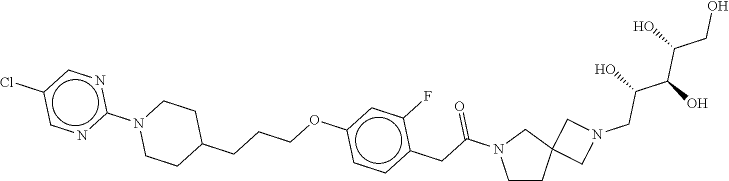 | 2-(4-(3-(1-(5-chloropyrimidin-2-yl)piperidin-4- yl)propoxy)-2-fluorophenyl)-1-(2-((2S,3S,4R)- 2,3,4,5-tetrahydroxypentyl)-2,6- diazaspiro[3.4]octan-6-yl)ethan-1-one |
| 29 | 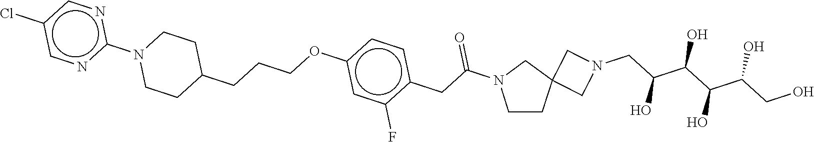 | 2-(4-(3-(1-(5-chloropyrimidin-2-yl)piperidin-4- yl)propoxy)-2-fluorophenyl)-1-(2- ((2S,3R,4R,5R)-2,3,4,5,6-pentahydroxyhexyl)- 2,6-diazaspiro[3.4]octan-6-yl)ethan-1-one |
| 30 | 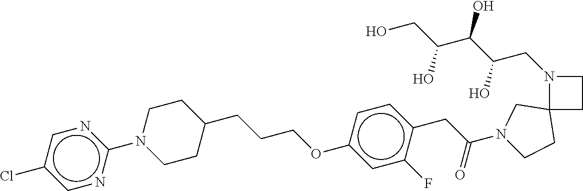 | 2-(4-(3-(1-(5-chloropyrimidin-2-yl)piperidin-4- yl)propoxy)-2-fluorophenyl)-1-(1-((2S,3S,4R)- 2,3,4,5-tetrahydroxypentyl)-1,6- diazaspiro[3.4]octan-6-yl)ethan-1-one |
| 31 | 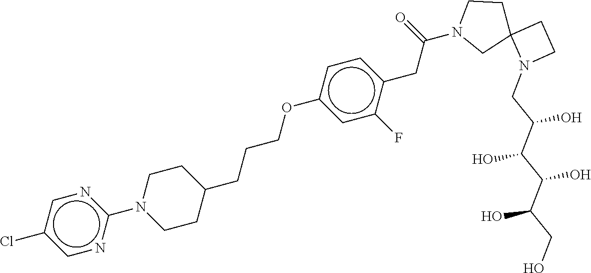 | 2-(4-(3-(1-(5-chloropyrimidin-2-yl)piperidin-4- yl)propoxy)-2-fluorophenyl)-1-(1- ((2S,3R,4R,5R)-2,3,4,5,6-pentahydroxyhexyl)- 1,6-diazaspiro[3.4]octan-6-yl)ethan-1-one |
| 32 | 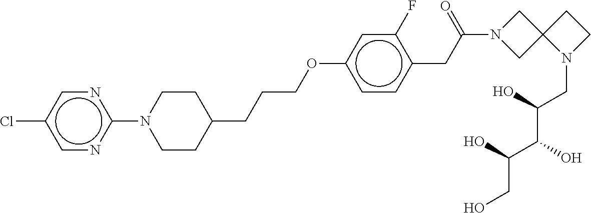 | 2-(4-(3-(1-(5-chloropyrimidin-2-yl)piperidin-4- yl)propoxy)-2-fluorophenyl)-1-(1-((2S,3S,4R)- 2,3,4,5-tetrahydroxypentyl)-1,6- diazaspiro[3.3]heptan-6-yl)ethan-1-one |
| 33 | 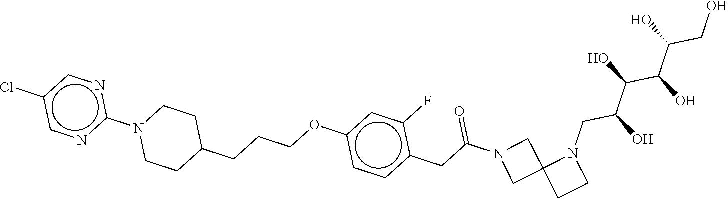 | 2-(4-(3-(1-(5-chloropyrimidin-2-yl)piperidin-4- yl)propoxy)-2-fluorophenyl)-1-(1- ((2S,3R,4R,5R)-2,3,4,5,6-pentahydroxyhexyl)- 1,6-diazaspiro[3.3]heptan-6-yl)ethan-1-one |
| 34 | 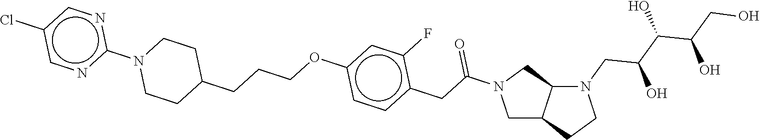 | 2-(4-(3-(1-(5-chloropyrimidin-2-yl)piperidin-4- yl)propoxy)-2-fluorophenyl)-1-((3aS,6aS)-1- ((2S,3S,4R)-2,3,4,5- tetrahydroxypentyl)hexahydropyrrolo[3,4- b]pyrrol-5(1H)-yl)ethan-1-one |
| 35 | 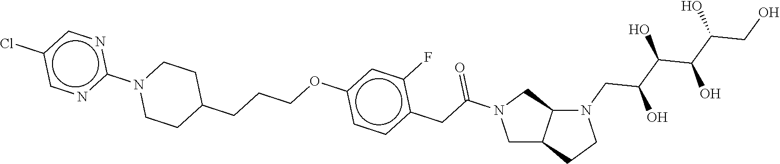 | 2-(4-(3-(1-(5-chloropyrimidin-2-yl)piperidin-4- yl)propoxy)-2-fluorophenyl)-1-((3aS,6aS)-1- ((2S,3R,4R,5R)-2,3,4,5,6- pentahydroxyhexyl)hexahydropyrrolo[3,4- b]pyrrol-5(1H)-yl)ethan-1-one |
| 36 | 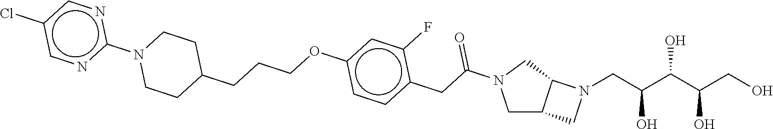 | 2-(4-(3-(1-(5-chloropyrimidin-2-yl)piperidin-4- yl)propoxy)-2-fluorophenyl)-1-((1R,5R)-6- ((2S,3S,4R)-2,3,4,5-tetrahydroxypentyl)-3,6- diazabicyclo[3.2.0]heptan-3-yl)ethan-1-one |
| 37 | 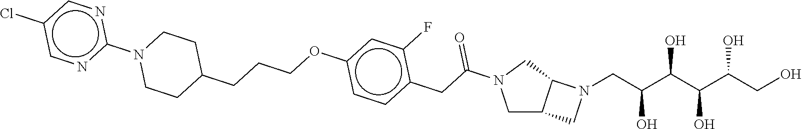 | 2-(4-(3-(1-(5-chloropyrimidin-2-yl)piperidin-4- yl)propoxy)-2-fluorophenyl)-1-((1R,5R)-6- ((2S,3R,4R,5R)-2,3,4,5,6-pentahydroxyhexyl)- 3,6-diazabicyclo[3.2.0]heptan-3-yl)ethan-1-one |
| 38 | 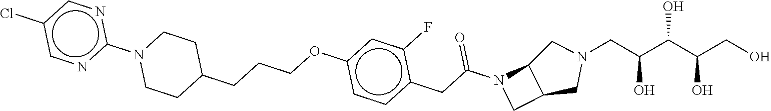 | 2-(4-(3-(1-(5-chloropyrimidin-2-yl)piperidin-4- yl)propoxy)-2-fluorophenyl)-1-((1S,5R)-3- ((2S,3S,4R)-2,3,4,5-tetrahydroxypentyl)-3,6- diazabicyclo[3.2.0]heptan-6-yl)ethan-1-one |
| 39 | 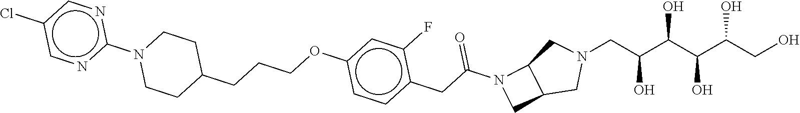 | 2-(4-(3-(1-(5-chloropyrimidin-2-yl)piperidin-4- yl)propoxy)-2-fluorophenyl)-1-((1S,5R)-3- ((2S,3R,4R,5R)-2,3,4,5,6-pentahydroxyhexyl)- 3,6-diazabicyclo[3.2.0]heptan-6-yl)ethan-1-one |
| 40 | 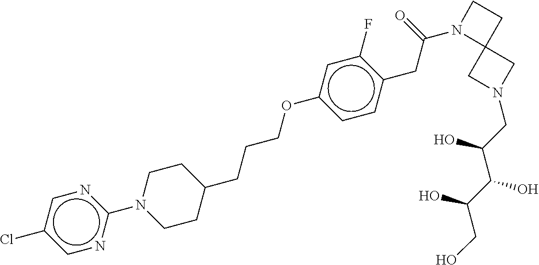 | 2-(4-(3-(1-(5-chloropyrimidin-2-yl)piperidin-4- yl)propoxy)-2-fluorophenyl)-1-(6-((2S,3S,4R)- 2,3,4,5-tetrahydroxypentyl)-1,6- diazaspiro[3.3]heptan-1-yl)ethan-1-one |
| 41 | 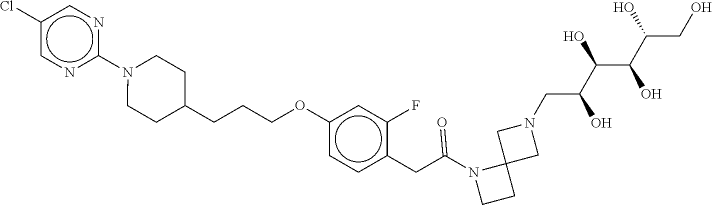 | 2-(4-(3-(1-(5-chloropyrimidin-2-yl)piperidin-4- yl)propoxy)-2-fluorophenyl)-1-(6- ((2S,3R,4R,5R)-2,3,4,5,6-pentahydroxyhexyl)- 1,6-diazaspiro[3.3]heptan-1-yl)ethan-1-one |
| 42 | 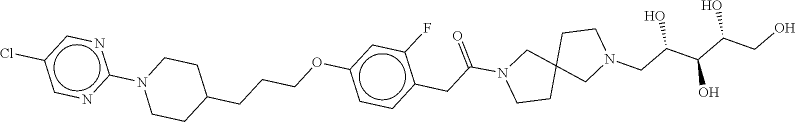 | 2-(4-(3-(1-(5-chloropyrimidin-2-yl)piperidin-4- yl)propoxy)-2-fluorophenyl)-1-(7-((2S,3S,4R)- 2,3,4,5-tetrahydroxypentyl)-2,7- diazaspiro[4.4]nonan-2-yl)ethan-1-one |
| 43 | 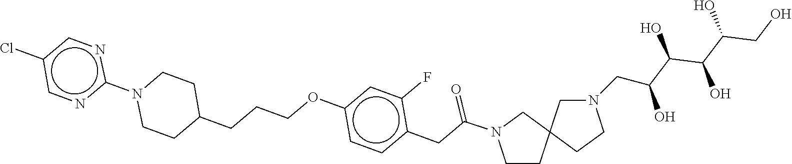 | 2-(4-(3-(1-(5-chloropyrimidin-2-yl)piperidin-4- yl)propoxy)-2-fluorophenyl)-1-(7- ((2S,3R,4R,5R)-2,3,4,5,6-pentahydroxyhexyl)- 2,7-diazaspiro[4.4]nonan-2-yl)ethan-1-one |
| 44 | 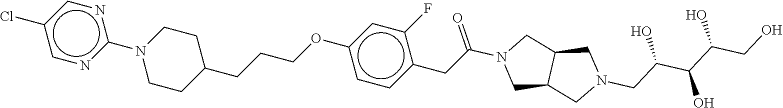 | 2-(4-(3-(1-(5-chloropyrimidin-2-yl)piperidin-4- yl)propoxy)-2-fluorophenyl)-1-((3aR,6aS)-5- ((2S,3S,4R)-2,3,4,5- tetrahydroxypentyl)hexahydropyrrolo[3,4- c]pyrrol-2(1H)-yl)ethan-1-one |
| 45 | 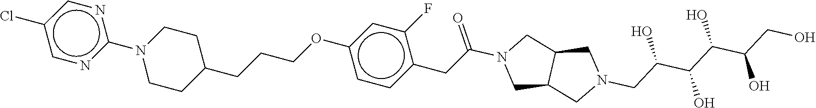 | 2-(4-(3-(1-(5-chloropyrimidin-2-yl)piperidin-4- yl)propoxy)-2-fluorophenyl)-1-((3aR,6aS)-5- ((2S,3R,4R,5R)-2,3,4,5,6- pentahydroxyhexyl)hexahydropyrrolo[3,4- c]pyrrol-2(1H)-yl)ethan-1-one |
| 46 | 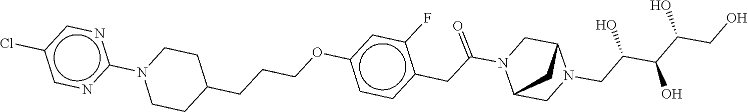 | 2-(4-(3-(1-(5-chloropyrimidin-2-yl)piperidin-4- yl)propoxy)-2-fluorophenyl)-1-((1R,4R)-5- ((2S,3S,4R)-2,3,4,5-tetrahydroxypentyl)-2,5- diazabicyclo[2.2.1]heptan-2-yl)ethan-1-one |
| 47 | 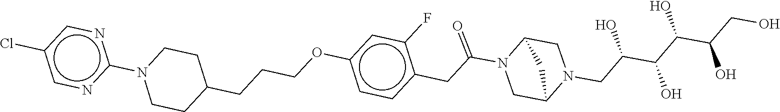 | 2-(4-(3-(1-(5-chloropyrimidin-2-yl)piperidin-4- yl)propoxy)-2-fluorophenyl)-1-((1R,4R)-5- ((2S,3R,4R,5R)-2,3,4,5,6-pentahydroxyhexyl)- 2,5-diazabicyclo[2.2.1]heptan-2-yl)ethan-1-one |
| 48 | 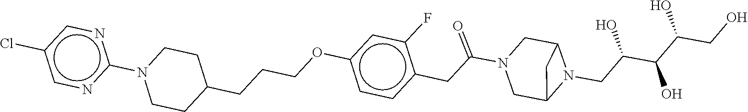 | 2-(4-(3-(1-(5-chloropyrimidin-2-yl)piperidin-4- yl)propoxy)-2-fluorophenyl)-1-(6-((2S,3S,4R)- 2,3,4,5-tetrahydroxypentyl)-3,6- diazabicyclo[3.1.1]heptan-3-yl)ethan-1-one |
| 49 | 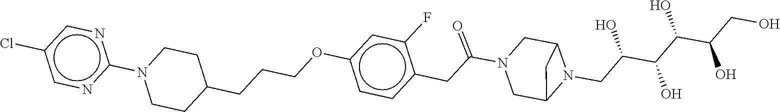 | 2-(4-(3-(1-(5-chloropyrimidin-2-yl)piperidin-4- yl)propoxy)-2-fluorophenyl)-1-(6- ((2S,3R,4R,5R)-2,3,4,5,6-pentahydroxyhexyl)- 3,6-diazabicyclo[3.1.1]heptan-3-yl)ethan-1-one |
| 50 | 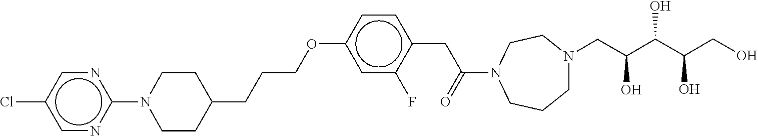 | 2-(4-(3-(1-(5-chloropyrimidin-2-yl)piperidin-4- yl)propoxy)-2-fluorophenyl)-1-(4-((2S,3S,4R)- 2,3,4,5-tetrahydroxypentyl)-1,4-diazepan-1- yl)ethan-1-one |
| 51 | 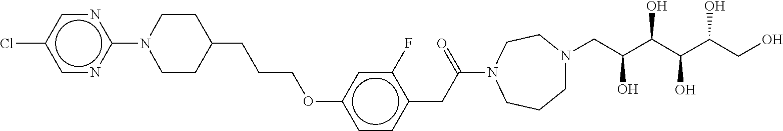 | 2-(4-(3-(1-(5-chloropyrimidin-2-yl)piperidin-4- yl)propoxy)-2-fluorophenyl)-1-(4- ((2S,3R,4R,5R)-2,3,4,5,6-pentahydroxyhexyl)- 1,4-diazepan-1-yl)ethan-1-one |
| 52 | 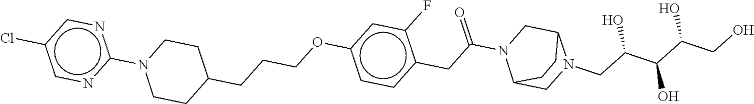 | 2-(4-(3-(1-(5-chloropyrimidin-2-yl)piperidin-4- yl)propoxy)-2-fluorophenyl)-1-(5-((2S,3S,4R)- 2,3,4,5-tetrahydroxypentyl)-2,5- diazabicyclo[2.2.2]octan-2-yl)ethan-1-one |
| 53 | 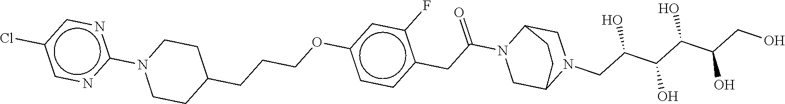 | 2-(4-(3-(1-(5-chloropyrimidin-2-yl)piperidin-4- yl)propoxy)-2-fluorophenyl)-1-(5- ((2S,3R,4R,5R)-2,3,4,5,6-pentahydroxyhexyl)- 2,5-diazabicyclo[2.2.2]octan-2-yl)ethan-1-one |
| 54 | 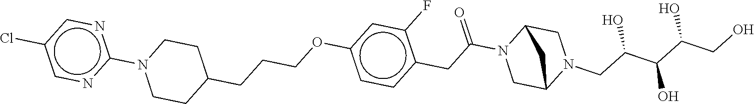 | 2-(4-(3-(1-(5-chloropyrimidin-2-yl)piperidin-4- yl)propoxy)-2-fluorophenyl)-1-((1S,4S)-5- ((2S,3S,4R)-2,3,4,5-tetrahydroxypentyl)-2,5- diazabicyclo[2.2.1]heptan-2-yl)ethan-1-one |
| 55 | 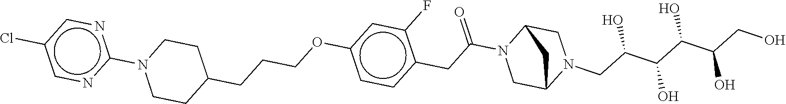 | 2-(4-(3-(1-(5-chloropyrimidin-2-yl)piperidin-4- yl)propoxy)-2-fluorophenyl)-1-((1S,4S)-5- ((2S,3R,4R,5R)-2,3,4,5,6-pentahydroxyhexyl)- 2,5-diazabicyclo[2.2.1]heptan-2-yl)ethan-1-one |
| 56 | 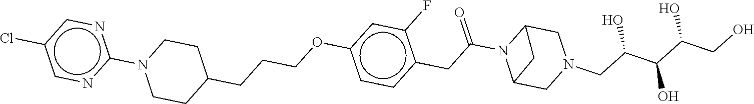 | 2-(4-(3-(1-(5-chloropyrimidin-2-yl)piperidin-4- yl)propoxy)-2-fluorophenyl)-1-(3-((2S,3S,4R)- 2,3,4,5-tetrahydroxypentyl)-3,6- diazabicyclo[3.1.1]heptan-6-yl)ethan-1-one |
| 57 | 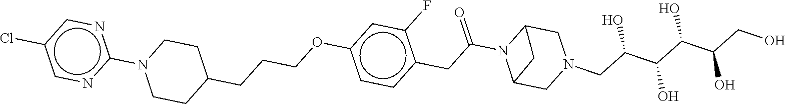 | 2-(4-(3-(1-(5-chloropyrimidin-2-yl)piperidin-4- yl)propoxy)-2-fluorophenyl)-1-(3- ((2S,3R,4R,5R)-2,3,4,5,6-pentahydroxyhexyl)- 3,6-diazabicyclo[3.1.1]heptan-6-yl)ethan-1-one |
| 58 | 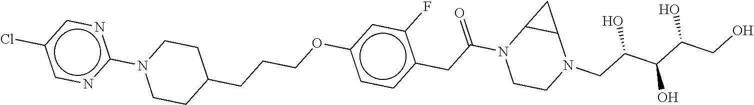 | 2-(4-(3-(1-(5-chloropyrimidin-2-yl)piperidin-4- yl)propoxy)-2-fluorophenyl)-1-(5-((2S,3S,4R)- 2,3,4,5-tetrahydroxypentyl)-2,5- diazabicyclo[4.1.0]heptan-2-yl)ethan-1-one |
| 59 | 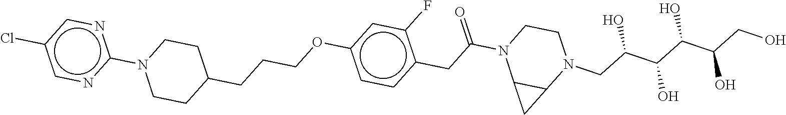 | 2-(4-(3-(1-(5-chloropyrimidin-2-yl)piperidin-4- yl)propoxy)-2-fluorophenyl)-1-(5- ((2S,3R,4R,5R)-2,3,4,5,6-pentahydroxyhexyl)- 2,5-diazabicyclo[4.1.0]heptan-2-yl)ethan-1-one |
| 60 | 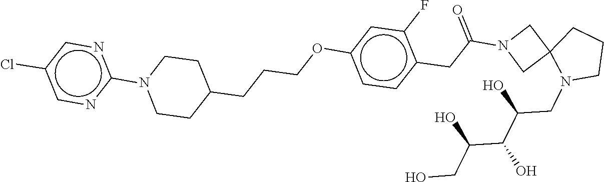 | 2-(4-(3-(1-(5-chloropyrimidin-2-yl)piperidin-4- yl)propoxy)-2-fluorophenyl)-1-(5-((2S,3S,4R)- 2,3,4,5-tetrahydroxypentyl)-2,5- diazaspiro[3.4]octan-2-yl)ethan-1-one |
| 61 | 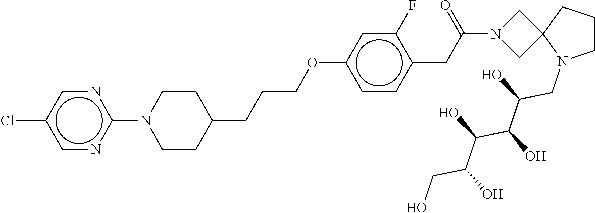 | 2-(4-(3-(1-(5-chloropyrimidin-2-yl)piperidin-4- yl)propoxy)-2-fluorophenyl)-1-(5- ((2S,3R,4R,5R)-2,3,4,5,6-pentahydroxyhexyl)- 2,5-diazaspiro[3.4]octan-2-yl)ethan-1-one |
| 62 | 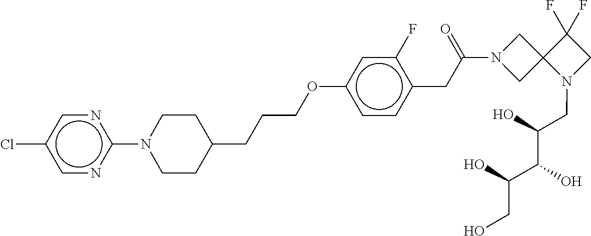 | 2-(4-(3-(1-(5-chloropyrimidin-2-yl)piperidin-4- yl)propoxy)-2-fluorophenyl)-1-(3,3-difluoro-1- ((2S,3S,4R)-2,3,4,5-tetrahydroxypentyl)-1,6- diazaspiro[3.3]heptan-6-yl)ethan-1-one |
| 63 | 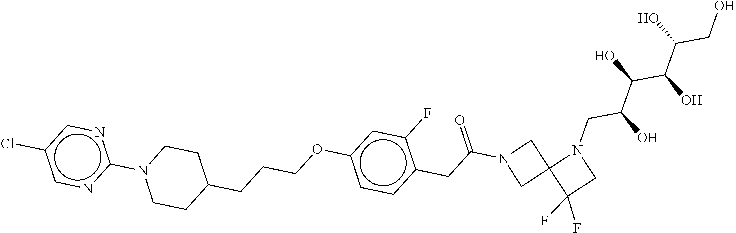 | 2-(4-(3-(1-(5-chloropyrimidin-2-yl)piperidin-4- yl)propoxy)-2-fluorophenyl)-1-(3,3-difluoro-1- ((2S,3R,4R,5R)-2,3,4,5,6-pentahydroxyhexyl)- 1,6-diazaspiro[3.3]heptan-6-yl)ethan-1-one |
| 64 | 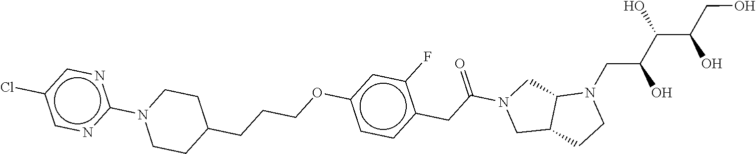 | 2-(4-(3-(1-(5-chloropyrimidin-2-yl)piperidin-4- yl)propoxy)-2-fluorophenyl)-1-((3aR,6aR)-1- ((2S,3S,4R)-2,3,4,5- tetrahydroxypentyl)hexahydropyrrolo[3,4- b]pyrrol-5(1H)-yl)ethan-1-one |
| 65 | 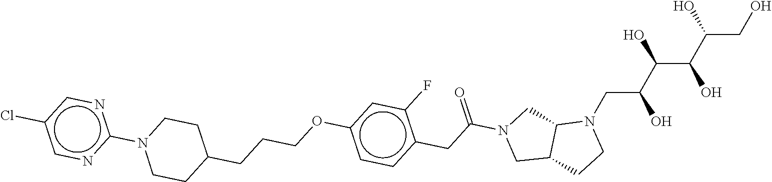 | 2-(4-(3-(1-(5-chloropyrimidin-2-yl)piperidin-4- yl)propoxy)-2-fluorophenyl)-1-((3aR,6aR)-1- ((2S,3R,4R,5R)-2,3,4,5,6- pentahydroxyhexyl)hexahydropyrrolo[3,4- b]pyrrol-5(1H)-yl)ethan-1-one |
| 66 | 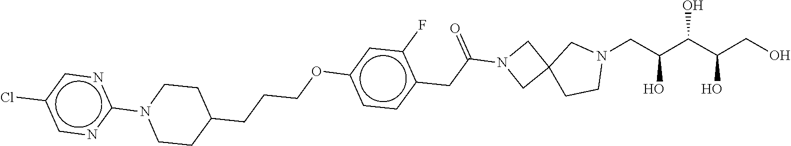 | 2-(4-(3-(1-(5-chloropyrimidin-2-yl)piperidin-4- yl)propoxy)-2-fluorophenyl)-1-(6-((2S,3S,4R)- 2,3,4,5-tetrahydroxypentyl)-2,6- diazaspiro[3.4]octan-2-yl)ethan-1-one |
| 67 | 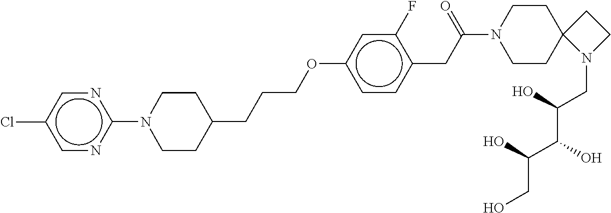 | 2-(4-(3-(1-(5-chloropyrimidin-2-yl)piperidin-4- yl)propoxy)-2-fluorophenyl)-1-(1-((2S,3S,4R)- 2,3,4,5-tetrahydroxypentyl)-1,7- diazaspiro[3.5]nonan-7-yl)ethan-1-one |
| 68 | 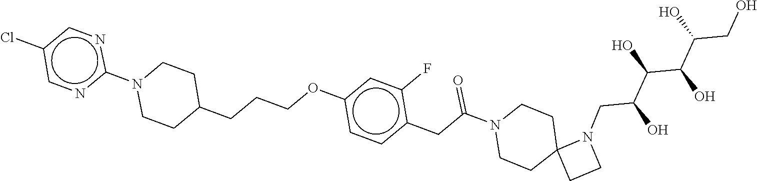 | 2-(4-(3-(1-(5-chloropyrimidin-2-yl)piperidin-4- yl)propoxy)-2-fluorophenyl)-1-(1- ((2S,3R,4R,5R)-2,3,4,5,6-pentahydroxyhexyl)- 1,7-diazaspiro[3.5]nonan-7-yl)ethan-1-one |
| 69 | 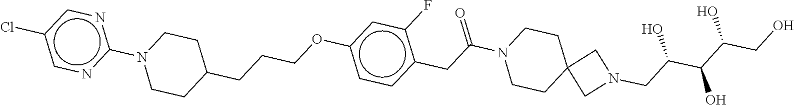 | 2-(4-(3-(1-(5-chloropyrimidin-2-yl)piperidin-4- yl)propoxy)-2-fluorophenyl)-1-(2-((2S,3S,4R)- 2,3,4,5-tetrahydroxypentyl)-2,7- diazaspiro[3.5]nonan-7-yl)ethan-1-one |
| 70 | 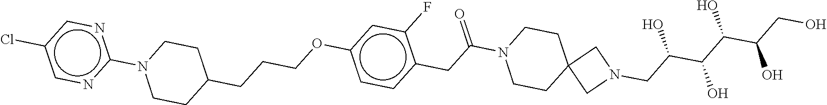 | 2-(4-(3-(1-(5-chloropyrimidin-2-yl)piperidin-4- yl)propoxy)-2-fluorophenyl)-1-(2- ((2S,3R,4R,5R)-2,3,4,5,6-pentahydroxyhexyl)- 2,7-diazaspiro[3.5]nonan-7-yl)ethan-1-one |
| 71 | 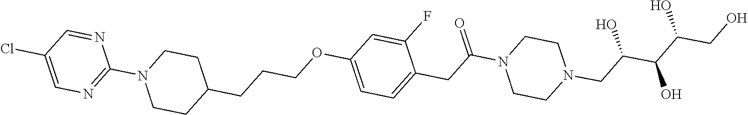 [SMILES: O=C(Cc1ccc(OCCCC2CCN(c3ncc(Cl)cn3)CC2)cc1F)N1CCN(C[C@H](O)[C@H](O)[C@H](O)CO)CC1] | 2-(4-(3-(1-(5-chloropyrimidin-2-yl)piperidin-4- yl)propoxy)-2-fluorophenyl)-1-(4-((2S,3S,4R)- 2,3,4,5-tetrahydroxypentyl)piperazin-1-yl)ethan- 1-one |
| 72 | 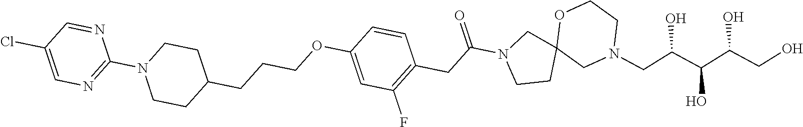 [SMILES: O=C(Cc1ccc(OCCCC2CCN(c3ncc(Cl)cn3)CC2)cc1F)N1CCC2(CN(C[C@H](O)[C@H](O)[C@H](O)CO)CCO2)C1] | 2-(4-(3-(1-(5-chloropyrimidin-2-yl)piperidin-4- yl)propoxy)-2-fluorophenyl)-1-(9-((2S,3S,4R)- 2,3,4,5-tetrahydroxypentyl)-6-oxa-2,9- diazaspiro[4.5]decan-2-yl)ethan-1-one |
| 73 | 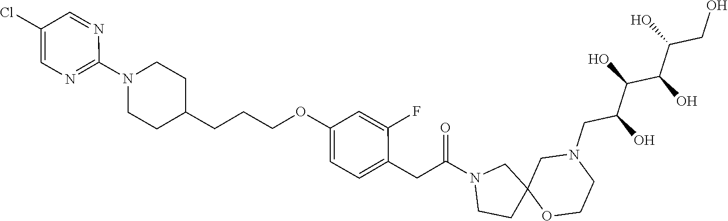 [SMILES: O=C(Cc1ccc(OCCCC2CCN(c3ncc(Cl)cn3)CC2)cc1F)N1CCC2(CN(C[C@H](O)[C@@H](O)[C@H](O)[C@H](O)CO)CCO2)C1] | 2-(4-(3-(1-(5-chloropyrimidin-2-yl)piperidin-4- yl)propoxy)-2-fluorophenyl)-1-(9- ((2S,3R,4R,5R)-2,3,4,5,6-pentahydroxyhexyl)-6- oxa-2,9-diazaspiro[4.5]decan-2-yl)ethan-1-one |
| 74 | 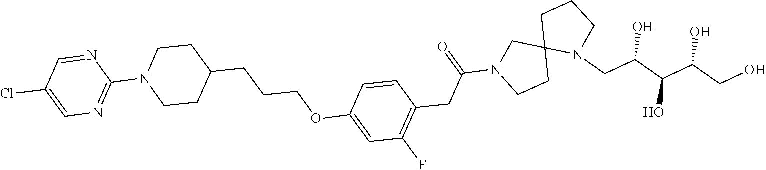 [SMILES: O=C(Cc1ccc(OCCCC2CCN(c3ncc(Cl)cn3)CC2)cc1F)N1CCC2(CCCN2C[C@H](O)[C@H](O)[C@H](O)CO)C1] | 2-(4-(3-(1-(5-chloropyrimidin-2-yl)piperidin-4- yl)propoxy)-2-fluorophenyl)-1-(1-((2S,3S,4R)- 2,3,4,5-tetrahydroxypentyl)-1,7- diazaspiro[4.4]nonan-7-yl)ethan-1-one |
| 75 | 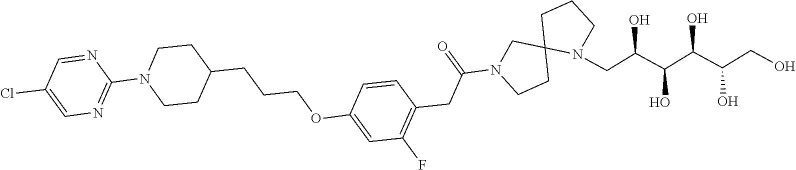 [SMILES: O=C(Cc1ccc(OCCCC2CCN(c3ncc(Cl)cn3)CC2)cc1F)N1CCC2(CCCN2C[C@@H](O)[C@H](O)[C@@H](O)[C@@H](O)CO)C1] | 2-(4-(3-(1-(5-chloropyrimidin-2-yl)piperidin-4- yl)propoxy)-2-fluorophenyl)-1-(1- ((2S,3R,4R,5R)-2,3,4,5,6-pentahydroxyhexyl)- 1,7-diazaspiro[4.4]nonan-7-yl)ethan-1-one |
| 76 | 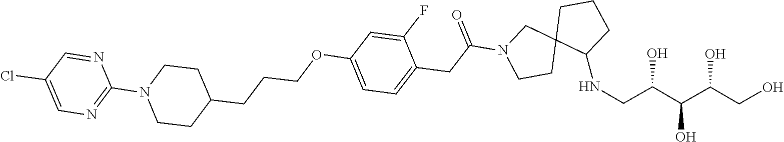 [SMILES: O=C(Cc1ccc(OCCCC2CCN(c3ncc(Cl)cn3)CC2)cc1F)N1CCC2(CCCC2NC[C@H](O)[C@H](O)[C@H](O)CO)C1] | 2-(4-(3-(1-(5-chloropyrimidin-2-yl)piperidin-4- yl)propoxy)-2-fluorophenyl)-1-(6-(((2S,3S,4R)- 2,3,4,5-tetrahydroxypentyl)amino)-2- azaspiro[4.4]nonan-2-yl)ethan-1-one |
| 77 | 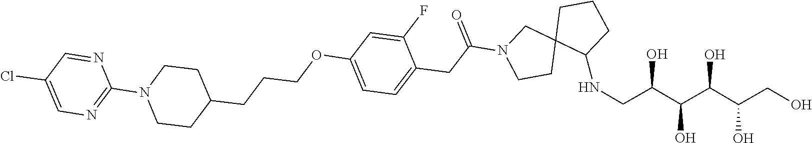 [SMILES: O=C(Cc1ccc(OCCCC2CCN(c3ncc(Cl)cn3)CC2)cc1F)N1CCC2(CCCC2NC[C@@H](O)[C@H](O)[C@@H](O)[C@@H](O)CO)C1] | 2-(4-(3-(1-(5-chloropyrimidin-2-yl)piperidin-4- yl)propoxy)-2-fluorophenyl)-1-(6- (((2S,3R,4R,5R)-2,3,4,5,6- pentahydroxyhexyl)amino)-2- azaspiro[4.4]nonan-2-yl)ethan-1-one |
| 78 | 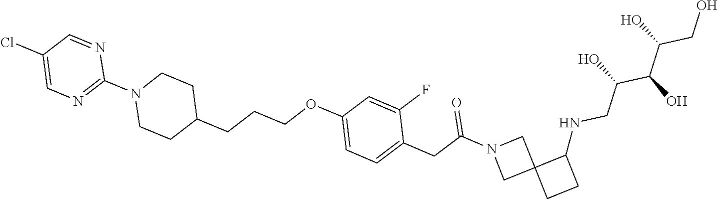 [SMILES: O=C(Cc1ccc(OCCCC2CCN(c3ncc(Cl)cn3)CC2)cc1F)N1CC2(CCC2NC[C@H](O)[C@H](O)[C@H](O)CO)C1] | 2-(4-(3-(1-(5-chloropyrimidin-2-yl)piperidin-4- yl)propoxy)-2-fluorophenyl)-1-(5-(((2S,3S,4R)- 2,3,4,5-tetrahydroxypentyl)amino)-2- azaspiro[3.3]heptan-2-yl)ethan-1-one |
| 79 | 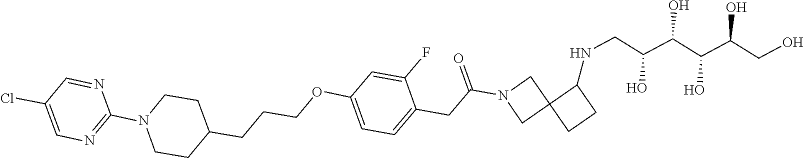 [SMILES: O=C(Cc1ccc(OCCCC2CCN(c3ncc(Cl)cn3)CC2)cc1F)N1CC2(CCC2NC[C@@H](O)[C@H](O)[C@@H](O)[C@@H](O)CO)C1] | 2-(4-(3-(1-(5-chloropyrimidin-2-yl)piperidin-4- yl)propoxy)-2-fluorophenyl)-1-(5- (((2S,3R,4R,5R)-2,3,4,5,6- pentahydroxyhexyl)amino)-2- azaspiro[3.3]heptan-2-yl)ethan-1-one |
| 80 | 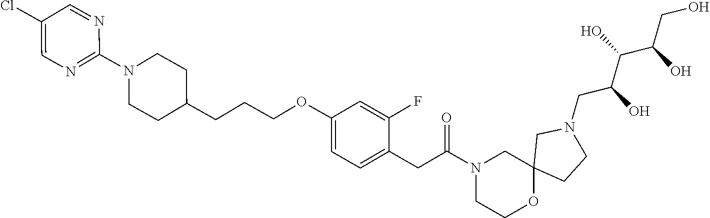 [SMILES: O=C(Cc1ccc(OCCCC2CCN(c3ncc(Cl)cn3)CC2)cc1F)N1CCOC2(CCN(C[C@H](O)[C@H](O)[C@H](O)CO)C2)C1] | 2-(4-(3-(1-(5-chloropyrimidin-2-yl)piperidin-4- yl)propoxy)-2-fluorophenyl)-1-(2-((2S,3S,4R)- 2,3,4,5-tetrahydroxypentyl)-6-oxa-2,9- diazaspiro[4.5]decan-9-yl)ethan-1-one |
| 81 | 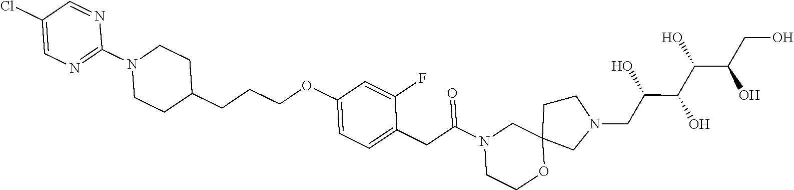 [SMILES: O=C(Cc1ccc(OCCCC2CCN(c3ncc(Cl)cn3)CC2)cc1F)N1CCOC2(CCN(C[C@H](O)[C@@H](O)[C@H](O)[C@H](O)CO)C2)C1] | 2-(4-(3-(1-(5-chloropyrimidin-2-yl)piperidin-4- yl)propoxy)-2-fluorophenyl)-1-(2- ((2S,3R,4R,5R)-2,3,4,5,6-pentahydroxyhexyl)-6- oxa-2,9-diazaspiro[4.5]decan-9-yl)ethan-1-one |
| 82 | 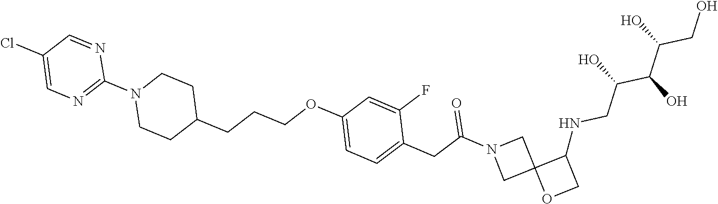 [SMILES: O=C(Cc1ccc(OCCCC2CCN(c3ncc(Cl)cn3)CC2)cc1F)N1CC2(C1)OCC2NC[C@H](O)[C@H](O)[C@H](O)CO] | 2-(4-(3-(1-(5-chloropyrimidin-2-yl)piperidin-4- yl)propoxy)-2-fluorophenyl)-1-(3-(((2S,3S,4R)- 2,3,4,5-tetrahydroxypentyl)amino)-1-oxa-6- azaspiro[3.3]heptan-6-yl)ethan-1-one |
| 83 | 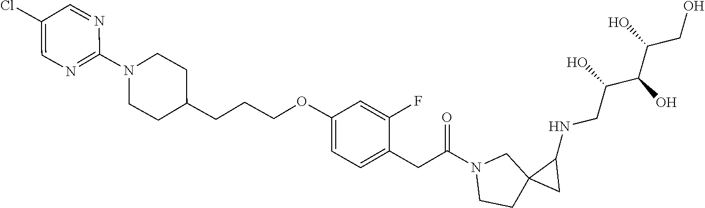 [SMILES: O=C(Cc1ccc(OCCCC2CCN(c3ncc(Cl)cn3)CC2)cc1F)N1CCC2(CC2NC[C@H](O)[C@H](O)[C@H](O)CO)C1] | 2-(4-(3-(1-(5-chloropyrimidin-2-yl)piperidin-4- yl)propoxy)-2-fluorophenyl)-1-(1-(((2S,3S,4R)- 2,3,4,5-tetrahydroxypentyl)amino)-5- azaspiro[2.4]heptan-5-yl)ethan-1-one |
| 84 | 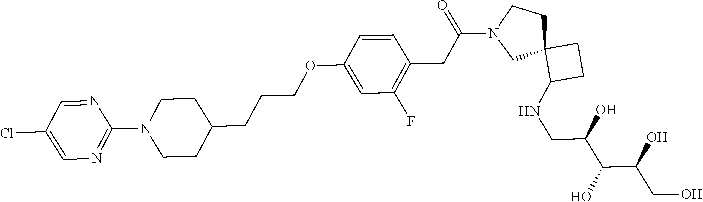 [SMILES: O=C(Cc1ccc(OCCCC2CCN(c3ncc(Cl)cn3)CC2)cc1F)N1CC[C@]2(CCC2NC[C@@H](O)[C@@H](O)[C@@H](O)CO)C1] | 2-(4-(3-(1-(5-chloropyrimidin-2-yl)piperidin-4- yl)propoxy)-2-fluorophenyl)-1-(1-(((2S,3S,4R)- 2,3,4,5-tetrahydroxypentyl)amino)-6- azaspiro[3.4]octan-6-yl)ethan-1-one |
| 85 | 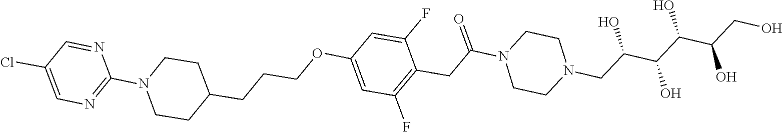 [SMILES: O=C(Cc1c(F)cc(OCCCC2CCN(c3ncc(Cl)cn3)CC2)cc1F)N1CCN(C[C@H](O)[C@@H](O)[C@H](O)[C@H](O)CO)CC1] | 2-(4-(3-(1-(5-chloropyrimidin-2-yl)piperidin-4- yl)propoxy)-2,6-difluorophenyl)-1-(4- ((2S,3R,4R,5R)-2,3,4,5,6- pentahydroxyhexyl)piperazin-1-yl)ethan-1-one |
| 86 | 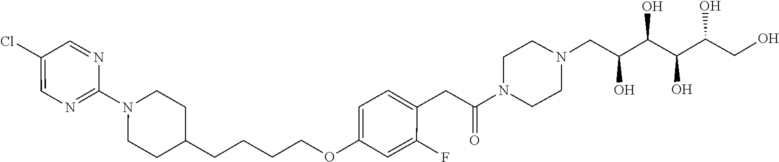 [SMILES: O=C(Cc1ccc(OCCCCC2CCN(c3ncc(Cl)cn3)CC2)cc1F)N1CCN(C[C@H](O)[C@@H](O)[C@H](O)[C@H](O)CO)CC1] | 2-(4-(4-(1-(5-chloropyrimidin-2-yl)piperidin-4- yl)butoxy)-2-fluorophenyl)-1-(4- ((2S,3R,4R,5R)-2,3,4,5,6- pentahydroxyhexyl)piperazin-1-yl)ethan-1-one |
| 87 | 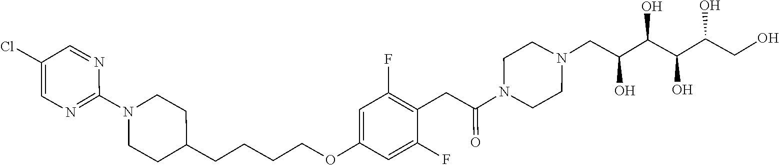 [SMILES: O=C(Cc1c(F)cc(OCCCCC2CCN(c3ncc(Cl)cn3)CC2)cc1F)N1CCN(C[C@H](O)[C@@H](O)[C@H](O)[C@H](O)CO)CC1] | 2-(4-(4-(1-(5-chloropyrimidin-2-yl)piperidin-4- yl)butoxy)-2,6-difluorophenyl)-1-(4- ((2S,3R,4R,5R)-2,3,4,5,6- pentahydroxyhexyl)piperazin-1-yl)ethan-1-one |
| 88 | 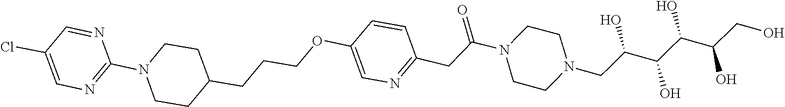 [SMILES: O=C(Cc1ccc(OCCCC2CCN(c3ncc(Cl)cn3)CC2)cn1)N1CCN(C[C@H](O)[C@@H](O)[C@H](O)[C@H](O)CO)CC1] | 2-(5-(3-(1-(5-chloropyrimidin-2-yl)piperidin-4- yl)propoxy)pyridin-2-yl)-1-(4-((2S,3R,4R,5R)- 2,3,4,5,6-pentahydroxyhexyl)piperazin-1- yl)ethan-1-one |
| 89 | 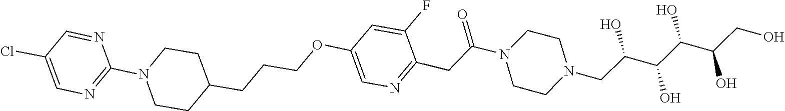 [SMILES: O=C(Cc1ncc(OCCCC2CCN(c3ncc(Cl)cn3)CC2)cc1F)N1CCN(C[C@H](O)[C@@H](O)[C@H](O)[C@H](O)CO)CC1] | 2-(5-(3-(1-(5-chloropyrimidin-2-yl)piperidin-4- yl)propoxy)-3-fluoropyridin-2-yl)-1-(4- ((2S,3R,4R,5R)-2,3,4,5,6- pentahydroxyhexyl)piperazin-1-yl)ethan-1-one |
| 90 | 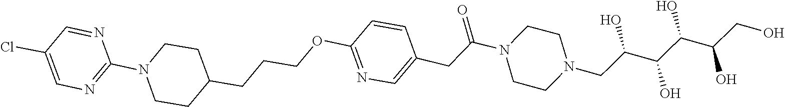 [SMILES: O=C(Cc1ccc(OCCCC2CCN(c3ncc(Cl)cn3)CC2)nc1)N1CCN(C[C@H](O)[C@@H](O)[C@H](O)[C@H](O)CO)CC1] | 2-(6-(3-(1-(5-chloropyrimidin-2-yl)piperidin-4- yl)propoxy)pyridin-3-yl)-1-(4-((2S,3R,4R,5R)- 2,3,4,5,6-pentahydroxyhexyl)piperazin-1- yl)ethan-1-one |
| 91 | 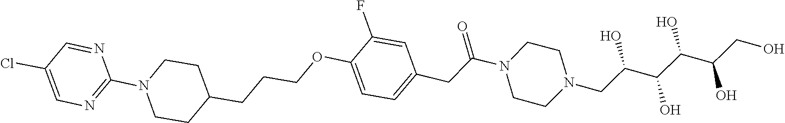 [SMILES: O=C(Cc1ccc(OCCCC2CCN(c3ncc(Cl)cn3)CC2)c(F)c1)N1CCN(C[C@H](O)[C@@H](O)[C@H](O)[C@H](O)CO)CC1] | 2-(4-(3-(1-(5-chloropyrimidin-2-yl)piperidin-4- yl)propoxy)-3-fluorophenyl)-1-(4- ((2S,3R,4R,5R)-2,3,4,5,6- pentahydroxyhexyl)piperazin-1-yl)ethan-1-one |
| 92 | 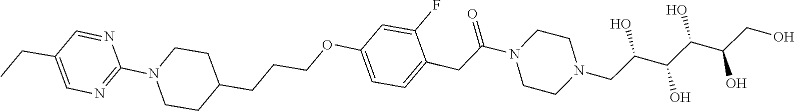 [SMILES: CCc1cnc(N2CCC(CCCOc3ccc(CC(=O)N4CCN(C[C@H](O)[C@@H](O)[C@H](O)[C@H](O)CO)CC4)c(F)c3)CC2)nc1] | 2-(4-(3-(1-(5-ethylpyrimidin-2-yl)piperidin-4- yl)propoxy)-2-fluorophenyl)-1-(4- ((2S,3R,4R,5R)-2,3,4,5,6- pentahydroxyhexyl)piperazin-1-yl)ethan-1-one |
| 93 | 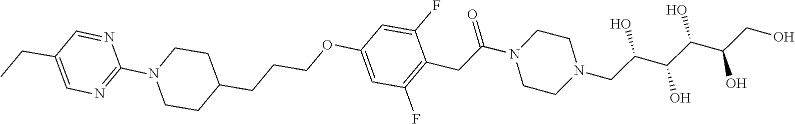 [SMILES: CCc1cnc(N2CCC(CCCOc3cc(F)c(CC(=O)N4CCN(C[C@H](O)[C@@H](O)[C@H](O)[C@H](O)CO)CC4)c(F)c3)CC2)nc1] | 2-(4-(3-(1-(5-ethylpyrimidin-2-yl)piperidin-4- yl)propoxy)-2,6-difluorophenyl)-1-(4- ((2S,3R,4R,5R)-2,3,4,5,6- pentahydroxyhexyl)piperazin-1-yl)ethan-1-one |
| 94 | 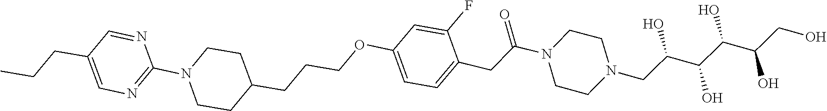 [SMILES: CCCc1cnc(N2CCC(CCCOc3ccc(CC(=O)N4CCN(C[C@H](O)[C@@H](O)[C@H](O)[C@H](O)CO)CC4)c(F)c3)CC2)nc1] | 2-(2-fluoro-4-(3-(1-(5-propylpyrimidin-2- yl)piperidin-4-yl)propoxy)phenyl)-1-(4- ((2S,3R,4R,5R)-2,3,4,5,6- pentahydroxyhexyl)piperazin-1-yl)ethan-1-one |
| 95 | 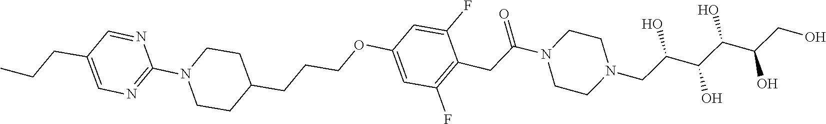 [SMILES: CCCc1cnc(N2CCC(CCCOc3cc(F)c(CC(=O)N4CCN(C[C@H](O)[C@@H](O)[C@H](O)[C@H](O)CO)CC4)c(F)c3)CC2)nc1] | 2-(2,6-difluoro-4-(3-(1-(5-propylpyrimidin-2- yl)piperidin-4-yl)propoxy)phenyl)-1-(4- ((2S,3R,4R,5R)-2,3,4,5,6- pentahydroxyhexyl)piperazin-1-yl)ethan-1-one |
| 96 | 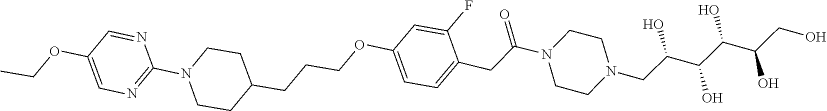 [SMILES: CCOc1cnc(N2CCC(CCCOc3ccc(CC(=O)N4CCN(C[C@H](O)[C@@H](O)[C@H](O)[C@H](O)CO)CC4)c(F)c3)CC2)nc1] | 2-(4-(3-(1-(5-ethoxypyrimidin-2-yl)piperidin-4- yl)propoxy)-2-fluorophenyl)-1-(4- ((2S,3R,4R,5R)-2,3,4,5,6- pentahydroxyhexyl)piperazin-1-yl)ethan-1-one |
| 97 | 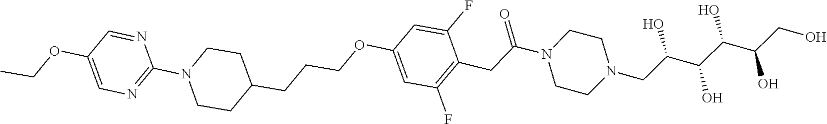 [SMILES: CCOc1cnc(N2CCC(CCCOc3cc(F)c(CC(=O)N4CCN(C[C@H](O)[C@@H](O)[C@H](O)[C@H](O)CO)CC4)c(F)c3)CC2)nc1] | 2-(4-(3-(1-(5-ethoxypyrimidin-2-yl)piperidin-4- yl)propoxy)-2,6-difluorophenyl)-1-(4- ((2S,3R,4R,5R)-2,3,4,5,6- pentahydroxyhexyl)piperazin-1-yl)ethan-1-one |
| 98 | 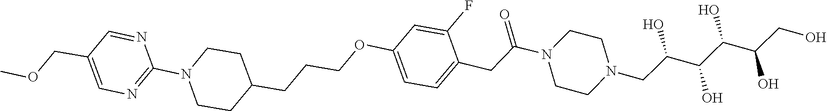 [SMILES: COCc1cnc(N2CCC(CCCOc3ccc(CC(=O)N4CCN(C[C@H](O)[C@@H](O)[C@H](O)[C@H](O)CO)CC4)c(F)c3)CC2)nc1] | 2-(2-fluoro-4-(3-(1-(5- (methoxymethyl)pyrimidin-2-yl)piperidin-4- yl)propoxy)phenyl)-1-(4-((2S,3R,4R,5R)- 2,3,4,5,6-pentahydroxyhexyl)piperazin-1- yl)ethan-1-one |
| 99 | 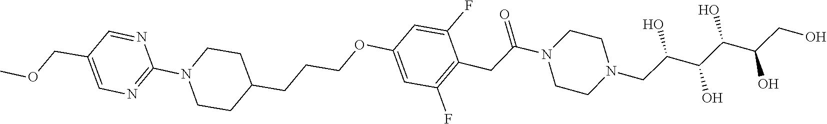 [SMILES: COCc1cnc(N2CCC(CCCOc3cc(F)c(CC(=O)N4CCN(C[C@H](O)[C@@H](O)[C@H](O)[C@H](O)CO)CC4)c(F)c3)CC2)nc1] | 2-(2,6-difluoro-4-(3-(1-(5- (methoxymethyl)pyrimidin-2-yl)piperidin-4- yl)propoxy)phenyl)-1-(4-((2S,3R,4R,5R)- 2,3,4,5,6-pentahydroxyhexyl)piperazin-1- yl)ethan-1-one |
| 100 | 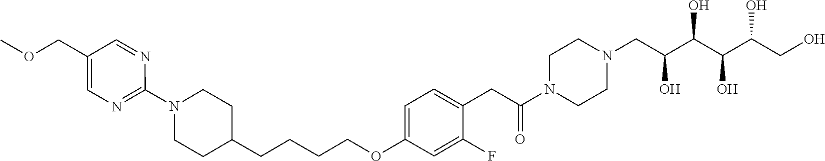 [SMILES: COCc1cnc(N2CCC(CCCCOc3ccc(CC(=O)N4CCN(C[C@H](O)[C@@H](O)[C@H](O)[C@H](O)CO)CC4)c(F)c3)CC2)nc1] | 2-(2-fluoro-4-(4-(1-(5- (methoxymethyl)pyrimidin-2-yl)piperidin-4- yl)butoxy)phenyl)-1-(4-((2S,3R,4R,5R)- 2,3,4,5,6-pentahydroxyhexyl)piperazin-1- yl)ethan-1-one |
| 101 | 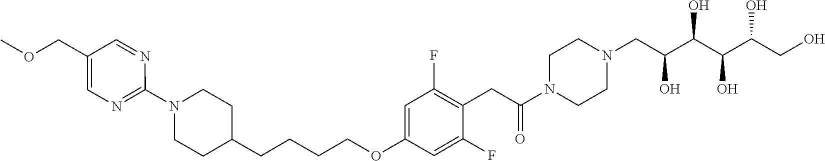 [SMILES: COCc1cnc(N2CCC(CCCCOc3cc(F)c(CC(=O)N4CCN(C[C@H](O)[C@@H](O)[C@H](O)[C@H](O)CO)CC4)c(F)c3)CC2)nc1] | 2-(2,6-difluoro-4-(4-(1-(5- (methoxymethyl)pyrimidin-2-yl)piperidin-4- yl)butoxy)phenyl)-1-(4-((2S,3R,4R,5R)- 2,3,4,5,6-pentahydroxyhexyl)piperazin-1- yl)ethan-1-one |
| 102 | 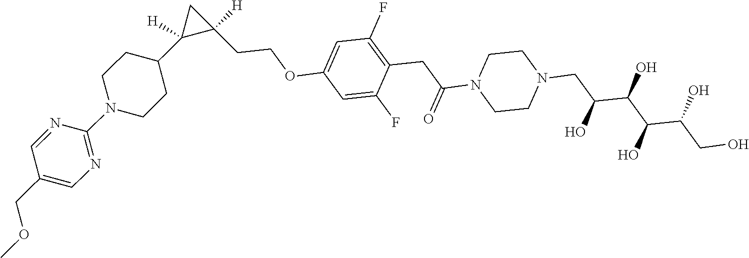 [SMILES: COCc1cnc(N2CCC([C@H]3C[C@H]3CCOc3cc(F)c(CC(=O)N4CCN(C[C@H](O)[C@@H](O)[C@H](O)[C@H](O)CO)CC4)c(F)c3)CC2)nc1] | 2-(2-fluoro-4-(2-((1S,2R)-2-(1-(5- (methoxymethyl)pyrimidin-2-yl)piperidin-4- yl)cyclopropyl)ethoxy)phenyl)-1-(4- ((2S,3R,4R,5R)-2,3,4,5,6- pentahydroxyhexyl)piperazin-1-yl)ethan-1-one |
| 103 | 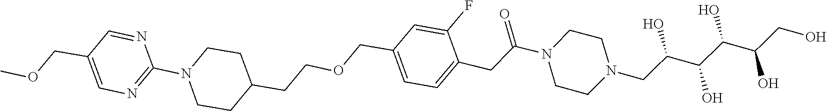 [SMILES: COCc1cnc(N2CCC(CCOCc3ccc(CC(=O)N4CCN(C[C@H](O)[C@@H](O)[C@H](O)[C@H](O)CO)CC4)c(F)c3)CC2)nc1] | 2-(2-fluoro-4-((2-(1-(5- (methoxymethyl)pyrimidin-2-yl)piperidin-4- yl)ethoxy)methyl)phenyl)-1-(4-((2S,3R,4R,5R)- 2,3,4,5,6-pentahydroxyhexyl)piperazin-1- yl)ethan-1-one |
| 104 | 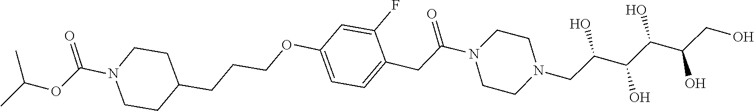 [SMILES: CC(C)OC(=O)N1CCC(CCCOc2ccc(CC(=O)N3CCN(C[C@H](O)[C@@H](O)[C@H](O)[C@H](O)CO)CC3)c(F)c2)CC1] | isopropyl 4-(3-(3-fluoro-4-(2-oxo-2-(4- ((2S,3R,4R,5R)-2,3,4,5,6- pentahydroxyhexyl)piperazin-1- yl)ethyl)phenoxy)propyl)piperidine-1- carboxylate |
| 105 | 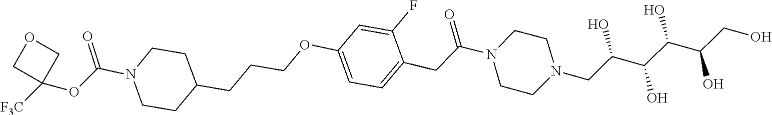 [SMILES: O=C(Cc1ccc(OCCCC2CCN(C(=O)OC3(C(F)(F)F)COC3)CC2)cc1F)N1CCN(C[C@H](O)[C@@H](O)[C@H](O)[C@H](O)CO)CC1] | 3-(trifluoromethyl)oxetan-3-yl 4-(3-(3-fluoro-4- (2-oxo-2-(4-((2S,3R,4R,5R)-2,3,4,5,6- pentahydroxyhexyl)piperazin-1- yl)ethyl)phenoxy)propyl)piperidine-1- carboxylate |
| 106 | 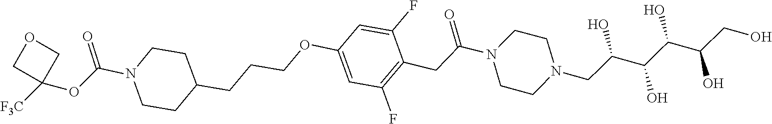 [SMILES: O=C(Cc1c(F)cc(OCCCC2CCN(C(=O)OC3(C(F)(F)F)COC3)CC2)cc1F)N1CCN(C[C@H](O)[C@@H](O)[C@H](O)[C@H](O)CO)CC1] | 3-(trifluoromethyl)oxetan-3-yl 4-(3-(3,5- difluoro-4-(2-oxo-2-(4-((2S,3R,4R,5R)- 2,3,4,5,6-pentahydroxyhexyl)piperazin-1- yl)ethyl)phenoxy)propyl)piperidine-1- carboxylate |
| 107 | 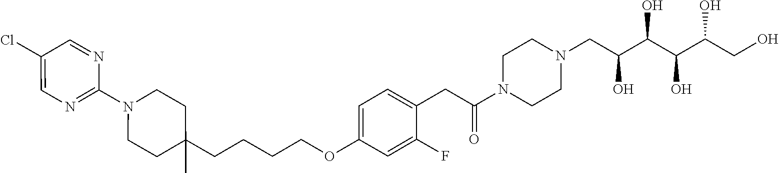 [SMILES: CC1(CCCCOc2ccc(CC(=O)N3CCN(C[C@H](O)[C@@H](O)[C@H](O)[C@H](O)CO)CC3)c(F)c2)CCN(c2ncc(Cl)cn2)CC1] | 2-(4-(4-(1-(5-chloropyrimidin-2-yl)-4- methylpiperidin-4-yl)butoxy)-2-fluorophenyl)-1- (4-((2S,3R,4R,5R)-2,3,4,5,6- pentahydroxyhexyl)piperazin-1-yl)ethan-1-one |
| 108 | 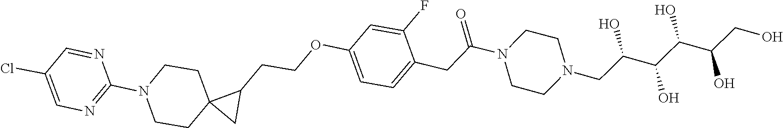 [SMILES: O=C(Cc1ccc(OCCC2CC23CCN(c2ncc(Cl)cn2)CC3)cc1F)N1CCN(C[C@H](O)[C@@H](O)[C@H](O)[C@H](O)CO)CC1] | 2-(4-(2-(6-(5-chloropyrimidin-2-yl)-6- azaspiro[2.5]octan-1-yl)ethoxy)-2-fluorophenyl)- 1-(4-((2S,3R,4R,5R)-2,3,4,5,6- pentahydroxyhexyl)piperazin-1-yl)ethan-1-one |
| 109 | 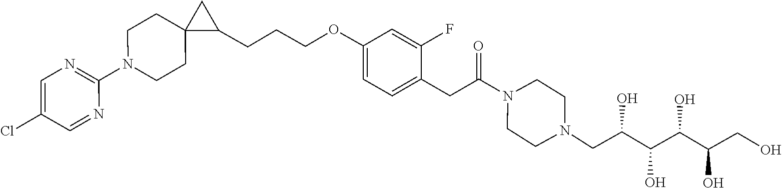 [SMILES: O=C(Cc1ccc(OCCCC2CC23CCN(c2ncc(Cl)cn2)CC3)cc1F)N1CCN(C[C@H](O)[C@@H](O)[C@H](O)[C@H](O)CO)CC1] | 2-(4-(3-(6-(5-chloropyrimidin-2-yl)-6- azaspiro[2.5]octan-1-yl)propoxy)-2- fluorophenyl)-1-(4-((2S,3R,4R,5R)-2,3,4,5,6- pentahydroxyhexyl)piperazin-1-yl)ethan-1-one |
| 110 | 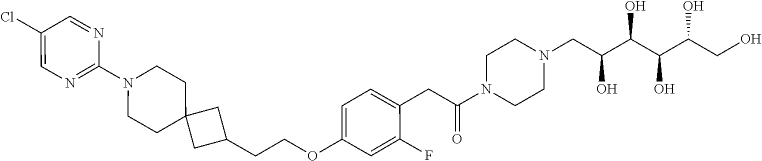 [SMILES: O=C(Cc1ccc(OCCC2CC3(CCN(c4ncc(Cl)cn4)CC3)C2)cc1F)N1CCN(C[C@H](O)[C@@H](O)[C@H](O)[C@H](O)CO)CC1] | 2-(4-(2-(7-(5-chloropyrimidin-2-yl)-7- azaspiro[3.5]nonan-2-yl)ethoxy)-2- fluorophenyl)-1-(4-((2S,3R,4R,5R)-2,3,4,5,6- pentahydroxyhexyl)piperazin-1-yl)ethan-1-one |
| 111 | 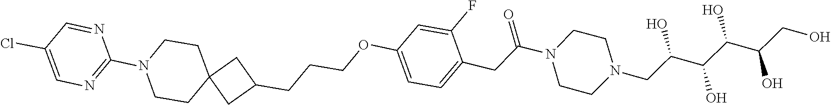 [SMILES: O=C(Cc1ccc(OCCCC2CC3(CCN(c4ncc(Cl)cn4)CC3)C2)cc1F)N1CCN(C[C@H](O)[C@@H](O)[C@H](O)[C@H](O)CO)CC1] | 2-(4-(3-(7-(5-chloropyrimidin-2-yl)-7- azaspiro[3.5]nonan-2-yl)propoxy)-2- fluorophenyl)-1-(4-((2S,3R,4R,5R)-2,3,4,5,6- pentahydroxyhexyl)piperazin-1-yl)ethan-1-one |
| 112 | 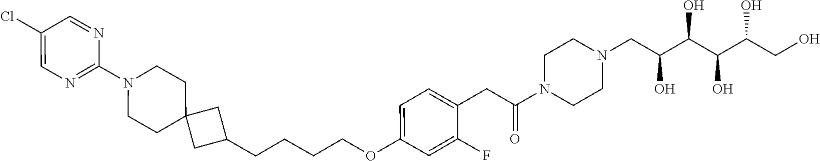 [SMILES: O=C(Cc1ccc(OCCCCC2CC3(CCN(c4ncc(Cl)cn4)CC3)C2)cc1F)N1CCN(C[C@H](O)[C@@H](O)[C@H](O)[C@H](O)CO)CC1] | 2-(4-(4-(7-(5-chloropyrimidin-2-yl)-7- azaspiro[3.5]nonan-2-yl)butoxy)-2- fluorophenyl)-1-(4-((2S,3R,4R,5R)-2,3,4,5,6- pentahydroxyhexyl)piperazin-1-yl)ethan-1-one |
| 113 | 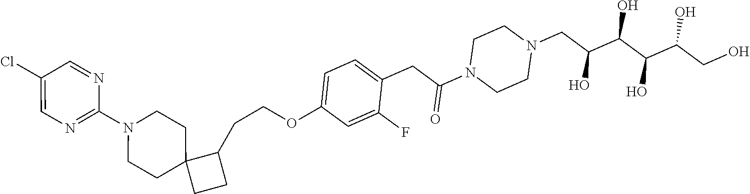 [SMILES: O=C(Cc1ccc(OCCC2CCC23CCN(c2ncc(Cl)cn2)CC3)cc1F)N1CCN(C[C@H](O)[C@@H](O)[C@H](O)[C@H](O)CO)CC1] | 2-(4-(2-(7-(5-chloropyrimidin-2-yl)-7- azaspiro[3.5]nonan-1-yl)ethoxy)-2- fluorophenyl)-1-(4-((2S,3R,4R,5R)-2,3,4,5,6- pentahydroxyhexyl)piperazin-1-yl)ethan-1-one |
| 114 | 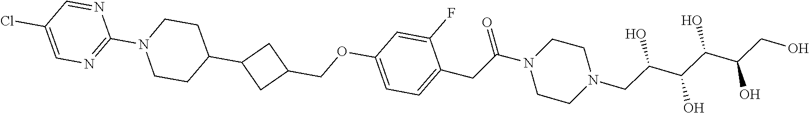 [SMILES: O=C(Cc1ccc(OCC2CC(C3CCN(c4ncc(Cl)cn4)CC3)C2)cc1F)N1CCN(C[C@H](O)[C@@H](O)[C@H](O)[C@H](O)CO)CC1] | 2-(4-((3-(1-(5-chloropyrimidin-2-yl)piperidin-4- yl)cyclobutyl)methoxy)-2-fluorophenyl)-1-(4- ((2S,3R,4R,5R)-2,3,4,5,6- pentahydroxyhexyl)piperazin-1-yl)ethan-1-one |
| 115 | 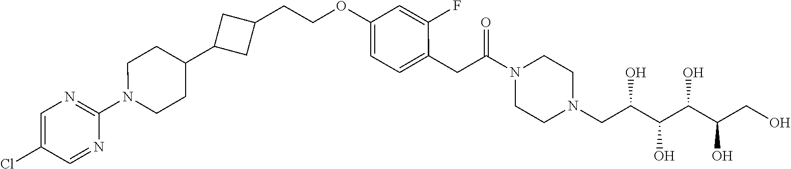 [SMILES: O=C(Cc1ccc(OCCC2CC(C3CCN(c4ncc(Cl)cn4)CC3)C2)cc1F)N1CCN(C[C@H](O)[C@@H](O)[C@H](O)[C@H](O)CO)CC1] | 2-(4-(2-(3-(1-(5-chloropyrimidin-2-yl)piperidin- 4-yl)cyclobutyl)ethoxy)-2-fluorophenyl)-1-(4- ((2S,3R,4R,5R)-2,3,4,5,6- pentahydroxyhexyl)piperazin-1-yl)ethan-1-one |

In some embodiments, provided herein is a pharmaceutically acceptable salt of a compound that is described in Table 1.

### Further Forms of Compounds

Furthermore, in some embodiments, the compounds described herein exist as “geometric isomers.” In some embodiments, the compounds described herein possess one or more double bonds. The compounds presented herein include all cis, trans, syn, anti, entgegen (E), and zusammen (Z) isomers as well as the corresponding mixtures thereof. In some situations, compounds exist as tautomers.

A “tautomer” refers to a molecule wherein a proton shift from one atom of a molecule to another atom of the same molecule is possible. In certain embodiments, the compounds presented herein exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

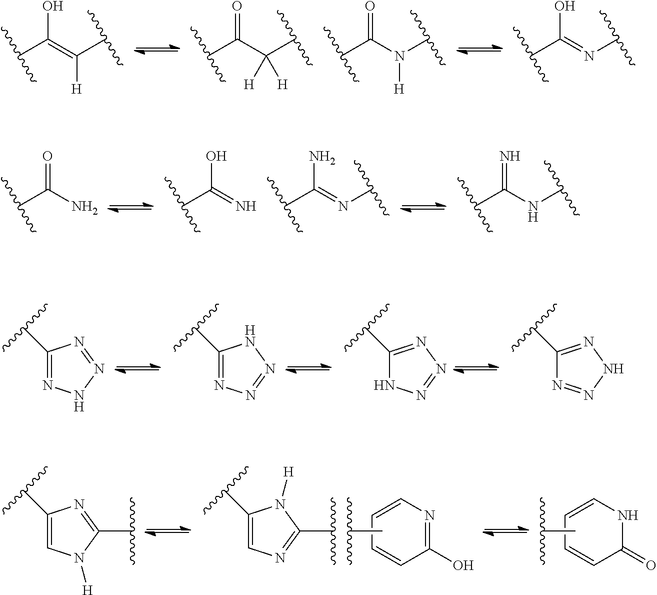

In some situations, the compounds described herein possess one or more chiral centers and each center exists in the (R)-configuration or (S)-configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as optically pure enantiomers by chiral chromatographic resolution of the racemic mixture. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred (e.g., crystalline diastereomeric salts). In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

The term “positional isomer” refers to structural isomers around a central ring, such as ortho-, meta-, and para-isomers around a benzene ring.

The methods and formulations described herein include the use of N-oxides (if appropriate), crystalline forms (also known as polymorphs), or pharmaceutically acceptable salts of compounds described herein, as well as active metabolites of these compounds having the same type of activity.

“Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997). Acid addition salts of basic compounds are prepared by contacting the free base forms with a sufficient amount of the desired acid to produce the salt.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. In some embodiments, pharmaceutically acceptable base addition salts are formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

“Prodrug” is meant to indicate a compound that is, in some embodiments, converted under physiological conditions or by solvolysis to an active compound described herein. Thus, the term prodrug refers to a precursor of an active compound that is pharmaceutically acceptable. A prodrug is typically inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam).

A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987.

The term “prodrug” is also meant to include any covalently bonded carriers, which release the active compound in vivo when such prodrug is administered to a mammalian subject. Prodrugs of an active compound, as described herein, are prepared by modifying functional groups present in the active compound in such a way that the modifications are cleaved, either in routine manipulation or in vivo, to the parent active compound. Prodrugs include compounds wherein a hydroxy, amino, carboxy, or mercapto group is bonded to any group that, when the prodrug of the active compound is administered to a mammalian subject, cleaves to form a free hydroxy, free amino, free carboxy, or free mercapto group, respectively. Examples of prodrugs include, but are not limited to, acetate, formate and benzoate derivatives of alcohol or amine functional groups in the active compounds and the like.

“Pharmaceutically acceptable solvate” refers to a composition of matter that is the solvent addition form. In some embodiments, solvates contain either stoichiometric or non-stoichiometric amounts of a solvent, and are formed during the process of making with pharmaceutically acceptable solvents such as water, ethanol, and the like. “Hydrates” are formed when the solvent is water, or “alcoholates” are formed when the solvent is alcohol. Solvates of compounds described herein are conveniently prepared or formed during the processes described herein. The compounds provided herein optionally exist in either unsolvated as well as solvated forms.

The compounds disclosed herein, in some embodiments, are used in different enriched isotopic forms, e.g., enriched in the content of ²H, ³H, ¹¹C, ¹³C and/or ¹⁴C. In some embodiments, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (2H), tritium (H), iodine-125 (¹²⁵I) or carbon-14 (¹⁴C). Isotopic substitution with ²H, ³H, ¹¹C, ¹³C, ¹⁴C, ¹⁵C, ²N, ¹³N, ¹⁵N, ¹⁶N, ¹⁷O, ¹⁸O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹²⁵I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art. In some embodiments deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

In some embodiments, the compounds described herein are labeled by other means, including, but not limited to, the use of chromophores or fluorescent moieties, bioluminescent labels, or chemiluminescent labels.

In certain embodiments, the compounds described herein, or a pharmaceutically acceptable salt, solvate, stereoisomer, or prodrug thereof, as described herein are substantially pure, in that it contains less than about 5%, or less than about 1%, or less than about 0.1%, of other organic small molecules, such as contaminating intermediates or by-products that are created, for example, in one or more of the steps of a synthesis method.

### Definitions

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulas, all combinations and subcombinations of ranges and specific embodiments therein are intended to be included.

The term “about” when referring to a number or a numerical range means that the number or numerical range referred to is an approximation within experimental variability (or within statistical experimental error), and thus the number or numerical range, in some instances, will vary between 1% and 15% of the stated number or numerical range.

The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

As used in the specification and appended claims, unless specified to the contrary, the following terms have the meaning indicated below:

As used herein, C₁-Cₓ includes C₁-C₂, C₁-C₃ . . . C₁-Cₓ. By way of example only, a group designated as “C₁-C₄” indicates that there are one to four carbon atoms in the moiety, i.e., groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl.

“Alkyl” refers to an optionally substituted straight-chain, or optionally substituted branched-chain saturated hydrocarbon monoradical having from one to about ten carbon atoms, or more preferably, from one to six carbon atoms, wherein an sp³-hybridized carbon of the alkyl residue is attached to the rest of the molecule by a single bond. Examples include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl, and the like. Whenever it appears herein, a numerical range such as “C₁-C₆ alkyl” means that the alkyl group consists of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁-C₁₀ alkyl, a C₁-C₉ alkyl, a C₁-C₈ alkyl, a C₁-C₇ alkyl, a C₁-C₆ alkyl, a C₁-C₅ alkyl, a C₁-C₄ alkyl, a C₁-C₃ alkyl, a C₁-C₂ alkyl, or a C₁ alkyl. Unless stated otherwise specifically in the specification, an alkyl group is optionally substituted as described below by one or more of the following substituents: halo, cyano, nitro, oxo, thioxo, imino, oximo, trimethylsilanyl, —OR^{a}, —SR^{a}, —OC(O)R^{a}, —OC(O)—OR^{f}, —N(R^{a})₂, —N⁺(R^{a})₃, —C(O)R^{a}, —C(O)OR^{a}, —C(O)N(R^{a})₂, —N(R^{a})C(O)OR^{f}, —OC(O)—N(R^{a})₂, —N(R^{a})C(O)R^{a}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, haloalkyl, cycloalkyl, aryl, aralkyl, heterocycloalkyl, heteroaryl or heteroarylalkyl, and each R^{f} is independently alkyl, haloalkyl, cycloalkyl, aryl, aralkyl, heterocycloalkyl, heteroaryl or heteroarylalkyl.

“Alkenyl” refers to an optionally substituted straight-chain, or optionally substituted branched-chain hydrocarbon monoradical having one or more carbon-carbon double-bonds and having from two to about ten carbon atoms, more preferably two to about six carbon atoms, wherein an sp²-hybridized carbon or an sp³-hybridized carbon of the alkenyl residue is attached to the rest of the molecule by a single bond. The group may be in either the cis or trans conformation about the double bond(s), and should be understood to include both isomers. Examples include, but are not limited to ethenyl (—CH═CH₂), 1-propenyl (—CH₂CH═CH₂), isopropenyl (—C(CH₃)═CH₂), butenyl, 1,3-butadienyl and the like. Whenever it appears herein, a numerical range such as “C₂-C₆ alkenyl” means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. In some embodiments, the alkenyl is a C₂-C₁₀ alkenyl, a C₂-C₉ alkenyl, a C₂-C₈ alkenyl, a C₂-C₇ alkenyl, a C₂-C₆ alkenyl, a C₂-C₅ alkenyl, a C₂-C₄ alkenyl, a C₂-C₃ alkenyl, or a C₂ alkenyl. Unless stated otherwise specifically in the specification, an alkenyl group is optionally substituted as described below, for example, with oxo, halogen, amino, nitrile, nitro, hydroxyl, haloalkyl, alkoxy, aryl, cycloalkyl, heterocycloalkyl, heteroaryl, and the like. Unless stated otherwise specifically in the specification, an alkenyl group is optionally substituted as described below by one or more of the following substituents: halo, cyano, nitro, oxo, thioxo, imino, oximo, trimethylsilanyl, —OR^{a}, —SR^{a}, —OC(O)—R^{f}, —OC(O)—OR^{f}, —N(R^{a})₂, —N⁺(R^{a})₃, —C(O)R^{a}, —C(O)OR^{a}, —C(O)N(R^{a})₂, —N(R^{a})C(O)OR^{f}, —OC(O)—N(R^{a})₂, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)N(R^{a})₂ (where t is 1 or 2) where each R^{a} is independently hydrogen, alkyl, haloalkyl, cycloalkyl, aryl, aralkyl, heterocycloalkyl, heteroaryl or heteroarylalkyl, and each R^{f} is independently alkyl, haloalkyl, cycloalkyl, aryl, aralkyl, heterocycloalkyl, heteroaryl or heteroarylalkyl.

“Alkynyl” refers to an optionally substituted straight-chain or optionally substituted branched-chain hydrocarbon monoradical having one or more carbon-carbon triple-bonds and having from two to about ten carbon atoms, more preferably from two to about six carbon atoms, wherein an sp-hybridized carbon or an sp³-hybridized carbon of the alkynyl residue is attached to the rest of the molecule by a single bond. Examples include, but are not limited to ethynyl, 2-propynyl, 2-butynyl, 1,3-butadiynyl and the like. Whenever it appears herein, a numerical range such as “C₂-C₆ alkynyl” means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. In some embodiments, the alkynyl is a C₂-C₁₀ alkynyl, a C₂-C₉ alkynyl, a C₂-C₈ alkynyl, a C₂-C₇ alkynyl, a C₂-C₆ alkynyl, a C₂-C₅ alkynyl, a C₂-C₄ alkynyl, a C₂-C₃ alkynyl, or a C₂ alkynyl. Unless stated otherwise specifically in the specification, an alkynyl group is optionally substituted as described below by one or more of the following substituents: halo, cyano, nitro, oxo, thioxo, imino, oximo, trimethylsilanyl, —OR^{a}, —SR^{a}, —OC(O)R^{a}, —OC(O)—OR^{f}, —N(R^{a})₂, —N⁺(R^{a})₃, —C(O)R^{a}, —C(O)OR^{a}, —C(O)N(R^{a})₂, —N(R^{a})C(O)OR^{f}, —OC(O)—N(R^{a})₂, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, haloalkyl, cycloalkyl, aryl, aralkyl, heterocycloalkyl, heteroaryl or heteroarylalkyl, and each R^{f} is independently alkyl, haloalkyl, cycloalkyl, aryl, aralkyl, heterocycloalkyl, heteroaryl or heteroarylalkyl.

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. Unless stated otherwise specifically in the specification, an alkylene group is optionally substituted as described below by one or more of the following substituents: halo, cyano, nitro, oxo, thioxo, imino, oximo, trimethylsilanyl, —OR^{a}, —SR^{a}, —OC(O)R^{a}, —OC(O)—OR^{f}, —N(R^{a})₂, —N⁺(R^{a})₃, —C(O)R^{a}, —C(O)OR^{a}, —C(O)N(R^{a})₂, —N(R^{a})C(O)OR^{f}, —OC(O)—N(R^{a})₂, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, haloalkyl, cycloalkyl, aryl, aralkyl, heterocycloalkyl, heteroaryl or heteroarylalkyl, and each R^{f} is independently alkyl, haloalkyl, cycloalkyl, aryl, aralkyl, heterocycloalkyl, heteroaryl or heteroarylalkyl.

“Alkenylene” or “alkenylene chain” refers to a straight or branched divalent hydrocarbon chain linking the rest of the molecule to a radical group, consisting solely of carbon and hydrogen, containing at least one carbon-carbon double bond, and having from two to twelve carbon atoms. The alkenylene chain is attached to the rest of the molecule through a single bond and to the radical group through a single bond. Unless stated otherwise specifically in the specification, an alkenylene group is optionally substituted as described below by one or more of the following substituents: halo, cyano, nitro, oxo, thioxo, imino, oximo, trimethylsilanyl, —OR^{a}, —SR^{a}, —OC(O)—R^{f}, —OC(O)—OR^{f}, —N(R^{a})₂, —N⁺(R^{a})₃, —C(O)R^{a}, —C(O)OR^{a}, —C(O)N(R^{a})₂, —N(R^{a})C(O)OR^{f}, —OC(O)—N(R^{a})₂, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, haloalkyl, cycloalkyl, aryl, aralkyl, heterocycloalkyl, heteroaryl or heteroarylalkyl, and each R^{f} is independently alkyl, haloalkyl, cycloalkyl, aryl, aralkyl, heterocycloalkyl, heteroaryl or heteroarylalkyl.

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and having from two to twelve carbon atoms. The alkynylene chain is attached to the rest of the molecule through a single bond and to the radical group through a single bond. Unless stated otherwise specifically in the specification, an alkynylene group is optionally substituted as described below by one or more of the following substituents: halo, cyano, nitro, oxo, thioxo, imino, oximo, trimethylsilanyl, —OR^{a}, —SR^{a}, —OC(O)R^{a}, —OC(O)—OR^{f}, —N(R^{a})₂, —N⁺(R^{a})₃, —C(O)R^{a}, —C(O)OR^{a}, —C(O)N(R^{a})₂, —N(R^{a})C(O)OR^{f}, —OC(O)—N(R^{a})₂, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, haloalkyl, cycloalkyl, aryl, aralkyl, heterocycloalkyl, heteroaryl or heteroarylalkyl, and each R^{f} is independently alkyl, haloalkyl, cycloalkyl, aryl, aralkyl, heterocycloalkyl, heteroaryl or heteroarylalkyl.

“Alkoxy” or “alkoxyl” refers to a radical bonded through an oxygen atom of the formula —O-alkyl, where alkyl is an alkyl chain as defined above.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from 6 to 18 carbon atoms, where at least one of the rings in the ring system is fully unsaturated, i.e., it contains a cyclic, delocalized (4n+2) π-electron system in accordance with the Hückel theory.

The ring system from which aryl groups are derived include, but are not limited to, groups such as benzene, fluorene, indane, indene, tetralin and naphthalene. In some embodiments, the aryl is a C₆-C₁₀ aryl. In some embodiments, the aryl is a phenyl. Unless stated otherwise specifically in the specification, the term “aryl” or the prefix “ar-” (such as in “aralkyl”) is meant to include aryl radicals optionally substituted as described below by one or more substituents independently selected from alkyl, alkenyl, alkynyl, halo, haloalkyl, cyano, nitro, aryl, aralkyl, aralkenyl, aralkynyl, cycloalkyl, heterocycloalkyl, heteroaryl, heteroarylalkyl, —R^{b}—OR^{a}, —R^{b}—SR^{a}, —R^{b}—OC(O)—R^{a}, —R^{b}—OC(O)—OR^{f}, —R^{b}—OC(O)—N(R^{a})₂, —R^{b}—N(R^{a})₂, —R^{b}—N⁺(R^{a})₃, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{f}, —R^{b}—N(R^{a})C(O)R^{a}, —R^{b}—N(R^{a})S(O)ₜR^{f} (where t is 1 or 2), —R^{b}—S(O)ₜOR^{a} (where t is 1 or 2), —R^{b}—S(O)ₜR^{f} (where t is 1 or 2) and —R^{b}—S(O)ₜN(R^{a})₂ (where t is 1 or 2), where each R^{a} is independently hydrogen, alkyl, haloalkyl, cycloalkyl, cycloalkylalkyl, aryl (optionally substituted with one or more halo groups), aralkyl, heterocycloalkyl, heteroaryl or heteroarylalkyl, R^{f} is independently alkyl, haloalkyl, cycloalkyl, cycloalkylalkyl, aryl (optionally substituted with one or more halo groups), aralkyl, heterocycloalkyl, heteroaryl or heteroarylalkyl, each R^{b} is independently a direct bond or a straight or branched alkylene or alkenylene chain, and R^{c} is a straight or branched alkylene or alkenylene chain.

An “arylene” refers to a divalent radical derived from an “aryl” group as described above linking the rest of the molecule to a radical group. The arylene is attached to the rest of the molecule through a single bond and to the radical group through a single bond. In some embodiments, the arylene is a phenylene. Unless stated otherwise specifically in the specification, an arylene group is optionally substituted as described above for an aryl group.

“Cycloalkyl” refers to a stable, partially or fully saturated, monocyclic or polycyclic carbocyclic ring, which may include fused (when fused with an aryl or a heteroaryl ring, the cycloalkyl is bonded through a non-aromatic ring atom) or bridged ring systems. Representative cycloalkyls include, but are not limited to, cycloalkyls having from three to fifteen carbon atoms (C₃-C₁₅ cycloalkyl), from three to ten carbon atoms (C₃-C₁₀ cycloalkyl), from three to eight carbon atoms (C₃-C₅ cycloalkyl), from three to six carbon atoms (C₃-C₆ cycloalkyl), from three to five carbon atoms (C₃-C₅ cycloalkyl), or three to four carbon atoms (C₃-C₄ cycloalkyl). In some embodiments, the cycloalkyl is a 3- to 6-membered cycloalkyl. In some embodiments, the cycloalkyl is a 5- to 6-membered cycloalkyl. Monocyclic cycloalkyls include, for example, cyclopropyl, cyclobutyl, cyclopentyl, cyclohexyl, cycloheptyl, and cyclooctyl. Polycyclic cycloalkyls or carbocycles include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, 7,7-dimethyl-bicyclo[2.2.1]heptanyl, and the like. Unless otherwise stated specifically in the specification, the term “cycloalkyl” is meant to include cycloalkyl radicals optionally substituted as described below by one or more substituents independently selected from alkyl, alkenyl, alkynyl, halo, haloalkyl, cyano, nitro, aryl, aralkyl, aralkenyl, aralkynyl, cycloalkyl, heterocycloalkyl, heteroaryl, heteroarylalkyl, —R^{b}—OR^{a}, —R^{b}—SR^{a}, —R^{b}—OC(O)—R^{a}, —R^{b}—OC(O)—OR^{f}, —R^{b}—OC(O)—N(R^{a})₂, —R^{b}—N(R^{a})₂, —R^{b}—N⁺(R^{a})₃, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{f}, —R^{b}—N(R^{a})C(O)R^{a}, —R^{b}—N(R^{a})S(O)ₜR^{f} (where t is 1 or 2), —R^{b}—S(O)ₜOR^{a} (where t is 1 or 2), —R^{b}—S(O)ₜR^{f} (where t is 1 or 2) and —R^{b}—S(O)ₜN(R^{a})₂ (where t is 1 or 2), where each R^{a} is independently hydrogen, alkyl, haloalkyl, cycloalkyl, cycloalkylalkyl, aryl (optionally substituted with one or more halo groups), aralkyl, heterocycloalkyl, heteroaryl or heteroarylalkyl, R^{f} is independently alkyl, haloalkyl, cycloalkyl, cycloalkylalkyl, aryl (optionally substituted with one or more halo groups), aralkyl, heterocycloalkyl, heteroaryl or heteroarylalkyl, each R^{b} is independently a direct bond or a straight or branched alkylene or alkenylene chain, and R^{c} is a straight or branched alkylene or alkenylene chain.

A “cycloalkylene” refers to a divalent radical derived from a “cycloalkyl” group as described above linking the rest of the molecule to a radical group. The cycloalkylene is attached to the rest of the molecule through a single bond and to the radical group through a single bond. Unless stated otherwise specifically in the specification, a cycloalkylene group is optionally substituted as described above for a cycloalkyl group.

“Halo” or “halogen” refers to bromo, chloro, fluoro or iodo. In some embodiments, halogen is fluoro or chloro. In some embodiments, halogen is fluoro.

“Haloalkyl” refers to an alkyl radical, as defined above, that is substituted by one or more halo radicals, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like.

“Haloalkoxy” or “haloalkoxyl” refers to an alkoxyl radical, as defined above, that is substituted by one or more halo radicals, as defined above.

“Fluoroalkoxy” or “fluoroalkoxyl” refers to an alkoxy radical, as defined above, that is substituted by one or more fluoro radicals, as defined above, for example, trifluoromethoxy, difluoromethoxy, fluoromethoxy, and the like.

“Hydroxyalkyl” refers to an alkyl radical, as defined above, that is substituted by one or more hydroxy radicals, as defined above, e.g., hydroxymethyl, 1-hydroxyethyl, 2-hydroxyethyl, 2-hydroxypropyl, 3-hydroxypropyl, 1,2-dihydroxyethyl, 2,3-dihydroxypropyl, 2,3,4,5,6-pentahydroxyhexyl, and the like.

“Heterocycloalkyl” refers to a stable 3- to 24-membered partially or fully saturated ring radical comprising 2 to 23 carbon atoms and from one to 8 heteroatoms selected from the group consisting of nitrogen, oxygen, and sulfur. Unless stated otherwise specifically in the specification, the heterocycloalkyl radical may be a monocyclic, bicyclic, tricyclic or tetracyclic ring system, which may include fused (when fused with an aryl or a heteroaryl ring, the heterocycloalkyl is bonded through a non-aromatic ring atom) or bridged ring systems; and the nitrogen, carbon or sulfur atoms in the heterocycloalkyl radical may be optionally oxidized; the nitrogen atom may be optionally quaternized. In some embodiments, the heterocycloalkyl is a 3- to 8-membered heterocycloalkyl. In some embodiments, the heterocycloalkyl is a 3- to 6-membered heterocycloalkyl. In some embodiments, the heterocycloalkyl is a 5- to 6-membered heterocycloalkyl. Examples of such heterocycloalkyl radicals include, but are not limited to, aziridinyl, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides and the oligosaccharides. More preferably, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e., skeletal atoms of the heterocycloalkyl ring). Unless stated otherwise specifically in the specification, the term “heterocycloalkyl” is meant to include heterocycloalkyl radicals as defined above that are optionally substituted by one or more substituents selected from alkyl, alkenyl, alkynyl, halo, fluoroalkyl, oxo, thioxo, cyano, nitro, aryl, aralkyl, aralkenyl, aralkynyl, cycloalkyl, heterocycloalkyl, heteroaryl, heteroarylalkyl, —R^{b}—OR^{a}, —R^{b}—SR^{a}, —R^{b}—OC(O)—R^{a}, —R^{b}—OC(O)—OR^{f}, —R^{b}—OC(O)—N(R^{a})₂, —R^{b}—N(R^{a})₂, —R^{b}—N⁺(R^{a})₃, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{f}, —R^{b}—N(R^{a})C(O)R^{a}, —R^{b}—N(R^{a})S(O)ₜR^{f} (where t is 1 or 2), —R^{b}—S(O)ₜOR^{a} (where t is 1 or 2), —R^{b}—S(O)ₜR^{f} (where t is 1 or 2) and —R^{b}—S(O)ₜN(R^{a})₂ (where t is 1 or 2), where each R^{a} is independently hydrogen, alkyl, haloalkyl, cycloalkyl, cycloalkylalkyl, aryl (optionally substituted with one or more halo groups), aralkyl, heterocycloalkyl, heteroaryl or heteroarylalkyl, R^{f} is independently alkyl, haloalkyl, cycloalkyl, cycloalkylalkyl, aryl (optionally substituted with one or more halo groups), aralkyl, heterocycloalkyl, heteroaryl or heteroarylalkyl, each R^{b} is independently a direct bond or a straight or branched alkylene or alkenylene chain, and R^{c} is a straight or branched alkylene or alkenylene chain.

“N-heterocycloalkyl” refers to a heterocycloalkyl radical as defined above containing at least one nitrogen and where the point of attachment of the heterocycloalkyl radical to the rest of the molecule is through a nitrogen atom in the heterocycloalkyl radical. An N-heterocycloalkyl radical is optionally substituted as described above for heterocycloalkyl radicals.

“C-heterocycloalkyl” refers to a heterocycloalkyl radical as defined above and where the point of attachment of the heterocycloalkyl radical to the rest of the molecule is through a carbon atom in the heterocycloalkyl radical. A C-heterocycloalkyl radical is optionally substituted as described above for heterocycloalkyl radicals.

A “heterocycloalkylene” refers to a divalent radical derived from a “heterocycloalkyl” group as described above linking the rest of the molecule to a radical group.

The heterocycloalkylene is attached to the rest of the molecule through a single bond and to the radical group through a single bond. Unless stated otherwise specifically in the specification, a heterocycloalkylene group is optionally substituted as described above for a heterocycloalkyl group.

“Heteroaryl” refers to a radical derived from a 5- to 18-membered aromatic ring radical that comprises one to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. In some embodiments, the heteroaryl is a 5- to 10-membered heteroaryl.

In some embodiments, the heteroaryl is a monocyclic heteroaryl, or a monocyclic 5- or 6-membered heteroaryl. In some embodiments, the heteroaryl is a 6,5-fused bicyclic heteroaryl.

The heteroatom(s) in the heteroaryl radical is optionally oxidized. One or more nitrogen atoms, if present, are optionally quaternized. The heteroaryl is attached to the rest of the molecule through any atom of the ring(s). Unless stated otherwise specifically in the specification, the term “heteroaryl” is meant to include heteroaryl radicals as defined above that are optionally substituted by one or more substituents selected from alkyl, alkenyl, alkynyl, halo, haloalkyl, oxo, thioxo, cyano, nitro, aryl, aralkyl, aralkenyl, aralkynyl, cycloalkyl, heterocycloalkyl, heteroaryl, heteroarylalkyl, —R^{b}—OR^{a}, —R^{b}—SR^{a}, —R^{b}—OC(O)—R^{a}, —R^{b}—OC(O)—OR^{f}, —R^{b}—OC(O)—N(R^{a})₂, —R^{b}—N(R^{a})₂, —R^{b}—N(R^{a})₃, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{f}, —R^{b}—N(R^{a})C(O)R^{a}, —R^{b}—N(R^{a})S(O)ₜR^{f} (where t is 1 or 2), —R^{b}—S(O)OR^{a} (where t is 1 or 2), —R^{b}—S(O)ₜR^{f} (where t is 1 or 2) and —R^{b}—S(O)ₜN(R^{a})₂ (where t is 1 or 2), where each R^{a} is independently hydrogen, alkyl, haloalkyl, cycloalkyl, cycloalkylalkyl, aryl (optionally substituted with one or more halo groups), aralkyl, heterocycloalkyl, heteroaryl or heteroarylalkyl, R^{f} is independently alkyl, haloalkyl, cycloalkyl, cycloalkylalkyl, aryl (optionally substituted with one or more halo groups), aralkyl, heterocycloalkyl, heteroaryl or heteroarylalkyl, each R^{b} is independently a direct bond or a straight or branched alkylene or alkenylene chain, and R^{c} is a straight or branched alkylene or alkenylene chain.

A “heteroarylene” refers to a divalent radical derived from a “heteroaryl” group as described above linking the rest of the molecule to a radical group. The heteroarylene is attached to the rest of the molecule through a single bond and to the radical group through a single bond.

Unless stated otherwise specifically in the specification, a heteroarylene group is optionally substituted as described above for a heteroaryl group.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be unsubstituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.). It will be understood by those skilled in the art with respect to any group containing one or more substituents that such groups are not intended to introduce any substitution or substitution patterns (e.g., substituted alkyl includes optionally substituted cycloalkyl groups, which in turn are defined as including optionally substituted alkyl groups, potentially ad infinitum) that are sterically impractical and/or synthetically non-feasible.

The term “modulate” or “modulating” or “modulation” refers to an increase or decrease in the amount, quality, or effect of a particular activity, function or molecule. By way of illustration and not limitation, agonists, partial agonists, inverse agonists, antagonists, and allosteric modulators of a G protein-coupled receptor are modulators of the receptor.

The term “agonism” as used herein refers to the activation of a receptor or enzyme by a modulator, or agonist, to produce a biological response.

The term “agonist” as used herein refers to a modulator that binds to a receptor or target enzyme and activates the receptor or enzyme to produce a biological response. By way of example, “GPR119 agonist” can be used to refer to a compound that exhibits an EC₅₀ with respect to GPR119 activity of no more than about 100 μM, as measured in the as measured in the inositol phosphate accumulation assay. In some embodiments, the term “agonist” includes full agonists or partial agonists.

The term “full agonist” refers to a modulator that binds to and activates a receptor or target enzyme with the maximum response that an agonist can elicit at the receptor or enzyme.

The term “partial agonist” refers to a modulator that binds to and activates a receptor or target enzyme, but has partial efficacy, that is, less than the maximal response, at the receptor or enzyme relative to a full agonist.

The term “positive allosteric modulator” refers to a modulator that binds to a site distinct from the orthosteric binding site and enhances or amplifies the effect of an agonist.

The term “antagonism” as used herein refers to the inactivation of a receptor or target enzyme by a modulator, or antagonist. Antagonism of a receptor, for example, is when a molecule binds to the receptor or target enzyme and does not allow activity to occur.

The term “antagonist” or “neutral antagonist” as used herein refers to a modulator that binds to a receptor or target enzyme and blocks a biological response. An antagonist has no activity in the absence of an agonist or inverse agonist but can block the activity of either, causing no change in the biological response.

The term “inverse agonist” refers to a modulator that binds to the same receptor or target enzyme as an agonist but induces a pharmacological response opposite to that agonist, i.e., a decrease in biological response.

The term “negative allosteric modulator” refers to a modulator that binds to a site distinct from the orthosteric binding site and reduces or dampens the effect of an agonist.

As used herein, “EC₅₀” is intended to refer to the concentration of a substance (e.g., a compound or a drug) that is required for 50% activation or enhancement of a biological process. In some instances, EC₅₀ refers to the concentration of agonist that provokes a response halfway between the baseline and maximum response in an in vitro assay. In some embodiments as used herein, EC₅₀ refers to the concentration of an agonist (e.g., a GPR119 agonist) that is required for 50% activation of GPR119.

As used herein, “IC₅₀” is intended to refer to the concentration of a substance (e.g., a compound or a drug) that is required for 50% inhibition of a biological process. For example, IC₅₀ refers to the half maximal (50%) inhibitory concentration (IC) of a substance as determined in a suitable assay. In some instances, an IC₅₀ is determined in an in vitro assay system. In some embodiments as used herein, IC₅₀ refers to the concentration of a modulator (e.g., an antagonist or inhibitor) that is required for 50% inhibition of a receptor or a target enzyme.

The terms “subject,” “individual,” and “patient” are used interchangeably. These terms encompass mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like.

The term “gut-restricted” as used herein refers to a compound, e.g., a GPR119 agonist, that is predominantly active in the gastrointestinal system. In some embodiments, the biological activity of the gut-restricted compound, e.g., a gut-restricted GPR119 agonist, is restricted to the gastrointestinal system. In some embodiments, gastrointestinal concentration of a gut-restricted modulator, e.g., a gut-restricted GPR119 agonist, is higher than the IC₅₀ value or the EC₅₀ value of the gut-restricted modulator against its receptor or target enzyme, e.g., GPR119, while the plasma levels of said gut-restricted modulator, e.g., gut-restricted GPR119 agonist, are lower than the IC₅₀ value or the EC₅₀ value of the gut-restricted modulator against its receptor or target enzyme, e.g., GPR119. In some embodiments, the gut-restricted compound, e.g., a gut-restricted GPR119 agonist, is non-systemic. In some embodiments, the gut-restricted compound, e.g., a gut-restricted GPR119 agonist, is a non-absorbed compound. In other embodiments, the gut-restricted compound, e.g., a gut-restricted GPR119 agonist, is absorbed, but is rapidly metabolized to metabolites that are significantly less active than the modulator itself toward the target receptor or enzyme, i.e., a “soft drug.” In other embodiments, the gut-restricted compound, e.g., a gut-restricted GPR119 agonist, is minimally absorbed and rapidly metabolized to metabolites that are significantly less active than the modulator itself toward the target receptor or enzyme.

In some embodiments, the gut-restricted modulator, e.g., a gut-restricted GPR119 agonist, is non-systemic but is instead localized to the gastrointestinal system. For example, the modulator, e.g., a gut-restricted GPR119 agonist, may be present in high levels in the gut, but low levels in serum. In some embodiments, the systemic exposure of a gut-restricted modulator, e.g., a gut-restricted GPR119 agonist, is, for example, less than 100, less than 50, less than 20, less than 10, or less than 5 nM, bound or unbound, in blood serum. In some embodiments, the intestinal exposure of a gut-restricted modulator, e.g., a gut-restricted GPR119 agonist, is, for example, greater than 1000, 5000, 10000, 50000, 100000, or 500000 nM. In some embodiments, a modulator, e.g., a GPR119 agonist, is gut-restricted due to poor absorption of the modulator itself, or because of absorption of the modulator which is rapidly metabolized in serum resulting in low systemic circulation, or due to both poor absorption and rapid metabolism in the serum.

In some embodiments, a modulator, e.g., a GPR119 agonist, is covalently bonded to a kinetophore, optionally through a linker, which changes the pharmacokinetic profile of the modulator.

In particular embodiments, the gut-restricted GPR119 agonist is a soft drug. The term “soft drug” as used herein refers to a compound that is biologically active but is rapidly metabolized to metabolites that are significantly less active than the compound itself toward the target receptor. In some embodiments, the gut-restricted GPR119 agonist is a soft drug that is rapidly metabolized in the blood to significantly less active metabolites. In some embodiments, the gut-restricted GPR119 agonist is a soft drug that is rapidly metabolized in the liver to significantly less active metabolites. In some embodiments, the gut-restricted GPR119 agonist is a soft drug that is rapidly metabolized in the blood and the liver to significantly less active metabolites. In some embodiments, the gut-restricted GPR119 agonist is a soft drug that has low systemic exposure. In some embodiments, the biological activity of the metabolite(s) is/are 10-fold, 20-fold, 50-fold, 100-fold, 500-fold, or 1000-fold lower than the biological activity of the soft drug gut-restricted GPR119 agonist.

The term “kinetophore” as used herein refers to a structural unit tethered to a small molecule modulator, e.g., a GPR119 agonist, optionally through a linker, which makes the whole molecule larger and increases the polar surface area while maintaining biological activity of the small molecule modulator. The kinetophore influences the pharmacokinetic properties, for example solubility, absorption, distribution, rate of elimination, and the like, of the small molecule modulator, e.g., a GPR119 agonist, and has minimal changes to the binding to or association with a receptor or target enzyme. The defining feature of a kinetophore is not its interaction with the target, for example a receptor, but rather its effect on specific physiochemical characteristics of the modulator to which it is attached, e.g., a GPR119 agonist. In some instances, kinetophores are used to restrict a modulator, e.g., a GPR119 agonist, to the gut.

The term “linked” as used herein refers to a covalent linkage between a modulator, e.g., a GPR119 agonist, and a kinetophore. The linkage can be through a covalent bond, or through a “linker.” As used herein, “linker” refers to one or more bifunctional molecules which can be used to covalently bond to the modulator, e.g., a GPR119 agonist, and kinetophore. In some embodiments, the linker is attached to any part of the modulator, e.g., a GPRJ 19 agonist, so long as the point of attachment does not interfere with the binding of the modulator to its receptor or target enzyme. In some embodiments, the linker is non-cleavable. In some embodiments, the linker is cleavable. In some embodiments, the linker is cleavable in the gut. In some embodiments, cleaving the linker releases the biologically active modulator, e.g., a GPR119 agonist, in the gut.

The term “gastrointestinal system” (GI system) or “gastrointestinal tract” (GI tract) as used herein, refers to the organs and systems involved in the process of digestion. The gastrointestinal tract includes the esophagus, stomach, small intestine, which includes the duodenum, jejunum, and ileum, and large intestine, which includes the cecum, colon, and rectum. In some embodiments herein, the GI system refers to the “gut,” meaning the stomach, small intestines, and large intestines or to the small and large intestines, including, for example, the duodenum, jejunum, and/or colon.

### Preparation of the Compounds

Compounds described herein are synthesized using standard synthetic techniques or using methods known in the art in combination with methods described herein.

Unless otherwise indicated, conventional methods of mass spectroscopy, NMR, HPLC, protein chemistry, biochemistry, recombinant DNA techniques and pharmacology are employed.

Compounds are prepared using standard organic chemistry techniques such as those described in, for example, March's Advanced Organic Chemistry, 6th Edition, John Wiley and Sons, Inc. Alternative reaction conditions for the synthetic transformations described herein may be employed such as variation of solvent, reaction temperature, reaction time, as well as different chemical reagents and other reaction conditions.

In some embodiments, compounds described herein are prepared as described as outlined in the Examples.

### Pharmaceutical Compositions

In some embodiments, disclosed herein is a pharmaceutical composition comprising a GPR119 agonist described herein, or a pharmaceutically acceptable salt, solvate, stereoisomer, or prodrug thereof, and a pharmaceutically acceptable excipient. In some embodiments, the GPR119 agonist is combined with a pharmaceutically suitable (or acceptable) carrier (also referred to herein as a pharmaceutically suitable (or acceptable) excipient, physiologically suitable (or acceptable) excipient, or physiologically suitable (or acceptable) carrier) selected on the basis of a chosen route of administration, e.g., oral administration, and standard pharmaceutical practice as described, for example, in Remington: The Science and Practice of Pharmacy (Gennaro, 21^{st} Ed. Mack Pub. Co., Easton, PA (2005)).

Accordingly, provided herein is a pharmaceutical composition comprising a compound described herein, or a pharmaceutically acceptable salt or solvate thereof, together with a pharmaceutically acceptable excipient.

Examples of suitable aqueous and non-aqueous carriers which are employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity is maintained, for example, by the use of coating materials, such as lecithin, by the maintenance of the required particle size in the case of dispersions, and by the use of surfactants.

### Combination Therapies

In certain embodiments, it is appropriate to administer at least one compound described herein, or a pharmaceutically acceptable salt, solvate, stereoisomer, or prodrug thereof, in combination with one or more other therapeutic agents. In some embodiments, a compound described herein, or a pharmaceutically acceptable salt, solvate, stereoisomer, or prodrug thereof, is administered in combination with a TGR5 agonist, a GPR40 agonist, an SSTR5 antagonist, an SSTR5 inverse agonist, a CCK1 agonist, a PDE4 inhibitor, a DPP-4 inhibitor, a GLP-1 receptor agonist, metformin, or combinations thereof. In certain embodiments, the pharmaceutical composition further comprises one or more anti-diabetic agents. In certain embodiments, the pharmaceutical composition further comprises one or more anti-obesity agents. In certain embodiments, the pharmaceutical composition further comprises one or more agents to treat nutritional disorders.

Examples of a TGR5 agonist to be used in combination with a compound described herein, or a pharmaceutically acceptable salt, solvate, stereoisomer, or prodrug thereof, include: INT-777, XL-475, SRX-1374, RDX-8940, RDX-98940, SB-756050, and those disclosed in WO-2008091540, WO-2010059853, WO-2011071565, WO-2018005801, WO-2010014739, WO-2018005794, WO-2016054208, WO-2015160772, WO-2013096771, WO-2008067222, WO-2008067219, WO-2009026241, WO-2010016846, WO-2012082947, WO-2012149236, WO-2008097976, WO-2016205475, WO-2015183794, WO-2013054338, WO-2010059859, WO-2010014836, WO-2016086115, WO-2017147159, WO-2017147174, WO-2017106818, WO-2016161003, WO-2014100025, WO-2014100021, WO-2016073767, WO-2016130809, WO-2018226724, WO-2018237350, WO-2010093845, WO-2017147137, WO-2015181275, WO-2017027396, WO-2018222701, WO-2018064441, WO-2017053826, WO-2014066819, WO-2017079062, WO-2014200349, WO-2017180577, WO-2014085474.

Examples of a GPR40 agonist to be used in combination with a compound described herein, or a pharmaceutically acceptable salt, solvate, stereoisomer, or prodrug thereof, include: fasiglifam, MR-1704, SCO-267, SHR-0534, HXP-0057-SS, LY-2922470, P-11187, JTT-851, ASP-4178, AMG-837, ID-11014A, HD-C715, CNX-011-67, JNJ-076, TU-5113, HD-6277, MK-8666, LY-2881835, CPL-207-280, ZYDG-2, and those described in U.S. Ser. No. 07/750,048, WO-2005051890, WO-2005095338, WO-2006011615, WO-2006083612, WO-2006083781, WO-2007088857, WO-2007123225, WO-2007136572, WO-2008054674, WO-2008054675, WO-2008063768, WO-2009039942, WO-2009039943, WO-2009054390, WO-2009054423, WO-2009054468, WO-2009054479, WO-2009058237, WO-2010085522, WO-2010085525, WO-2010085528, WO-2010091176, WO-2010123016, WO-2010123017, WO-2010143733, WO-2011046851, WO-2011052756, WO-2011066183, WO-2011078371, WO-2011161030, WO-2012004269, WO-2012004270, WO-2012010413, WO-2012011125, WO-2012046869, WO-2012072691, WO-2012111849, WO-2012147518, WO-2013025424, WO-2013057743, WO-2013104257, WO-2013122028, WO-2013122029, WO-2013128378, WO-2013144097, WO-2013154163, WO-2013164292, WO-2013178575, WO-2014019186, WO-2014073904, WO-2014082918, WO-2014086712, WO-2014122067, WO-2014130608, WO-2014146604, WO-2014169817, WO-2014170842, WO-2014187343, WO-2015000412, WO-2015010655, WO-2015020184, WO-2015024448, WO-2015024526, WO-2015028960, WO-2015032328, WO-2015044073, WO-2015051496, WO-2015062486, WO-2015073342, WO-2015078802, WO-2015084692, WO-2015088868, WO-2015089809, WO-2015097713, WO-2015105779, WO-2015105786, WO-2015119899, WO-2015176267, WO-201600771, WO-2016019587, WO-2016022446, WO-2016022448, WO-2016022742, WO-2016032120, WO-2016057731, WO-2017025368, WO-2017027309, WO-2017027310, WO-2017027312, WO-2017042121, WO-2017172505, WO-2017180571, WO-2018077699, WO-2018081047, WO-2018095877, WO-2018106518, WO-2018111012, WO-2018118670, WO-2018138026, WO-2018138027, WO-2018138028, WO-2018138029, WO-2018138030, WO-2018146008, WO-2018172727, WO-2018181847, WO-2018182050, WO-2018219204, WO-2019099315, and WO-2019134984.

Examples of a SSTR5 antagonist or inverse agonist to be used in combination with a compound described herein, or a pharmaceutically acceptable salt, solvate, stereoisomer, or prodrug thereof, include those described in: WO-03104816, WO-2009050309, WO-2015052910, WO-2011146324, WO-2006128803, WO-2010056717, WO-2012024183, and WO-2016205032.

Examples of a CCK1 agonist to be used in combination with a compound described herein, or a pharmaceutically acceptable salt, solvate, stereoisomer, or prodrug thereof, include: A-70874, A-71378, A-71623, A-74498, CE-326597, GI-248573, GSKI-181771X, NN-9056, PD-149164, PD-134308, PD-135158, PD-170292, PF-04756956, SR-146131, SSR-125180, and those described in EP-00697403, US-20060177438, WO-2000068209, WO-2000177108, WO-2000234743, WO-2000244150, WO-2009119733, WO-2009314066, WO-2009316982, WO-2009424151, WO-2009528391, WO-2009528399, WO-2009528419, WO-2009611691, WO-2009611940, WO-2009851686, WO-2009915525, WO-2005035793, WO-2005116034, WO-2007120655, WO-2007120688, WO-2008091631, WO-2010067233, WO-2012070554, and WO-2017005765.

Examples of a PDE4 inhibitor to be used in combination with a compound described herein, or a pharmaceutically acceptable salt, solvate, stereoisomer, or prodrug thereof, include: apremilast, cilomilast, crisaborole, diazepam, luteolin, piclamilast, and roflumilast.

Examples of a DPP-4 inhibitor to be used in combination with a compound described herein, or a pharmaceutically acceptable salt, solvate, stereoisomer, or prodrug thereof, include: sitagliptin, vildagliptin, saxagliptin, linagliptin, gemigliptin, teneligliptin, alogliptin, trelagliptin, omarigliptin, evogliptin, gosogliptin, and dutogliptin.

Examples of a GLP-1 receptor agonist to be used in combination with a compound described herein, or a pharmaceutically acceptable salt, solvate, stereoisomer, or prodrug thereof, include: albiglutide, dulaglutide, exenatide, extended-release exenatide, liraglutide, lixisenatide, and semaglutide.

Examples of anti-diabetic agents to be used in combination with a compound described herein, or a pharmaceutically acceptable salt, solvate, stereoisomer, or prodrug thereof, include: GLP-1 receptor agonists such as exenatide, liraglutide, taspoglutide, lixisenatide, albiglutide, dulaglutide, semaglutide, OWL833 and ORMD 0901; SGLT2 inhibitors such as dapagliflozin, canagliflozin, empagliflozin, ertugliflozin, ipragliflozin, luseogliflozin, remogliflozin, sergliflozin, sotagliflozin, and tofogliflozin; biguinides such as metformin; insulin and insulin analogs.

Examples of anti-obesity agents to be used in combination with a compound described herein, or a pharmaceutically acceptable salt, solvate, stereoisomer, or prodrug thereof, include: GLP-1 receptor agonists such as liraglutide, semaglutide; SGLT1/2 inhibitors such as LIK066, pramlintide and other amylin analogs such as AM-833, AC2307, and BI 473494; PYY analogs such as NN-9747, NN-9748, AC-162352, AC-163954, GT-001, GT-002, GT-003, and RHS-08; GIP receptor agonists such as APD-668 and APD-597; GLP-1/GIP co-agonists such as tirzepatide (LY329176), BHM-089, LBT-6030, CT-868, SCO-094, NNC-0090-2746, RG-7685, NN-9709, and SAR-438335; GLP-1/glucagon co-agonist such as cotadutide (MEDI0382), BI 456906, TT-401, G-49, H&D-001A, ZP-2929, and HM-12525A; GLP-1/GIP/glucagon triple agonist such as SAR-441255, HM-15211, and NN-9423; GLP-1/secretin co-agonists such as GUB06-046; leptin analogs such as metreleptin; GDF15 modulators such as those described in WO2012138919, WO2015017710, WO2015198199, WO-2017147742 and WO-2018071493; FGF21 receptor modulators such as NN9499, NGM386, NGM313, BFKB8488A (RG7992), AKR-001, LLF-580, CVX-343, LY-2405319, B1089-100, and BMS-986036; MC4 agonists such as setmelanotide; MetAP2 inhibitors such as ZGN-1061; ghrelin receptor modulators such as HMO4 and AZP-531; ghrelin O-acyltransferase inhibitors such as T-3525770 (RM-852) and GLWL-01; and oxytocin analogs such as carbetocin.

Examples of agents for nutritional disorders to be used in combination with a compound described herein, or a pharmaceutically acceptable salt, solvate, stereoisomer, or prodrug thereof, include: GLP-2 receptor agonists such as tedaglutide, glepaglutide (ZP1848), elsiglutide (ZP1846), apraglutide (FE 203799), HM-15912, NB-1002, GX-G8, PE-0503, SAN-134, and those described in WO-2011050174, WO-2012028602, WO-2013164484, WO-2019040399, WO-2018142363, WO-2019090209, WO-2006117565, WO-2019086559, WO-2017002786, WO-2010042145, WO-2008056155, WO-2007067828, WO-2018229252, WO-2013040093, WO-2002066511, WO-2005067368, WO-2009739031, WO-2009632414, and WO2008028117; and GLP-1/GLP-2 receptor co-agonists such as ZP-GG-72 and those described in WO-2018104561, WO-2018104558, WO-2018103868, WO-2018104560, WO-2018104559, WO-2018009778, WO-2016066818, and WO-2014096440.

In one embodiment, the therapeutic effectiveness of one of the compounds described herein is enhanced by administration of an adjuvant (i.e., by itself the adjuvant has minimal therapeutic benefit, but in combination with another therapeutic agent, the overall therapeutic benefit to the patient is enhanced). Or, in some embodiments, the benefit experienced by a patient is increased by administering one of the compounds described herein with another agent (which also includes a therapeutic regimen) that also has therapeutic benefit.

In one specific embodiment, a compound described herein, or a pharmaceutically acceptable salt, solvate, stereoisomer, or prodrug thereof, is co-administered with one or more additional therapeutic agents, wherein the compound described herein, or a pharmaceutically acceptable salt, solvate, stereoisomer, or prodrug thereof, and the additional therapeutic agent(s) modulate different aspects of the disease, disorder or condition being treated, thereby providing a greater overall benefit than administration of either therapeutic agent alone. In some embodiments, the additional therapeutic agent(s) is a TGR5 agonist, a GPR40 agonist, an SSTR5 antagonist, an SSTR5 inverse agonist, a CCK1 agonist, a PDE4 inhibitor, a DPP-4 inhibitor, a GLP-1 receptor agonist, metformin, or combinations thereof. In some embodiments, the additional therapeutic agent is an anti-diabetic agent. In some embodiments, the additional therapeutic agent is an anti-obesity agent. In some embodiments, the additional therapeutic agent is an agent to treat nutritional disorders.

In combination therapies, the multiple therapeutic agents (one of which is one of the compounds described herein) are administered in any order or even simultaneously. If administration is simultaneous, the multiple therapeutic agents are, by way of example only, provided in a single, unified form, or in multiple forms (e.g., as a single pill or as two separate pills).

The compounds described herein, or pharmaceutically acceptable salts, solvates, stereoisomers, or prodrugs thereof, as well as combination therapies, are administered before, during or after the occurrence of a disease or condition, and the timing of administering the composition containing a compound varies. Thus, in one embodiment, the compounds described herein are used as a prophylactic and are administered continuously to subjects with a propensity to develop conditions or diseases in order to prevent the occurrence of the disease or condition.

In another embodiment, the compounds and compositions are administered to a subject during or as soon as possible after the onset of the symptoms. In specific embodiments, a compound described herein is administered as soon as is practicable after the onset of a disease or condition is detected or suspected, and for a length of time necessary for the treatment of the disease.

In some embodiments, a compound described herein, or a pharmaceutically acceptable salt thereof, is administered in combination with anti-inflammatory agent, anti-cancer agent, immunosuppressive agent, steroid, non-steroidal anti-inflammatory agent, antihistamine, analgesic, hormone blocking therapy, radiation therapy, monoclonal antibodies, or combinations thereof.

### EXAMPLES

### List of Abbreviations

As used above, and throughout the description of the invention, the following abbreviations, unless otherwise indicated, shall be understood to have the following meanings:
ACN or MeCN acetonitrile
BF₃—OEt₂ or BF₃-Et₂O boron trifluoride etherate
Boc or BOC tert-butyloxycarbonyl
DCM dichloromethane (CH₂Cl₂)
DEAD diethyl azodicarboxylate
DIAD diisopropyl azodicarboxylate
DIPEA or DIEA diisopropylethylamine
DMF dimethylformamide
DMSO dimethylsulfoxide
eq equivalent(s)
Et ethyl
EtOH ethanol
EtOAc ethyl acetate
FA formic acid
h, hr(s) hour(s)
HATU 1-[bis(dimethylamino)methylene]-1H-1,2,3-triazolo[4,5-b]pyridinium 3-oxid hexafluorophosphate
HPLC high performance liquid chromatography
Hunig's base diisopropylethylamine (DIPEA, DIEA)
i-PrOH iso-propanol
LCMS liquid chromatography-mass spectrometry
Me methyl
MeOH methanol
min(s) minute(s)
MS mass spectroscopy
NaHMDS sodium bis(trimethylsilyl)amide
n-BuLi N-butyllithium
NMO N-methyl-morpholine-N-oxide
NMR nuclear magnetic resonance
Pd₂(dba)₃ tris(dibenzylideneacetone)dipalladium(0)
PPh₃ triphenylphosphine
Rt or RT room temperature
sat. saturated
super-hydride lithium triethylborohydride (LiEt₃BH)
TBAI tetrabutylammonium iodide
TEA triethylamine
TFA trifluoroacetic acid
THE tetrahydrofuran
TLC thin layer chromatography
Tol or tol toluene
TPAP tetrapropylammonium perruthenate
tR retention time
X-phos 2-dicyclohexylphosphino-2′,4′,6′-triisopropylbiphenyl

### I. Chemical Synthesis

Unless otherwise noted, reagents and solvents were used as received from commercial suppliers. Anhydrous solvents and oven-dried glassware were used for synthetic transformations sensitive to moisture and/or oxygen. Yields were not optimized. Reaction times are approximate and were not optimized. Column chromatography and thin layer chromatography (TLC) were performed on silica gel unless otherwise noted.

### Intermediate 1: 2-(4-(3-(1-(5-chloropyrimidin-2-yl)piperidin-4-yl)propoxy)-2-fluorophenyl)acetic acid

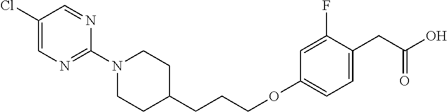 [SMILES: O=C(O)Cc1ccc(OCCCC2CCN(c3ncc(Cl)cn3)CC2)cc1F]

### Step 1: 3-(1-(5-chloropyrimidin-2-yl)piperidin-4-yl)propan-1-ol

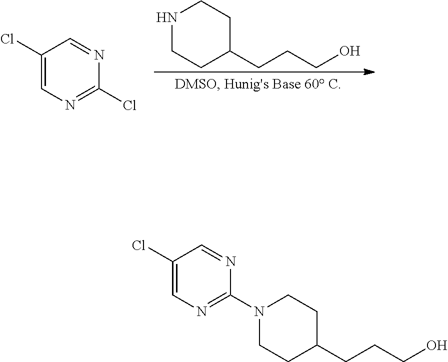 [SMILES: Clc1cnc(Cl)nc1 ; OCCCC1CCNCC1 ; OCCCC1CCN(c2ncc(Cl)cn2)CC1]

A mixture of 2,5-dichloropyrimidine (2.5 g, 16.78 mmol), 3-(piperidin-4-yl)propan-1-ol (2.4 g, 16.78 mmol) and Hunig's base (5.85 mL, 33.56 mmol) in DMSO (30 mL) was heated at 60° C. overnight. Mixture cooled and poured into water (150 mL) and extracted with EtOAc (3×50 mL); combined EtOAc layers washed with sat. NaCl (50 ML), dried over Na₂SO₄, filtered and evaporated. The residue was purified by silica gel column chromatography (Teledyne Isco: SNAP 80 g GOLD) eluent: gradient 0-100% EtOAc in Hexanes to give 3-(1-(5-chloropyrimidin-2-yl)piperidin-4-yl)propan-1-ol (3.56 g, 82%) as a white solid. ¹H NMR (500 MHz, Chloroform-d) δ 8.19 (s, 2H), 4.67 (ddt, J=13.4, 4.3, 1.9 Hz, 2H), 3.65 (t, J=6.6 Hz, 2H), 2.85 (ddd, J=13.3, 12.3, 2.8 Hz, 2H), 1.80-1.74 (m, 2H), 1.65-1.58 (m, 3H), 1.53 (th, J=10.7, 3.5 Hz, 1H), 1.36-1.30 (m, 3H), 1.16 (tdd, J=13.3, 11.6, 4.2 Hz, 2H). LCMS: tR=0.64, (ES⁺) m/z (M+H)⁺=256.2.

### Step 2: methyl 2-(4-(3-(1-(5-chloropyrimidin-2-yl)piperidin-4-yl)propoxy)-2-fluorophenyl)acetate

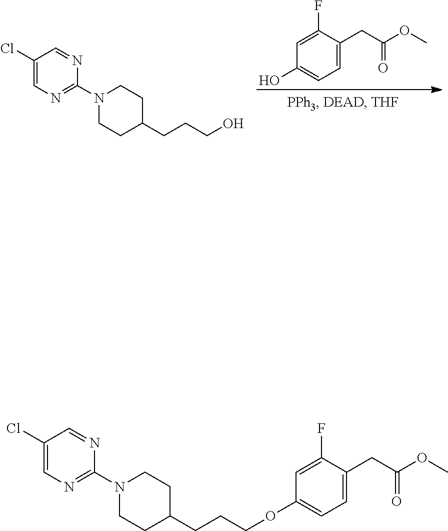 [SMILES: OCCCC1CCN(c2ncc(Cl)cn2)CC1 ; COC(=O)Cc1ccc(O)cc1F ; COC(=O)Cc1ccc(OCCCC2CCN(c3ncc(Cl)cn3)CC2)cc1F]

To a mixture of 3-[1-(5-chloropyrimidin-2-yl)-4-piperidyl]propan-1-ol (1.13 g, 4.4 mmol) and methyl-2-fluoro-4-hydroxyphenyl acetate (814 mg, 4.4 mmol) and triphenyl phosphine (2.5 g of polymer bound ˜3 mmol/g, 6.6 mmol) in DCM (20 mL) was added DEAD (2.98 mL of a 40% wt solution in toluene, 6.6 mmol) and the resulting mixture stirred at room temperature overnight. Mixture filtered through celite and the filtrate evaporated. The residue was purified by silica gel column chromatography (Teledyne Isco: SNAP 40 g GOLD) eluent: gradient 0-30% EtOAc in Heptane to give 2-(4-(3-(1-(5-chloropyrimidin-2-yl)piperidin-4-yl)propoxy)-2-fluorophenyl)acetate (1.14 g, 61%) as a white solid. ¹H NMR (500 MHz, Chloroform-d) δ 8.20 (s, 2H), 7.13 (t, J=8.5 Hz, 1H), 6.66-6.63 (m, 1H), 6.61 (dd, J=11.6, 2.5 Hz, 1H), 4.68 (dp, J=13.2, 1.9 Hz, 2H), 3.93 (t, J=6.4 Hz, 2H), 3.70 (s, 3H), 3.61-3.58 (m, 2H), 2.86 (ddd, J=13.3, 12.3, 2.8 Hz, 2H), 1.86-1.77 (m, 4H), 1.56 (ddd, J=11.1, 8.5, 4.8 Hz, 1H), 1.42 (dddd, J=9.3, 7.2, 5.6, 2.5 Hz, 2H), 1.23-1.13 (m, 2H). LCMS: tR=1.59, (ES⁺) m/z (M+H)⁺=422.2.

### Step 3: 2-(4-(3-(1-(5-chloropyrimidin-2-yl)piperidin-4-yl)propoxy)-2-fluorophenyl)acetic acid

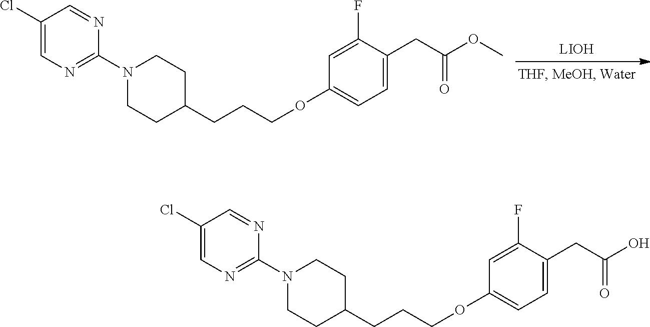 [SMILES: COC(=O)Cc1ccc(OCCCC2CCN(c3ncc(Cl)cn3)CC2)cc1F ; O=C(O)Cc1ccc(OCCCC2CCN(c3ncc(Cl)cn3)CC2)cc1F]

To a solution of methyl 2-[4-[3-[1-(5-chloropyrimidin-2-yl)-4-piperidyl]propoxy]-2-fluoro-phenyl]acetate (1.14 g, 2.7 mmol) in THF (15 mL) and MeOH (5 mL) was added lithium hydroxide (5.4 mL of a 1M aqueous soln, 5.4 mmol) and the resulting mixture stirred at room temperature for 1 hour. Mixture evaporated to remove organic solvents and remaining aqueous diluted with water (20 mL) and acidified by the addition of 1N HCl and extracted with DCM (2×15 mL); combined DCM layers dried over Na₂SO₄, filtered and evaporated to give 2-(4-(3-(1-(5-chloropyrimidin-2-yl)piperidin-4-yl)propoxy)-2-fluorophenyl)acetic acid (1.1 g, 99%) as a white solid. ¹H NMR (500 MHz, Chloroform-d) δ 8.20 (s, 2H), 7.13 (t, J=8.5 Hz, 1H), 6.67-6.63 (m, 1H), 6.62 (dd, J=11.5, 2.5 Hz, 1H), 4.67 (dp, J=13.2, 1.9 Hz, 2H), 3.93 (t, J=6.4 Hz, 2H), 3.63 (d, J=1.2 Hz, 2H), 2.86 (ddd, J=13.3, 12.3, 2.7 Hz, 2H), 1.86-1.75 (m, 4H), 1.57 (ddt, J=14.7, 7.0, 3.7 Hz, 1H), 1.45-1.38 (m, 2H), 1.23-1.12 (m, 2H). LCMS: tR=1.28, (ES⁺) m/z (M+H)⁺=408.2.

The following intermediates in Table P1 were prepared using procedures similar to those described in Intermediate 1 using appropriate starting materials.

TABLE P1

| Int | | |
|---|---|---|
| # | Structure | ¹HNMR/Mass [M + H]⁺ |
| 2 | 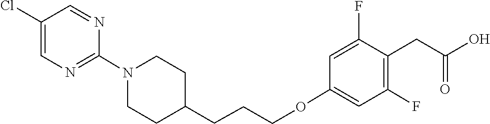 [SMILES: O=C(O)Cc1c(F)cc(OCCCC2CCN(c3ncc(Cl)cn3)CC2)cc1F] | ¹H NMR (500 MHz, Chloroform-d) δ 8.20 (s, 2H), 6.49- 6.41 (m, 2H), 4.68 (dp, J = 13.4, 2.0 Hz, 2H), 3.91 (t, J = 6.4 Hz, 2H), 3.67 (d, J = 1.3 Hz, 2H), 2.92-2.81 (m, 2H), 1.86-1.75 (m, 4H), 1.56 (ddt, J = 14.5, 7.0, 3.6 Hz, 1H), 1.46-1.37 (m, 3H), 1.18 (qd, J = 12.5, 4.2 Hz, 2H). LCMS: tR = 1.51, (ES⁺) m/z (M + H)⁺ 426.1. |
| 3 | 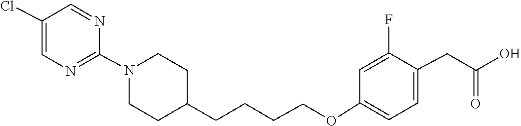 [SMILES: O=C(O)Cc1ccc(OCCCCC2CCN(c3ncc(Cl)cn3)CC2)cc1F] | ¹H NMR (500 MHz, DMSO-d₆) δ 8.36 (s, 2H), 7.14 (t, J = 8.7 Hz, 1H), 6.68-6.62 (m, 2H), 4.57 (dt, J = 13.8, 3.0 Hz, 2H), 3.93 (t, J = 6.5 Hz, 2H), 3.22 (s, 2H), 2.87 (td, J = 12.8, 2.7 Hz, 2H), 1.76-1.65 (m, 4H), 1.54 (ddp, J = 10.8, 6.8, 3.5 Hz, 1H), 1.48-1.40 (m, 2H), 1.27 (q, J = 7.2 Hz, 2H), 1.03 (qd, J = 12.6, 4.2 Hz, 2H). LCMS: tR = 1.48, (ES⁺) m/z (M + H)⁺ = 422.2. |
| 4 | 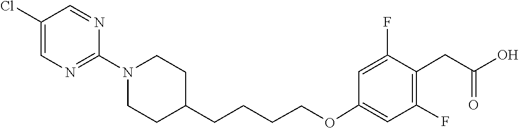 [SMILES: O=C(O)Cc1c(F)cc(OCCCCC2CCN(c3ncc(Cl)cn3)CC2)cc1F] | ¹H NMR (500 MHz, Chloroform-d) δ 8.20 (s, 2H), 6.49- 6.41 (m, 2H), 4.65 (dq, J = 13.4, 2.3 Hz, 2H), 3.92 (t, J = 6.3 Hz, 2H), 3.66 (s, 2H), 2.90-2.80 (m, 2H), 1.80-1.73 (m, 4H), 1.57-1.44 (m, 3H), 1.35-1.28 (m, 2H), 1.19- 1.09 (m, 2H). LCMS: tR = 1.49, (ES⁺) m/z (M + H)⁺ = 440.1. |
| 5 | 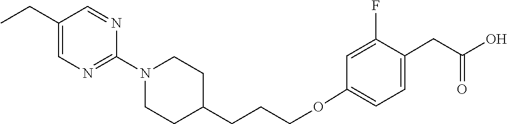 [SMILES: CCc1cnc(N2CCC(CCCOc3ccc(CC(=O)O)c(F)c3)CC2)nc1] | ¹H NMR (500 MHz, Chloroform-d) δ 8.20 (s, 2H), 6.49- 6.41 (m, 2H), 4.65 (dq, J = 13.4, 2.3 Hz, 2H), 3.92 (t, J = 6.3 Hz, 2H), 3.66 (s, 2H), 2.90-2.80 (m, 2H), 1.80-1.73 (m, 4H), 1.57-1.44 (m, 3H), 1.35-1.28 (m, 2H), 1.19- 1.09 (m, 2H). LCMS: tR = 1.07, (ES⁺) m/z (M + H)⁺ = 402.3. |
| 6 | 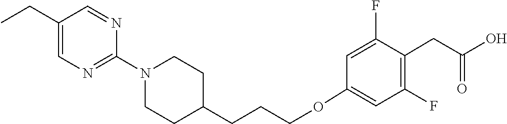 [SMILES: CCc1cnc(N2CCC(CCCOc3cc(F)c(CC(=O)O)c(F)c3)CC2)nc1] | ¹H NMR (500 MHz, Chloroform-d) δ 8.20 (s, 2H), 6.49- 6.41 (m, 2H), 4.65 (dq, J = 13.4, 2.3 Hz, 2H), 3.92 (t, J = 6.3 Hz, 2H), 3.66 (s, 2H), 2.90-2.80 (m, 2H), 1.80-1.73 (m, 4H), 1.57-1.44 (m, 3H), 1.35-1.28 (m, 2H), 1.19- 1.09 (m, 2H). LCMS: tR = 0.96, (ES⁺) m/z (M + H)⁺ = 420.3. |
| 7 | 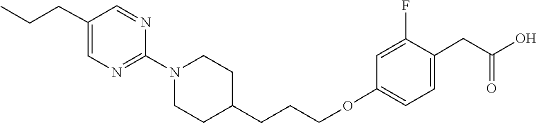 [SMILES: CCCc1cnc(N2CCC(CCCOc3ccc(CC(=O)O)c(F)c3)CC2)nc1] | ¹H NMR (500 MHz, Chloroform-d) δ 8.20 (s, 2H), 7.22 (t, J = 8.6 Hz, 1H), 6.64 (dd, J = 8.5, 2.5 Hz, 1H), 6.58 (dd, J = 11.7, 2.5 Hz, 1H), 4.68 (dp, J = 13.2, 1.9 Hz, 2H), 4.29-4.21 (m, 1H), 4.16-4.09 (m, 1H), 3.92 (t, J = 6.5 Hz, 2H), 3.81 (dd, J = 8.5, 5.6 Hz, 1H), 3.73-3.62 (m, 3H), 3.38 (s, 2H), 2.91-2.82 (m, 2H), 2.73 (ddt, J = 10.3, 7.9, 4.0 Hz, 1H), 1.86 (dt, J = 7.7, 6.3 Hz, 2H), 1.83-1.76 (m, 4H), 1.60-1.53 (m, 1H), 1.45-1.38 (m, |
| | | 3H), 1.23-1.13 (m, 2H). LCMS: tR = 1.26, (ES⁺) m/z |
| | | (M + H)⁺ = 416.3. |
| 8 | 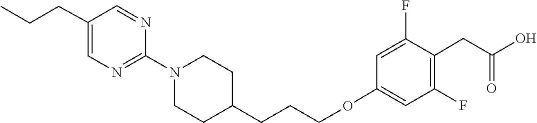 [SMILES: CCCc1cnc(N2CCC(CCCOc3cc(F)c(CC(=O)O)c(F)c3)CC2)nc1] | ¹H NMR (500 MHz, Chloroform-d) δ 8.10 (s, 2H), 6.38 (d, J = 9.1 Hz, 2H), 4.61 (dt, J = 13.4, 2.8 Hz, 2H), 3.84 (t, J = 6.4 Hz, 2H), 3.59 (s, 2H), 2.87-2.76 (m, 2H), 2.32 (t, J = 7.5 Hz, 2H), 1.73 (ddd, J = 10.5, 8.3, 4.1 Hz, 4H), 1.49 (h, J = 7.4 Hz, 3H), 1.34 (ddt, J = 12.3, 7.0, 3.4 Hz, 2H), 1.13 (qd, J = 12.5, 4.2 Hz, 2H), 0.86 (t, J = 7.3 Hz, 3H). LCMS: tR = 1.32, (ES⁺) m/z (M + H)⁺ = 434.3. |
| 9 | 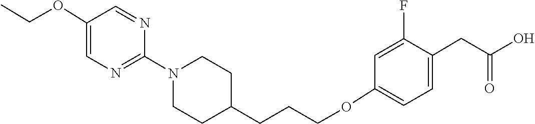 [SMILES: CCOc1cnc(N2CCC(CCCOc3ccc(CC(=O)O)c(F)c3)CC2)nc1] | ¹H NMR (500 MHz, Chloroform-d) δ 8.09 (s, 2H), 7.14 (t, J = 8.6 Hz, 1H), 6.65 (dd, J = 8.4, 2.6 Hz, 1H), 6.62 (dd, J = 11.5, 2.5 Hz, 1H), 4.61 (dt, J = 12.5, 2.8 Hz, 2H), 3.99 (q, J = 7.0 Hz, 2H), 3.93 (t, J = 6.5 Hz, 2H), 3.63 (d, J = 1.1 Hz, 2H), 2.85 (td, J = 12.8, 2.6 Hz, 2H), 1.86-1.76 (m, 4H), 1.54 (dddt, J = 14.8, 10.8, 7.0, 3.6 Hz, 1H), 1.45- 1.34 (m, 5H), 1.24-1.16 (m, 2H). LCMS: tR = 0.96, |
| | | (ES⁺) m/z (M + H)⁺ = 418.3. |
| 10 | 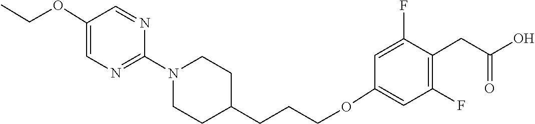 [SMILES: CCOc1cnc(N2CCC(CCCOc3cc(F)c(CC(=O)O)c(F)c3)CC2)nc1] | LCMS: tR = 1.18, (ES⁺) m/z (M + H)⁺ = 436.3. |
| 11 | 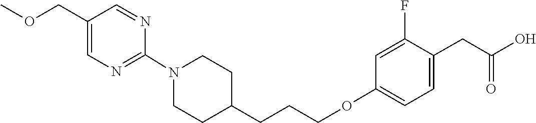 [SMILES: COCc1cnc(N2CCC(CCCOc3ccc(CC(=O)O)c(F)c3)CC2)nc1] | ¹H NMR (500 MHz, Chloroform-d) δ 8.29 (s, 2H), 7.14 (t, J = 8.6 Hz, 1H), 6.67-6.64 (m, 1H), 6.62 (dd, J = 11.5, 2.5 Hz, 1H), 6.47 (s, 1H), 4.78-4.70 (m, 2H), 4.26 (s, 2H), 4.21 (q, J = 7.1 Hz, 2H), 3.93 (t, J = 6.4 Hz, 2H), 3.63 (d, J = 1.2 Hz, 2H), 3.34 (s, 3H), 2.89 (td, J = 12.9, 2.7 Hz, 2H), 1.86-1.77 (m, 4H), 1.58 (ddt, J = 11.3, 7.8, 4.3 Hz, 1H), 1.45-1.39 (m, 2H), 1.28 (t, J = 7.1 Hz, 3H), 1.18 (qd, J = 12.3, 4.2 Hz, 2H). LCMS: |
| | | tR = 0.96, (ES⁺) m/z (M + H)⁺ = 418.3. |
| 12 | 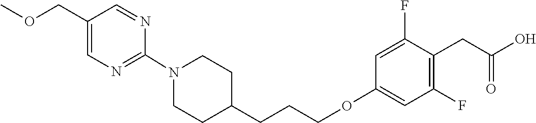 [SMILES: COCc1cnc(N2CCC(CCCOc3cc(F)c(CC(=O)O)c(F)c3)CC2)nc1] | ¹H NMR (500 MHz, Chloroform-d) δ 8.28 (s, 2H), 6.49- 6.41 (m, 2H), 4.78-4.70 (m, 2H), 4.26 (s, 2H), 3.91 (t, J = 6.4 Hz, 2H), 3.66 (s, 2H), 3.34 (s, 3H), 2.88 (td, J = 12.9, 2.7 Hz, 2H), 1.87-1.75 (m, 4H), 1.57 (dtq, J = 14.7, 7.0, 3.4 Hz, 1H), 1.45-1.36 (m, 2H), 1.23-1.13 (m, 2H). LCMS: tR = 0.86, (ES⁺) m/z (M + H)⁺ = 436.5. |
| 13 | 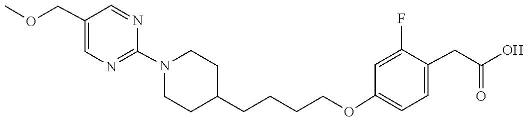 [SMILES: COCc1cnc(N2CCC(CCCCOc3ccc(CC(=O)O)c(F)c3)CC2)nc1] | ¹H NMR (500 MHz, Chloroform-d) δ 8.27 (s, 2H), 7.22 (t, J = 8.6 Hz, 1H), 6.68-6.63 (m, 1H), 6.59 (dd, J = 11.7, 2.5 Hz, 1H), 4.76-4.69 (m, 2H), 4.26 (s, 2H), 4.20 (t, J = 8.5 Hz, 1H), 4.11-4.03 (m, 1H), 3.98-3.88 (m, 3H), 3.82-3.72 (m, 3H), 3.40 (d, J = 1.5 Hz, 2H), 3.34 (s, 3H), 2.87 (td, J = 12.9, 2.7 Hz, 2H), 2.82-2.73 (m, 1H), 1.81-1.74 (m, 4H), 1.56-1.46 (m, 2H), 1.32 (dt, J = 9.1, 6.8 Hz, 2H), 1.20-1.12 (m, 2H). LCMS: |
| | | tR = 0.86, (ES⁺) m/z (M + H)⁺ = 432.3. |
| 14 | 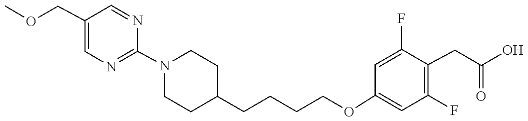 [SMILES: COCc1cnc(N2CCC(CCCCOc3cc(F)c(CC(=O)O)c(F)c3)CC2)nc1] | ¹H NMR (500 MHz, Chloroform-d) δ 8.27 (s, 2H), 6.44 (d, J = 9.2 Hz, 2H), 4.73 (dq, J = 13.3, 2.2 Hz, 2H), 4.25 (d, J = 8.8 Hz, 3H), 4.08 (dd, J = 10.0, 8.5 Hz, 1H), 3.98 (dd, J = 8.6, 5.2 Hz, 1H), 3.90 (t, J = 6.4 Hz, 2H), 3.79 (ddd, J = 11.5, 7.9, 4.9 Hz, 3H), 3.40 (s, 2H), 3.34 (s, 3H), 2.91-2.84 (m, 2H), 2.83-2.76 (m, 1H), 1.81-1.73 (m, 2H), 1.57-1.44 (m, 1H), 1.20-1.11 (m, 1H). LCMS: tR = 0.92, (ES⁺) m/z (M + H)⁺ = 450.3. |
| 15 | 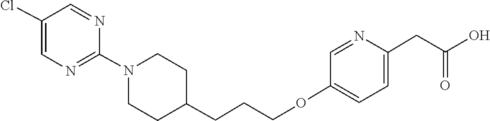 [SMILES: O=C(O)Cc1ccc(OCCCC2CCN(c3ncc(Cl)cn3)CC2)cn1] | ¹H NMR (500 MHz, Chloroform-d) δ 8.20 (s, 2H), 8.17 (d, J = 2.9 Hz, 1H), 7.30 (dd, J = 8.6, 2.9 Hz, 1H), 7.17 (d, J = 8.6 Hz, 1H), 4.69 (dp, J = 13.2, 1.9 Hz, 2H), 4.02 (t, J = 6.4 Hz, 2H), 3.84 (s, 2H), 2.87 (ddd, J = 13.3, 12.3, 2.7 Hz, 2H), 1.91-1.83 (m, 2H), 1.83-1.77 (m, 2H), 1.59 (ttt, J = 10.8, 7.0, 3.7 Hz, 1H), 1.48-1.40 (m, 2H), 1.24-1.13 (m, 2H). LCMS: tR = 1.00, (ES⁺) m/z (M + H)⁺ = 391.3. |
| 16 | 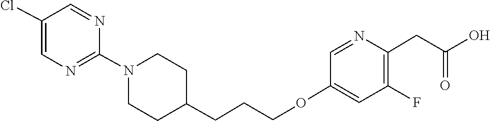 [SMILES: O=C(O)Cc1ncc(OCCCC2CCN(c3ncc(Cl)cn3)CC2)cc1F] | ¹H NMR (500 MHz, Chloroform-d) δ 8.20 (d, J = 4.9 Hz, 2H), 8.07 (d, J = 2.4 Hz, 1H), 7.05 (dd, J = 10.4, 2.2 Hz, 1H), 4.69 (ddt, J = 13.6, 4.6, 2.1 Hz, 2H), 4.02 (t, J = 6.4 Hz, 2H), 3.88 (s, 2H), 2.87 (td, J = 12.9, 2.7 Hz, 2H), 1.91-1.83 (m, 2H), 1.83-1.75 (m, 2H), 1.59 (tdd, J = 11.2, 8.7, 5.4 Hz, 1H), 1.44 (ddd, J = 12.0, 8.4, 5.9 Hz, 2H), 1.18 (pd, J = 13.4, 12.9, 4.2 Hz, 2H). LCMS: tR = 1.36, (ES⁺) m/z (M + H)⁺ = 409.2. |
| 17 | 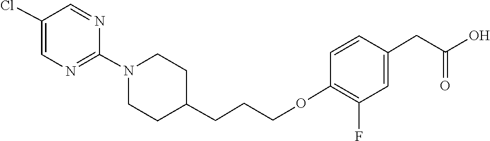 [SMILES: O=C(O)Cc1ccc(OCCCC2CCN(c3ncc(Cl)cn3)CC2)c(F)c1] | LCMS: tR = 1.37, (ES⁺) m/z (M + H)⁺ = 408.2. |

### Intermediate 18: 2-(2-fluoro-4-(2-((1S,2R)-2-(1-(5-(methoxymethyl)pyrimidin-2-yl)piperidin-4-yl)cyclopropyl)ethoxy) phenyl)acetic acid

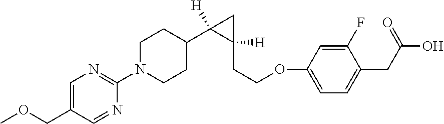 [SMILES: COCc1cnc(N2CCC([C@H]3C[C@H]3CCOc3ccc(CC(=O)O)c(F)c3)CC2)nc1]

### Step 1: tert-butyl 4-(2,2-dibromovinyl)piperidine-1-carboxylate

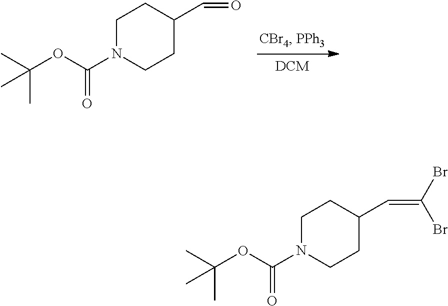 [SMILES: CC(C)(C)OC(=O)N1CCC(C=O)CC1 ; CC(C)(C)OC(=O)N1CCC(C=C(Br)Br)CC1]

Carbon tetrabromide (11.6 g, 35.1 mmol) in DCM (150 mL) was cooled in an ice bath and triphenylphosphine (18.4 g, 70.2 mmol) added and stirring at 0° C. continued for 25 mins then tert-butyl 4-formylpiperidine-1-carboxylate (5 g, 23.4 mmol) added in one portion. After stirring at ice bath temperature for 50 mins the mixture was evaporated to about ⅓ the original volume to give a suspension. Cyclopentylmethyl ether (150 mL) added causing more precipitation and the mixture filtered washing with more cyclopentylmethyl ether. The filtrate was washed with water (200 mL), 10% aqueous sodium bisulfite, dried over Na₂SO₄, filtered and evaporated. The residue was triturated with 40% EtOAc in Heptane and filtered through a pad of silica (washing with further 40% EtOAc in Heptane and filtrate evaporated to give tert-butyl 4-(2,2-dibromovinyl)piperidine-1-carboxylate (7.84 g, 90%) as a white solid. ¹H NMR (500 MHz, Chloroform-d) δ 6.23 (d, J=8.9 Hz, 1H), 4.06 (s, 2H), 2.88-2.65 (m, 2H), 2.44 (tdt, J=11.4, 8.9, 3.9 Hz, 1H), 1.75-1.67 (m, 2H), 1.46 (s, 9H), 1.37-1.27 (m, 2H).

### Step 2 tert-butyl 4-(3-hydroxyprop-1-yn-1-yl)piperidine-1-carboxylate

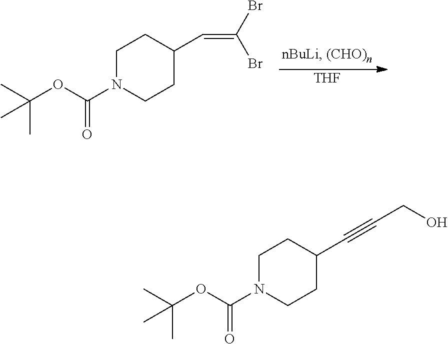 [SMILES: CC(C)(C)OC(=O)N1CCC(C=C(Br)Br)CC1 ; CC(C)(C)OC(=O)N1CCC(C#CCO)CC1 ; "THF"]

To a solution of tert-butyl 4-(2,2-dibromovinyl)piperidine-1-carboxylate (7.84 g, 21.2 mmol) in THE (100 mL) cooled at −45° C. was added n-butyl lithium (17.4 mL of a 2.5M soln in Hexanes, 43.5 mmol) slowly over 10 mins. After complete addition mixture stirred at −45° C. for 45 minutes then paraformaldehyde (1.91 g, 63.6 mmol) added and mixture allowed to warm slowly to warm to room temperature and stirred overnight. Mixture quenched by the addition of sat.NH₄Cl (200 mL) and extracted with EtOAc (300 mL); organic layer washed with water (200 mL), sat. NaCl (100 mL), dried over MgSO₄, filtered and evaporated. The residue was purified by silica gel column chromatography (Teledyne Isco: SNAP 80 g GOLD) eluent: gradient 0-100% EtOAc in Heptane (7cv) to give tert-butyl 4-(3-hydroxyprop-1-yn-1-yl)piperidine-1-carboxylate (3.77 g, 74%) as a light yellow oil. ¹H NMR (500 MHz, Chloroform-d) δ 4.27 (dd, J=6.0, 2.0 Hz, 2H), 3.75-3.66 (m, 2H), 3.14 (ddd, J=13.5, 8.8, 3.4 Hz, 2H), 2.60 (ttq, J=8.2, 4.0, 2.0 Hz, 1H), 1.77 (ddt, J=13.7, 6.3, 3.5 Hz, 2H), 1.56 (dtt, J=12.7, 8.6, 3.7 Hz, 2H), 1.45 (s, 9H).

### Step 3: tert-butyl (Z)-4-(3-hydroxyprop-1-en-1-yl)piperidine-1-carboxylate

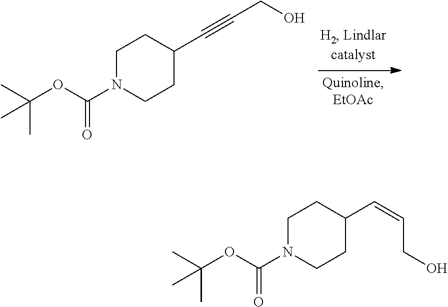 [SMILES: CC(C)(C)OC(=O)N1CCC(C#CCO)CC1 ; CC(C)(C)OC(=O)N1CCC(/C=C\CO)CC1]

To a solution of alkyne (CM-781, 6.6 g, 27.6 mmol) in EtOAc (120 mL) was added quinoline (0.55 mL) and Lindlar catalyst (750 mg) and the resulting mixture stirred under a balloon of hydrogen for 1 hour. Mixture filtered through celite and the filtrate evaporated. The residue was purified by silica gel column chromatography (Teledyne Isco: SNAP 120 g GOLD) eluent: gradient 0-100% EtOAc in Heptane to give tert-butyl (Z)-4-(3-hydroxyprop-1-en-1-yl)piperidine-1-carboxylate (5 g, 75%) as a light yellow oil. ¹H NMR (500 MHz, Chloroform-d) δ 5.58 (dtd, J=11.0, 6.8, 1.0 Hz, 1H), 5.37 (ddt, J=11.0, 9.5, 1.4 Hz, 1H), 4.22 (td, J=5.4, 2.7 Hz, 2H), 4.08 (s, 2H), 2.73 (d, J=13.6 Hz, 2H), 2.50-2.40 (m, 1H), 1.59-1.55 (m, 2H), 1.46 (s, 9H), 1.35-1.22 (m, 2H).

### Step 4: tert-butyl 4-((1R,2R)-2-(hydroxymethyl)cyclopropyl)piperidine-1-carboxylate

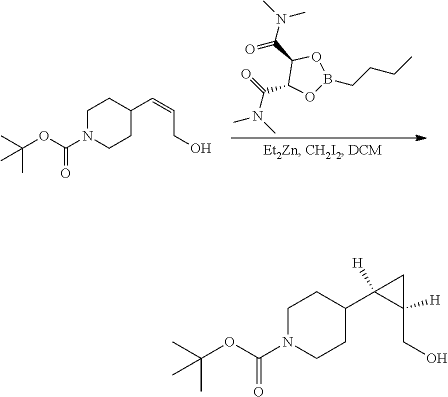 [SMILES: CC(C)(C)OC(=O)N1CCC(/C=C\CO)CC1 ; CCCCB1O[C@H](C(=O)N(C)C)[C@@H](C(=O)N(C)C)O1 ; CC(C)(C)OC(=O)N1CCC([C@H]2C[C@H]2CO)CC1]

In a 100 mL flask was added dichloromethane (20 mL) cooled to −30° C. and diethyl zinc (10.3 mL of a 1M soln in hexane, 10.3 mmol) added followed by 1,2-dimethoxyethane (1.07 mL, 10.3 mmol) and the resulting mixture stirred at −20° C. for 20 min then diiodomethane (1.67 mL, 20.7 mmol) added slowly over 10 min and the resulting mixture stirred at −20° C. for 45 minutes. To this mixture was added slowly over 45 min a mixture of tert-butyl (Z)-4-(3-hydroxyprop-1-en-1-yl)piperidine-1-carboxylate (1 g, 4.14 mmol) and (4S,5S)-2-butyl-N⁴,N⁴,N⁵,N⁵-tetramethyl-1,3,2-dioxaborolane-4,5-dicarboxamide (1.22 mL, 4.97 mmol) in DCM (12 mL) and the resulting mixture allowed to warm to room temperature overnight. Mixture quenched by the addition of sat. NH₄Cl (30 mL) and mixture decanted into a separating funnel and remaining solids treated with DCM (30 mL) and sat. NH₄Cl (30 mL) and stirred until all solids had dissolved, mixture added to separating funnel and organic layer separated and dried over MgSO₄, filtered and evaporated. The residue was purified by silica gel column chromatography (Teledyne Isco: SNAP 24g GOLD) eluent: gradient 0-100% EtOAc in Heptane to give an oil which partially solidified on standing. Mixture treated with heptane and solid filtered and dried to give tert-butyl 4-((1R,2R)-2-(hydroxymethyl)cyclopropyl)piperidine-1-carboxylate (660 mg, 62%) as a white solid. ¹H NMR (500 MHz, Chloroform-d) δ 4.09 (s, 2H), 3.67 (dd, J=7.5, 3.7 Hz, 2H), 2.68 (s, 2H), 1.84-1.68 (m, 2H), 1.48 (s, 9H), 1.38-1.24 (m, 2H), 1.17 (dddd, J=15.9, 8.5, 7.5, 5.5 Hz, 1H), 0.98 (tdd, J=11.2, 8.2, 4.9 Hz, 1H), 0.77-0.68 (m, 2H), 0.05-0.01 (m, 1H).

### Step 5: tert-butyl 4-((1R,2R)-2-formylcyclopropyl)piperidine-1-carboxylate

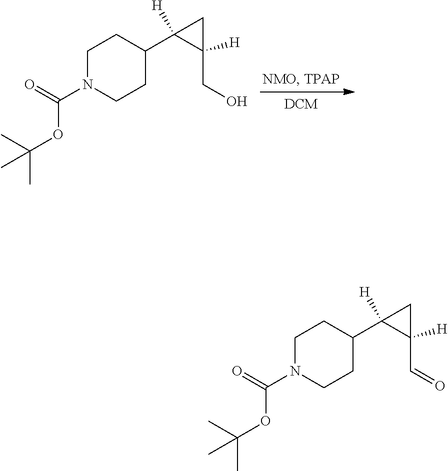 [SMILES: CC(C)(C)OC(=O)N1CCC([C@H]2C[C@H]2CO)CC1 ; CC(C)(C)OC(=O)N1CCC([C@H]2C[C@H]2C=O)CC1]

To a solution of tert-butyl 4-((1R,2R)-2-(hydroxymethyl)cyclopropyl)piperidine-1-carboxylate (2 g, 7.8 mmol) in DCM (40 mL) was treated with N-methyl-morpholine-N-oxide (2.8 g, 24 mmol) and the resulting mixture stirred at room temperature for 15 mins. The mixture was cooled to 0° C. and tetrapropylammonium perruthenate (28 mg, 0.078 mmol) and molecular sieves (2 g) added and the resulting mixture stirred at room temperature for 1 hour. The mixture was filtered and the filtrate washed with water (50 mL), DCM layer evaporated and the residue purified by silica gel column chromatography eluting with petroleum ether:ethyl acetate 3:1 to give tert-butyl 4-((1R,2R)-2-formylcyclopropyl)piperidine-1-carboxylate (1.6 g, 81%) as a yellow solid.

### Step 6: tert-butyl 4-((1R,2R)-2-(oxiran-2-yl)cyclopropyl)piperidine-1-carboxylate

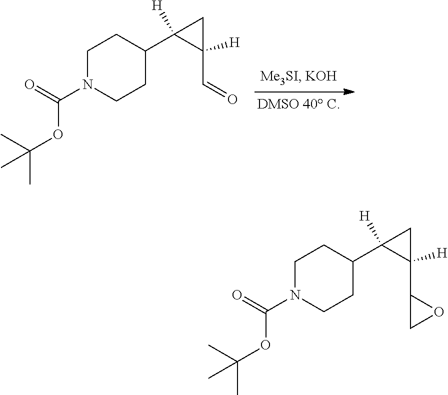 [SMILES: CC(C)(C)OC(=O)N1CCC([C@H]2C[C@H]2C=O)CC1 ; CC(C)(C)OC(=O)N1CCC([C@H]2C[C@H]2C2CO2)CC1]

To a solution of tert-butyl 4-((1R,2R)-2-formylcyclopropyl)piperidine-1-carboxylate (1.6 g, 6.3 mmol) and trimethylsulfonium iodide (1.8, 8.8 mmol) in DMSO (20 mL) was added KOH (0.5 g, 8.8 mmol) and the resulting mixture stirred at 40° C. for 3 hours. Water (30 mL) was added and extracted with EtOAc (2×40 mL); combined EtOAc layers dried over MgSO₄, filtered and evaporated. The residue was purified by silica gel column chromatography eluent: petroleum ether:ethyl acetate 5:1 to give tert-butyl 4-((1R,2R)-2-(oxiran-2-yl)cyclopropyl)piperidine-1-carboxylate (0.7 g, 41%) as a yellow solid. LCMS: tR=0.176, (ES⁺) m/z (M−55)⁺=212.1.

### Step 7: tert-butyl 4-((1R,2S)-2-(2-hydroxyethyl)cyclopropyl)piperidine-1-carboxylate

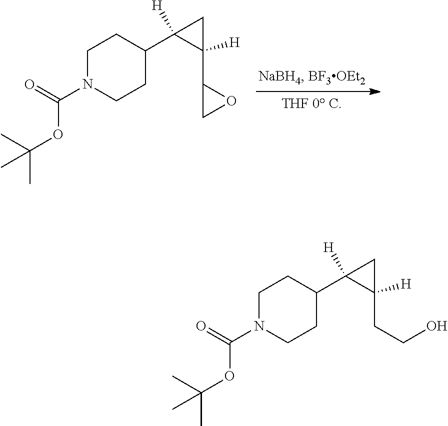 [SMILES: CC(C)(C)OC(=O)N1CCC([C@H]2C[C@H]2C2CO2)CC1 ; CC(C)(C)OC(=O)N1CCC([C@H]2C[C@H]2CCO)CC1]

To a solution of NaBH₄ (57 mg, 1.5 mmol) in THE (5 mL) was added BF₃·Et₂O (0.24 mL, 2.0 mmol) and the resulting mixture stirred at room temperature for 30 mins. The mixture was cooled to 0° C. and a solution of tert-butyl 4-((1R,2R)-2-(oxiran-2-yl)cyclopropyl)piperidine-1-carboxylate (0.8 g, 3.0 mmol) in THE (5 ml) added dropwise over 10 mins. After complete addition the mixture was stirred at room temperature for 3 hours. The mixture was quenched by the addition of water (30 mL) and extracted with EtOAc (2×40 mL); combined EtOAc layers washed with sat. NaCl (20 mL), dried over MgSO₄, filtered and evaporated. The residue was purified by silica gel column chromatography eluent petroleum ether: ethyl acetate 4:1 to give tert-butyl 4-((1R,2S)-2-(2-hydroxyethyl)cyclopropyl)piperidine-1-carboxylate (680 mg, 84%) as a yellow solid. ¹H NMR (400 MHz, MeOD-d) δ 4.22-4.16 (m, 2H), 3.81-3.77 (m, 2H), 2.75 (m, 1H), 1.96-1.88 (m, 3H), 1.60 (s, 9H), 1.44-1.37 (m, 3H), 1.20 (m, 1H), 1.00-0.98 (m, 1H) 0.86-0.70 (m, 2H), 0.01-0.02 (m, 1H).

### Step 8: tert-butyl 4-((1R,2S)-2-(2-(3-fluoro-4-(2-methoxy-2-oxoethyl)phenoxy)ethyl)cyclopropyl) piperidine-1-carboxylate

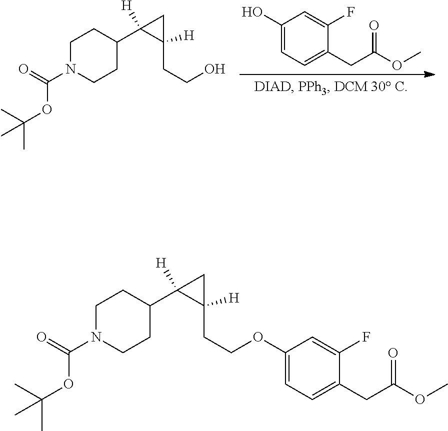 [SMILES: CC(C)(C)OC(=O)N1CCC([C@H]2C[C@H]2CCO)CC1 ; COC(=O)Cc1ccc(O)cc1F ; COC(=O)Cc1ccc(OCC[C@@H]2C[C@@H]2C2CCN(C(=O)OC(C)(C)C)CC2)cc1F]

To a mixture of tert-butyl 4-((1R,2S)-2-(2-hydroxyethyl)cyclopropyl)piperidine-1-carboxylate (680 mg, 2.5 mmol) and methyl (2-fluoro-4-hydroxy-phenyl)acetate (470 mg, 2.5 mmol) in DCM (60 mL) was added di-isopropylazodicarboxylate (0.74 mL, 3.8 mmol) and triphenylphosphine (990 mg, 3.8 mmol) and the resulting mixture stirred at 30° C. for 12 hours. The mixture was quenched by the addition of water (50 mL) and extracted with EtOAc (2×70 mL); combined EtOAc layers washed with sat. NaCl (30 mL), dried over MgSO₄, filtered and evaporated. The residue was purified by silica gel column chromatography eluent petroleum ether: ethyl acetate 5:1 to give tert-butyl 4-((1R,2S)-2-(2-(3-fluoro-4-(2-methoxy-2-oxoethyl)phenoxy)ethyl)cyclopropyl) piperidine-1-carboxylate (740 mg, 66%) as a yellow solid. LCMS: tR=1.121, (ES⁺) m/z (M−55)⁺=380.2.

### Step 9: methyl 2-(2-fluoro-4-(2-((1S,2R)-2-(1-(5-(methoxymethyl)pyrimidin-2-yl)piperidin-4-yl)cyclopropyl)ethoxy)phenyl)acetate

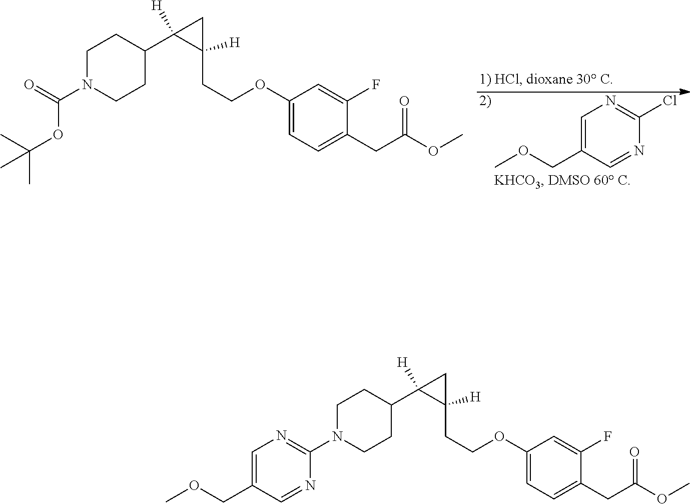

A mixture of tert-butyl 4-((1R,2S)-2-(2-(3-fluoro-4-(2-methoxy-2-oxoethyl)phenoxy)ethyl)cyclopropyl) piperidine-1-carboxylate (730 mg, 1.7 mmol) and 4M HCl in dioxane (20 mL) was stirred at 30° C. for 3 hours and then evaporated. The residue was mixed with 2-chloro-5-(methoxymethyl)pyrimidine (270 mg, 1.7 mmol), KHCO₃ (330 mg, 3.3 mmol) in DMSO (30 mL) and heated at 60° C. for 12 hours. The cooled mixture was treated with water (50 mL) and extracted with EtOAc (2×80 mL); combined EtOAc layers washed with sat. NaCl (20 mL), dried over MgSO₄, filtered and evaporated. The residue was purified by silica gel column chromatography eluent petroleum ether:ethyl acetate 3:1 to give methyl 2-(2-fluoro-4-(2-((1S,2R)-2-(1-(5-(methoxymethyl)pyrimidin-2-yl)piperidin-4-yl)cyclopropyl)ethoxy)phenyl)acetate 680 mg, 89%) as a yellow solid. ¹H NMR (400 MHz, MeOD-d) δ 8.30 (d, J=4.0 Hz, 2H), 7.23-7.19 (m, 1H), 6.76-670 (m, 2H), 4.76-4.73 (m, 2H), 4.31 (s, 2H), 4.11-4.09 (m, 2H), 3.72 (s, 3H), 3.65 (s, 2H), 3.47 (s, 3H), 2.95-2.93 (m, 2H), 2.16-2.14 (m, 1H), 1.89-1.86 (m, 2H), 1.62 (m, 1H), 1.39-1.23 (m, 3H), 1.02-1.00 (m, 1H) 0.72-0.65 (m, 2H), 0.01-0.01 (m, 1H). LCMS: tR=1.014, (ES⁺) m/z (M+H)⁺=458.2.

### Step 10: 2-(2-fluoro-4-(2-((1S,2R)-2-(1-(5-(methoxymethyl)pyrimidin-2-yl)piperidin-4-yl)cyclopropyl)ethoxy) phenyl)acetic acid

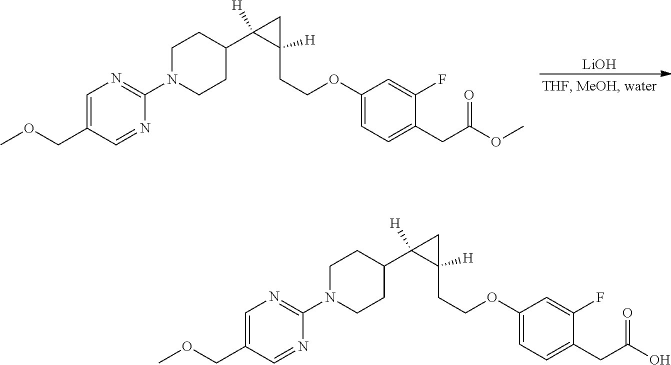

To a mixture of methyl 2-(2-fluoro-4-(2-((1S,2R)-2-(1-(5-(methoxymethyl)pyrimidin-2-yl)piperidin-4-yl)cyclopropyl)ethoxy)phenyl)acetate (300 mg, 0.66 mmol) in THF (10 mL), MeOH (10 mL) and water (10 mL) was added lithium hydroxide monohydrate (55 mg, 1.3 mmol) and stirred at 30° C. for 5 hours. Mixture evaporated to remove organic solvents and diluted with water (20 ml) and extracted with EtOAc (2×20 mL). The aqueous layer was acidified to pH ˜2 by the addition of HCl and extracted with EtOAc (2×20 mL). The combined EtOAc layers were washed with sat. NaCl (20 mL0, dried over MgSO₄, filtered and evaporated to give 2-(2-fluoro-4-(2-((1S,2R)-2-(1-(5-(methoxymethyl)pyrimidin-2-yl)piperidin-4-yl)cyclopropyl)ethoxy) phenyl)acetic acid (200 mg, 68%) as a yellow solid. LCMS: tR=1.85, (ES⁺) m/z (M+H)⁺=444.2.

### Intermediate 19: 2-(6-(3-(1-(5-chloropyrimidin-2-yl)piperidin-4-yl)propoxy)pyridin-3-yl)acetic acid

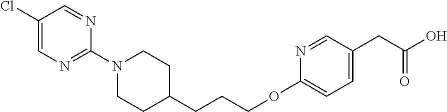 [SMILES: O=C(O)Cc1ccc(OCCCC2CCN(c3ncc(Cl)cn3)CC2)nc1]

### Step 1: 2-(4-(3-((5-bromopyridin-2-yl)oxy)propyl)piperidin-1-yl)-5-chloropyrimidine

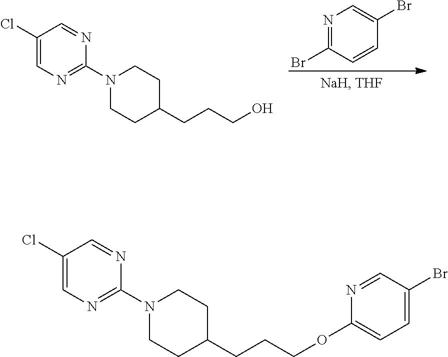 [SMILES: OCCCC1CCN(c2ncc(Cl)cn2)CC1 ; Brc1ccc(Br)nc1 ; Clc1cnc(N2CCC(CCCOc3ccc(Br)cn3)CC2)nc1]

To a solution of 3-[1-(5-chloropyrimidin-2-yl)-4-piperidyl]propan-1-ol (Intermediate 1 step 1, 1 g, 3.91 mmol) in THF (20 mL) was added sodium hydride (188 mg of a 60% dispersion in oil, 4.69 mmol) and the resulting mixture stirred at room temperature for 30 mins then a solution of 2,5-dibromo-pyrimidine (926 mg, 3.91 mmol) in THF (5 mL) added and the resulting mixture stirred at 50° C. overnight. The mixture was cooled and quenched by the addition of water (50 mL) and extracted with EtOAc (50 mL), the organic layer washed with sat. NaCl (20 mL), dried over Na₂SO₄, filtered and evaporated. The residue was purified by silica gel column chromatography (Teledyne Isco: SNAP 40 g GOLD) eluent: gradient 0-20% EtOAc in Heptane to give 2-(4-(3-((5-bromopyridin-2-yl)oxy)propyl)piperidin-1-yl)-5-chloropyrimidine (340 mg, 21%) as a white solid. LCMS: tR=2.30, (ES⁺) m/z (M+H)⁺=413.0.

### Step 2: tert-butyl 2-(6-(3-(1-(5-chloropyrimidin-2-yl)piperidin-4-yl)propoxy)pyridin-3-yl)acetate

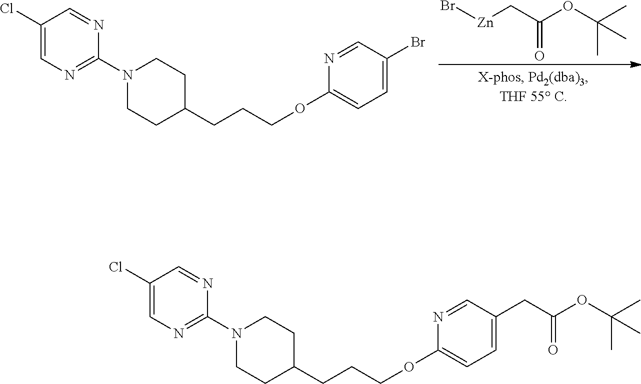

A mixture of 2-[4-[3-[(5-bromo-2-pyridyl)oxy]propyl]-1-piperidyl]-5-chloro-pyrimidine (330 mg, 0.802 mmol) and 2-tert-butoxy-2-oxoethyline bromide (9.6 mL of a 0.5M solution in ether, 4.81 mmol) in THF (10 mL) was degassed by bubbling nitrogen gas through for 10 mins then Pd₂(dba)₃ (36 mg, 0.04 mmol) and X-phos (38 mg, 0.080 mmol) added and de-gassing continued for 10 mins. Mixture heated at 55° C. overnight. Mixture cooled to room temp and quenched by the addition of MeOH (3 mL) and evaporated. The residue purified by silica gel column chromatography (Teledyne Isco: SNAP 24g GOLD) eluent: gradient 0-20% EtOAc in Heptane to give tert-butyl 2-(6-(3-(1-(5-chloropyrimidin-2-yl)piperidin-4-yl)propoxy)pyridin-3-yl)acetate (200 mg, 55%) as a colorless oil. LCMS: tR=2.22, (ES⁺) m/z (M+H)⁺=447.3.

### Step 3: 2-(6-(3-(1-(5-chloropyrimidin-2-yl)piperidin-4-yl)propoxy)pyridin-3-yl)acetic acid hydrochloride

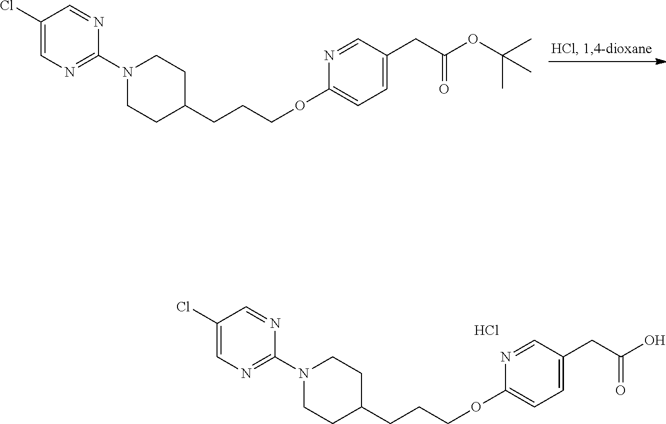 [SMILES: CC(C)(C)OC(=O)Cc1ccc(OCCCC2CCN(c3ncc(Cl)cn3)CC2)nc1 ; O=C(O)Cc1ccc(OCCCC2CCN(c3ncc(Cl)cn3)CC2)nc1 ; Cl]

To tert-butyl 2-[6-[3-[1-(5-chloropyrimidin-2-yl)-4-piperidyl]propoxy]-3-pyridyl]acetate (200 mg, 0.448 mmol) was added hydrogen chloride (4.5 mL of a 4M solution in 1,4-dioxane, 17.9 mmol) and the resulting mixture stirred at room temperature for 2 hours. Mixture evaporated and the residue triturated with diethyl ether, filtered and dried to give 2-(6-(3-(1-(5-chloropyrimidin-2-yl)piperidin-4-yl)propoxy)pyridin-3-yl)acetic acid hydrochloride (155 mg, 88%) as an off white solid. ¹H NMR (500 MHz, DMSO-d₆) δ 8.38 (s, 2H), 8.07 (d, J=2.4 Hz, 1H), 7.76 (dd, J=8.6, 2.4 Hz, 1H), 6.93 (d, J=8.6 Hz, 1H), 4.58 (dq, J=13.2, 2.7, 2.3 Hz, 2H), 4.27 (t, J=6.6 Hz, 2H), 2.88 (td, J=12.9, 2.7 Hz, 2H), 1.76 (ddd, J=12.0, 9.6, 5.1 Hz, 4H), 1.57 (ddp, J=11.0, 7.1, 3.5 Hz, 1H), 1.39-1.29 (m, 2H), 1.05 (qd, J=12.5, 4.1 Hz, 2H). LCMS: tR=1.48, (ES⁺) m/z (M+H)⁺=391.2.

### Intermediate 20: 2-(2-fluoro-4-((2-(1-(5-(methoxymethyl)pyrimidin-2-yl)piperidin-4-yl)ethoxy)methyl)phenyl)acetic acid

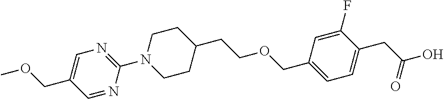 [SMILES: COCc1cnc(N2CCC(CCOCc3ccc(CC(=O)O)c(F)c3)CC2)nc1]

### Step 1: 2-(1-(5-(methoxymethyl)pyrimidin-2-yl)piperidin-4-yl)ethan-1-ol

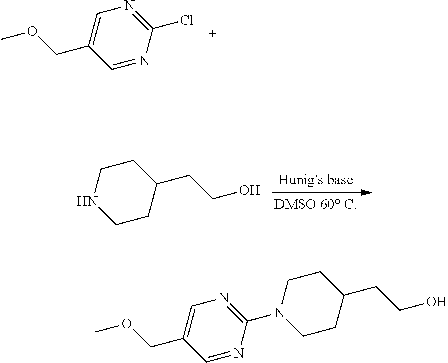

A mixture of 2-chloro-5-(methoxymethyl)pyrimidine (385 mg, 2.43 mmol) and 4-piperidineethanol (314 mg, 2.43 mmol) in DMSO (3 mL) was heated at 60° C. overnight. Mixture cooled and poured into water (30 mL) and extracted with EtOAc (3×15 mL); combined EtOAc layers washed with sat. NaCl (25 mL), dried over Na₂SO₄, filtered and evaporated. The residue was purified by silica gel column chromatography (Teledyne Isco: SNAP 24g GOLD) eluent: gradient 50-100% EtOAc in Heptane then hold 100% EtOAc to give 2-(1-(5-(methoxymethyl)pyrimidin-2-yl)piperidin-4-yl)ethan-1-ol (575 mg, 94%) as a clear oil.

### Step 2: 2-(4-(2-((4-bromo-3-fluorobenzyl)oxy)ethyl)piperidin-1-yl)-5-(methoxymethyl)pyrimidine

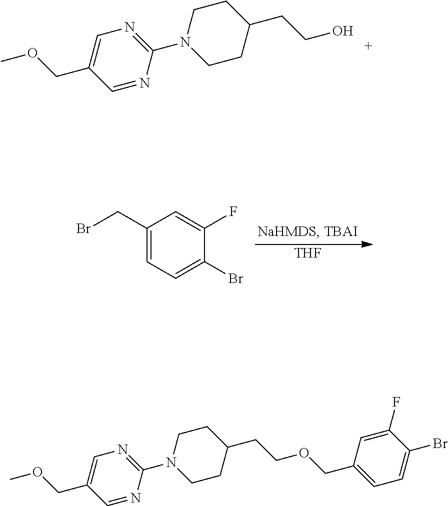 [SMILES: COCc1cnc(N2CCC(CCO)CC2)nc1 ; Fc1cc(CBr)ccc1Br ; COCc1cnc(N2CCC(CCOCc3ccc(Br)c(F)c3)CC2)nc1 ; "THF"]

To a solution of 2-[1-[5-(methoxymethyl)pyrimidin-2-yl]-4-piperidyl]ethanol (575 mg, 2.29 mmol) in THF (15 mL) cooled at −78° C. was added slowly NaHMDS (2.5 mL of a 1M soln in THF, 2.5 mmol), after complete addition mixture stirred at −78° C. for 30 mins then 4-bromo-3-fluorobenzyl bromide (796 mg, 2.97 mmol) and tetrabutylammonium iodide (93 mg, 0.25 mmol) and the resulting mixture allowed to warm to room temperature and stirred for 3 days (weekend). Mixture quenched by the addition of sat. NH₄Cl (50 mL) and mixture extracted with EtOAc (3×20 mL); combined EtOAc layers washed with sat. NaCl (25 mL), dried over Na₂SO₄ filtered and evaporated. The residue was purified by silica gel column chromatography (Teledyne Isco: SNAP 24 g GOLD) eluent: gradient 0-100% EtOAc in Heptane to give 2-(4-(2-((4-bromo-3-fluorobenzyl)oxy)ethyl)piperidin-1-yl)-5-(methoxymethyl)pyrimidine (560 mg, 55%) as a clear oil. LCMS: tR=1.23, (ES⁺) m/z (M+H)⁺=438.2/440.2.

### Step 3: tert-butyl 2-(2-fluoro-4-((2-(1-(5-(methoxymethyl)pyrimidin-2-yl)piperidin-4-yl)ethoxy)methyl) phenyl)acetate

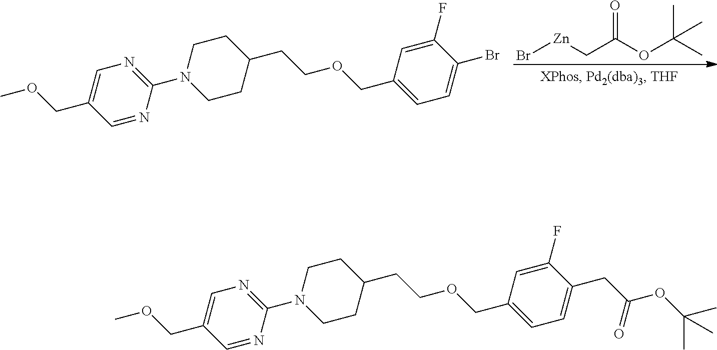

To a solution of 2-[4-[2-[(4-bromo-3-fluoro-phenyl)methoxy]ethyl]-1-piperidyl]-5-(methoxymethyl) pyrimidine (560 mg, 1.28 mmol) in THF (5 mL) was added 2-tert butoxy-2-oxoethylzinc bromide (7.7 mL of a 0.5M soln in Et₂O, 3.83 mmol) and followed by Xphos (61 mg, 0.128 mmol) and Pd₂(dba)₃ (58 mg, 0.064 mmol) and the resulting mixture de-gassed by bubbling nitrogen gas through for 15 mins and the resulting mixture heated at 50° C. overnight. Mixture evaporated and the residue purified by silica gel column chromatography (Teledyne Isco: SNAP 24g GOLD) eluent: gradient 0-100% EtOAc in Heptane to give tert-butyl 2-(2-fluoro-4-((2-(1-(5-(methoxymethyl)pyrimidin-2-yl)piperidin-4-yl)ethoxy)methyl) phenyl)acetate (507 mg, 83%) as a clear oil. LCMS: tR=1.33, (ES⁺) m/z (M+H)⁺=474.4.

### Step 4: 2-(2-fluoro-4-((2-(1-(5-(methoxymethyl)pyrimidin-2-yl)piperidin-4-yl)ethoxy)methyl)phenyl) acetic acid

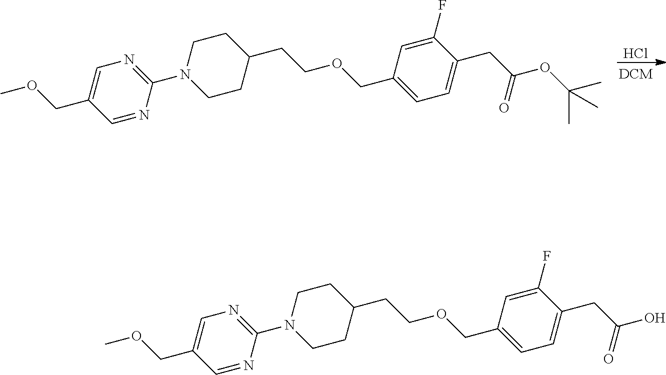

To a solution of tert-butyl 2-[2-fluoro-4-[2-[1-[5-(methoxymethyl)pyrimidin-2-yl]-4-piperidyl] ethoxymethyl]phenyl]acetate (507 mg, 1.07 mmol) in DCM (5 mL) was added hydrogen chloride (2.7 mL of a 4M soln in 1,4-dioxane, 10.7 mmol) and the resulting mixture heated at 35° C. for 4 hours. Mixture evaporated to give 2-(2-fluoro-4-((2-(1-(5-(methoxy methyl)pyrimidin-2-yl)piperidin-4-yl)ethoxy) methyl)phenyl) acetic acid (450 mg, 100%) as a yellow oil. LCMS: tR=0.59, (ES⁺) m/z (M+H)⁺=418.3.

### Intermediate 21: 2-(2-fluoro-4-(3-(1-(((3-(trifluoromethyl)oxetan-3-yl)oxy)carbonyl)piperidin-4-yl)propoxy)phenyl)acetic acid

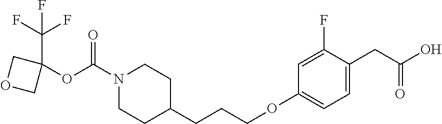 [SMILES: O=C(O)Cc1ccc(OCCCC2CCN(C(=O)OC3(C(F)(F)F)COC3)CC2)cc1F]

### Step 1: perfluorophenyl (3-(trifluoromethyl)oxetan-3-yl) carbonate

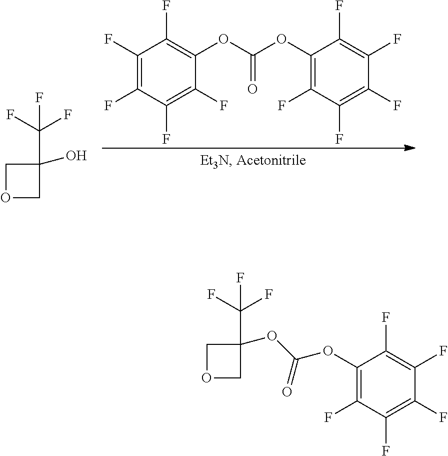

To a solution of 3-(trifluoromethyl)oxetan-3-ol (500 mg, 3.52 mmol) and bis(pentafluorophenyl)carbonate (1.66 g, 4.22 mmol) in acetonitrile (4 mL) was added dropwise triethylamine (1.47 mL, 10.56 mmol) and the resulting mixture stirred at room temperature overnight. Mixture evaporated and the residue purified by silica gel column chromatography (Teledyne Isco: SNAP 24g GOLD) eluent: gradient 0-100% EtOAc in Heptane to give perfluorophenyl (3-(trifluoromethyl)oxetan-3-yl) carbonate (1.05 g, 84%) as a colorless oil used as such in the subsequent step.

### Step 2: 3-(trifluoromethyl)oxetan-3-yl 4-(3-hydroxypropyl)piperidine-1-carboxylate

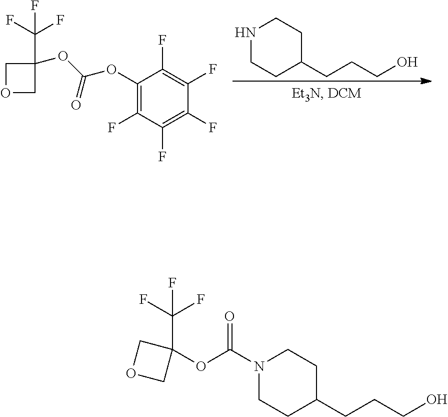

A mixture of (2,3,4,5,6-pentafluorophenyl) [3-(trifluoromethyl)oxetan-3-yl]carbonate (1.05 g, 2.98 mmol) and 4-piperidinepropanol (427 mg, 2.98 mmol) in DCM (10 mL) was treated with triethylamine 0.83 mL, 5.96 mmol) and the resulting mixture stirred at room temperature overnight. Mixture diluted with DCM (20 mL) and washed with water (30 mL), sat. NaCl (15 mL), dried over Na₂SO₄, filtered and evaporated. The residue was purified by silica gel column chromatography (Teledyne Isco: SNAP 24g GOLD) eluent: gradient 0-100% EtOAc in Heptane to give 3-(trifluoromethyl)oxetan-3-yl 4-(3-hydroxypropyl)piperidine-1-carboxylate (530 mg, 57%) as a colorless oil. ¹H NMR (500 MHz, Chloroform-d) δ 4.99 (t, J=9.0 Hz, 2H), 4.87-4.81 (m, 2H), 4.08 (dd, J=33.3, 13.4 Hz, 2H), 3.65 (t, J=6.7 Hz, 2H), 2.86 (td, J=13.0, 2.7 Hz, 1H), 2.75 (td, J=12.9, 2.8 Hz, 1H), 1.80-1.69 (m, 2H), 1.62-1.56 (m, 2H), 1.45 (tdt, J=13.8, 7.0, 3.7 Hz, 1H), 1.38-1.29 (m, 2H), 1.15 (qd, J=12.6, 10.1, 4.4 Hz, 2H).

### Step 3: 3-(trifluoromethyl)oxetan-3-yl 4-(3-(3-fluoro-4-(2-methoxy-2-oxoethyl)phenoxy)propyl) piperidine-1-carboxylate

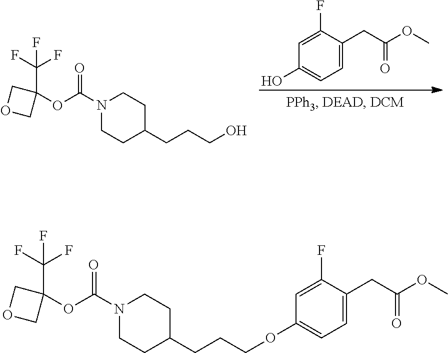 [SMILES: O=C(OC1(C(F)(F)F)COC1)N1CCC(CCCO)CC1 ; COC(=O)Cc1ccc(O)cc1F ; COC(=O)Cc1ccc(OCCCC2CCN(C(=O)OC3(C(F)(F)F)COC3)CC2)cc1F]

Prepared using procedures outlined in the preparation of intermediate 1 (step 2); replacing 3-(1-(5-chloropyrimidin-2-yl)piperidin-4-yl)propan-1-ol with 3-(trifluoromethyl)oxetan-3-yl 4-(3-hydroxypropyl)piperidine-1-carboxylate to give 3-(trifluoromethyl)oxetan-3-yl 4-(3-(3-fluoro-4-(2-methoxy-2-oxoethyl)phenoxy)propyl)piperidine-1-carboxylate. ¹H NMR (500 MHz, Chloroform-d) δ 7.14 (t, J=8.6 Hz, 1H), 6.66-6.63 (m, 1H), 6.61 (dd, J=11.5, 2.5 Hz, 1H), 5.00 (t, J=8.7 Hz, 2H), 4.89-4.81 (m, 2H), 4.17-4.02 (m, 2H), 3.92 (t, J=6.4 Hz, 2H), 3.70 (s, 3H), 3.61-3.59 (m, 2H), 2.88 (t, J=13.0 Hz, 1H), 2.76 (t, J=12.5 Hz, 1H), 1.86-1.70 (m, 4H), 1.49 (dtd, J=14.3, 7.4, 3.5 Hz, 1H), 1.46-1.39 (m, 2H), 1.17 (q, J=12.6 Hz, 2H). LCMS: tR=1.29, (ES⁺) m/z (M+H)⁺=478.3.

### Step 4: 2-(2-fluoro-4-(3-(1-(((3-(trifluoromethyl)oxetan-3-yl)oxy)carbonyl)piperidin-4-yl)propoxy) phenyl)acetic acid

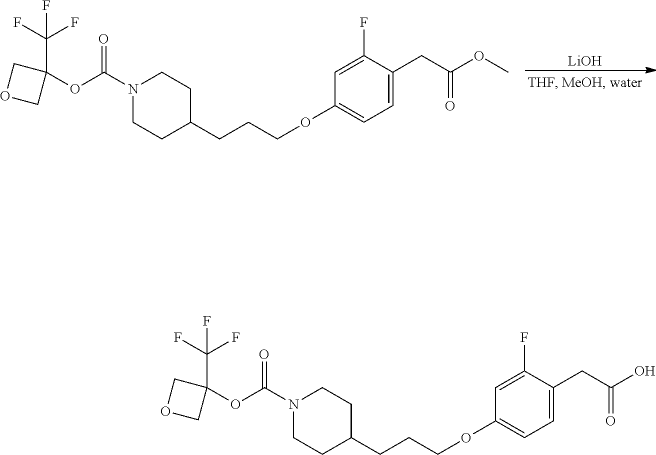 [SMILES: COC(=O)Cc1ccc(OCCCC2CCN(C(=O)OC3(C(F)(F)F)COC3)CC2)cc1F ; "LiOH" ; O=C(O)Cc1ccc(OCCCC2CCN(C(=O)OC3(C(F)(F)F)COC3)CC2)cc1F]

Prepared using procedure outlined in the preparation of intermediate 1 (step 3); replacing 2-(4-(3-(1-(5-chloropyrimidin-2-yl)piperidin-4-yl)propoxy)-2-fluorophenyl)acetate with 3-(trifluoromethyl)oxetan-3-yl 4-(3-(3-fluoro-4-(2-methoxy-2-oxoethyl)phenoxy)propyl) piperidine-1-carboxylate to give 2-(2-fluoro-4-(3-(1-(((3-(trifluoromethyl)oxetan-3-yl)oxy)carbonyl)piperidin-4-yl)propoxy) phenyl)acetic acid. LCMS: tR=0.98, (ES⁺) m/z (M+H)⁺=464.3.

### Intermediate 22: 2-(2,6-difluoro-4-(3-(1-(((3-(trifluoromethyl)oxetan-3-yl)oxy)carbonyl)piperidin-4-yl)propoxy) phenyl)acetic acid

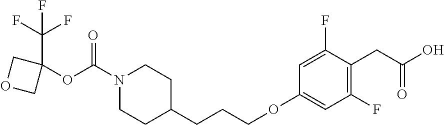 [SMILES: O=C(O)Cc1c(F)cc(OCCCC2CCN(C(=O)OC3(C(F)(F)F)COC3)CC2)cc1F]

Prepared using procedures outlined in the preparation of intermediate 21 replacing methyl 2-fluoro-4-hydroxyphenyl acetate with methyl 2-(2,6-difluoro-4-hydroxyphenyl)acetate in step 3 to give 2-(2,6-difluoro-4-(3-(1-(((3-(trifluoromethyl)oxetan-3-yl)oxy)carbonyl)piperidin-4-yl)propoxy)phenyl)acetic acid. LCMS: tR=1.05, (ES⁺) m/z (M+H)⁺=482.3.

### Intermediate 23: 2-(2-fluoro-4-(3-(1-(isopropoxycarbonyl)piperidin-4-yl)propoxy)phenyl)acetic acid

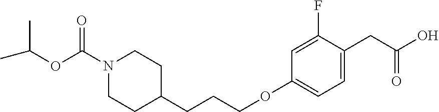 [SMILES: CC(C)OC(=O)N1CCC(CCCOc2ccc(CC(=O)O)c(F)c2)CC1]

### Step 1: isopropyl 4-(3-hydroxypropyl)piperidine-1-carboxylate

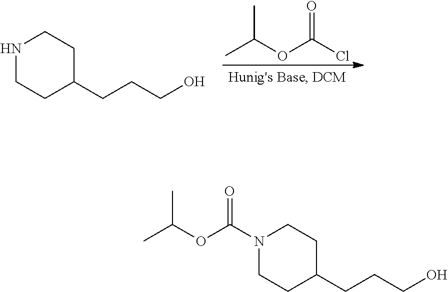

To a solution of 4-piperidinepropanol (300 mg, 2.09 mmol) and Hunig's base (0.73 mL, 4.19 mmol) in DCM (10 mL) was added isopropylchloroformate (1.05 mL of a 2M soln in xylenes, 2.09 mmol) and the resulting mixture stirred at room temperature overnight. Mixture diluted with DCM (20 mL) and washed with water (30 mL), sat. NaCl (20 mL), dried over Na₂SO₄, filtered and evaporated. The residue purified by silica gel column chromatography (Teledyne Isco: SNAP 24g GOLD) eluent: gradient 0-100% EtOAc in Heptane to give 458 mg (Yield 95%).

### Step 2: isopropyl 4-(3-(3-fluoro-4-(2-methoxy-2-oxoethyl)phenoxy)propyl)piperidine-1-carboxylate

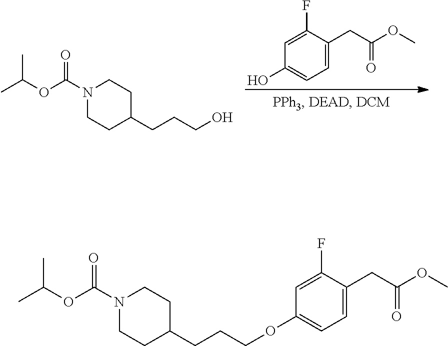 [SMILES: CC(C)OC(=O)N1CCC(CCCO)CC1 ; COC(=O)Cc1ccc(O)cc1F ; COC(=O)Cc1ccc(OCCCC2CCN(C(=O)OC(C)C)CC2)cc1F]

Prepared using procedures outlined in the preparation of intermediate 1 (step 2); replacing 3-(1-(5-chloropyrimidin-2-yl)piperidin-4-yl)propan-1-ol with isopropyl 4-(3-hydroxypropyl)piperidine-1-carboxylate to give isopropyl 4-(3-(3-fluoro-4-(2-methoxy-2-oxoethyl)phenoxy)propyl)piperidine-1-carboxylate ¹H NMR (500 MHz, Chloroform-d) δ 7.13 (t, J=8.6 Hz, 1H), 6.66-6.63 (m, 1H), 6.61 (dd, J=11.6, 2.5 Hz, 1H), 4.91 (h, J=6.3 Hz, 1H), 4.12 (m, 2H), 3.92 (t, J=6.4 Hz, 2H), 3.70 (s, 3H), 3.61-3.58 (m, 2H), 2.71 (t, J=12.6 Hz, 2H), 1.84-1.76 (m, 2H), 1.69 (d, J=13.1 Hz, 2H), 1.50-1.36 (m, 3H), 1.24 (d, J=6.3 Hz, 6H), 1.12 (dd, J=12.0, 4.1 Hz, 2H). LCMS: tR=1.27, (ES⁺) m/z (M+H)⁺=396.4.

### Step 3: 2-(2-fluoro-4-(3-(1-(isopropoxycarbonyl)piperidin-4-yl)propoxy)phenyl)acetic acid

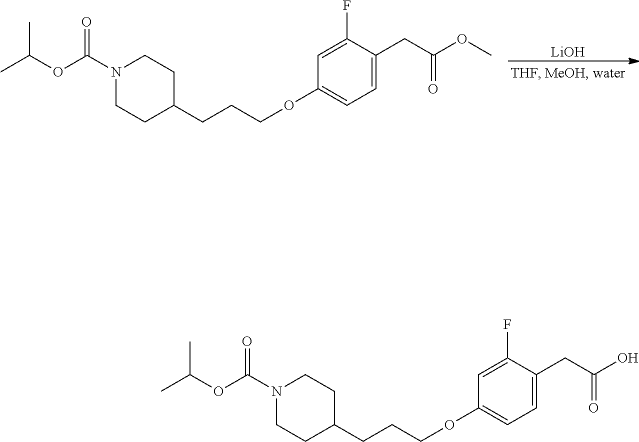

Prepared using procedures outlined in the preparation of intermediate 1 (step 3); replacing 2-(4-(3-(1-(5-chloropyrimidin-2-yl)piperidin-4-yl)propoxy)-2-fluorophenyl)acetate with isopropyl 4-(3-(3-fluoro-4-(2-methoxy-2-oxoethyl)phenoxy)propyl)piperidine-1-carboxylate to give 2-(2-fluoro-4-(3-(1-(isopropoxycarbonyl)piperidin-4-yl)propoxy)phenyl)acetic acid. ¹H NMR (500 MHz, Chloroform-d) δ 7.14 (t, J=8.5 Hz, 1H), 6.65 (dd, J=8.5, 2.8 Hz, 1H), 6.61 (dd, J=11.5, 2.5 Hz, 1H), 4.91 (p, J=6.2 Hz, 1H), 4.15 (m, 2H), 3.91 (t, J=6.4 Hz, 2H), 3.63 (d, J=1.2 Hz, 2H), 2.71 (t, J=12.8 Hz, 2H), 1.83-1.75 (m, 2H), 1.69 (d, J=13.1 Hz, 2H), 1.51-1.35 (m, 3H), 1.24 (d, J=6.2 Hz, 6H), 1.17-1.06 (m, 2H). LCMS: tR=0.95, (ES⁺) m/z (M+H)⁺=382.4.

### Intermediate 24: 2-(4-(2-(7-(5-chloropyrimidin-2-yl)-7-azaspiro[3.5]nonan-2-yl)ethoxy)-2-fluorophenyl)acetic acid

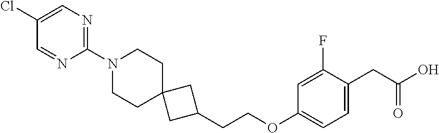 [SMILES: O=C(O)Cc1ccc(OCCC2CC3(CCN(c4ncc(Cl)cn4)CC3)C2)cc1F]

### Step 1: tert-butyl 2-(2-ethoxy-2-oxoethylidene)-7-azaspiro[3.5]nonane-7-carboxylate

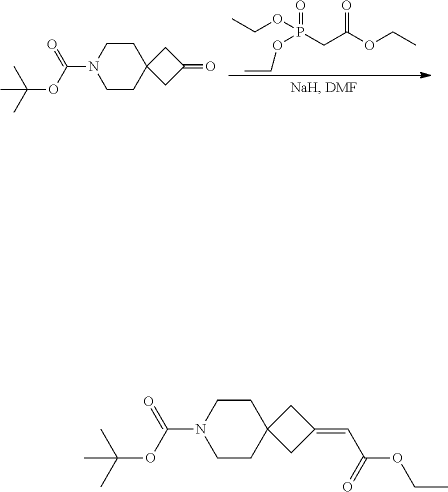

To a solution of triethyl phosphonoacetate (3.7 mL, 18.8 mmol) in DMF (50 mL) cooled in an ice bath was added sodium hydride (752 mg of a 60% dispersion, 18.8 mmol) and the resulting mixture stirred at ice bath temperature for 30 minutes after which a solution of tert-butyl 2-oxo-7-azaspiro[3.5]nonane-7-carboxylate (3 g, 12.5 mmol) in DMF (15 mL) was added. The mixture was then allowed to warm to room temperature and stirred for 5 hours. Mixture quenched by the addition of sat. NH₄Cl (100 mL) and diluted with water (100 mL) then extracted with EtOAc (2×50 mL), combined EtOAc layers washed with water (100 mL), sat. NaCl (50 mL), dried over Na₂SO₄, filtered and evaporated. The residue was purified by silica gel column chromatography (Teledyne Isco: SNAP 40 g GOLD) eluent: gradient 0-50% EtOAc in Heptane to give tert-butyl 2-(2-ethoxy-2-oxoethylidene)-7-azaspiro[3.5]nonane-7-carboxylate (2.99 g, 77%) as a colorless oil. ¹H NMR (500 MHz, Chloroform-d) δ 5.70 (q, J=2.3 Hz, 1H), 4.15 (q, J=7.1 Hz, 2H), 3.41-3.27 (m, 4H), 2.88 (dq, J=3.0, 1.6 Hz, 2H), 2.57 (t, J=1.8 Hz, 2H), 1.62-1.55 (m, 4H), 1.46 (s, 9H), 1.27 (t, J=7.1 Hz, 3H).

### Step 2: tert-butyl 2-(2-ethoxy-2-oxoethyl)-7-azaspiro[3.5]nonane-7-carboxylate

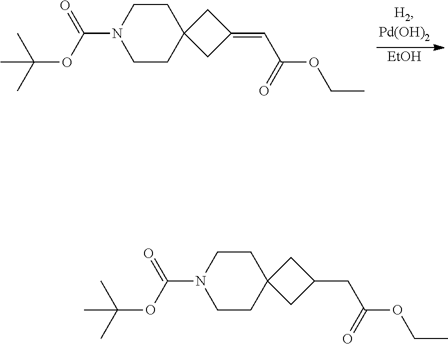

To a nitrogen flushed solution of tert-butyl 2-(2-ethoxy-2-oxo-ethylidene)-7-azaspiro[3.5]nonane-7-carboxylate (2.99 g, 9.66 mmol) in EtOH (50 mL) was added palladium hydroxide (475 mg) and the resulting mixture stirred under a balloon of hydrogen overnight. Mixture filtered through celite and the filtrate evaporated to give tert-butyl 2-(2-ethoxy-2-oxoethyl)-7-azaspiro[3.5]nonane-7-carboxylate (2.98 g, 99%) as a colorless oil. ¹H NMR (500 MHz, Chloroform-d) δ 4.11 (q, J=7.1 Hz, 2H), 3.36-3.32 (m, 2H), 3.28-3.23 (m, 2H), 2.69-2.58 (m, 1H), 2.41 (d, J=7.6 Hz, 2H), 2.07-2.00 (m, 2H), 1.57 (t, J=5.4 Hz, 2H), 1.44 (s, 12H), 1.25 (t, J=7.1 Hz, 3H).

### Step 3: tert-butyl 2-(2-hydroxyethyl)-7-azaspiro[3.5]nonane-7-carboxylate

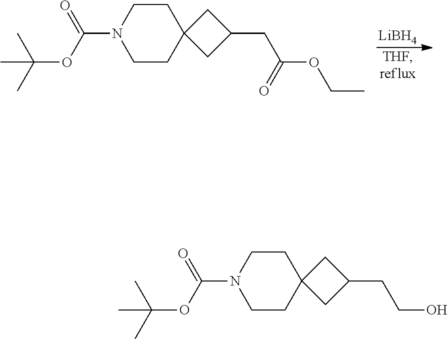

To a solution of tert-butyl 2-(2-ethoxy-2-oxo-ethyl)-7-azaspiro[3.5]nonane-7-carboxylate (N32-58, 2.98 g, 9.57 mmol) in THF (50 mL) was added lithium borohydride (792 mg, 36.36 mmol) and the resulting mixture heated at reflux for 5 hours. Mixture quenched with water (100 mL) under ice-bath cooling and extracted with EtOAc (2×30 mL); combined EtOAc layers washed with sat. NaCl (30 mL), dried over Na₂SO₄, filtered and evaporated to give tert-butyl 2-(2-hydroxyethyl)-7-azaspiro[3.5]nonane-7-carboxylate (2.6 g, 100%) as a colorless oil. ¹H NMR (500 MHz, Chloroform-d) δ 3.59 (td, J=5.9, 3.4 Hz, 2H), 3.37-3.31 (m, 2H), 3.29-3.21 (m, 2H), 2.40-2.29 (m, 1H), 1.98 (ddd, J=9.8, 8.5, 2.5 Hz, 2H), 1.73-1.65 (m, 3H), 1.59-1.53 (m, 2H), 1.45 (s, 11H).

### Step 4: 2-(7-azaspiro[3.5]nonan-2-yl)ethan-1-ol hydrochloride

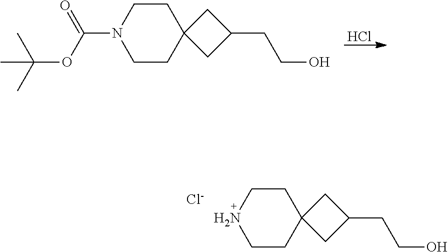 [SMILES: CC(C)(C)OC(=O)N1CCC2(CC1)CC(CCO)C2 ; Cl ; [Cl-] ; OCCC1CC2(CCNCC2)C1]

A mixture of tert-butyl 2-(2-hydroxyethyl)-7-azaspiro[3.5]nonane-7-carboxylate (N32-61-1, 2.6 g, 9.65 mmol) was treated with hydrogen chloride (48 mL of a 4M solution in dioxane, 193 mmol) and stirred for 12 hours. Mixture evaporated to give 2-(7-azaspiro[3.5]nonan-2-yl)ethan-1-ol hydrochloride (1.94 g, 97%) as a white solid. ¹H NMR (500 MHz, DMSO-d₆) δ 8.75 (s, 2H), 4.33 (s, 1H), 3.31 (t, J=6.6 Hz, 2H), 2.96 (dq, J=9.0, 5.2 Hz, 2H), 2.87 (dq, J=8.3, 4.6 Hz, 2H), 2.27 (p, J=8.1 Hz, 1H), 1.98-1.89 (m, 2H), 1.76-1.70 (m, 2H), 1.62 (dd, J=6.6, 4.9 Hz, 2H), 1.53 (q, J=6.8 Hz, 2H), 1.44-1.36 (m, 2H).

### Step 5: 2-(7-(5-chloropyrimidin-2-yl)-7-azaspiro[3.5]nonan-2-yl)ethan-1-ol

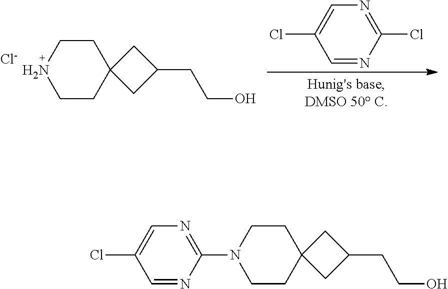 [SMILES: [Cl-] ; OCCC1CC2(CCNCC2)C1 ; Clc1cnc(Cl)nc1 ; OCCC1CC2(CCN(c3ncc(Cl)cn3)CC2)C1]

Prepared using procedures outlined in the preparation of intermediate 1 (step 1); replacing 3-(piperidin-4-yl)propan-1-ol with 2-(7-azaspiro[3.5]nonan-2-yl)ethan-1-ol hydrochloride to give 2-(7-(5-chloropyrimidin-2-yl)-7-azaspiro[3.5]nonan-2-yl)ethan-1-ol. ¹H NMR (500 MHz, Chloroform-d) δ 8.15 (s, 2H), 3.72-3.66 (m, 2H), 3.65-3.59 (m, 2H), 3.53 (t, J=6.6 Hz, 2H), 2.32 (ddd, J=16.0, 8.5, 7.5 Hz, 1H), 2.01-1.92 (m, 2H), 1.64 (dt, J=7.7, 6.7 Hz, 2H), 1.61-1.58 (m, 2H), 1.51-1.45 (m, 2H), 1.45-1.37 (m, 2H). LCMS: tR=0.86, (ES⁺) m/z (M+H)⁺-282.1.

### Step 6: methyl 2-(4-(2-(7-(5-chloropyrimidin-2-yl)-7-azaspiro[3.5]nonan-2-yl)ethoxy)-2-fluorophenyl) acetate

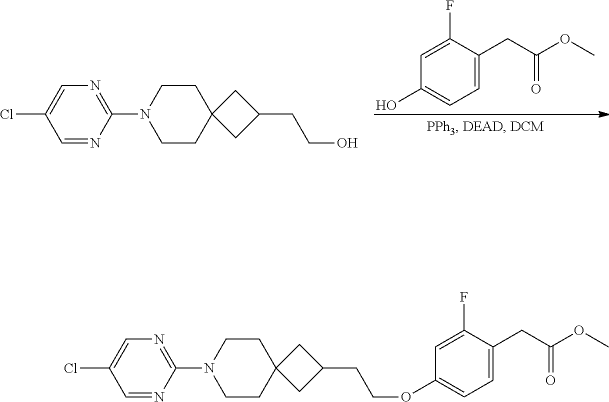 [SMILES: OCCC1CC2(CCN(c3ncc(Cl)cn3)CC2)C1 ; COC(=O)Cc1ccc(O)cc1F ; COC(=O)Cc1ccc(OCCC2CC3(CCN(c4ncc(Cl)cn4)CC3)C2)cc1F]

Prepared using procedures outlined in the preparation of intermediate 1 (step 2); replacing 3-(1-(5-chloropyrimidin-2-yl)piperidin-4-yl)propan-1-ol with 2-(7-(5-chloropyrimidin-2-yl)-7-azaspiro[3.5]nonan-2-yl)ethan-1-ol to give methyl 2-(4-(2-(7-(5-chloropyrimidin-2-yl)-7-azaspiro[3.5]nonan-2-yl)ethoxy)-2-fluorophenyl) acetate. ¹H NMR (500 MHz, Chloroform-d) δ 8.24 (s, 2H), 7.15 (t, J=8.6 Hz, 1H), 6.66 (d, J=8.6 Hz, 1H), 6.63 (dd, J=11.6, 2.4 Hz, 1H), 3.90 (t, J=6.3 Hz, 2H), 3.79-3.76 (m, 2H), 3.72 (s, 3H), 3.72-3.68 (m, 2H), 3.62 (s, 2H), 2.53-2.45 (m, 1H), 2.12-2.05 (m, 2H), 1.92 (q, J=6.7 Hz, 2H), 1.73-1.66 (m, 2H), 1.51-1.45 (m, 4H). LCMS: tR=1.72, (ES⁺) m/z (M+H)⁺=448.3.

### Step 7: 2-(4-(2-(7-(5-chloropyrimidin-2-yl)-7-azaspiro[3.5]nonan-2-yl)ethoxy)-2-fluorophenyl)acetic acid

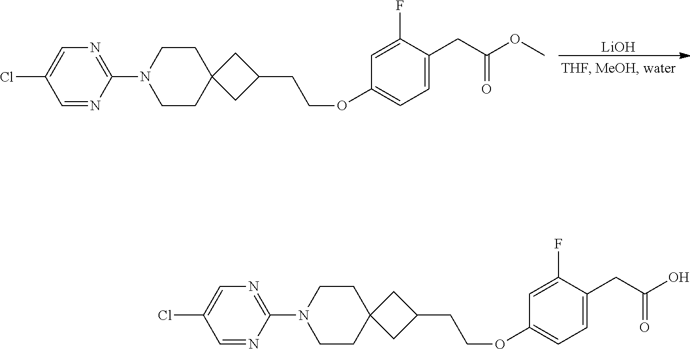

Prepared using procedures outlined in the preparation of intermediate 1 (step 3); replacing 2-(4-(3-(1-(5-chloropyrimidin-2-yl)piperidin-4-yl)propoxy)-2-fluorophenyl)acetate with methyl 2-(4-(2-(7-(5-chloropyrimidin-2-yl)-7-azaspiro[3.5]nonan-2-yl)ethoxy)-2-fluorophenyl) acetate to give 2-(7-(5-chloropyrimidin-2-yl)-7-azaspiro[3.5]nonan-2-yl)ethan-1-ol. ¹H NMR (500 MHz, DMSO-d₆) δ 8.35 (s, 2H), 7.20 (t, J=8.7 Hz, 1H), 6.75 (dd, J=12.0, 2.5 Hz, 1H), 6.70 (dd, J=8.4, 2.5 Hz, 1H), 3.90 (t, J=6.4 Hz, 2H), 3.71-3.67 (m, 2H), 3.65-3.58 (m, 2H), 3.51 (d, J=1.3 Hz, 2H), 2.41 (p, J=8.1 Hz, 1H), 2.03-1.96 (m, 2H), 1.84 (q, J=6.7 Hz, 2H), 1.61-1.54 (m, 2H), 1.53-1.44 (m, 4H). LCMS: tR=1.44, (ES⁺) m/z (M+H)⁺=434.3.

### Intermediate 25: 2-(4-(3-(7-(5-chloropyrimidin-2-yl)-7-azaspiro[3.5]nonan-2-yl)propoxy)-2-fluorophenyl)acetic acid

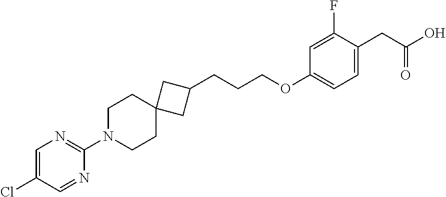 [SMILES: O=C(O)Cc1ccc(OCCCC2CC3(CCN(c4ncc(Cl)cn4)CC3)C2)cc1F]

Prepared using procedures outlined in the preparation of intermediate 24; replacing tert-butyl 2-oxo-7-azaspiro[3.5]nonane-7-carboxylate with tert-butyl 2-formyl-7-azaspiro[3.5]nonane-7-carboxylate in step 1 to give 2-(4-(3-(7-(5-chloropyrimidin-2-yl)-7-azaspiro[3.5]nonan-2-yl)propoxy)-2-fluorophenyl)acetic acid. ¹H NMR (500 MHz, DMSO-d₆) δ 12.37 (s, 1H), 8.36 (s, 2H), 7.20 (t, J=8.7 Hz, 1H), 6.77 (dd, J=12.0, 2.5 Hz, 1H), 6.71 (dd, J=8.4, 2.5 Hz, 1H), 3.94 (t, J=6.4 Hz, 2H), 3.71-3.66 (m, 2H), 3.63-3.57 (m, 2H), 3.52 (d, J=1.3 Hz, 2H), 2.27 (p, J=8.0 Hz, 1H), 2.00-1.93 (m, 2H), 1.67-1.59 (m, 2H), 1.59-1.49 (m, 4H), 1.48-1.43 (m, 2H), 1.43-1.36 (m, 2H). LCMS: tR=1.61, (ES⁺) m/z (M+H)⁺=448.3.

### Intermediate 26: 2-(4-(2-(7-(5-chloropyrimidin-2-yl)-7-azaspiro[3.5]nonan-1-yl)ethoxy)-2-fluorophenyl)acetic acid

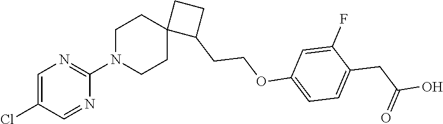 [SMILES: O=C(O)Cc1ccc(OCCC2CCC23CCN(c2ncc(Cl)cn2)CC3)cc1F]

### Step 1: tert-butyl (E)-1-(2-ethoxy-2-oxoethylidene)-7-azaspiro[3.5]nonane-7-carboxylate

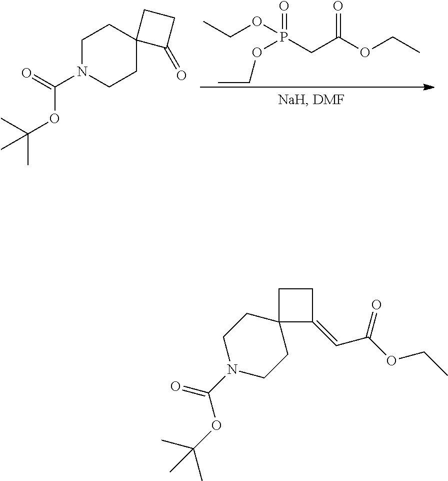

Prepared using procedure outlined in the preparation of intermediate 24 (step 1); replacing tert-butyl 2-oxo-7-azaspiro[3.5]nonane-7-carboxylate with tert-butyl 1-oxo-7-azaspiro[3.5]nonane-7-carboxylate to give tert-butyl (E)-1-(2-ethoxy-2-oxoethylidene)-7-azaspiro[3.5]nonane-7-carboxylate. ¹H NMR (500 MHz, Chloroform-d) δ 5.66 (t, J=2.6 Hz, 1H), 4.15 (q, J=7.1 Hz, 2H), 3.80 (s, 2H), 3.12-3.03 (m, 2H), 2.97 (ddd, J=14.4, 10.0, 3.5 Hz, 2H), 1.95 (t, J=8.1 Hz, 2H), 1.70-1.57 (m, 5H), 1.46 (s, 9H), 1.28 (t, J=7.1 Hz, 3H).

### Step 2: tert-butyl 1-(2-ethoxy-2-oxoethyl)-7-azaspiro[3.5]nonane-7-carboxylate

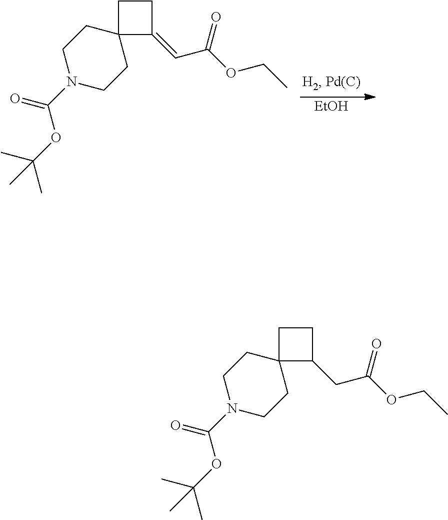

To a nitrogen flushed solution of tert-butyl (3E)-3-(2-ethoxy-2-oxo-ethylidene)-7-azaspiro[3.5]nonane-7-carboxylate (2.1 g, 6.79 mmol) in EtOH (60 mL) was added 10% palladium on carbon (200 mg) and the resulting mixture stirred under a balloon of hydrogen overnight. Mixture filtered through celite and the filtrate evaporated to give tert-butyl 1-(2-ethoxy-2-oxoethyl)-7-azaspiro[3.5]nonane-7-carboxylate (2 g, 94%) as a colorless oil. ¹H NMR (500 MHz, Chloroform-d) δ 4.10 (q, J=7.1 Hz, 2H), 3.89 (t, J=34.7 Hz, 2H), 2.78 (t, J=17.4 Hz, 2H), 2.47-2.36 (m, 2H), 2.33-2.23 (m, 1H), 2.13-2.05 (m, 1H), 1.85-1.73 (m, 1H), 1.72-1.53 (m, 4H), 1.51-1.38 (m, 11H), 1.24 (t, J=7.1 Hz, 3H).

### Step 3: tert-butyl 1-(2-hydroxyethyl)-7-azaspiro[3.5]nonane-7-carboxylate

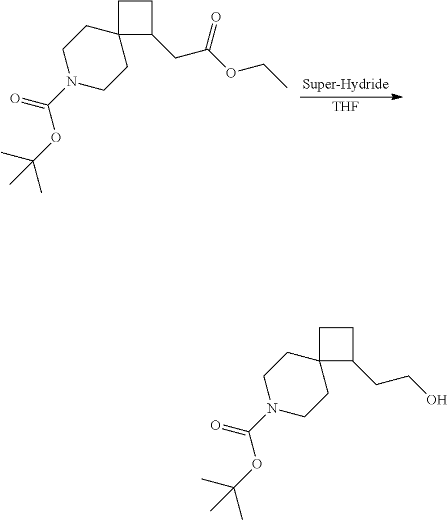

To a solution of tert-butyl 3-(2-ethoxy-2-oxo-ethyl)-7-azaspiro[3.5]nonane-7-carboxylate (2 g, 6.42 mmol) in THF (25 mL) cooled at ice bath temperature was added super-Hydride (19 mL of a 1M soln in THF, 1 mmol) and the resulting mixture stirred at room temperature overnight. Reaction quenched by the addition of MeOH (40 mL) and sat. NH₄Cl (75 mL) and extracted with DCM (3×50 mL); combined DCM layers washed with sat. NaCl (50 mL), dried over Na₂SO₄, filtered and evaporated. The residue was purified by silica gel column chromatography (Teledyne Isco: SNAP 24 g GOLD) eluent: gradient 0-100% EtOAc in Heptane to give tert-butyl 1-(2-hydroxyethyl)-7-azaspiro[3.5]nonane-7-carboxylate (1.7 g, 98%). ¹H NMR (500 MHz, Chloroform-d) δ 3.97-3.73 (m, 2H), 3.64-3.51 (m, 2H), 2.91-2.71 (m, 2H), 2.10-1.96 (m, 2H), 1.85-1.74 (m, 1H), 1.74-1.48 (m, 7H), 1.46 (s, 9H), 1.25 (d, J=5.1 Hz, 1H).

### Step 4: 2-(7-azaspiro[3.5]nonan-1-yl)ethan-1-ol hydrochloride

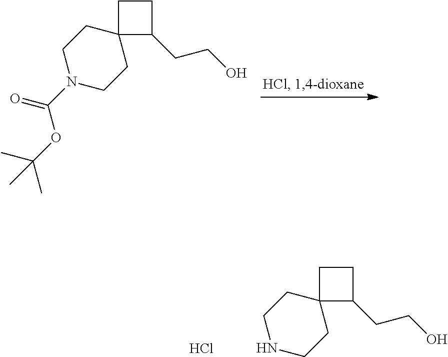 [SMILES: CC(C)(C)OC(=O)N1CCC2(CCC2CCO)CC1 ; Cl ; OCCC1CCC12CCNCC2]

Prepared using procedure outlined in the preparation of intermediate 24 (step 4); replacing tert-butyl 2-oxo-7-azaspiro[3.5]nonane-7-carboxylate with tert-butyl 1-oxo-7-azaspiro[3.5]nonane-7-carboxylate to give 2-(7-azaspiro[3.5]nonan-1-yl)ethan-1-ol hydrochloride.

### Step 5: 2-(7-(5-chloropyrimidin-2-yl)-7-azaspiro[3.5]nonan-1-yl)ethan-1-ol

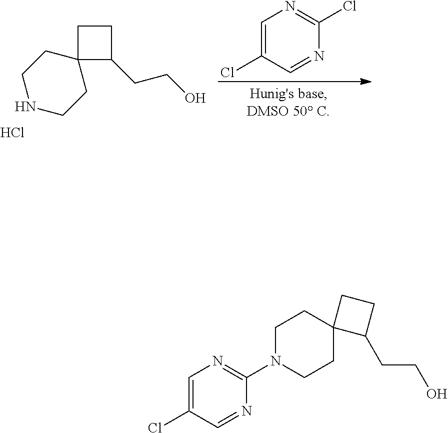 [SMILES: OCCC1CCC12CCNCC2 ; Clc1cnc(Cl)nc1 ; Cl ; OCCC1CCC12CCN(c1ncc(Cl)cn1)CC2]

Prepared using procedures outlined in the preparation of intermediate 1 (step 1) replacing 3-(piperidin-4-yl)propan-1-ol with tert-butyl 1-oxo-7-azaspiro[3.5]nonane-7-carboxylate to give 2-(7-azaspiro[3.5]nonan-1-yl)ethan-1-ol hydrochloride to give 2-(7-(5-chloropyrimidin-2-yl)-7-azaspiro[3.5]nonan-1-yl)ethan-1-ol. ¹H NMR (500 MHz, Chloroform-d) δ 8.19 (s, 2H), 4.44 (dtd, J=13.3, 4.0, 1.7 Hz, 1H), 4.36 (dtd, J=13.4, 4.0, 1.8 Hz, 1H), 3.63-3.52 (m, 2H), 3.05 (ddd, J=13.3, 11.7, 3.0 Hz, 1H), 2.99 (ddd, J=13.3, 10.1, 4.8 Hz, 1H), 2.06 (dddd, J=17.0, 15.0, 8.0, 4.5 Hz, 2H), 1.91-1.84 (m, 1H), 1.78-1.67 (m, 3H), 1.66-1.48 (m, 7H). LCMS: tR=1.06, (ES⁺) m/z (M+H)=282.1.

### Step 6: methyl 2-(4-(2-(7-(5-chloropyrimidin-2-yl)-7-azaspiro[3.5]nonan-1-yl)ethoxy)-2-fluorophenyl) acetate

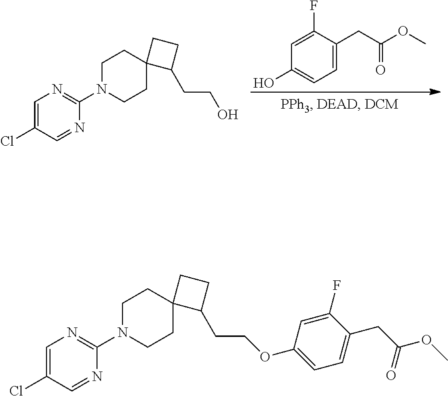 [SMILES: OCCC1CCC12CCN(c1ncc(Cl)cn1)CC2 ; COC(=O)Cc1ccc(O)cc1F ; COC(=O)Cc1ccc(OCCC2CCC23CCN(c2ncc(Cl)cn2)CC3)cc1F]

Prepared using procedures outlined in the preparation of intermediate 1 (step 2); replacing 3-(1-(5-chloropyrimidin-2-yl)piperidin-4-yl)propan-1-ol with 2-(7-(5-chloropyrimidin-2-yl)-7-azaspiro[3.5]nonan-1-yl)ethan-1-ol to give methyl 2-(4-(2-(7-(5-chloropyrimidin-2-yl)-7-azaspiro[3.5]nonan-1-yl)ethoxy)-2-fluorophenyl)acetate LCMS: tR=1.91, (ES⁺) m/z (M+H)⁺=448.2.

### Step 7: 2-(4-(2-(7-(5-chloropyrimidin-2-yl)-7-azaspiro[3.5]nonan-1-yl)ethoxy)-2-fluorophenyl)acetic acid

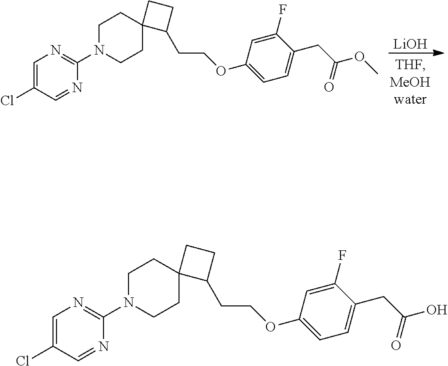

Prepared using procedures outlined in the preparation of intermediate 1 (step 3); replacing methyl 2-[4-[3-[1-(5-chloropyrimidin-2-yl)-4-piperidyl]propoxy]-2-fluoro-phenyl]acetate with methyl 2-(4-(2-(7-(5-chloropyrimidin-2-yl)-7-azaspiro[3.5]nonan-1-yl)ethoxy)-2-fluorophenyl)acetate to give 2-(4-(2-(7-(5-chloropyrimidin-2-yl)-7-azaspiro[3.5]nonan-1-yl)ethoxy)-2-fluorophenyl)acetic acid. LCMS: tR=1.62, (ES⁺) m/z (M+H)⁺=434.2.

### Intermediate 27: 2-(4-(4-(7-(5-chloropyrimidin-2-yl)-7-azaspiro[3.5]nonan-2-yl)butoxy)-2-fluorophenyl)acetic acid

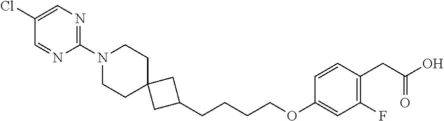 [SMILES: O=C(O)Cc1ccc(OCCCCC2CC3(CCN(c4ncc(Cl)cn4)CC3)C2)cc1F]

Prepared according to the procedures outlined in intermediate 24 replacing triethyl phosphonoacetate with triethyl 4-phosphonocrotonate in step 1 to give 2-(4-(4-(7-(5-chloropyrimidin-2-yl)-7-azaspiro[3.5]nonan-2-yl)butoxy)-2-fluorophenyl)acetic acid. ¹H NMR (500 MHz, DMSO-d₆) δ 12.38 (s, 1H), 8.35 (s, 2H), 7.20 (t, J=8.7 Hz, 1H), 6.76 (dd, J=12.0, 2.5 Hz, 1H), 6.71 (dd, J=8.4, 2.5 Hz, 1H), 3.94 (t, J=6.5 Hz, 2H), 3.71-3.66 (m, 2H), 3.62-3.57 (m, 2H), 3.52 (d, J=1.3 Hz, 2H), 2.22 (p, J=8.0 Hz, 1H), 2.00-1.92 (m, 2H), 1.68 (p, J=6.8 Hz, 2H), 1.59-1.53 (m, 2H), 1.48-1.41 (m, 4H), 1.41-1.26 (m, 4H). LCMS: tR=1.72, (ES⁺) m/z (M+H)⁺=462.3.

### Intermediate 28: 2-(4-((3-(1-(5-chloropyrimidin-2-yl)piperidin-4-yl)cyclobutyl)methoxy)-2-fluorophenyl)acetic acid

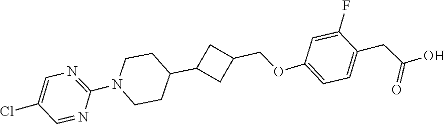 [SMILES: O=C(O)Cc1ccc(OCC2CC(C3CCN(c4ncc(Cl)cn4)CC3)C2)cc1F]

### Step 1: benzyl 4-(3-oxocyclobutyl)piperidine-1-carboxylate

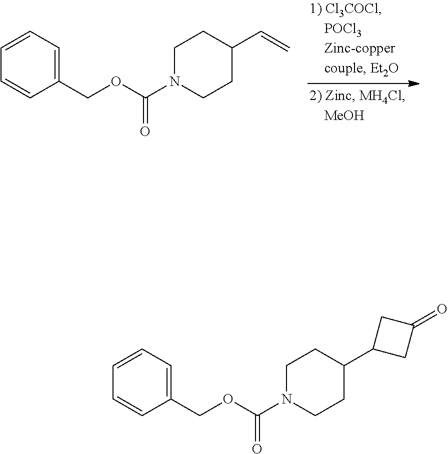 [SMILES: C=CC1CCN(C(=O)OCc2ccccc2)CC1 ; O=C1CC(C2CCN(C(=O)OCc3ccccc3)CC2)C1]

To a suspension of zinc-copper couple (9.1 g, 142 mmol), benzyl 4-vinylpiperidine-1-carboxylate (3.5 g, 14.3 mmol) and POCl₃ (1.46 mL, 15.7 mmol) in anhydrous Et₂O (100 mL) was added dropwise trichloroacetyl chloride (7.96 mL, 71.3 mmol). The resulting mixture stirred at room temperature overnight then quenched by pouring into sat. NaHCO₃ (200 mL) at 0° C. The mixture was filtered and the filtrate extracted with EtOAc (2×200 mL); combined EtOAc layers washed with sat. NaCl (2×50 mL), dried over MgSO₄, filtered and evaporated. The residue was dissolved in sat. NH₄Cl-MeOH (100 mL) and zinc (5 g, 75 mmol) added and stirred at room temperature overnight. The mixture was filtered and the filtrate evaporated. The residue was suspended in DCM and filtered and the filtrate evaporated. The residue was purified by silica gel column chromatography (Teledyne Isco: SNAP 40 g GOLD) eluent: 20% EtOAc in Heptane to give benzyl 4-(3-oxocyclobutyl)piperidine-1-carboxylate (1.87 g, 45%) as a white solid. ¹H NMR (500 MHz, Chloroform-d) δ 7.39-7.30 (m, 5H), 5.13 (s, 2H), 4.24 (s, 2H), 3.14-3.05 (m, 2H), 2.84-2.72 (m, 4H), 2.16-2.06 (m, 1H), 1.74 (s, 2H), 1.49-1.38 (m, 1H), 1.17 (d, J=11.2 Hz, 2H).

### Step 2: benzyl 4-(3-(methoxymethylene)cyclobutyl)piperidine-1-carboxylate

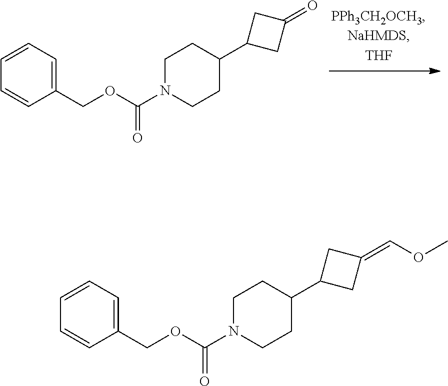 [SMILES: O=C1CC(C2CCN(C(=O)OCc3ccccc3)CC2)C1 ; COC=C1CC(C2CCN(C(=O)OCc3ccccc3)CC2)C1]

To a suspension of methoxymethyl(triphenyl)phosphonium;chloride (1.77 g, 5.17 mmol) in THE (50 mL) cooled in an ice bath was added sodium bis(trimethylsilylamide) (5.18 mL of a 1M solution in THF, 5.17 mmol) and the resulting mixture stirred at ice bath temperature for 1 hour then a solution of benzyl 4-(3-oxocyclobutyl)piperidine-1-carboxylate (990 mg, 3.44 mmol) in THE (10 mL) and the resulting mixture stirred at room temperature overnight. Reaction diluted by the addition of EtOAc (200 mL) and washed with water (2×100 mL), sat. NaCl (100 mL), dried over Na₂SO₄, filtered and evaporated. The residue was purified by silica gel column chromatography (Teledyne Isco: SNAP 24g GOLD) eluent: gradient 0-20% EtOAc in Heptane to give benzyl 4-(3-(methoxymethylene)cyclobutyl)piperidine-1-carboxylate (430 mg, 39%). ¹H NMR (500 MHz, Chloroform-d) δ 7.36 (d, J=4.1 Hz, 4H), 7.33-7.29 (m, 1H), 5.78 (p, J=2.3 Hz, 1H), 5.15-5.09 (m, 2H), 4.18 (s, 2H), 3.54 (s, 3H), 2.83-2.69 (m, 3H), 2.65 (dddt, J=14.7, 8.6, 3.2, 1.6 Hz, 1H), 2.40-2.24 (m, 2H), 2.02 (dtt, J=9.9, 8.5, 6.9 Hz, 1H), 1.7-1.6 (m, 2H), 1.38 (tdt, J=11.4, 9.7, 3.6 Hz, 1H), 1.01 (d, J=14.4 Hz, 2H).

### Step 3: benzyl 4-(3-(hydroxymethyl)cyclobutyl)piperidine-1-carboxylate

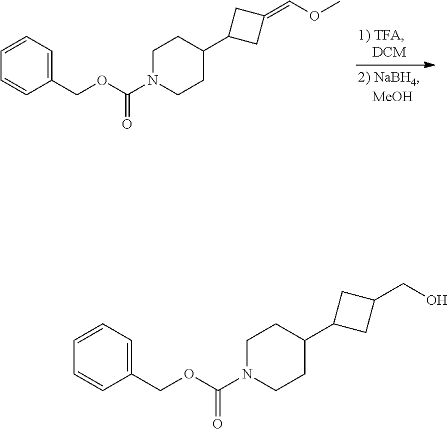 [SMILES: COC=C1CC(C2CCN(C(=O)OCc3ccccc3)CC2)C1 ; O=C(OCc1ccccc1)N1CCC(C2CC(CO)C2)CC1]

To a solution of benzyl 4-[3-(methoxymethylene)cyclobutyl]piperidine-1-carboxylate (430 mg, 1.36 mmol) in DCM (10 mL) cooled at 0° C. was added TFA (10 mL, 136 mmol) and the resulting mixture stirred at 0° C. for 1 hour. The mixture was evaporated and the residue neutralized by the addition of sat. NaHCO₃ and extracted with DCM (2×15 mL); combined DCM layers dried over Na₂SO₄, filtered and evaporated. The residue was taken up in MeOH (5 mL) and treated with sodium borohydride (103 mg, 2.73 mmol) and the resulting mixture stirred at room temperature for 1 hour. Mixture evaporated and the residue partitioned between water (10 mL) and DCM (10 mL); organic layer dried over Na₂SO₄, filtered and evaporated. The residue was purified by silica gel column chromatography (Teledyne Isco: SNAP 12 g GOLD) eluent: gradient 0-100% EtOAc in Heptane to give benzyl 4-(3-(hydroxymethyl) cyclobutyl)piperidine-1-carboxylate (313 mg, 75%) as a white solid. ¹H NMR (500 MHz, Chloroform-d) δ 7.36 (d, J=4.5 Hz, 4H), 7.34-7.29 (m, 1H), 5.12 (s, 2H), 4.14 (d, J=20.8 Hz, 2H), 3.67 (d, J=7.3 Hz, 1H), 3.53 (d, J=6.3 Hz, 1H), 2.73 (s, 2H), 2.40-2.29 (m, 1H), 2.14-2.07 (m, 1H), 1.92-1.85 (m, 1H), 1.81 (dd, J=7.8, 6.6 Hz, 2H), 1.45 (d, J=9.6 Hz, 4H), 1.42-1.33 (m, 2H), 0.97 (s, 2H). LCMS: tR=0.78, (ES⁺) m/z (M+H)⁺=304.2.

### Step 4: (3-(piperidin-4-yl)cyclobutyl)methanol

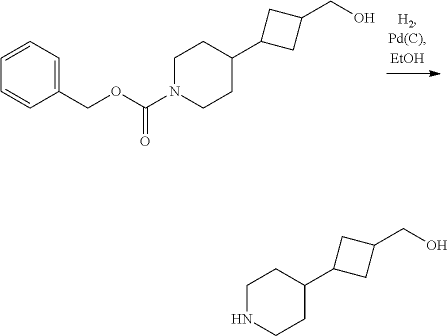 [SMILES: O=C(OCc1ccccc1)N1CCC(C2CC(CO)C2)CC1 ; OCC1CC(C2CCNCC2)C1]

A nitrogen flushed solution of benzyl 4-[3-(hydroxymethyl)cyclobutyl]piperidine-1-carboxylate (310 mg, 1.02 mmol) in EtOH (5 mL) was added 10% palladium on carbon (50 mg) and the resulting mixture stirred under a balloon of hydrogen for 1 hour. The mixture was filtered through celite and the filtrate evaporated to give (3-(piperidin-4-yl)cyclobutyl)methanol (140 mg, 82%) as a colorless oil. ¹H NMR (500 MHz, Chloroform-d) δ 3.66 (dd, J=7.4, 2.8 Hz, 1H), 3.52 (dd, J=6.4, 3.0 Hz, 1H), 3.16 (dq, J=9.6, 3.6 Hz, 2H), 2.95-2.75 (m, 2H), 2.61 (tdd, J=12.2, 9.5, 2.7 Hz, 2H), 2.33 (tdt, J=9.3, 7.7, 4.7 Hz, 1H), 2.10 (dddd, J=10.4, 9.1, 5.2, 2.1 Hz, 1H), 1.84-1.78 (m, 2H), 1.76-1.64 (m, 2H), 1.43-1.30 (m, 2H), 1.30-1.18 (m, 1H), 1.17-0.99 (m, 2H).

### Step 5: (3-(1-(5-chloropyrimidin-2-yl)piperidin-4-yl)cyclobutyl)methanol

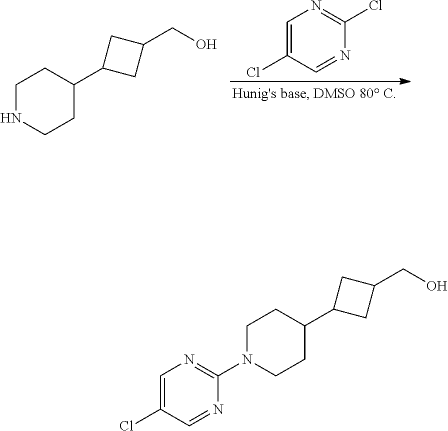 [SMILES: OCC1CC(C2CCNCC2)C1 ; Clc1cnc(Cl)nc1 ; OCC1CC(C2CCN(c3ncc(Cl)cn3)CC2)C1]

Prepared using procedure outlined in the preparation of intermediate 1 step 1; replacing 3-(piperidin-4-yl)propan-1-ol with (3-(piperidin-4-yl)cyclobutyl)methanol to give (3-(1-(5-chloropyrimidin-2-yl)piperidin-4-yl)cyclobutyl)methanol. ¹H NMR (500 MHz, Chloroform-d) δ 8.19 (d, J=1.1 Hz, 2H), 4.70-4.60 (m, 2H), 3.68 (d, J=7.3 Hz, 1H), 3.55 (d, J=6.2 Hz, 1H), 2.83 (dddd, J=13.2, 12.2, 8.0, 2.7 Hz, 2H), 2.42-2.30 (m, 1H), 2.12 (dddd, J=10.4, 9.1, 5.2, 2.1 Hz, 1H), 2.08-1.80 (m, 3H), 1.73 (dddd, J=17.9, 13.8, 4.1, 1.9 Hz, 2H), 1.54-1.45 (m, 1H), 1.45-1.34 (m, 2H), 1.07-0.94 (m, 2H). LCMS: tR=1.97, (ES⁺) m/z (M+H)⁺=282.2.

### Step 6: methyl 2-(4-(2-(3-(1-(5-chloropyrimidin-2-yl)piperidin-4-yl)cyclobutyl)ethyl)-2-fluorophenyl) acetate

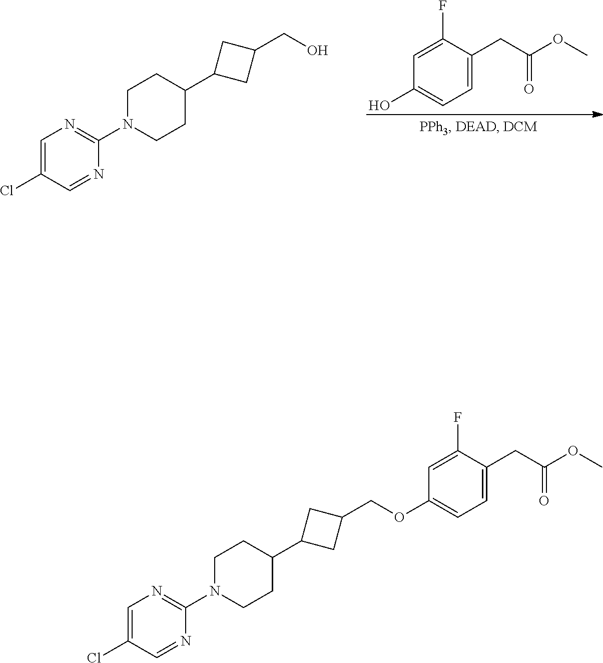 [SMILES: OCC1CC(C2CCN(c3ncc(Cl)cn3)CC2)C1 ; COC(=O)Cc1ccc(O)cc1F ; COC(=O)Cc1ccc(OCC2CC(C3CCN(c4ncc(Cl)cn4)CC3)C2)cc1F]

Prepared using procedures outlined in the preparation of intermediate 1 step 2; replacing 3-(1-(5-chloropyrimidin-2-yl)piperidin-4-yl)propan-1-ol with (3-(1-(5-chloropyrimidin-2-yl)piperidin-4-yl)cyclobutyl)methanol to give methyl 2-(4-(2-(3-(1-(5-chloropyrimidin-2-yl)piperidin-4-yl)cyclobutyl)ethyl)-2-fluorophenyl) acetate. 1H NMR (500 MHz, Chloroform-d) δ 8.20 (d, J=0.7 Hz, 2H), 7.13 (td, J=8.7, 1.5 Hz, 1H), 6.69-6.58 (m, 2H), 4.67 (tdd, J=10.8, 4.1, 1.8 Hz, 2H), 3.96 (d, J=7.2 Hz, 1H), 3.83 (d, J=6.1 Hz, 1H), 3.70 (d, J=0.9 Hz, 3H), 3.62-3.58 (m, 2H), 2.84 (dddd, J=13.2, 12.2, 5.1, 2.8 Hz, 2H), 2.68-2.52 (m, 1H), 2.19 (dddd, J=10.5, 9.2, 5.2, 2.1 Hz, 1H), 2.10-1.89 (m, 3H), 1.80-1.68 (m, 2H), 1.60-1.36 (m, 4H), 1.02 (dddd, J=13.3, 12.0, 8.8, 6.1 Hz, 2H). LCMS: tR=1.87, (ES⁺) m/z (M+H)⁺=448.3.

### Step 7: 2-(4-((3-(1-(5-chloropyrimidin-2-yl)piperidin-4-yl)cyclobutyl)methoxy)-2-fluorophenyl)acetic acid

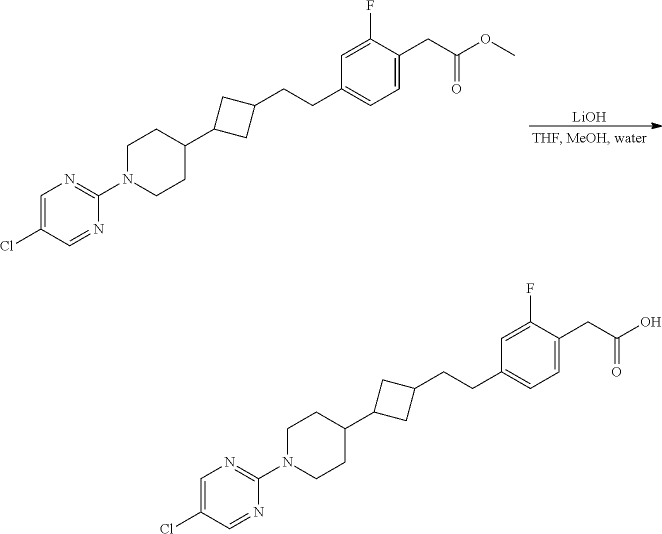 [SMILES: COC(=O)Cc1ccc(CCC2CC(C3CCN(c4ncc(Cl)cn4)CC3)C2)cc1F ; "LiOH" ; O=C(O)Cc1ccc(CCC2CC(C3CCN(c4ncc(Cl)cn4)CC3)C2)cc1F]

Prepared using procedure outlined in the preparation of intermediate 1 step 3; replacing methyl 2-[4-[3-[1-(5-chloropyrimidin-2-yl)-4-piperidyl]propoxy]-2-fluoro-phenyl]acetate with methyl 2-(4-(2-(3-(1-(5-chloropyrimidin-2-yl)piperidin-4-yl)cyclobutyl)ethyl)-2-fluorophenyl) acetate to give 2-(4-((3-(1-(5-chloropyrimidin-2-yl)piperidin-4-yl)cyclobutyl)methoxy)-2-fluorophenyl)acetic acid. ¹H NMR (500 MHz, DMSO-d6) δ 12.38 (s, 1H), 8.38 (s, 2H), 7.20 (t, J=8.8 Hz, 1H), 6.79 (ddd, J=11.9, 10.6, 2.5 Hz, 1H), 6.72 (td, J=8.2, 2.5 Hz, 1H), 4.58 (ddt, J=13.0, 10.6, 2.4 Hz, 2H), 4.01 (d, J=7.3 Hz, 1H), 3.88 (d, J=6.3 Hz, 1H), 3.52 (d, J=1.1 Hz, 2H), 3.39 (s, 1H), 2.86 (tdd, J=12.6, 7.1, 2.6 Hz, 2H), 2.61-2.53 (m, 1H), 2.17-2.05 (m, 2H), 1.97-1.82 (m, 3H), 1.74-1.63 (m, 2H), 1.60-1.39 (m, 2H), 0.91 (qt, J=12.5, 3.8 Hz, 2H). LCMS: tR=1.59, (ES⁺) m/z (M+H)⁺=434.3.

### Intermediate 29: 2-(4-(2-(3-(1-(5-chloropyrimidin-2-yl)piperidin-4-yl)cyclobutyl)ethoxy)-2-fluorophenyl)acetic acid

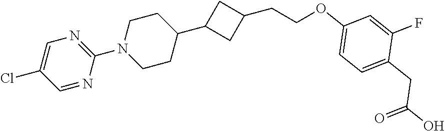 [SMILES: O=C(O)Cc1ccc(OCCC2CC(C3CCN(c4ncc(Cl)cn4)CC3)C2)cc1F]

### Step 1: benzyl 4-(3-(2-ethoxy-2-oxoethylidene)cyclobutyl)piperidine-1-carboxylate

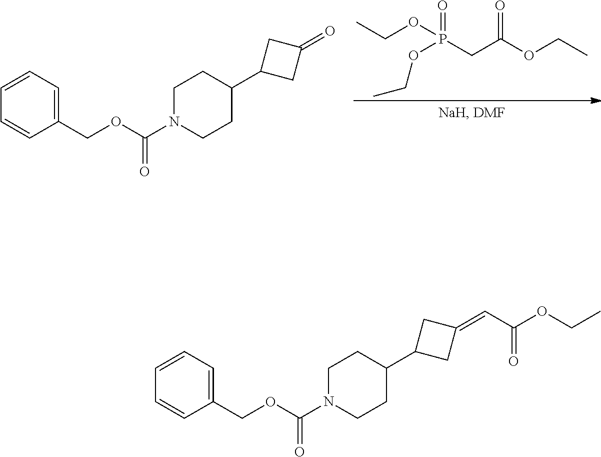

To a solution of triethyl phosphonoacetate (0.829 mL, 4.18 mmol) in DMF (10 mL) cooled in an ice bath was added sodium hydride (167 mg of a 60% dispersion, 4.17 mmol) and the resulting mixture stirred at ice bath temperature for 30 mins then a solution of benzyl 4-(3-oxocyclobutyl)piperidine-1-carboxylate (Intermediate 28 step 1, 800 mg, 2.78 mmol) in DMF (4 mL) and the resulting mixture stirred at room temperature for 3 hours. Mixture quenched by the addition of sat. Nh₄Cl (50 mL). Mixture extracted with EtOAc (2×30 mL); combined EtOAc layers washed with sat. NaCl (50 mL), dried over Na₂SO₄, filtered and evaporated. The residue purified by silica gel column chromatography (Teledyne Isco: SNAP 24 g GOLD) eluent: gradient 0-50% EtOAc in Heptane to give 630 mg (Yield 63%) as a colorless oil. ¹H NMR (500 MHz, Chloroform-d) δ 7.36 (d, J=4.1 Hz, 4H), 7.34-7.28 (m, 1H), 5.64-5.59 (m, 1H), 5.17-5.10 (m, 2H), 4.14 (qd, J=7.1, 0.8 Hz, 4H), 3.28-3.18 (m, 1H), 2.85 (d, J=6.5 Hz, 1H), 2.75 (ddt, J=17.8, 6.6, 3.1 Hz, 3H), 2.56-2.46 (m, 1H), 2.11 (dtt, J=9.8, 8.4, 6.9 Hz, 1H), 1.68 (s, 2H), 1.41 (ddt, J=14.8, 7.2, 5.6 Hz, 1H), 1.27 (t, J=7.1 Hz, 3H), 1.05 (s, 2H).

### Step 2: ethyl 2-(3-(piperidin-4-yl)cyclobutyl)acetate

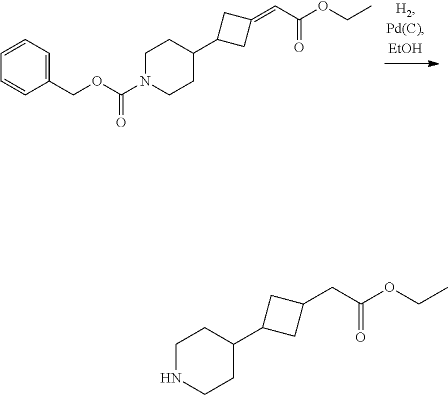 [SMILES: CCOC(=O)C=C1CC(C2CCN(C(=O)OCc3ccccc3)CC2)C1 ; CCOC(=O)CC1CC(C2CCNCC2)C1]

To a nitrogen flushed solution of benzyl 4-[3-(2-ethoxy-2-oxo-ethylidene)cyclobutyl]piperidine-1-carboxylate (630 mg, 1.78 mmol) in ethanol (20 mL) was added 10% palladium on carbon (100 mg) and the resulting mixture stirred under a balloon of hydrogen overnight. Mixture filtered through celite and the filtrate evaporated to give ethyl 2-(3-(piperidin-4-yl)cyclobutyl)acetate (410 mg, 100%) as a colorless oil. ¹H NMR (500 MHz, Chloroform-d) δ 4.11 (qd, J=7.1, 4.0 Hz, 2H), 3.11-3.00 (m, 2H), 2.55 (td, J=12.2, 2.6 Hz, 2H), 2.51-2.43 (m, 1H), 2.34 (d, J=7.5 Hz, 1H), 2.20 (dddd, J=10.4, 9.0, 5.2, 2.4 Hz, 1H), 2.10-1.80 (m, 2H), 1.80-1.70 (m, 2H), 1.69-1.55 (m, 2H), 1.37-1.15 (m, 5H), 1.00-0.88 (m, 2H).

### Step 3: tert-butyl 4-(3-(2-ethoxy-2-oxoethyl)cyclobutyl)piperidine-1-carboxylate

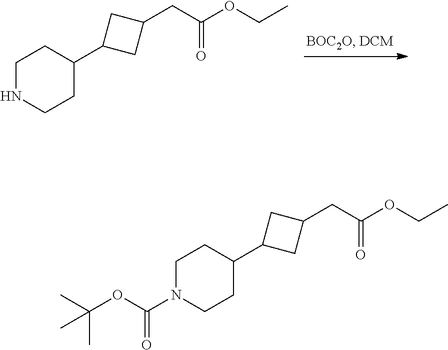

To a solution of ethyl 2-(3-(piperidin-4-yl)cyclobutyl)acetate (410 mg, 1.81 mmol) in DCM (10 mL) was added di-tert butydicarbonate (436 mg, 2.0 mmol) and the resulting mixture stirred at room temperature overnight. Mixture evaporated and purified by silica gel column chromatography (Teledyne Isco: SNAP 24g GOLD) eluent: gradient 0-50% EtOAc in Heptane to give tert-butyl 4-(3-(2-ethoxy-2-oxoethyl)cyclobutyl)piperidine-1-carboxylate (590 mg, 99%) as a colorless oil. ¹H NMR (500 MHz, Chloroform-d) δ 4.11 (qd, J=7.2, 4.0 Hz, 4H), 2.71-2.44 (m, 4H), 2.34 (d, J=7.4 Hz, 1H), 2.21 (dddd, J=10.4, 9.0, 5.1, 2.4 Hz, 1H), 2.06 and 1.82 (m, 1H), 1.96-1.88 (m, 1H), 1.81-1.75 (m, 1H), 1.61 (m, 2H), 1.45 (d, J=1.7 Hz, 9H), 1.40-1.24 (m, 5H), 0.98-0.86 (m, 2H).

### Step 4: tert-butyl 4-(3-(2-hydroxyethyl)cyclobutyl)piperidine-1-carboxylate

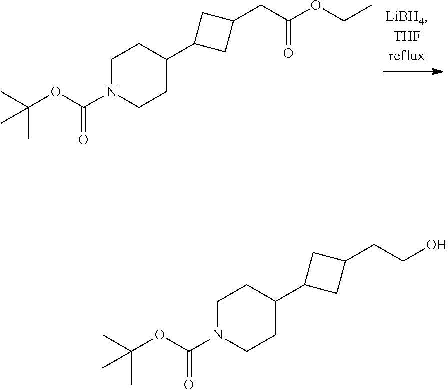

To a solution of tert-butyl 4-[3-(2-ethoxy-2-oxo-ethyl)cyclobutyl]piperidine-1-carboxylate (590 mg, 1.81 mmol) in THE (10 mL) was added lithium borohydride (146 mg, 6.7 mmol) and the resulting mixture heated at reflux for 5 hours. Mixture cooled and quenched by the addition of water (50 mL) and extracted with EtOAc (2×20 mL), combined EtOAc layers washed with sat. NaCl (20 mL), dried over Na₂SO₄, filtered and evaporated to give tert-butyl 4-(3-(2-hydroxyethyl)cyclobutyl)piperidine-1-carboxylate (500 mg, 97%) as a colorless oil. ¹H NMR (500 MHz, Chloroform-d) δ 4.16-3.97 (m, 2H), 3.65-3.53 (m, 2H), 2.72-2.57 (m, 2H), 2.30-2.12 (m, 2H), 2.08-1.79 (m, 2H), 1.73 (ddt, J=12.5, 8.7, 5.8 Hz, 2H), 1.69-1.51 (m, 2H), 1.45 (d, J=1.5 Hz, 9H), 1.40-1.31 (m, 1H), 1.40-1.17 (m, 3H), 0.93 (qd, J=12.2, 4.3 Hz, 2H).

### Step 5: 2-(3-(1-(5-chloropyrimidin-2-yl)piperidin-4-yl)cyclobutyl)ethan-1-ol

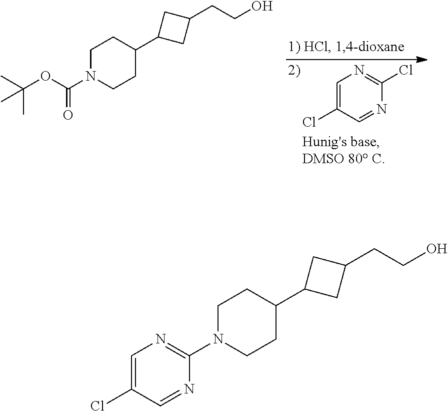

tert-butyl 4-[3-(2-hydroxyethyl)cyclobutyl]piperidine-1-carboxylate (500 mg, 1.76 mmol) was treated with hydrogen chloride (8.8 mL of a 4M solution in 1,4-dioxane, 35 mmol) and the resulting mixture stirred at room temperature overnight. Mixture evaporated and the residue triturated with acetonitrile, filtered and dried to give 2-(3-(piperidin-4-yl)cyclobutyl)ethan-1-ol hydrochloride (240 mg, 61%) as a white solid. A mixture of 2-(3-(piperidin-4-yl)cyclobutyl)ethan-1-ol hydrochloride (240 mg, 1.09 mmol), 2,5-dichloropyrimidine (163 mg, 1.09 mmol) and Hunig's base (0.57 mL, 3.28 mmol)) in DMSO (3 mL) was heated at 80° C. overnight. The mixture was cooled and poured into water (40 mL) and extracted with EtOAc (3×15 mL); combined EtOAc layers washed with sat. NaCl (2×30 mL), dried over Na₂SO₄, filtered and evaporated. The residue was purified by silica gel column chromatography (Teledyne Isco: SNAP 12g GOLD) eluent: gradient 0-100% EtOAc in Heptane to give 2-(3-(1-(5-chloropyrimidin-2-yl)piperidin-4-yl)cyclobutyl)ethan-1-ol (280 mg, 80%) as a white solid. LCMS: tR=1.20, (ES⁺) m/z (M+H)⁺=296.2.

### Step 6: methyl 2-(4-(2-(3-(1-(5-chloropyrimidin-2-yl)piperidin-4-yl)cyclobutyl)ethoxy)-2-fluorophenyl) acetate

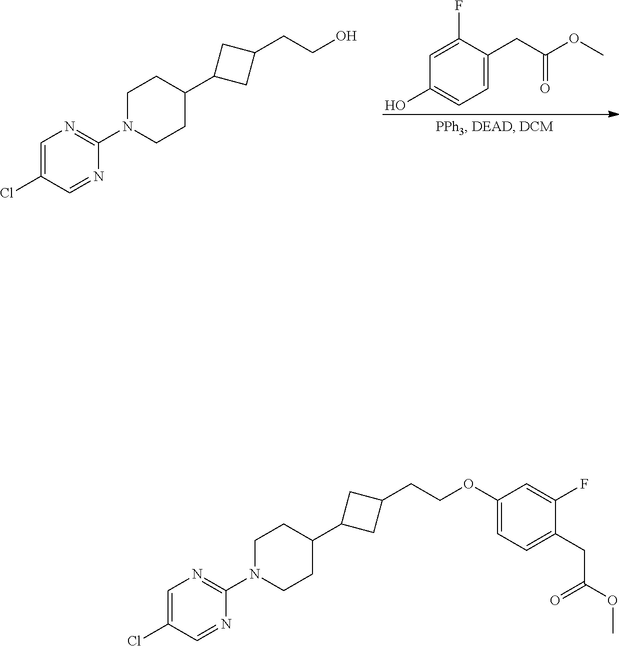 [SMILES: OCCC1CC(C2CCN(c3ncc(Cl)cn3)CC2)C1 ; COC(=O)Cc1ccc(O)cc1F ; COC(=O)Cc1ccc(OCCC2CC(C3CCN(c4ncc(Cl)cn4)CC3)C2)cc1F]

Prepared using procedures outlined in the preparation of intermediate 1 step 2; replacing 3-(1-(5-chloropyrimidin-2-yl)piperidin-4-yl)propan-1-ol with 2-(3-(1-(5-chloropyrimidin-2-yl)piperidin-4-yl)cyclobutyl)ethan-1-ol to give methyl 2-(4-(2-(3-(1-(5-chloropyrimidin-2-yl)piperidin-4-yl)cyclobutyl)ethoxy)-2-fluorophenyl) acetate. LCMS: tR=1.99, (ES⁺) m/z (M+H)⁺=462.3.

### Step 7: 2-(4-(2-(3-(1-(5-chloropyrimidin-2-yl)piperidin-4-yl)cyclobutyl)ethoxy)-2-fluorophenyl)acetic acid

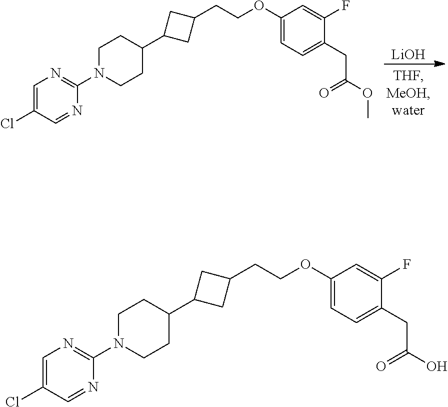

Prepared using procedure outlined in the preparation of intermediate 1 step 3; replacing methyl 2-[4-[3-[1-(5-chloropyrimidin-2-yl)-4-piperidyl]propoxy]-2-fluoro-phenyl]acetate with methyl 2-(4-(2-(3-(1-(5-chloropyrimidin-2-yl)piperidin-4-yl)cyclobutyl)ethoxy)-2-fluorophenyl) acetate to give 2-(4-(2-(3-(1-(5-chloropyrimidin-2-yl)piperidin-4-yl)cyclobutyl)ethoxy)-2-fluorophenyl)acetic acid. LCMS: tR=1.73, (ES⁺) m/z (M+H)⁺=448.2.

### Intermediate 30: 2-(4-(2-(6-(5-chloropyrimidin-2-yl)-6-azaspiro[2.5]octan-1-yl)ethoxy)-2-fluorophenyl)acetic acid

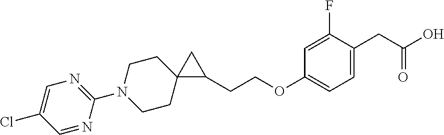 [SMILES: O=C(O)Cc1ccc(OCCC2CC23CCN(c2ncc(Cl)cn2)CC3)cc1F]

### Step 1: tert-butyl 1-(2-hydroxyethyl)-6-azaspiro[2.5]octane-6-carboxylate

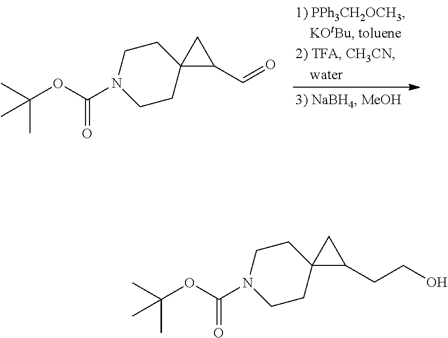 [SMILES: CC(C)(C)OC(=O)N1CCC2(CC1)CC2C=O ; CC(C)(C)OC(=O)N1CCC2(CC1)CC2CCO]

To a suspension of methoxymethyl(triphenyl)phosphonium;chloride (2.23 g, 6.52 mmol) in anhydrous toluene (10 mL) was added potassium tert-butoxide (6.52 mL of a 1M soln in THF, 6.52 mmol) and the resulting mixture stirred at room temperature for 1 hour. Mixture cooled in an ice bath and a solution of tert-butyl 2-formyl-6-azaspiro[2.5]octane-6-carboxylate (1.2 g, 5.01 mmol) in toluene (6 mL), the cooling bath removed and stirred at room temperature for 2 hours. The mixture was quenched by the addition of sat. NH₄Cl (50 mL) and extracted with EtOAc (2×20 mL); combined EtOAc layers washed with sat. NaCl (20 mL), dried over Na₂SO₄, filtered and evaporated. The residue was purified by silica gel column chromatography (Teledyne Isco: SNAP 40 g GOLD) eluent: gradient 0-60% EtOAc in Heptane to give tert-butyl (E)-1-(2-methoxyvinyl)-6-azaspiro[2.5]octane-6-carboxylate (845 mg, 63%). To a solution of tert-butyl 2-[(E)-2-methoxyvinyl]-6-azaspiro[2.5]octane-6-carboxylate (845 mg, 3.16 mmol) in a mixture of CH₃CN (31 mL) and water (8 mL) was added TFA (0.726 mL, 9.48 mmol) and the resulting mixture stirred at room temperature for 7 hours. Mixture quenched by the addition of sat. NaHCO₃ (50 mL) and the mixture evaporated to remove organic solvents. The remaining aqueous was extracted with DCM (2×40 mL); combined DCM layers washed with sat. NaCl (30 mL), dried over Na₂SO₄ filtered and evaporated. The residue was dissolved in MeOH (10 mL) and treated with sodium borohydride (108 mg, 9.48 mmol) and the resulting mixture stirred at room temperature for 1 hour. Mixture evaporated and the residue purified by silica gel column chromatography (Teledyne Isco: SNAP 24g GOLD) eluent: gradient 20-100% EtOAc in Heptane to give tert-butyl 1-(2-hydroxyethyl)-6-azaspiro[2.5]octane-6-carboxylate (422 mg, 52%) as a colorless oil. ¹H NMR (500 MHz, Chloroform-d) δ 3.72 (dq, J=10.4, 6.1, 4.3 Hz, 2H), 3.62 (s, 2H), 3.23 (dddd, J=12.8, 9.0, 3.5, 1.5 Hz, 2H), 1.82-1.73 (m, 1H), 1.57 (ddd, J=13.2, 8.9, 3.8 Hz, 1H), 1.57-1.40 (m, 12H), 1.36-1.26 (m, 1H), 1.12 (dt, J=13.1, 4.6 Hz, 1H), 0.65 (tt, J=8.4, 5.7 Hz, 1H), 0.52 (dd, J=8.5, 4.4 Hz, 1H), 0.10-0.05 (m, 1H).

### Step 2: 2-(6-azaspiro[2.5]octan-1-yl)ethan-1-ol hydrochloride

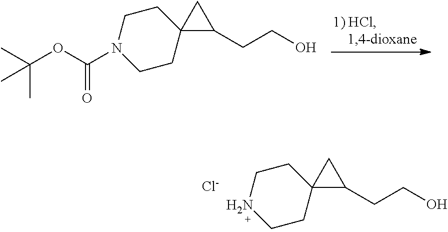 [SMILES: CC(C)(C)OC(=O)N1CCC2(CC1)CC2CCO ; [Cl-] ; OCCC1CC12CCNCC2]

A mixture of tert-butyl 2-(2-hydroxyethyl)-6-azaspiro[2.5]octane-6-carboxylate (422 mg, 1.65 mmol) and hydrogen chloride (8.26 mL of a 4M solution in dioxane, 33 mmol) was stirred at room temperature overnight. Mixture evaporated to give 2-(6-azaspiro[2.5]octan-1-yl)ethan-1-ol hydrochloride (316 mg, 99%) as colorless oil. ¹H NMR (500 MHz, DMSO-d₆) δ 9.12 (d, J=32.5 Hz, 2H), 4.33 (s, 1H), 3.45-3.31 (m, 2H), 3.08-2.94 (m, 2H), 2.88 (qd, J=8.2, 3.6 Hz, 2H), 1.67 (ddd, J=13.2, 8.3, 3.3 Hz, 1H), 1.63-1.54 (m, 1H), 1.48 (dq, J=16.7, 9.9, 8.4 Hz, 2H), 1.26 (tdd, J=13.6, 7.8, 5.4 Hz, 2H), 0.67-0.57 (m, 1H), 0.40 (dd, J=8.5, 4.2 Hz, 1H), 0.00 (t, J=4.9 Hz, 1H).

### Step 3: 2-(6-(5-chloropyrimidin-2-yl)-6-azaspiro[2.5]octan-1-yl)ethan-1-ol

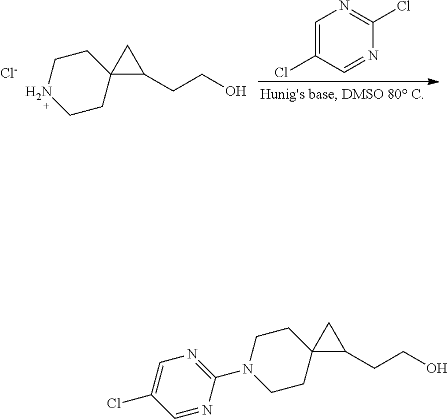 [SMILES: [Cl-] ; OCCC1CC12CCNCC2 ; Clc1cnc(Cl)nc1 ; OCCC1CC12CCN(c1ncc(Cl)cn1)CC2]

Prepared using procedure outlined in the preparation of intermediate 1 step 1; replacing 3-(piperidin-4-yl)propan-1-ol with 2-(6-azaspiro[2.5]octan-1-yl)ethan-1-ol hydrochloride to give 2-(6-(5-chloropyrimidin-2-yl)-6-azaspiro[2.5]octan-1-yl)ethan-1-ol. ¹H NMR (500 MHz, Chloroform-d) δ 8.21 (s, 2H), 4.14-4.03 (m, 2H), 3.74 (td, J=6.7, 2.2 Hz, 2H), 3.55 (dddd, J=12.7, 9.1, 3.4, 1.9 Hz, 2H), 1.87-1.74 (m, 1H), 1.65 (dt, J=13.7, 4.9 Hz, 1H), 1.57 (dddd, J=13.2, 9.1, 3.8, 1.1 Hz, 1H), 1.46 (ddt, J=13.1, 8.4, 6.6 Hz, 2H), 1.39 (dddd, J=13.5, 6.1, 3.5, 1.3 Hz, 1H), 1.25-1.16 (m, 1H), 0.75-0.65 (m, 1H), 0.62-0.54 (m, 1H), 0.14 (td, J=4.9, 4.4, 1.1 Hz, 1H). LCMS: tR=1.73, (ES⁺) m/z (M+H)⁺=268.1.

### Step 4: methyl 2-(4-(2-(6-(5-chloropyrimidin-2-yl)-6-azaspiro[2.5]octan-1-yl)ethoxy)-2-fluorophenyl) acetate

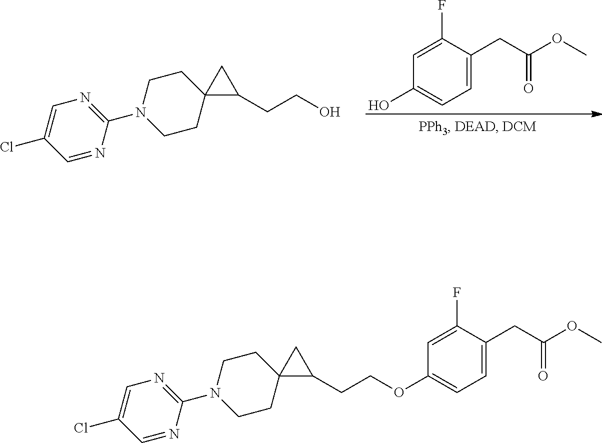 [SMILES: OCCC1CC12CCN(c1ncc(Cl)cn1)CC2 ; COC(=O)Cc1ccc(O)cc1F ; COC(=O)Cc1ccc(OCCC2CC23CCN(c2ncc(Cl)cn2)CC3)cc1F]

Prepared using procedures outlined in the preparation of intermediate 1 step 2; replacing 3-(1-(5-chloropyrimidin-2-yl)piperidin-4-yl)propan-1-ol with 2-(6-(5-chloropyrimidin-2-yl)-6-azaspiro[2.5]octan-1-yl)ethan-1-ol to give methyl 2-(4-(2-(6-(5-chloropyrimidin-2-yl)-6-azaspiro[2.5]octan-1-yl)ethoxy)-2-fluorophenyl) acetate. ¹H NMR (500 MHz, Chloroform-d) δ 8.21 (s, 2H), 7.13 (t, J=8.5 Hz, 1H), 6.67-6.64 (m, 1H), 6.62 (dd, J=11.5, 2.5 Hz, 1H), 4.18-4.06 (m, 2H), 4.03-3.97 (m, 2H), 3.70 (s, 3H), 3.60 (d, J=1.1 Hz, 2H), 3.55 (dddd, J=12.8, 9.2, 3.5, 2.1 Hz, 2H), 1.99 (dq, J=13.7, 6.9 Hz, 1H), 1.73-1.65 (m, 2H), 1.59 (dddd, J=13.2, 9.2, 3.9, 1.0 Hz, 1H), 1.46-1.39 (m, 1H), 1.21 (dddd, J=13.4, 5.9, 3.5, 1.2 Hz, 1H), 0.83-0.75 (m, 1H), 0.60 (ddd, J=8.6, 4.6, 0.8 Hz, 1H), 0.19-0.14 (m, 1H). LCMS: tR=1.63, (ES⁺) m/z (M+H)⁺=434.2.

### Step 5: 2-(4-(2-(6-(5-chloropyrimidin-2-yl)-6-azaspiro[2.5]octan-1-yl)ethoxy)-2-fluorophenyl)acetic acid

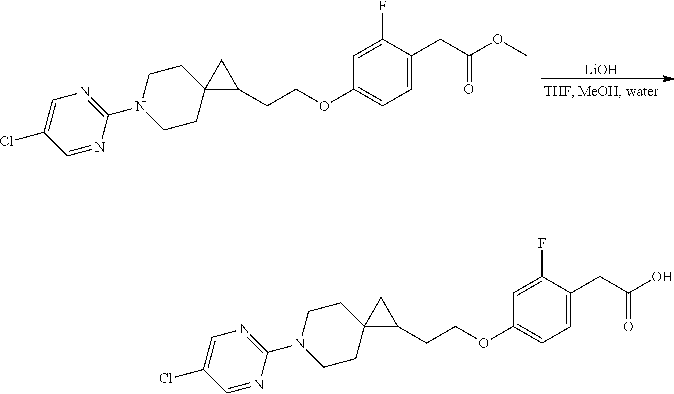 [SMILES: COC(=O)Cc1ccc(OCCC2CC23CCN(c2ncc(Cl)cn2)CC3)cc1F ; "LiOH" ; O=C(O)Cc1ccc(OCCC2CC23CCN(c2ncc(Cl)cn2)CC3)cc1F]

Prepared using procedure outlined in the preparation of intermediate 1 step 3; replacing methyl 2-[4-[3-[1-(5-chloropyrimidin-2-yl)-4-piperidyl]propoxy]-2-fluoro-phenyl]acetate with methyl 2-(4-(2-(6-(5-chloropyrimidin-2-yl)-6-azaspiro[2.5]octan-1-yl)ethoxy)-2-fluorophenyl) acetate to give 2-(4-(2-(6-(5-chloropyrimidin-2-yl)-6-azaspiro[2.5]octan-1-yl)ethoxy)-2-fluorophenyl)acetic acid. ¹H NMR (500 MHz, Chloroform-d) δ 8.22 (s, 2H), 7.13 (t, J=8.5 Hz, 1H), 6.66 (dd, J=8.1, 2.5 Hz, 1H), 6.63 (dd, J=11.4, 2.4 Hz, 1H), 4.17-4.06 (m, 2H), 4.03-3.97 (m, 2H), 3.65-3.62 (m, 2H), 3.54 (dddd, J=12.8, 9.2, 3.4, 2.1 Hz, 2H), 1.98 (dq, J=13.7, 6.9 Hz, 1H), 1.68 (dddd, J=14.1, 7.9, 5.4, 2.6 Hz, 2H), 1.63-1.55 (m, 1H), 1.41 (dddd, J=13.4, 6.1, 3.5, 1.2 Hz, 1H), 1.25-1.17 (m, 1H), 0.84-0.75 (m, 1H), 0.60 (ddd, J=8.5, 4.6, 0.8 Hz, 1H), 0.19-0.14 (m, 1H). LCMS: tR=1.33, (ES⁺) m/z (M+H)⁺=420.2.

### Intermediate 31: 2-(4-(3-(6-(5-chloropyrimidin-2-yl)-6-azaspiro[2.5]octan-1-yl)propoxy)-2-fluorophenyl)acetic acid

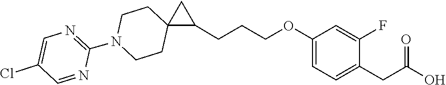 [SMILES: O=C(O)Cc1ccc(OCCCC2CC23CCN(c2ncc(Cl)cn2)CC3)cc1F]

### Step 1: tert-butyl (E)-1-(3-ethoxy-3-oxoprop-1-en-1-yl)-6-azaspiro[2.5]octane-6-carboxylate

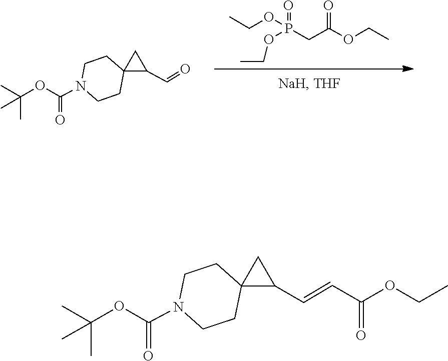 [SMILES: CC(C)(C)OC(=O)N1CCC2(CC1)CC2C=O ; CCOC(=O)CP(=O)(OCC)OCC ; CCOC(=O)/C=C/C1CC12CCN(C(=O)OC(C)(C)C)CC2]

To a solution of triethylphosphonoacetate (0.51 mL, 2.57 mmol) in THF (10 mL) cooled in an ice bath was added sodium hydride (103 mg of a 60% dispersion, 2.57 mmol) and the resulting mixture stirred at ice bath temperature for 1 hour after which a solution of tert-butyl 2-formyl-6-azaspiro[2.5]octane-6-carboxylate (410 mg, 1.71 mmol) in THF (4 mL) added and the resulting mixture stirred at room temperature overnight. Mixture quenched by the addition of sat. NH₄Cl (50 mL) and extracted with EtOAc (2×30 mL); combined EtOAc layers washed with sat. NaCl (30 mL), dried over Na₂SO₄, filtered and evaporated. The residue was purified by silica gel column chromatography (Teledyne Isco: SNAP 24g GOLD) eluent: gradient 0-100% EtOAc in Heptane to give tert-butyl (E)-1-(3-ethoxy-3-oxoprop-1-en-1-yl)-6-azaspiro[2.5]octane-6-carboxylate (1.01 g, 94%) as a colorless oil. ¹H NMR (500 MHz, Chloroform-d) δ 6.69 (dd, J=15.3, 10.2 Hz, 1H), 5.92 (dd, J=15.3, 0.5 Hz, 1H), 4.18 (qd, J=7.1, 2.3 Hz, 2H), 3.43 (ddt, J=11.0, 7.4, 5.1 Hz, 3H), 3.36 (dt, J=12.8, 6.1 Hz, 1H), 1.56 (ddq, J=7.0, 4.9, 2.8, 2.2 Hz, 2H), 1.53-1.48 (m, 1H), 1.45 (s, 9H), 1.43-1.39 (m, 2H), 1.28 (t, J=7.2 Hz, 3H), 1.00 (dd, J=8.2, 4.8 Hz, 1H), 0.78 (t, J=5.0 Hz, 1H).

### Step 2: tert-butyl 1-(3-ethoxy-3-oxopropyl)-6-azaspiro[2.5]octane-6-carboxylate

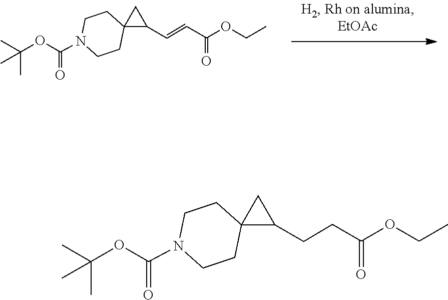 [SMILES: CCOC(=O)/C=C/C1CC12CCN(C(=O)OC(C)(C)C)CC2 ; CCOC(=O)CCC1CC12CCN(C(=O)OC(C)(C)C)CC2]

To a nitrogen flushed solution of tert-butyl 2-[(E)-3-ethoxy-3-oxo-prop-1-enyl]-6-azaspiro[2.5]octane-6-carboxylate (N53-64-1, 440 mg, 1.42 mmol) in EtOAc (15 mL) was added 5% Rhodium on alumina (100 mg) and the resulting mixture stirred under a balloon of hydrogen overnight. Mixture filtered through celite and the filtrate evaporated to give tert-butyl 1-(3-ethoxy-3-oxopropyl)-6-azaspiro[2.5]octane-6-carboxylate (860 mg, 81%) as a colorless oil.

### Step 3: 3-(6-(5-chloropyrimidin-2-yl)-6-azaspiro[2.5]octan-1-yl)propan-1-ol

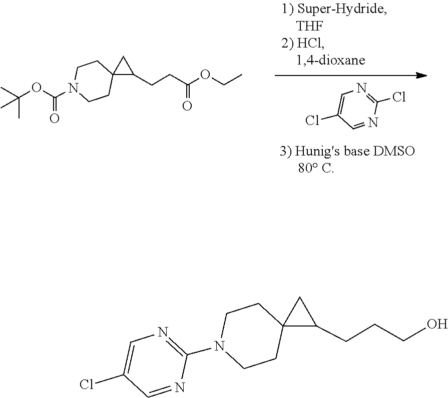 [SMILES: CCOC(=O)CCC1CC12CCN(C(=O)OC(C)(C)C)CC2 ; Clc1cnc(Cl)nc1 ; OCCCC1CC12CCN(c1ncc(Cl)cn1)CC2]

To a solution of tert-butyl 1-(3-ethoxy-3-oxopropyl)-6-azaspiro[2.5]octane-6-carboxylate (860 mg, 2.63 mmol) in THE (30 mL) cooled at 0° C. was added super-Hydride (6.6 mL of a 1M solution in THE, 6.6 mmol) and the resulting mixture stirred at room temperature overnight. The reaction was quenched by the addition of MeOH (15 mL) and sat. NH₄Cl (100 mL) and extracted with DCM (2×30 mL); combined DCM layers washed with sat. NaCl (30 mL), dried over Na₂SO₄, filtered and evaporated. The residue was treated with hydrogen chloride (13.9 mL of a 4M solution in dioxane, 55.6 mmol) and stirred at room temperature overnight. Mixture evaporated and residue azeotroped with acetonitrile (2×20 ml). To a solution of the residue in DMSO (5 mL) was added 2,5-dichloropyrimidine (201 mg, 1.35 mmol) and Hunig's base (0.709 mL, 4.06 mmol) and the resulting mixture heated at 80° C. overnight. The cooled mixture was diluted with water (100 mL) and extracted with EtOAc (3×15 mL); combined EtOAc layers washed with sat. NaCl (100 mL), dried over Na₂SO₄, filtered and evaporated. The residue was purified by silica gel column chromatography (Teledyne Isco: SNAP 24g GOLD) eluent: gradient 0-80% EtOAc in Heptane and further purified by reverse phase PREP-HPLC (C18 column) to give 3-(6-(5-chloropyrimidin-2-yl)-6-azaspiro[2.5]octan-1-yl)propan-1-ol (130 mg, 34%). ¹H NMR (500 MHz, Chloroform-d) δ 8.20 (s, 2H), 4.14-4.03 (m, 2H), 3.69 (t, J=6.6 Hz, 2H), 3.54 (ddt, J=12.5, 9.1, 3.2 Hz, 2H), 1.74-1.62 (m, 3H), 1.62-1.51 (m, 2H), 1.39 (dddd, J=13.4, 6.0, 3.5, 1.3 Hz, 1H), 1.36-1.24 (m, 2H), 1.19 (dddd, J=13.4, 5.9, 3.4, 1.3 Hz, 1H), 0.63 (tt, J=8.0, 5.7 Hz, 1H), 0.57-0.52 (m, 1H), 0.09-0.05 (m, 1H).

### Step 4: methyl 2-(4-(3-(6-(5-chloropyrimidin-2-yl)-6-azaspiro[2.5]octan-1-yl)propoxy)-2-fluorophenyl) acetate

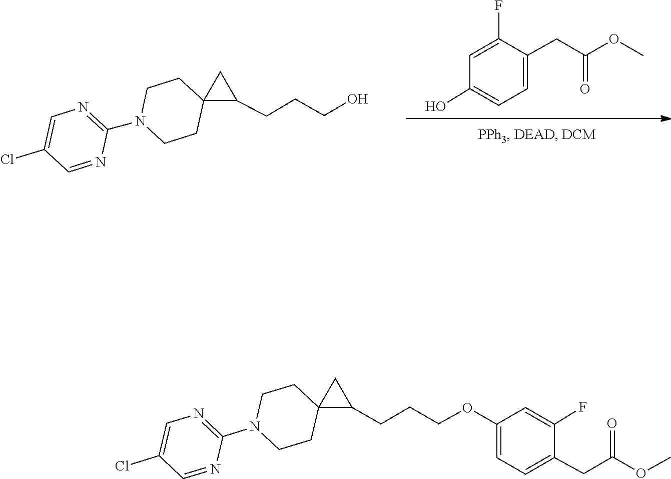 [SMILES: OCCCC1CC12CCN(c1ncc(Cl)cn1)CC2 ; COC(=O)Cc1ccc(O)cc1F ; COC(=O)Cc1ccc(OCCCC2CC23CCN(c2ncc(Cl)cn2)CC3)cc1F]

Prepared using procedures outlined in the preparation of intermediate 1 step 2; replacing 3-(1-(5-chloropyrimidin-2-yl)piperidin-4-yl)propan-1-ol with 3-(6-(5-chloropyrimidin-2-yl)-6-azaspiro[2.5]octan-1-yl)propan-1-ol to give methyl 2-(4-(3-(6-(5-chloropyrimidin-2-yl)-6-azaspiro[2.5]octan-1-yl)propoxy)-2-fluorophenyl) acetate. LCMS: tR=1.94, (ES⁺) m/z (M+H)⁺=448.3.

### Step 5: 2-(4-(3-(6-(5-chloropyrimidin-2-yl)-6-azaspiro[2.5]octan-1-yl)propoxy)-2-fluorophenyl)acetic acid

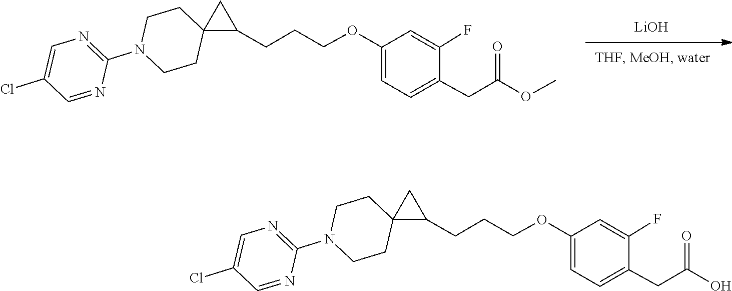 [SMILES: COC(=O)Cc1ccc(OCCCC2CC23CCN(c2ncc(Cl)cn2)CC3)cc1F ; "LiOH" ; O=C(O)Cc1ccc(OCCCC2CC23CCN(c2ncc(Cl)cn2)CC3)cc1F]

Prepared using procedure outlined in the preparation of intermediate 1 step 3; replacing methyl 2-[4-[3-[1-(5-chloropyrimidin-2-yl)-4-piperidyl]propoxy]-2-fluoro-phenyl]acetate with methyl 2-(4-(3-(6-(5-chloropyrimidin-2-yl)-6-azaspiro[2.5]octan-1-yl)propoxy)-2-fluorophenyl) acetate to give 2-(4-(3-(6-(5-chloropyrimidin-2-yl)-6-azaspiro[2.5]octan-1-yl)propoxy)-2-fluorophenyl)acetic acid. LCMS: tR=1.66, (ES⁺) m/z (M+H)⁺=434.2.

### Intermediate 32: 2-(4-(4-(1-(5-chloropyrimidin-2-yl)-4-methylpiperidin-4-yl)butoxy)-2-fluorophenyl)acetic acid

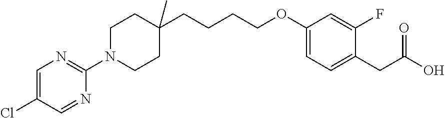 [SMILES: CC1(CCCCOc2ccc(CC(=O)O)c(F)c2)CCN(c2ncc(Cl)cn2)CC1]

### Step 1: tert-butyl 4-(4-ethoxy-4-oxobutyl)-4-methylpiperidine-1-carboxylate

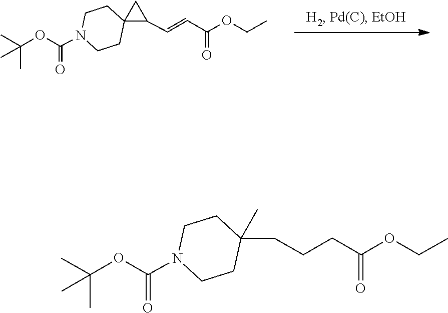 [SMILES: CCOC(=O)/C=C/C1CC12CCN(C(=O)OC(C)(C)C)CC2 ; CCOC(=O)CCCC1(C)CCN(C(=O)OC(C)(C)C)CC1]

To a nitrogen flushed solution of tert-butyl 2-[(E)-3-ethoxy-3-oxo-prop-1-enyl]-6-azaspiro[2.5]octane-6-carboxylate (Intermediate 31 step 1, 1.275 g, 4.12 mmol) in EtOH (20 mL) was added 10% palladium on carbon (100 mg) and the resulting mixture stirred under a balloon of hydrogen overnight. Mixture filtered through celite and the filtrate evaporated to give tert-butyl 4-(4-ethoxy-4-oxobutyl)-4-methylpiperidine-1-carboxylate (1.15 g, 89%) as a colorless oil. ¹H NMR (500 MHz, Chloroform-d) δ 4.13 (q, J =7.1 Hz, 2H), 3.52 (s, 2H), 3.20 (ddd, J=13.4, 9.1, 3.9 Hz, 2H), 2.28 (t, J=7.4 Hz, 2H), 1.64 (s, 3H), 1.62-1.54 (m, 2H), 1.45 (s, 9H), 1.40-1.26 (t, J=7.1 Hz, 9H).

### Step 2: tert-butyl 4-(4-hydroxybutyl)-4-methylpiperidine-1-carboxylate

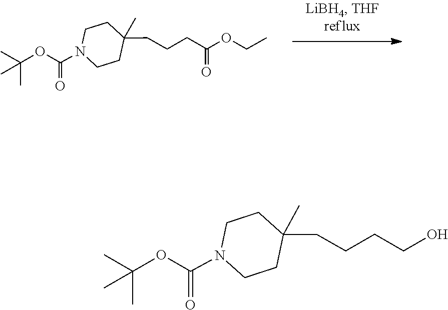

To a solution of tert-butyl 2-(3-ethoxy-3-oxo-propyl)-6-azaspiro[2.5]octane-6-carboxylate (1.15 g, 3.69 mmol) in THF (30 mL) was added lithium borohydride (201 mg, 9.23 mmol) and the resulting mixture heated at 60° C. for 5 hours. Mixture cooled in an ice bath and quenched by the addition of sat. NH₄Cl (50 mL) and extracted with EtOAc (40 mL); organic layer washed with sat. NaCl (40 mL), dried over Na₂SO₄, filtered and evaporated. The residue was purified by silica gel column chromatography (Teledyne Isco: SNAP 24g GOLD) eluent: gradient 20-100% EtOAc in Heptane to give tert-butyl 4-(4-hydroxybutyl)-4-methylpiperidine-1-carboxylate (894 mg, 89%) as a colorless oil. ¹H NMR (500 MHz, Chloroform-d) δ 3.66 (t, J=6.5 Hz, 2H), 3.53 (s, 2H), 3.19 (ddd, J=13.3, 9.2, 3.8 Hz, 2H), 1.68 (s, 1H), 1.59-1.50 (m, 2H), 1.45 (s, 9H), 1.43-1.23 (m, 8H), 0.92 (s, 3H).

### Step 3: 4-(4-methylpiperidin-4-yl)butan-1-ol hydrochloride

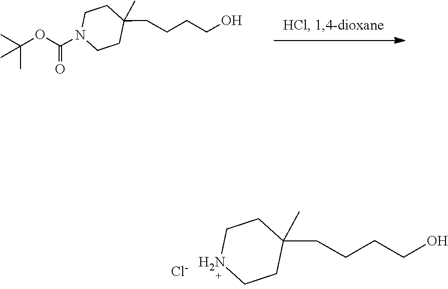 [SMILES: CC1(CCCCO)CCN(C(=O)OC(C)(C)C)CC1 ; [Cl-] ; CC1(CCCCO)CCNCC1]

A mixture of tert-butyl 2-(3-hydroxypropyl)-6-azaspiro[2.5]octane-6-carboxylate (890 mg, 3.3 mmol) and hydrogen chloride (16.5 mL of a 4M solution in 1,4-dioxane, 66 mmol) was stirred at room temperature overnight. Mixture evaporated to give 4-(4-methylpiperidin-4-yl)butan-1-ol hydrochloride (622 mg, 91%) as a white solid. ¹H NMR (500 MHz, DMSO-d₆) δ 8.87 (d, J=28.0 Hz, 2H), 4.36 (s, 1H), 3.35 (t, J=6.5 Hz, 2H), 3.03-2.86 (m, 4H), 1.49 (ddd, J=13.8, 9.1, 4.3 Hz, 2H), 1.45-1.31 (m, 4H), 1.19 (t, J=4.5 Hz, 4H), 0.87 (s, 3H).

### Step 4: 4-(1-(5-chloropyrimidin-2-yl)-4-methylpiperidin-4-yl)butan-1-ol

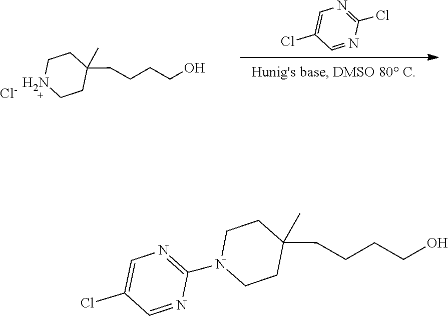 [SMILES: [Cl-] ; CC1(CCCCO)CCNCC1 ; Clc1cnc(Cl)nc1 ; CC1(CCCCO)CCN(c2ncc(Cl)cn2)CC1]

Prepared using procedure outlined in the preparation of intermediate 1 (step 1); replacing 3-(piperidin-4-yl)propan-1-ol with 4-(4-methylpiperidin-4-yl)butan-1-ol hydrochloride to give 4-(1-(5-chloropyrimidin-2-yl)-4-methylpiperidin-4-yl)butan-1-ol. ¹H NMR (500 MHz, Chloroform-d) δ 8.20 (s, 2H), 3.98 (dddd, J=13.5, 6.0, 4.3, 0.8 Hz, 2H), 3.66 (t, J=6.5 Hz, 2H), 3.50 (ddd, J=13.3, 9.1, 3.9 Hz, 2H), 1.61-1.53 (m, 2H), 1.50-1.28 (m, 9H), 0.99 (s, 3H). LCMS: tR=1.95, (ES⁺) m/z (M+H)⁺=284.2.

### Step 5: methyl 2-(4-(4-(1-(5-chloropyrimidin-2-yl)-4-methylpiperidin-4-yl)butoxy)-2-fluorophenyl)acetate

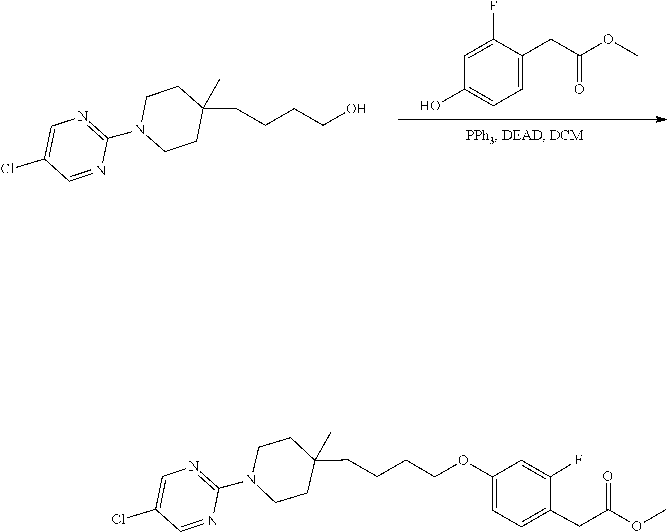 [SMILES: CC1(CCCCO)CCN(c2ncc(Cl)cn2)CC1 ; COC(=O)Cc1ccc(O)cc1F ; COC(=O)Cc1ccc(OCCCCC2(C)CCN(c3ncc(Cl)cn3)CC2)cc1F]

Prepared using procedures outlined in the preparation of intermediate 1 (step 2); replacing 3-(1-(5-chloropyrimidin-2-yl)piperidin-4-yl)propan-1-ol with 4-(1-(5-chloropyrimidin-2-yl)-4-methylpiperidin-4-yl)butan-1-ol to give methyl 2-(4-(4-(1-(5-chloropyrimidin-2-yl)-4-methylpiperidin-4-yl)butoxy)-2-fluorophenyl)acetate. ¹H NMR (500 MHz, Chloroform-d) δ 8.20 (s, 2H), 7.13 (t, J=8.5 Hz, 1H), 6.66-6.64 (m, 1H), 6.62 (dd, J=11.6, 2.5 Hz, 1H), 4.04-3.97 (m, 2H), 3.94 (t, J=6.4 Hz, 2H), 3.70 (s, 3H), 3.60 (d, J=1.2 Hz, 2H), 3.49 (ddd, J=13.4, 9.1, 3.9 Hz, 2H), 1.79-1.72 (m, 2H), 1.50-1.32 (m, 8H), 1.00 (s, 3H). LCMS: tR=1.80, (ES⁺) m/z (M+H)⁺=450.3.

### Step 6: 2-(4-(4-(1-(5-chloropyrimidin-2-yl)-4-methylpiperidin-4-yl)butoxy)-2-fluorophenyl)acetic acid

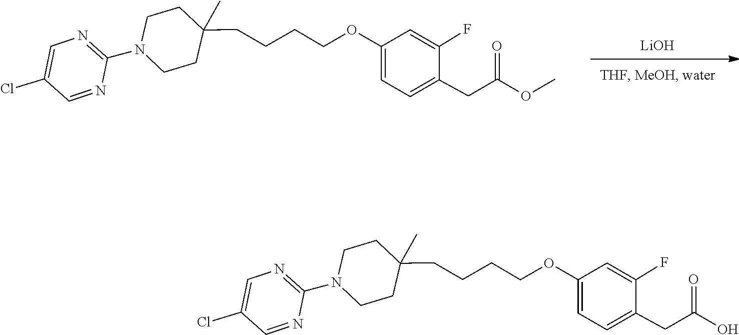 [SMILES: COC(=O)Cc1ccc(OCCCCC2(C)CCN(c3ncc(Cl)cn3)CC2)cc1F ; "LiOH" ; CC1(CCCCOc2ccc(CC(=O)O)c(F)c2)CCN(c2ncc(Cl)cn2)CC1]

Prepared using procedure outlined in the preparation of intermediate 1 (step 3); replacing methyl 2-[4-[3-[1-(5-chloropyrimidin-2-yl)-4-piperidyl]propoxy]-2-fluoro-phenyl]acetate with methyl 2-(4-(4-(1-(5-chloropyrimidin-2-yl)-4-methylpiperidin-4-yl)butoxy)-2-fluorophenyl)acetate to give 2-(4-(4-(1-(5-chloropyrimidin-2-yl)-4-methylpiperidin-4-yl)butoxy)-2-fluorophenyl)acetic acid. ¹H NMR (500 MHz, Chloroform-d) δ 8.21 (s, 2H), 7.13 (t, J=8.6 Hz, 1H), 6.65 (dd, J=8.2, 2.5 Hz, 1H), 6.62 (dd, J=11.6, 2.5 Hz, 1H), 4.03-3.96 (m, 2H), 3.94 (t, J=6.3 Hz, 2H), 3.63 (d, J=1.2 Hz, 2H), 3.48 (ddd, J=13.3, 9.0, 3.9 Hz, 2H), 1.80-1.71 (m, 2H), 1.49-1.37 (m, 6H), 1.37-1.31 (m, 2H), 1.00 (s, 3H). LCMS: tR=1.52, (ES⁺) m/z (M+H)⁺=436.3.

### Intermediate 33: 2-(4-(3-(1-(5-chloropyrimidin-2-yl)piperidin-4-yl)propoxy)-2-fluorophenyl)-1-(2,5-diazaspiro[3.4]octan-2-yl)ethan-1-one

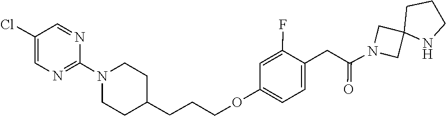 [SMILES: O=C(Cc1ccc(OCCCC2CCN(c3ncc(Cl)cn3)CC2)cc1F)N1CC2(CCCN2)C1]

### Step 1: tert-butyl 2-(2-(4-(3-(1-(5-chloropyrimidin-2-yl)piperidin-4-yl)propoxy)-2-fluorophenyl)acetyl)-2,5-diazaspiro[3.4]octane-5-carboxylate

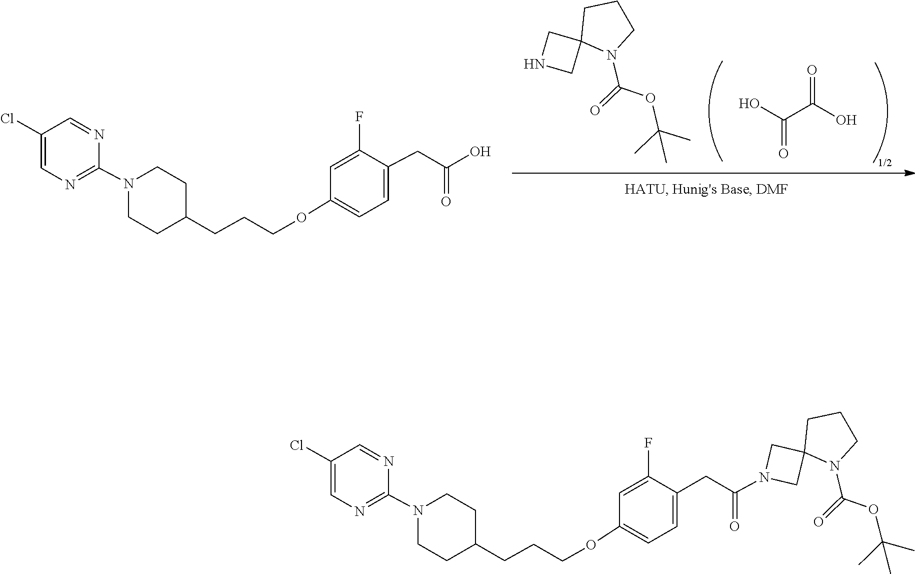

To a mixture of 2-[4-[3-[1-(5-chloropyrimidin-2-yl)-4-piperidyl]propoxy]-2-fluoro-phenyl]acetic acid (Intermediate 1, 85 mg, 0.208 mmol) and tert-butyl 2,5-diazaspiro[3.4]octane-5-carboxylate;oxalic acid (70 mg, 0.136 mmol) in DMF (0.5 mL) was added HATU (119 mg, 0.313 mmol) and Hunig's base (0.109 mL, 0.625 mmol) and the resulting mixture stirred at room temperature for 1 hour. Mixture diluted with EtOAc (10 mL) and washed with water (10 mL), sat. NaCl (10 mL), dried over Na₂SO₄, filtered and evaporated. The residue was purified by silica gel column chromatography (Teledyne Isco: SNAP 12g GOLD) eluent: gradient 1-5% MeOH in DCM to give tert-butyl 2-(2-(4-(3-(1-(5-chloropyrimidin-2-yl)piperidin-4-yl)propoxy)-2-fluorophenyl)acetyl)-2,5-diazaspiro[3.4]octane-5-carboxylate (120 mg, 93%) as a colorless oil. LCMS: tR=1.58, (ES⁺) m/z (M+H)⁺=602.5.

### Step 2: 2-(4-(3-(1-(5-chloropyrimidin-2-yl)piperidin-4-yl)propoxy)-2-fluorophenyl)-1-(2,5-diazaspiro[3.4]octan-2-yl)ethan-1-one

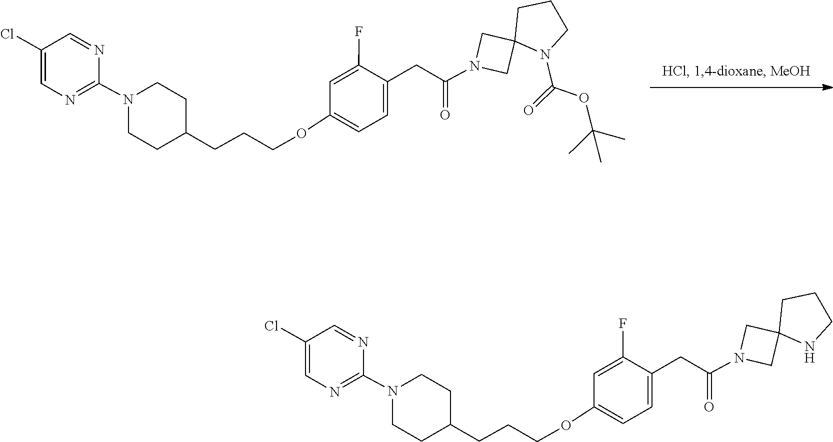 [SMILES: CC(C)(C)OC(=O)N1CCCC12CN(C(=O)Cc1ccc(OCCCC3CCN(c4ncc(Cl)cn4)CC3)cc1F)C2 ; O=C(Cc1ccc(OCCCC2CCN(c3ncc(Cl)cn3)CC2)cc1F)N1CC2(CCCN2)C1]

To a solution of tert-butyl 2-methyl-2,5-diazaspiro[3.4]octane-5-carboxylate;2-[4-[3-[1-(5-chloropyrimidin-2-yl)-4-piperidyl]propoxy]-2-fluoro-phenyl]acetaldehyde (120 mg, 0.194 mmol) in MeOH (1 mL) was added hydrogen chloride (0.97 ml of a 4M soln in dioxane, 3.88 mmol) and the resulting mixture stirred at room temperature for 2 hours after which UPLC_MS indicated complete loss of BOC group. Mixture evaporated and residue partitioned between DCM (15 mL) and sat. NaHCO₃ (20 mL); organic layer dried over Na₂SO₄, filtered and evaporated. The residue was purified by silica gel column chromatography (Teledyne Isco: SNAP 4g GOLD) eluent: gradient 2-15% MeOH in DCM+0.5% NH₄OH to give 2-(4-(3-(1-(5-chloropyrimidin-2-yl)piperidin-4-yl)propoxy)-2-fluorophenyl)-1-(2,5-diazaspiro[3.4]octan-2-yl)ethan-1-one_(75 mg, 74%) as a white solid. ¹H NMR (500 MHz, Chloroform-d) δ 8.20 (s, 2H), 7.21 (t, J=8.7 Hz, 1H), 6.67-6.62 (m, 1H), 6.58 (dd, J=11.8, 2.5 Hz, 1H), 4.68 (dp, J=13.4, 2.0 Hz, 2H), 4.09 (dd, J=8.7, 1.2 Hz, 1H), 4.06-3.98 (m, 2H), 3.95-3.87 (m, 3H), 3.41 (s, 2H), 3.07-2.94 (m, 2H), 2.91-2.82 (m, 2H), 1.96 (t, J=7.6 Hz, 2H), 1.81 (tdd, J=11.8, 5.8, 4.2 Hz, 6H), 1.57 (ttt, J=10.7, 7.0, 3.7 Hz, 1H), 1.45-1.37 (m, 2H), 1.23-1.12 (m, 2H). LCMS: tR=0.52, (ES⁺) m/z (M+H)⁺=502.5.

The following intermediates in Table P2 were prepared using procedures similar to those described in Intermediate 33 using appropriate starting materials.

TABLE P2

| Int | | |
|---|---|---|
| # | Structure | ¹HNMR/Mass [M + H]⁺ |
| 34 | 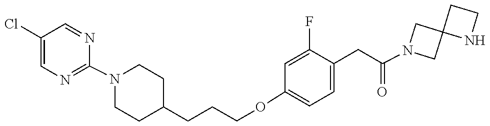 [SMILES: O=C(Cc1ccc(OCCCC2CCN(c3ncc(Cl)cn3)CC2)cc1F)N1CC2(CCN2)C1] | ¹H NMR (500 MHz, Chloroform-d) δ 8.20 (s, 2H), 7.20 (t, J = 8.6 Hz, 1H), 6.67-6.62 (m, 1H), 6.58 (dd, J = 11.7, 2.5 Hz, 1H), 4.72-4.64 (m, 2H), 4.24 (dd, J = 9.6, 1.6 Hz, 1H), 4.20 (dd, J = 10.9, 1.6 Hz, 1H), 4.13 (dd, J = 9.4, 1.5 Hz, 1H), 4.02 (dd, J = 10.8, 1.5 Hz, 1H), 3.92 (t, J = 6.5 Hz, 2H), 3.56-3.48 (m, 1H), 3.45-3.40 (m, 1H), 2.91-2.82 (m, 2H), 2.50 (qdd, J = 11.6, 8.3, 6.1 Hz, 2H), 1.85-1.74 (m, 4H), 1.57 (ttt, J = 10.7, 7.0, 3.7 Hz, 1H), 1.45-1.38 (m, 2H), 1.23-1.11 (m, |
| | | 2H). LCMS: tR = 0.46, (ES⁺) m/z (M + H)⁺ = 488.5. |
| 35 | 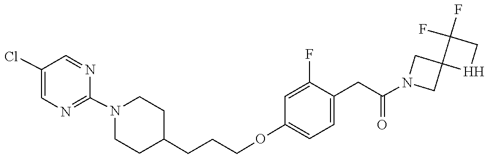 | ¹H NMR (500 MHz, Chloroform-d) δ 8.20 (s, 2H), 7.20 (t, J = 8.6 Hz, 1H), 6.69-6.63 (m, 1H), 6.60 (dd, J = 11.8, 2.5 Hz, 1H), 4.72-4.64 (m, 2H), 4.54 (dd, J = 10.1, 1.6 Hz, 1H), 4.48 (dd, J = 11.4, 1.7 Hz, 1H), 4.05 (d, J = 10.0 Hz, 1H), 3.95- 3.90 (m, 3H), 3.88 (d, J = 12.4 Hz, 1H), 3.83 (d, J = 11.3 Hz, 1H), 3.48-3.35 (m, 2H), 2.91-2.81 (m, 2H), 1.85-1.76 (m, 4H), 1.58 (ttt, J = 10.8, 7.0, 3.7 Hz, 1H), 1.45-1.37 (m, 2H), 1.23-1.13 (m, 2H). LCMS: tR = 0.95, (ES⁺) m/z (M + H)⁺ = 524.4. |
| 36 | 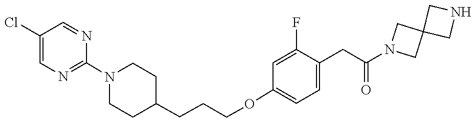 [SMILES: O=C(Cc1ccc(OCCCC2CCN(c3ncc(Cl)cn3)CC2)cc1F)N1CC2(CNC2)C1] | ¹H NMR (500 MHz, Chloroform-d) δ 8.20 (d, J = 1.4 Hz, 2H), 7.17 (dt, J = 18.9, 8.7 Hz, 1H), 6.64 (ddd, J = 8.5, 3.9, 2.5 Hz, 1H), 6.58 (ddd, J = 11.7, 2.6, 1.1 Hz, 1H), 4.68 (ddq, J = 13.3, 4.5, 2.2 Hz, 2H), 4.29 (s, 1H), 4.19 (s, 1H), 4.11 (s, 1H), 4.02 (d, J = 4.7 Hz, 3H), 3.91 (dt, J = 8.1, 6.5 Hz, 2H), 3.40-3.27 (m, 4H), 2.86 (tt, J = 12.8, 3.1 Hz, 2H), 1.86- 1.72 (m, 4H), 1.56 (dqt, J = 15.1, 6.5, 2.9 Hz, 1H), 1.44-1.35 (m, 2H), 1.17 (qt, J = 12.1, 4.1 Hz, 2H). LCMS: tR = 0.49, |
| | | (ES⁺) m/z (M + H)⁺ = 488.4. |
| 37 | 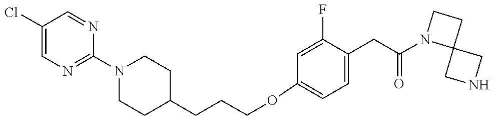 [SMILES: O=C(Cc1ccc(OCCCC2CCN(c3ncc(Cl)cn3)CC2)cc1F)N1CCC12CNC2] | ¹H NMR (500 MHz, DMSO-d6) δ 8.38 (d, J = 3.2 Hz, 2H), 7.17 (dt, J = 30.4, 8.8 Hz, 1H), 6.84-6.67 (m, 2H), 4.62- 4.55 (m, 2H), 4.46-4.41 (m, 1H), 4.05-3.92 (m, 5H), 3.39- 3.28 (m, 2H), 2.88 (td, J = 12.8, 2.7 Hz, 2H), 2.50-2.46 (m, 2H), 1.80-1.69 (m, 4H), 1.57 (dtd, J = 11.7, 7.2, 3.6 Hz, 1H), 1.40-1.31 (m, 2H), 1.05 (qd, J = 12.5, 4.1 Hz, 2H). LCMS: tR = 0.58, (ES⁺) m/z (M + H)⁺ = 488.4. |
| 38 | 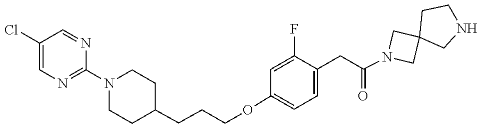 [SMILES: O=C(Cc1ccc(OCCCC2CCN(c3ncc(Cl)cn3)CC2)cc1F)N1CC2(CCNC2)C1] | LCMS: tR = 0.48, (ES⁺) m/z (M + H)⁺ = 502.4. |
| 39 | 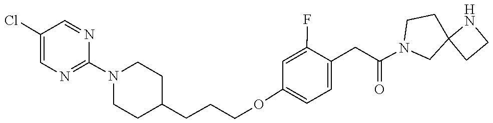 [SMILES: O=C(Cc1ccc(OCCCC2CCN(c3ncc(Cl)cn3)CC2)cc1F)N1CCC2(CCN2)C1] | ¹H NMR (500 MHz, Chloroform-d) δ 8.20 (s, 2H), 7.19 (dtd, J = 11.2, 8.6, 7.8, 2.5 Hz, 1H), 6.64 (dt, J = 8.5, 2.4 Hz, 1H), 6.62-6.56 (m, 1H), 4.68 (dp, J = 13.3, 2.0 Hz, 2H), 3.96- 3.80 (m, 3H), 3.73-3.50 (m, 6H), 3.50-2.99 (m, 2H), 2.89- 2.81 (m, 2H), 2.47-2.36 (m, 1H), 2.36-2.21 (m, 1H), 2.14- 1.90 (m, 2H), 1.80 (ddt, J = 14.1, 9.5, 4.9 Hz, 3H), 1.57 (ttt, J = 10.6, 6.9, 3.8 Hz, 1H), 1.45-1.37 (m, 2H), 1.17 (qd, J = 12.5, 4.2 Hz, 2H). LCMS: tR = 0.83, (ES⁺) m/z (M + H)⁺ = |
| | | 502.5. |
| 40 | 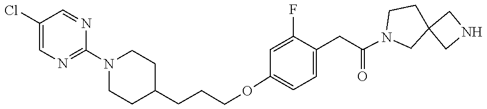 [SMILES: O=C(Cc1ccc(OCCCC2CCN(c3ncc(Cl)cn3)CC2)cc1F)N1CCC2(CNC2)C1] | ¹H NMR (500 MHz, Chloroform-d) δ 8.20 (s, 2H), 7.21- 7.11 (m, 1H), 6.64 (dt, J = 8.5, 2.2 Hz, 1H), 6.60 (ddd, J = 11.7, 3.8, 2.5 Hz, 1H), 4.72-4.64 (m, 2H), 4.01-3.83 (m, 4H), 3.71 (d, J = 18.6 Hz, 1H), 3.63-3.45 (m, 5H), 3.26- 3.12 (m, 3H), 2.86 (tt, J = 12.3, 2.6 Hz, 2H), 2.32 (t, J = 7.0 Hz, 1H), 2.19-2.10 (m, 1H), 2.00 (td, J = 7.1, 3.0 Hz, 1H), 1.80 (ddd, J = 14.6, 9.5, 4.5 Hz, 2H), 1.57 (ttt, J = 10.5, 6.9, |
| | | 3.5 Hz, 1H), 1.41 (qt, J = 9.1, 3.5 Hz, 2H), 1.17 (tdd, J = |
| | | 13.6, 7.6, 3.0 Hz, 2H). LCMS: tR = 0.54, (ES⁺) m/z |
| | | (M + H)⁺ = 502.5. |
| 41 | 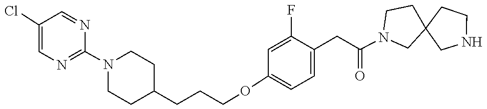 [SMILES: O=C(Cc1ccc(OCCCC2CCN(c3ncc(Cl)cn3)CC2)cc1F)N1CCC2(CCNC2)C1] | ¹H NMR (500 MHz, Chloroform-d) δ 8.20 (s, 2H), 7.18 (tdd, J = 8.7, 7.2, 1.9 Hz, 1H), 6.65 (ddd, J = 8.5, 5.7, 2.5 Hz, 1H), 6.59 (ddt, J = 11.6, 4.3, 2.2 Hz, 1H), 4.68 (dq, J = 13.2, 2.2, 1.8 Hz, 2H), 3.92 (td, J = 6.5, 2.6 Hz, 2H), 3.62-3.47 (m, 5H), 3.47-3.39 (m, 1H), 3.34-3.17 (m, 2H), 3.13-2.98 (m, 1H), 2.86 (td, J = 12.9, 2.7 Hz, 2H), 2.81-2.51 (m, 1H), 2.14- 1.96 (m, 1H), 1.91 (dq, J = 13.9, 6.7 Hz, 2H), 1.86-1.74 |
| | | (m, 5H), 1.57 (ttt, J = 10.7, 6.9, 3.7 Hz, 1H), 1.45-1.37 (m, |
| | | 2H), 1.17 (qd, J = 12.5, 4.2 Hz, 2H). LCMS: tR = 0.57, (ES⁺) |
| | | m/z (M + H)⁺ = 516.5. |
| 42 | 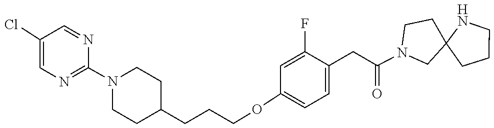 [SMILES: O=C(Cc1ccc(OCCCC2CCN(c3ncc(Cl)cn3)CC2)cc1F)N1CCC2(CCCN2)C1] | ¹H NMR (500 MHz, Chloroform-d) δ 8.20 (s, 2H), 7.20 (td, J = 8.6, 1.3 Hz, 1H), 6.67-6.62 (m, 1H), 6.59 (ddd, J = 11.7, 2.5, 0.9 Hz, 1H), 4.68 (dp, J = 13.3, 2.0 Hz, 2H), 3.92 (t, J = 6.4 Hz, 2H), 3.68-3.52 (m, 4H), 3.46-3.34 (m, 2H), 3.08- 2.91 (m, 2H), 2.91-2.81 (m, 2H), 1.95 (dd, J = 7.7, 6.4 Hz, 1H), 1.89-1.76 (m, 8H), 1.76-1.67 (m, 1H), 1.56 (ddt, J = 14.6, 6.9, 3.6 Hz, 1H), 1.45-1.38 (m, 2H), 1.23-1.12 (m, 2H). LCMS: tR = 0.52, (ES⁺) m/z (M + H)⁺ = 516.5. |
| 43 | 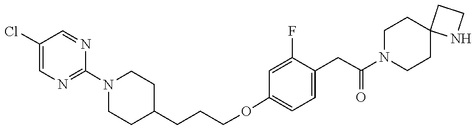 [SMILES: O=C(Cc1ccc(OCCCC2CCN(c3ncc(Cl)cn3)CC2)cc1F)N1CCC2(CCN2)CC1] | ¹H NMR (500 MHz, Chloroform-d) δ 8.20 (s, 2H), 7.16 (dt, J = 16.7, 8.6 Hz, 1H), 6.62 (ddt, J = 21.4, 11.6, 3.1 Hz, 2H), 5.51-5.37 (m, 1H), 4.68 (dp, J = 13.4, 1.9 Hz, 2H), 4.09- 4.04 (m, 1H), 3.96 (d, J = 3.2 Hz, 1H), 3.92 (td, J = 6.5, 1.8 Hz, 2H), 3.70 (t, J = 5.8 Hz, 1H), 3.67 (s, 1H), 3.64 (s, 1H), 3.54 (t, J = 5.7 Hz, 1H), 2.86 (td, J = 12.6, 2.7 Hz, 2H), 2.79 (dt, J = 8.1, 6.8 Hz, 2H), 2.21-2.11 (m, 2H), 2.07 (s, 1H), 2.00 (s, 1H), 1.85-1.75 (m, 5H), 1.57 (dtt, J = 11.0, 7.0, 3.6 Hz, 1H), 1.42 (qd, J = 7.6, 7.0, 5.3 Hz, 2H), 1.17 (qd, J = |
| | | 12.5, 4.2 Hz, 2H). LCMS: tR = 0.52, (ES⁺) m/z (M + H)⁺ = |
| | | 516.4. |
| 44 | 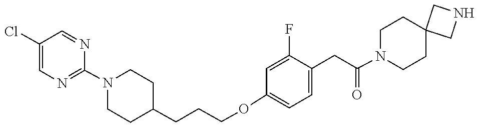 [SMILES: O=C(Cc1ccc(OCCCC2CCN(c3ncc(Cl)cn3)CC2)cc1F)N1CCC2(CC1)CNC2] | ¹H NMR (500 MHz, Chloroform-d) δ 8.20 (s, 2H), 7.15 (dd, J = 9.5, 7.9 Hz, 1H), 6.64 (dd, J = 8.5, 2.6 Hz, 1H), 6.59 (dt, J = 11.8, 2.7 Hz, 1H), 4.71-4.64 (m, 2H), 3.92 (td, J = 6.5, 1.7 Hz, 2H), 3.65-3.55 (m, 5H), 3.52 (t, J = 5.8 Hz, 2H), 3.37 (q, J = 8.8, 7.2 Hz, 2H), 3.04 (q, J = 7.1 Hz, 1H), 2.86 (td, J = 12.9, 2.7 Hz, 2H), 1.79 (dddt, J = 23.3, 17.7, 11.4, 5.7 Hz, 7H), 1.66 (dt, J = 15.9, 5.6 Hz, 1H), 1.57 (ddp, J = 10.9, 7.0, 3.5 Hz, 1H), 1.45-1.38 (m, 2H), 1.18 (qd, J = 12.5, 4.2 Hz, |
| | | 2H). LCMS: tR = 0.55, (ES⁺) m/z (M + H)⁺ = 516.5. |
| 45 | 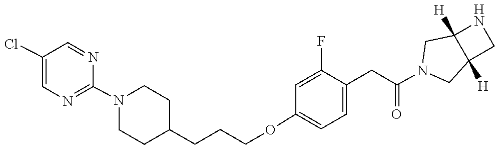 [SMILES: O=C(Cc1ccc(OCCCC2CCN(c3ncc(Cl)cn3)CC2)cc1F)N1C[C@H]2CN[C@H]2C1] | ¹H NMR (500 MHz, Chloroform-d) δ 8.20 (s, 2H), 7.30- 7.19 (m, 1H), 6.70-6.53 (m, 2H), 4.68 (dt, J = 12.5, 2.8 Hz, 2H), 4.53-4.40 and 4.15-4.03 (m, 1H), 3.96-3.74 (m, 4H), 3.74-3.59 (m, 2H), 3.50-3.42 and 3.36-3.30 (m, 1H), 3.26-2.90 (m, 3H), 2.86 (td, J = 12.8, 2.7 Hz, 2H), 1.81 (t, J = 11.9 Hz, 5H), 1.58 (dq, J = 7.1, 4.0 Hz, 1H), 1.41 (dd, J = 10.2, 5.5 Hz, 2H), 1.17 (qd, J = 12.4, 4.1 Hz, 2H). LCMS: tR = 0.51, (ES⁺) m/z (M + H)⁺ = 488.3. |
| 46 | 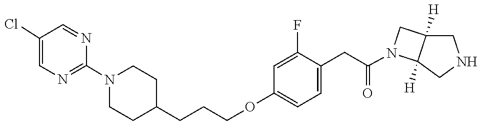 [SMILES: O=C(Cc1ccc(OCCCC2CCN(c3ncc(Cl)cn3)CC2)cc1F)N1C[C@H]2CNC[C@H]21] | ¹H NMR (500 MHz, Chloroform-d) δ 8.20 (s, 2H), 7.22 (dt, J = 13.6, 8.7 Hz, 1H), 6.65 (dt, J = 8.5, 2.6 Hz, 1H), 6.59 (dt, J = 11.7, 2.2 Hz, 1H), 4.80 (ddd, J = 9.8, 6.2, 3.7 Hz, 1H), 4.68 (dp, J = 13.2, 2.0 Hz, 2H), 4.20-4.03 (m, 1H), 3.92 (t, J = 6.5 Hz, 2H), 3.68-3.41 (m, 2H), 3.38-3.21 (m, 2H), 3.08 (dd, J = 12.4, 2.1 Hz, 1H), 2.98 (ddtd, J = 24.3, 8.0, 6.0, 4.2 Hz, 1H), 2.91-2.82 (m, 2H), 2.73 (td, J = 12.3, 5.8 Hz, 1H), 2.59 (ddd, J = 34.8, 13.0, 3.7 Hz, 1H), 1.81 (tdd, J = 10.1, 7.9, 3.9 |
| | | Hz, 4H), 1.57 (ttt, J = 10.7, 6.9, 3.6 Hz, 1H), 1.46-1.37 (m, |
| | | 2H), 1.24-1.12 (m, 2H). LCMS: tR = 0.51, (ES⁺) m/z |
| | | (M + H)⁺ = 488.4. |
| 47 | 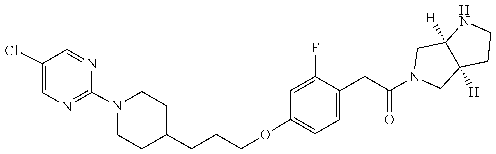 [SMILES: O=C(Cc1ccc(OCCCC2CCN(c3ncc(Cl)cn3)CC2)cc1F)N1C[C@@H]2CCN[C@@H]2C1] | ¹H NMR (500 MHz, Chloroform-d) δ 8.20 (s, 2H), 7.19 (q, J = 8.4 Hz, 1H), 6.64 (dt, J = 8.5, 2.4 Hz, 1H), 6.59 (ddd, J = 11.7, 2.5, 1.2 Hz, 1H), 4.68 (dp, J = 13.3, 1.9 Hz, 2H), 3.91 (ddt, J = 6.7, 5.0, 2.5 Hz, 3H), 3.76-3.69 (m, 1H), 3.68- 3.60 (m, 1H), 3.60-3.49 (m, 3H), 3.37 (dd, J = 12.6, 5.7 Hz, 1H), 3.06-2.96 (m, 1H), 2.90-2.81 (m, 3H), 2.77 (pd, J = 9.4, 8.5, 3.7 Hz, 1H), 2.05-1.91 (m, 1H), 11.81 (ddt, J = 14.4, 9.8, 4.8 Hz, 4H), 1.73-1.60 (m, 1H), 1.56 (dtt, J = 14.7, 7.0, 3.8 Hz, 1H), 1.45-1.38 (m, 2H), 1.23-1.11 (m, 2H). LCMS: tR = 0.52, (ES⁺) m/z (M + H)⁺ = 502.4. |
| 48 | 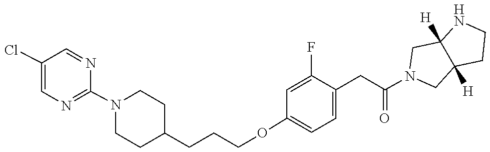 [SMILES: O=C(Cc1ccc(OCCCC2CCN(c3ncc(Cl)cn3)CC2)cc1F)N1C[C@H]2CCN[C@H]2C1] | ¹H NMR (500 MHz, Chloroform-d) δ 8.20 (s, 2H), 7.23- 7.14 (m, 1H), 6.64 (dt, J = 8.5, 2.9 Hz, 1H), 6.59 (ddd, J = 11.7, 2.5, 1.2 Hz, 1H), 4.72-4.63 (m, 2H), 4.00 and 3.75 (m, 1H), 3.91 (t, J = 6.4 Hz, 2H), 3.75-3.51 (m, 4H), 3.47-3.07 (m, 5H), 2.94-2.76 (m, 3H), 2.10-1.96 (m, 1H), 1.86-1.67 (m, 4H), 1.57 (dtq, J = 14.7, 6.8, 3.6 Hz, 1H), 1.46-1.37 (m, 2H), 1.23-1.11 (m, 2H). LCMS: tR = 0.45, (ES⁺) m/z (M + H)⁺ = 502.4. |
| 49 | 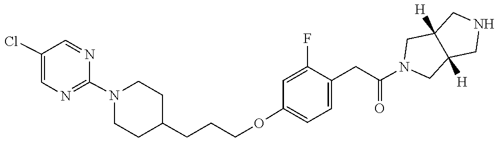 [SMILES: O=C(Cc1ccc(OCCCC2CCN(c3ncc(Cl)cn3)CC2)cc1F)N1C[C@H]2CNC[C@H]2C1] | ¹H NMR (500 MHz, Chloroform-d) δ 8.20 (s, 2H), 7.19 (t, J = 8.7 Hz, 1H), 6.67-6.62 (m, 1H), 6.59 (dd, J = 11.7, 2.5 Hz, 1H), 4.68 (dq, J = 13.2, 2.3 Hz, 2H), 3.92 (t, J = 6.4 Hz, 2H), 3.73-3.66 (m, 2H), 3.56 (s, 2H), 3.43 (dd, J = 12.7, 4.1 Hz, 1H), 3.35 (dd, J = 10.9, 4.4 Hz, 1H), 3.12 (dd, J = 11.8, 5.9 Hz, 2H), 2.86 (ddd, J = 15.2, 11.0, 2.7 Hz, 3H), 2.82-2.67 (m, 3H), 1.85-1.75 (m, 4H), 1.57 (ttt, J = 10.7, 7.0, 3.7 Hz, 1H), 1.46-1.37 (m, 2H), 1.17 (tdd, J = 13.3, 11.6, 4.2 Hz, 2H). LCMS: tR = 0.51, (ES⁺) m/z (M + H)⁺ = 502.5. |
| 50 | 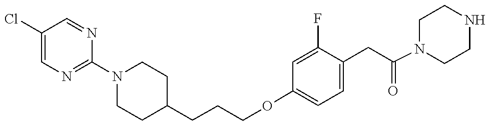 [SMILES: O=C(Cc1ccc(OCCCC2CCN(c3ncc(Cl)cn3)CC2)cc1F)N1CCNCC1] | ¹H NMR (500 MHz, Chloroform-d) δ 8.20 (s, 2H), 7.18 (t, J = 8.7 Hz, 1H), 6.67-6.63 (m, 1H), 6.60 (dd, J = 11.8, 2.5 Hz, 1H), 4.72-4.64 (m, 2H), 3.92 (t, J = 6.5 Hz, 2H), 3.63 (s, 2H), 3.63-3.59 (m, 2H), 3.45 (dd, J = 6.1, 4.0 Hz, 2H), 2.92- 2.73 (m, 6H), 1.84-1.75 (m, 4H), 1.58 (ttt, J = 10.8, 7.0, 3.7 Hz, 1H), 1.46-1.38 (m, 2H), 1.23-1.12 (m, 2H). LCMS: tR = 0.50, (ES⁺) m/z (M + H)⁺ = 476.3. |
| 51 | 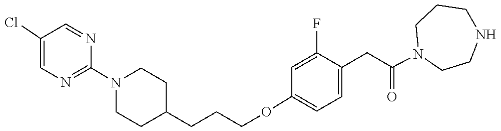 [SMILES: O=C(Cc1ccc(OCCCC2CCN(c3ncc(Cl)cn3)CC2)cc1F)N1CCCNCC1] | ¹H NMR (500 MHz, Chloroform-d) δ 8.20 (s, 2H), 7.20 (td, J = 8.7, 1.9 Hz, 1H), 6.68-6.63 (m, 1H), 6.60 (dt, J = 11.8, 2.2 Hz, 1H), 4.72-4.64 (m, 2H), 3.92 (td, J = 6.5, 1.1 Hz, 2H), 3.67-3.60 (m, 4H), 3.58 (t, J = 6.3 Hz, 1H), 3.56-3.50 (m, 1H), 2.95-2.92 (m, 1H), 2.91-2.80 (m, 5H), 1.85-1.75 (m, 6H), 1.57 (ttt, J = 10.7, 7.0, 3.7 Hz, 1H), 1.46-1.38 (m, 2H), 1.18 (tdd, J = 13.3, 11.6, 4.2 Hz, 2H). LCMS: tR = 0.52, (ES⁺) m/z (M + H)⁺ = 490.5. |
| 52 | 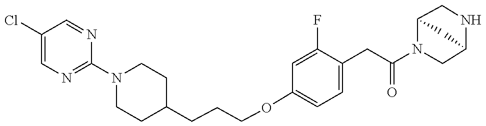 [SMILES: O=C(Cc1ccc(OCCCC2CCN(c3ncc(Cl)cn3)CC2)cc1F)N1C[C@H]2C[C@@H]1CN2] | ¹H NMR (500 MHz, Chloroform-d) δ 8.20 (s, 2H), 7.22 (dt, J = 14.6, 8.6 Hz, 1H), 6.69-6.62 (m, 1H), 6.59 (ddd, J = 11.7, 6.6, 2.5 Hz, 1H), 4.85 (dq, J = 2.0, 1.1 Hz, 1H), 4.72-4.64 (m, 2H), 3.92 (td, J = 6.5, 1.9 Hz, 2H), 3.81-3.74 (m, 1H), 3.62 (q, J = 15.0 Hz, 1H), 3.56-3.48 (m, 1H), 3.36-3.28 (m, 1H), 3.07-2.96 (m, 1H), 2.92-2.82 (m, 2H), 1.87-1.76 (m, 5H), 1.76-1.64 (m, 3H), 1.57 (th, J = 10.8, 3.5 Hz, 1H), 1.46- 1.38 (m, 2H), 1.23-1.12 (m, 2H). LCMS: tR = 0.49, (ES⁺) |
| | | m/z (M + H)⁺ = 488.5. |
| 53 | 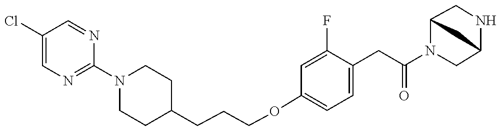 [SMILES: O=C(Cc1ccc(OCCCC2CCN(c3ncc(Cl)cn3)CC2)cc1F)N1C[C@@H]2C[C@H]1CN2] | ¹H NMR (500 MHz, Chloroform-d) δ 8.20 (s, 2H), 7.22 (dt, J = 14.7, 8.7 Hz, 1H), 6.68-6.63 (m, 1H), 6.59 (ddd, J = 11.7, 6.6, 2.5 Hz, 1H), 4.85 (tt, J = 1.9, 1.0 Hz, 1H), 4.68 (dp, J = 13.3, 1.9 Hz, 2H), 3.92 (td, J = 6.4, 1.9 Hz, 2H), 3.82-3.74 (m, 1H), 3.62 (q, J = 15.0 Hz, 1H), 3.56-3.46 (m, 2H), 3.36- 3.27 (m, 1H), 3.05-2.96 (m, 1H), 2.91-2.82 (m, 2H), 1.86- 1.76 (m, 5H), 1.76-1.69 (m, 1H), 1.57 (ddp, J = 11.1, 7.1, 3.6 Hz, 1H), 1.45-1.38 (m, 2H), 1.18 (qd, J = 12.4, 4.2 Hz, |
| | | 2H). LCMS: tR = 0.49, (ES⁺) m/z (M + H)⁺ = 488.5. |
| 54 | 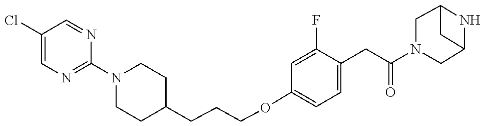 [SMILES: O=C(Cc1ccc(OCCCC2CCN(c3ncc(Cl)cn3)CC2)cc1F)N1CC2CC(C1)N2] | ¹H NMR (500 MHz, Chloroform-d) δ 8.20 (s, 2H), 7.20 (t, J = 8.6 Hz, 1H), 6.69-6.64 (m, 1H), 6.61 (dd, J = 11.7, 2.5 Hz, 1H), 4.68 (ddt, J = 13.5, 4.8, 2.2 Hz, 2H), 3.93 (t, J = 6.4 Hz, 2H), 3.83-3.73 (m, 5H), 3.69-3.60 (m, 3H), 2.91-2.83 (m, 2H), 2.76-2.67 (m, 1H), 1.86-1.76 (m, 4H), 1.57 (dtq, J = 14.8, 7.1, 3.5 Hz, 1H), 1.50-1.38 (m, 4H), 1.23-1.12 (m, 2H). LCMS: tR = 0.51, (ES⁺) m/z (M + H)⁺ = 488.5. |
| 55 | 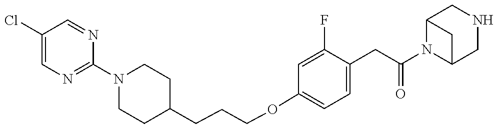 [SMILES: O=C(Cc1ccc(OCCCC2CCN(c3ncc(Cl)cn3)CC2)cc1F)N1C2CNCC1C2] | ¹H NMR (500 MHz, Chloroform-d) δ 8.20 (s, 2H), 7.32- 7.16 (m, 1H), 6.69-6.64 (m, 1H), 6.60 (td, J = 11.5, 2.5 Hz, 1H), 4.68 (dp, J = 13.3, 1.9 Hz, 2H), 4.34 (dddt, J = 21.3, 6.1, 3.9, 2.0 Hz, 1H), 3.92 (td, J = 6.4, 3.5 Hz, 2H), 3.84-3.74 (m, 2H), 3.67-3.61 (m, 1H), 3.54-3.48 (m, 1H), 3.44 (d, J = 1.0 Hz, 1H), 3.23 (ddd, J = 12.6, 2.4, 1.3 Hz, 1H), 3.05 (dd, J = 12.6, 1.7 Hz, 1H), 2.96 (dd, J = 12.5, 1.8 Hz, 1H), 2.90- 2.82 (m, 2H), 2.76-2.59 (m, 1H), 1.81 (ddtt, J = 14.4, 6.2, |
| | | 4.0, 2.1 Hz, 4H), 1.57 (th, J = 10.8, 3.5 Hz, 1H), 1.49-1.39 |
| | | (m, 2H), 1.22-1.13 (m, 2H). LCMS: tR = 0.50, (ES⁺) m/z |
| | | (M + H)⁺ = 488.5. |
| 56 | 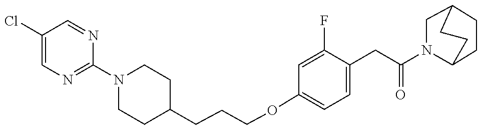 [SMILES: O=C(Cc1ccc(OCCCC2CCN(c3ncc(Cl)cn3)CC2)cc1F)N1CC2CCC1CC2] | ¹H NMR (500 MHz, Chloroform-d) δ 8.20 (s, 2H), 7.20 (td, J = 8.7, 4.2 Hz, 1H), 6.65 (dd, J = 8.5, 2.5 Hz, 1H), 6.60 (ddd, J = 11.8, 3.8, 2.5 Hz, 1H), 4.68 (dp, J = 13.3, 2.0 Hz, 2H), 3.92 (td, J = 6.5, 2.1 Hz, 2H), 3.84 (p, J = 2.6 Hz, 1H), 3.74 (dt, J = 10.4, 2.7 Hz, 1H), 3.66-3.53 (m, 4H), 3.26 (dt, J = 11.2, 2.8 Hz, 1H), 3.13 (ddt, J = 10.8, 3.7, 2.1 Hz, 2H), 3.10-3.05 (m, 1H), 2.86 (td, J = 12.9, 2.7 Hz, 2H), 2.00-1.90 (m, 2H), 1.89-1.75 (m, 6H), 1.75-1.68 (m, 1H), 1.58 (dqd, |
| | | J = 14.4, 7.0, 3.3 Hz, 3H), 1.46-1.38 (m, 2H), 1.23-1.12 |
| | | (m, 2H). LCMS: tR = 0.52, (ES⁺) m/z (M + H)⁺ = 502.5. |
| 57 | 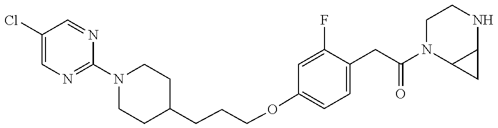 [SMILES: O=C(Cc1ccc(OCCCC2CCN(c3ncc(Cl)cn3)CC2)cc1F)N1CCNC2CC21] | ¹H NMR (500 MHz, Chloroform-d) δ 8.20 (s, 2H), 7.21 (t, J = 8.6 Hz, 1H), 6.68-6.63 (m, 1H), 6.63-6.56 (m, 1H), 4.68 (dq, J = 13.1, 2.3 Hz, 2H), 3.96-3.88 (m, 3H), 3.88-3.76 (m, 2H), 2.94-2.74 (m, 6H), 1.81 (dddd, J = 13.0, 11.0, 6.2, 4.1 Hz, 4H), 1.57 (dtt, J = 11.0, 7.1, 3.6 Hz, 2H), 1.45-1.37 (m, 2H), 1.18 (tdd, J = 13.1, 11.6, 4.2 Hz, 2H), 1.00 and 0.95 (dt, J = 7.4, 6.4 Hz, 1H), 0.64 and 0.52 (ddd, J = 6.3, 5.0, 4.2 Hz, 1H). LCMS: tR = 0.53, (ES⁺) m/z (M + H)⁺ = 488.5. |
| 58 | 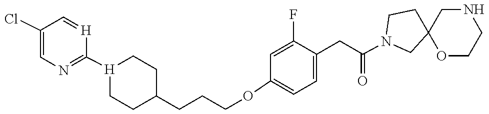 | ¹H NMR (500 MHz, Chloroform-d) δ 8.20 (s, 2H), 7.19 (td, J = 8.7, 3.3 Hz, 1H), 6.67-6.62 (m, 1H), 6.59 (dt, J = 11.7, 2.4 Hz, 1H), 4.68 (dp, J = 13.3, 1.9 Hz, 2H), 3.92 (t, J = 6.4 Hz, 2H), 3.85 and 3.41 (m, 1H), 3.75-3.69 (m, 2H), 3.69-3.62 (m, 1H), 3.62-3.51 (m, 4H), 2.90-2.82 (m, 5H), 2.20 and 2.15 (m, 1H), 2.14-2.07 (m, 1H), 1.94-1.75 (m, 5H), 1.57 (ttt, J = 10.7, 7.0, 3.7 Hz, 1H), 1.45-1.36 (m, 2H), 1.23- |
| | | 1.11 (m, 2H). LCMS: tR = 0.48, (ES⁺) m/z (M + H)⁺ = 532.5. |
| 59 | 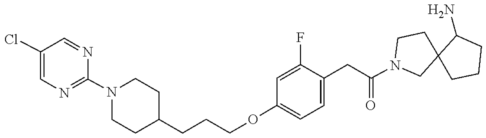 [SMILES: NC1CCCC12CCN(C(=O)Cc1ccc(OCCCC3CCN(c4ncc(Cl)cn4)CC3)cc1F)C2] | ¹H NMR (500 MHz, Chloroform-d) δ 8.20 (s, 2H), 7.23- 7.15 (m, 1H), 6.65 (dd, J = 8.5, 2.5 Hz, 1H), 6.59 (dt, J = 11.9, 2.1 Hz, 1H), 4.72-4.64 (m, 2H), 3.92 (td, J = 6.5, 1.4 Hz, 2H), 3.72-3.40 (m, 4H), 3.36-3.28 (m, 1H), 3.23 (dd, J = 10.7, 8.4 Hz, 1H), 3.12-3.00 (m, 1H), 2.91-2.80 (m, 2H), 2.09-1.87 (m, 2H), 1.85-1.48 (m, 12H), 1.44-1.38 (m, 3H), 1.23-1.12 (m, 2H). LCMS: tR = 0.56, (ES⁺) m/z (M + H)⁺ = 530.5. |
| 60 | 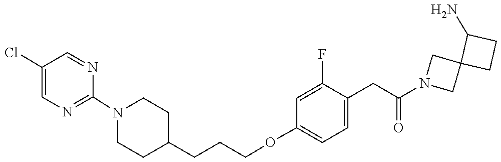 [SMILES: NC1CCC12CN(C(=O)Cc1ccc(OCCCC3CCN(c4ncc(Cl)cn4)CC3)cc1F)C2] | ¹H NMR (500 MHz, Chloroform-d) δ 8.20 (s, 2H), 7.22 (td, J = 8.6, 7.1 Hz, 1H), 6.67-6.62 (m, 1H), 6.59 (ddd, J = 11.7, 5.3, 2.5 Hz, 1H), 4.68 (dq, J = 13.3, 2.3 Hz, 2H), 4.38 (dd, J = 83.7, 9.2 Hz, 1H), 4.03-3.95 (m, 1H), 3.92 (td, J = 6.4, 1.2 Hz, 2H), 3.90-3.73 (m, 2H), 3.43-3.35 (m, 2H), 3.35- 3.27 (m, 1H), 2.91-2.81 (m, 2H), 2.18 (dtt, J = 10.8, 8.3, 2.5 Hz, 1H), 1.91 (tdt, J = 9.4, 2.7, 1.4 Hz, 1H), 1.81 (ddt, J = 12.7, 10.6, 4.9 Hz, 4H), 1.68-1.49 (m, 4H), 1.46-1.37 (m, 3H), 1.17 (qd, J = 12.4, 4.2 Hz, 2H). LCMS: tR = 1.53, (ES⁺) m/z (M + H)⁺ = 502.5. |
| 61 | 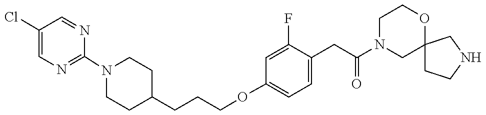 [SMILES: O=C(Cc1ccc(OCCCC2CCN(c3ncc(Cl)cn3)CC2)cc1F)N1CCOC2(CCNC2)C1] | ¹H NMR (500 MHz, Chloroform-d) δ 8.20 (s, 2H), 7.19 (t, J = 8.7 Hz, 1H), 6.67 (dt, J = 8.6, 2.6 Hz, 1H), 6.61 (dd, J = 11.8, 2.5 Hz, 1H), 4.68 (dp, J = 13.2, 1.9 Hz, 2H), 3.92 (td, J = 6.5, 2.4 Hz, 2H), 3.70-3.57 (m, 5H), 3.54 (q, J = 5.4, 4.5 Hz, 1H), 3.49-3.43 (m, 2H), 3.09 (dtd, J = 11.4, 7.4, 3.8 Hz, 1H), 2.96 (dd, J = 17.3, 6.2 Hz, 2H), 2.90-2.82 (m, 2H), 2.72 (dd, J = 40.5, 12.0 Hz, 1H), 1.90-1.70 (m, 6H), 1.58 (ddt, J = 18.4, 11.5, 6.7 Hz, 2H), 1.42 (tdd, J = 9.4, 6.2, 2.0 Hz, 2H), |
| | | 1.18 (qd, J = 12.5, 4.2 Hz, 2H). LCMS: tR = 0.51, (ES⁺) m/z |
| | | (M + H)⁺ = 532.4. |
| 62 | 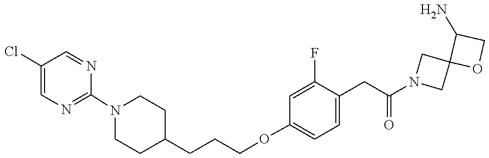 [SMILES: NC1COC12CN(C(=O)Cc1ccc(OCCCC3CCN(c4ncc(Cl)cn4)CC3)cc1F)C2] | ¹H NMR (500 MHz, Chloroform-d) δ 8.20 (s, 2H), 7.21 (td, J = 8.6, 4.7 Hz, 1H), 6.65 (dt, J = 8.5, 2.0 Hz, 1H), 6.59 (dt, J = 11.7, 2.9 Hz, 1H), 4.75-4.65 (m, 3H), 4.55 (ddd, J = 69.1, 10.8, 1.7 Hz, 1H), 4.29-4.18 (m, 1H), 4.18-4.10 (m, 2H), 4.09-3.94 (m, 2H), 3.92 (t, J = 6.5 Hz, 2H), 3.41 (d, J = 16.0 Hz, 2H), 2.91-2.82 (m, 2H), 1.85-1.75 (m, 4H), 1.62-1.52 (m, 3H), 1.45-1.38 (m, 2H), 1.23-1.12 (m, 2H). LCMS: tR = 1.49, (ES⁺) m/z (M + H)⁺ = 504.4. |
| 63 | 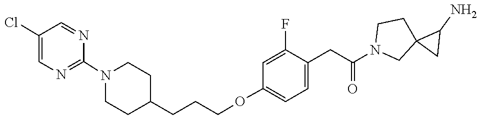 [SMILES: NC1CC12CCN(C(=O)Cc1ccc(OCCCC3CCN(c4ncc(Cl)cn4)CC3)cc1F)C2] | ¹H NMR (500 MHz, Chloroform-d) δ 8.20 (s, 2H), 7.25- 7.17 (m, 1H), 6.65 (dt, J = 8.5, 3.0 Hz, 1H), 6.62-6.57 (m, 1H), 4.68 (dp, J = 13.4, 2.0 Hz, 2H), 3.92 (td, J = 6.5, 1.7 Hz, 2H), 3.76 (dd, J = 18.0, 11.3 Hz, 1H), 3.71-3.60 (m, 1H), 3.57-3.47 (m, 1H), 3.45-3.20 (m, 1H), 2.91-2.81 (m, 2H), 2.42-2.30 (m, 1H), 1.99-1.76 (m, 6H), 1.70 (ddd, J = 12.1, 7.3, 4.6 Hz, 4H), 1.57 (dtd, J = 11.0, 7.2, 3.8 Hz, 2H), 1.46- 1.37 (m, 2H), 1.23-1.09 (m, 2H). LCMS: tR = 1.49, (ES⁺) |
| | | m/z (M + H)⁺ = 504.4. |
| 64 | 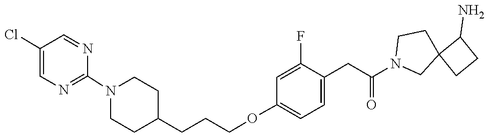 [SMILES: NC1CCC12CCN(C(=O)Cc1ccc(OCCCC3CCN(c4ncc(Cl)cn4)CC3)cc1F)C2] | ¹H NMR (500 MHz, Chloroform-d) δ 8.20 (s, 2H), 7.20 (q, J = 8.4 Hz, 1H), 6.68-6.55 (m, 2H), 4.68 (dp, J = 13.5, 1.9 Hz, 2H), 3.91 (td, J = 6.5, 2.6 Hz, 2H), 3.84 (t, J = 10.6 Hz, 1H), 3.61 (d, J = 6.1 Hz, 1H), 3.59-3.43 (m, 4H), 3.43-3.31 (m, 2H), 2.86 (m, 2H), 2.29-2.19 (m, 2H), 2.04 (dt, J = 13.7, 7.3 Hz, 1H), 1.90 (dq, J = 14.1, 7.2 Hz, 1H), 1.81 (tt, J = 11.9, 6.3 Hz, 5H), 1.71-1.52 (m, 3H), 1.45-1.38 (m, 2H), 1.17 (qd, J = 12.5, 4.2 Hz, 2H). |

### Intermediate 65: (2R,3R,4R,5S)-6-(piperazin-1-yl)hexane-1,2,3,4,5-pentaol dihydrochloride

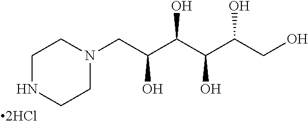 [SMILES: OC[C@@H](O)[C@@H](O)[C@H](O)[C@@H](O)CN1CCNCC1]

To a mixture of tert-butyl piperazine-1-carboxylate (1.5 g, 8.05 mmol) and D-glucose (1.74 g, 9.66 mmol) in a mixture of MeOH (15 mL) and acetic acid (0.74 mL, 12.89 mmol) was added sodium cyanoborohydride (1.01 g, 16.1 mmol) and the resulting mixture stirred at room temperature overnight. The mixture was evaporated and the residue was treated with a mixture of MeOH (10 mL) and 12M HCl (10 mL, 121 mmol) and the resulting mixture stirred at room temperature for 2 hours. Mixture filtered to remove a small amount of white solid and the filtrate evaporated to give (2R,3R,4R,5S)-6-(piperazin-1-yl)hexane-1,2,3,4,5-pentaol dihydrochloride (3g, 100%) as a white solid that was used crude in subsequent reactions. LCMS: tR=0.14, (ES⁺) m/z (M+H)⁺=251.2.

### Example 1: 5-(7-(2-(4-(3-(1-(5-chloropyrimidin-2-yl)piperidin-4-yl)propoxy)-2-fluorophenyl)acetyl)-2,7-diazaspiro[4.4]nonan-2-yl)-5-oxopentane-1-sulfonic acid

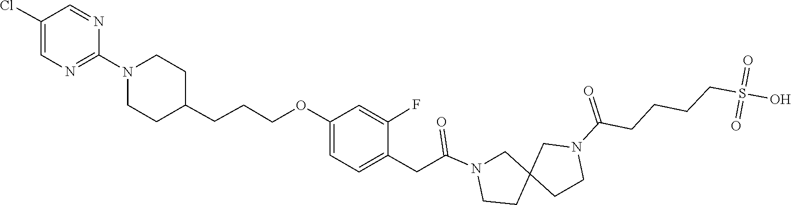 [SMILES: O=C(CCCCS(=O)(=O)O)N1CCC2(CCN(C(=O)Cc3ccc(OCCCC4CCN(c5ncc(Cl)cn5)CC4)cc3F)C2)C1]

To a mixture of 2-[4-[3-[1-(5-chloropyrimidin-2-yl)-4-piperidyl]propoxy]-2-fluoro-phenyl]-1-(2,7-diazaspiro[4.4]nonan-2-yl)ethanone Intermediate 41 (20 mg, 0.039 mmol) and 5-sulfopentanoic acid (9 mg, 0.05 mmol) in DMF (0.5 mL) was added HATU (22 mg, 0.058 mmol) and Hunig's base (0.034 mL, 0.194 mmol) and the resulting mixture stirred at room temperature for 1 hour. Mixture treated with formic acid (0.1 mL) and purified directly by reverse phase PREP-HPLC (C18 column: eluent gradient 15-85% CH₃CN in water+0.1% formic acid). Product containing fractions lyophilized to give 5-(7-(2-(4-(3-(1-(5-chloropyrimidin-2-yl)piperidin-4-yl)propoxy)-2-fluorophenyl)acetyl)-2,7-diazaspiro[4.4]nonan-2-yl)-5-oxopentane-1-sulfonic acid_(17.2 mg, 63%) as a fluffy white solid. LCMS: tR=1.01, (ES⁺) m/z (M+H)⁺=680.4.

The following compounds in Table P3 were prepared using procedures similar to those described in Example 1 using appropriate starting materials.

TABLE P3

| Example # | Intermediate used | Observed Exact Mass [M + H]⁺ |
|---|---|---|
| 2 | 40 | 666.4 |
| 3 | 36 | 652.3 |
| 4 | 39 | 666.4 |
| 5 | 34 | 652.3 |
| 6 | 47 | 666.4 |
| 7 | 45 | 652.3 |
| 8 | 46 | 652.3 |
| 9 | 37 | 652.3 |
| 10 | 49 | 666.4 |
| 11 | 52 | 652.3 |
| 12 | 54 | 652.4 |
| 13 | 51 | 654.4 |
| 14 | 56 | 666.4 |
| 15 | 53 | 652.4 |
| 16 | 55 | 652.3 |
| 17 | 57 | 652.3 |
| 18 | 33 | 666.3 |
| 19 | 35 | 688.3 |
| 20 | 48 | 666.4 |
| 21 | 43 | 680.4 |
| 22 | 44 | 680.4 |
| 23 | 50 | 640.4 |
| 24 | 58 | 696.4 |

### Example 25: 2-(4-(3-(1-(5-chloropyrimidin-2-yl)piperidin-4-yl)propoxy)-2-fluorophenyl)-1-(6-((2S,3S,4R)-2,3,4,5-tetrahydroxypentyl)-2,6-diazaspiro[3.3]heptan-2-yl)ethan-1-one formate

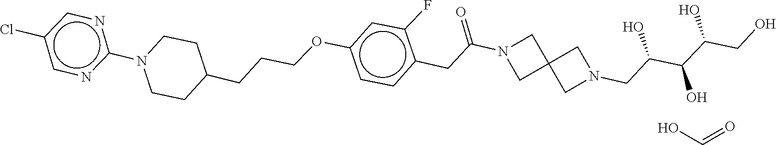

To a mixture of 1-(2-aza-6-azoniaspiro[3.3]heptan-2-yl)-2-[4-[3-[1-(5-chloropyrimidin-2-yl)-4-piperidyl]propoxy]-2-fluoro-phenyl]ethanone Intermediate 36 (30 mg, 0.057 mmol), D-ribose (17 mg, 0.114 mmol) and acetic acid (0.016 mL, 0.288 mmol) in MeOH (1 mL) was added sodium cyanoborohydride (11 mg, 0.172 mmol) and a spatula end of 3A° powdered molecular sieves and the resulting mixture stirred at room temperature overnight. Treated with formic acid (0.1 mL) and purified directly by reverse phase PREP-HPLC (C18 column: gradient 15-85% CH₃CN in water+0.1% formic acid). Product containing fractions lyophilized to give 2-(4-(3-(1-(5-chloropyrimidin-2-yl)piperidin-4-yl)propoxy)-2-fluorophenyl)-1-(6-((2S,3S,4R)-2,3,4,5-tetrahydroxypentyl)-2,6-diazaspiro[3.3]heptan-2-yl)ethan-1l-one-A7.5 mg, 18%) as a fluffy white solid. LCMS: tR=1.76, (ES⁺) m/z (M+H)⁺=622.4.

The following compounds in Table P4 were prepared using procedures similar to those described in Example 25 using appropriate starting materials.

TABLE P4

| | Intermediate | | Observed Exact Mass |
|---|---|---|---|
| Example # | used | Sugar used | [M + H]⁺ |
| 26 | 36 | D-glucose | 652.4 |
| 27 | 50 | D-glucose | 640.5 |
| 28 | 40 | D-ribose | 636.5 |
| 29 | 40 | D-glucose | 666.4 |
| 30 | 39 | D-ribose | 636.5 |
| 31 | 39 | D-glucose | 666.4 |
| 32 | 34 | D-ribose | 622.4 |
| 33 | 34 | D-glucose | 652.4 |
| 34 | 47 | D-ribose | 636.5 |
| 35 | 47 | D-glucose | 666.4 |
| 36 | 45 | D-ribose | 622.4 |
| 37 | 45 | D-glucose | 652.4 |
| 38 | 46 | D-ribose | 622.4 |
| 39 | 46 | D-glucose | 652.4 |
| 40 | 37 | D-ribose | 622.4 |
| 41 | 37 | D-glucose | 652.4 |
| 42 | 41 | D-ribose | 650.4 |
| 43 | 41 | D-glucose | 680.4 |
| 44 | 49 | D-ribose | 636.5 |
| 45 | 49 | D-glucose | 666.4 |
| 46 | 52 | D-ribose | 622.4 |
| 47 | 52 | D-glucose | 652.5 |
| 48 | 54 | D-ribose | 622.4 |
| 49 | 54 | D-glucose | 652.3 |
| 50 | 51 | D-ribose | 624.5 |
| 51 | 51 | D-glucose | 654.5 |
| 52 | 56 | D-ribose | 636.4 |
| 53 | 56 | D-glucose | 666.4 |
| 54 | 53 | D-ribose | 622.4 |
| 55 | 53 | D-glucose | 652.4 |
| 56 | 55 | D-ribose | 622.4 |
| 57 | 55 | D-glucose | 652.4 |
| 58 | 57 | D-ribose | 622.4 |
| 59 | 57 | D-glucose | 652.4 |
| 60 | 33 | D-ribose | 636.4 |
| 61 | 33 | D-glucose | 666.4 |
| 62 | 35 | D-ribose | 658.4 |
| 63 | 35 | D-glucose | 688.4 |
| 64 | 48 | D-ribose | 636.4 |
| 65 | 48 | D-glucose | 666.4 |
| 66 | 38 | D-ribose | 636.4 |
| 67 | 43 | D-ribose | 650.4 |
| 68 | 43 | D-glucose | 680.4 |
| 69 | 44 | D-ribose | 650.4 |
| 70 | 44 | D-glucose | 680.4 |
| 71 | 50 | D-ribose | 610.5 |
| 72 | 58 | D-ribose | 666.4 |
| 73 | 58 | D-glucose | 696.4 |
| 74 | 42 | D-ribose | 650.4 |
| 75 | 42 | D-glucose | 680.4 |
| 76 | 59 | D-ribose | 664.4 |
| 77 | 59 | D-glucose | 694.4 |
| 78 | 60 | D-ribose | 636.5 |
| 79 | 60 | D-glucose | 666.4 |
| 80 | 61 | D-ribose | 666.4 |
| 81 | 61 | D-glucose | 696.4 |
| 82 | 62 | D-ribose | 638.5 |
| 83 | 63 | D-ribose | 636.4 |
| 84 | 64 | D-ribose | 650.4 |

### Example 85: 2-(4-(3-(1-(5-chloropyrimidin-2-yl)piperidin-4-yl)propoxy)-2,6-difluorophenyl)-1-(4-((2S,3R,4R,5R)-2,3,4,5,6-pentahydroxyhexyl)piperazin-1-yl)ethan-1-one formate

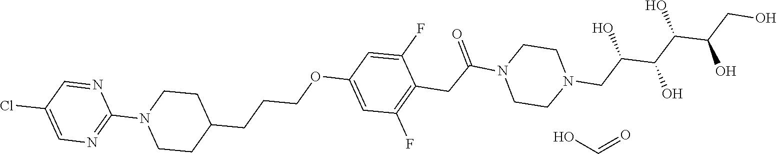

To a mixture of 2-[4-[3-[1-(5-chloropyrimidin-2-yl)-4-piperidyl]propoxy]-2,6-difluoro-phenyl]acetic acid Intermediate 2 (25 mg, 0.0587 mmol) and (2R,3R,4R,5S)-6-piperazine-1,4-diium-1-ylhexane-1,2,3,4,5-pentol dihydrochloride Intermediate 65 (29 mg, 0.088 mmol) in DMF (1 mL) was treated with HATU (34 mg, 0.088 mmol) and Hunig's base (0.051 mL, 0.294 mmol) and the resulting mixture stirred at room temperature for 30 mins. Mixture treated with formic acid (0.1 mL) and purified directly by reverse phase PREP-HPLC (C18 column: gradient 20-80% acetonitrile in water+0.1% formic acid). Product containing fractions lyophilized to give 2-(4-(3-(1-(5-chloropyrimidin-2-yl)piperidin-4-yl)propoxy)-2,6-difluorophenyl)-1-(4-((2S,3R,4R,5R)-2,3,4,5,6-pentahydroxyhexyl)piperazin-1-yl)ethan-1-one formate (34.8 mg, 82%) as a fluffy white solid. LCMS: tR=1.45, (ES⁺) m/z (M+H)⁺=658.4.

The following compounds in Table P5 were prepared using procedures similar to those described in Example 85 using appropriate starting materials.

TABLE P5

| Example # | Intermediate used | Observed Exact Mass [M + H]⁺ |
|---|---|---|
| 86 | 3 | 654.4 |
| 87 | 4 | 672.4 |
| 88 | 15 | 623.4 |
| 89 | 16 | 641.4 |
| 90 | 19 | 623.4 |
| 91 | 17 | 640.4 |
| 92 | 5 | 634.5 |
| 93 | 6 | 652.5 |
| 94 | 7 | 648.6 |
| 95 | 8 | 666.6 |
| 96 | 9 | 650.5 |
| 97 | 10 | 668.5 |
| 98 | 11 | 650.5 |
| 99 | 12 | 668.5 |
| 100 | 13 | 664.5 |
| 101 | 14 | 682.5 |
| 102 | 18 | 676.5 |
| 103 | 20 | 650.5 |
| 104 | 23 | 614.6 |
| 105 | 21 | 696.5 |
| 106 | 22 | 714.4 |
| 107 | 32 | 668.5 |
| 108 | 30 | 652.4 |
| 109 | 31 | 666.4 |
| 110 | 24 | 666.4 |
| 111 | 25 | 680.4 |
| 112 | 27 | 694.4 |
| 113 | 26 | 666.4 |
| 114 | 28 | 666.4 |
| 115 | 29 | 680.4 |

### II. Biological Evaluation

### Example A-1: In Vitro Activity Assay

### Cell Line Expressing GPR119

CHO-K1 cells stably expressing human GPR119 (hGPR119) were prepared by transfection of a GPR119-carrying plasmid using Lipofectamine 2000 (following manufacturer instructions). A stable cell line was established using the limiting dilution method with geneticine selection. Assay-ready frozen (ARF) cells were prepared and used throughout the study.

cAMP Accumulation Assay

The assay was performed in a 384-well plate format using the cAMP Gs dynamic assay kit from Cisbio. ARF cells expressing hGPR119 were thawed, washed and then resuspended in cAMP stimulation buffer at a cell density of 1.1×10⁶ cells/mL. Cells were plated at a density of ˜10,000 cells/well (9 μL/well). Dose response curves for the tested compounds were prepared in a cAMP stimulation buffer, containing 0.1% Tween 80 at 4 fold the final concentration. The compounds were then transferred to the cell plates using BRAVO (3 μL/well) and the plates were incubated for 60 minutes at 37° C./5% CO₂. Detection buffer (10 μL, prepared as described in the cAMP Gs dynamic kit) were added to each well, and the plates were incubated at ambient temperature for 1 hr.

RT-FRET was measured using a ClarioSTAR plate reader, calculating the ratio between emissions at 665 nm and 620 nm (HTRF ratio). The HTRF ratio for positive (Max) and negative (Min) controls were used to normalize HTRF data and generate values for % activity. EC₅₀ and Max activity values were determined using a standard 4-parameter fit.

Results for exemplary compounds are shown in Table 2.

TABLE 2

| Compound | Max Activityª | EC₅₀^{b} |
|---|---|---|
| 1 | ++ | A |
| 2 | +++ | A |
| 3 | ++ | A |
| 4 | ++ | A |
| 5 | ++ | A |
| 6 | +++ | A |
| 7 | ++ | B |
| 8 | ++ | A |
| 9 | ++ | C |
| 10 | ++ | A |
| 11 | ++ | A |
| 12 | ++ | A |
| 13 | ++ | B |
| 14 | ++ | A |
| 15 | ++ | A |
| 16 | ++ | A |
| 17 | ++ | B |
| 18 | ++ | A |
| 19 | ++ | A |
| 20 | ++ | A |
| 21 | ++ | A |
| 22 | ++ | B |
| 23 | ++ | A |
| 24 | ++ | A |
| 25 | + | C |
| 26 | ++ | A |
| 27 | ++ | A |
| 28 | ++ | A |
| 29 | ++ | A |
| 30 | ++ | A |
| 31 | ++ | A |
| 32 | ++ | A |
| 33 | ++ | A |
| 34 | +++ | A |
| 35 | ++ | A |
| 36 | ++ | B |
| 37 | ++ | B |
| 38 | ++ | A |
| 39 | ++ | A |
| 40 | ++ | B |
| 41 | ++ | C |
| 42 | ++ | A |
| 43 | ++ | A |
| 44 | ++ | A |
| 45 | ++ | A |
| 46 | +++ | A |
| 47 | ++ | A |
| 48 | +++ | A |
| 49 | ++ | A |
| 50 | ++ | A |
| 51 | ++ | A |
| 52 | +++ | A |
| 53 | ++ | A |
| 54 | ++ | A |
| 55 | ++ | A |
| 56 | ++ | A |
| 57 | ++ | A |
| 58 | ++ | A |
| 59 | ++ | A |
| 60 | +++ | A |
| 61 | +++ | A |
| 62 | ++ | A |
| 63 | +++ | A |
| 64 | ++ | A |
| 65 | ++ | A |
| 66 | ++ | A |
| 67 | ++ | A |
| 68 | ++ | A |
| 69 | ++ | B |
| 70 | ++ | B |
| 71 | ++ | A |
| 72 | ++ | A |
| 73 | ++ | A |
| 74 | +++ | A |
| 75 | +++ | A |
| 76 | +++ | A |
| 77 | +++ | A |
| 78 | +++ | A |
| 79 | +++ | A |
| 80 | ++ | A |
| 81 | ++ | A |
| 82 | ++ | A |
| 83 | ++ | A |
| 84 | +++ | A |
| 85 | +++ | A |
| 86 | ++ | A |
| 87 | +++ | A |
| 88 | ++ | C |
| 89 | ++ | B |
| 90 | ++ | B |
| 91 | ++ | B |
| 92 | ++ | A |
| 93 | ++ | A |
| 94 | ++ | A |
| 95 | ++ | A |
| 96 | ++ | B |
| 97 | ++ | A |
| 98 | ++ | B |
| 99 | ++ | A |
| 100 | ++ | B |
| 101 | ++ | A |
| 102 | ++ | B |
| 103 | + | C |
| 104 | ++ | B |
| 105 | ++ | B |
| 106 | ++ | B |
| 107 | ++ | B |
| 108 | ++ | B |
| 109 | ++ | B |
| 110 | ++ | B |
| 111 | ++ | A |
| 112 | ++ | B |
| 113 | + | C |
| 114 | ++ | A |
| 115 | ++ | B |

^{a}+++ ≥ 130%; 130% > ++ ≥ 100%; 100% > + ≥ 50%; − < 50%.
^{b}A ≤ 100 nM; 100 nM < B ≤ 1000 nM; C > 10000 nM.

We claim:

1. A compound of Formula (I): 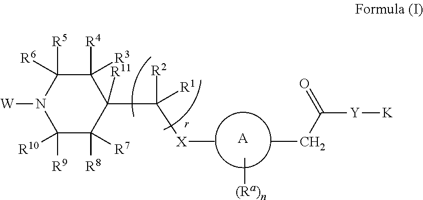
or a pharmaceutically acceptable salt, solvate, stereoisomer, or prodrug thereof, wherein:
Y is 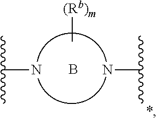
where Ring B is a heterocycloalkyl; wherein Ring B comprises 2 N atoms and 0 or 1 O or S atoms, and * represents the attachment point to K;
or Y is 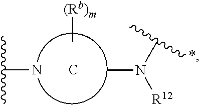
where Ring C is a bicyclic heterocycloalkyl; wherein Ring C comprises 1 or 2 N atoms and 0 or 1 O or S atoms, R¹² is hydrogen or C₁₋₄ alkyl, and * represents the attachment point to K;
each R^{b} is independently fluoro, C₁-6 alkyl, or C₁₋₆ fluoroalkyl;
K is C₁₋₈ alkyl, C₂₋₈ alkenyl, C₂₋₈ alkynyl, —C(═O)—C₁₋₈ alkyl, —C(═O)—C₂₋₈ alkenyl, —C(═O)—C₂₋₈ alkynyl, —[(CH₂)ₛ—Z]ₜ—R¹³, —[(CHR^{d})ₛ—Z]ₜ—R¹³, or —[(C(R^{d})₂)ₛ—Z]ₜ—R¹³; wherein each alkyl, alkenyl, or alkynyl is substituted by 1-6 R^{c} groups;
each Z is independently —CH₂O—, —CH₂NH—, —CH₂NR^{d}—, —CH₂N⁺(R^{d})₂—, —NH—C(═O)—NH—, —C(═O)NH—, —CH₂S(═O)₂—, or —CH₂S(═O)—;
each s is independently 1-6;
each t is independently 1-6;
R¹³ is hydrogen, C₁₋₈ alkyl, C₂₋₈alkenyl, or C₂₋₈ alkynyl, wherein the alkyl, alkenyl, or alkynyl is unsubstituted or substituted by 1-6 R^{c} groups;
each R^{c} is independently —OH, —CH₂OH, —CH₂CH₂OH, —NH₂, —CH₂NH₂, —NH(R^{d}), —CH₂NH(R^{d}), —N(R^{d})₂, —CH₂N(R^{d})₂, —N(R^{d})₃⁺, —C(═O)OH, —CH₂C(═O)OH, —CH₂CH₂C(═O)OH, —S(═O)₂OH, —S(═O)OH, —S(═O)₂NH₂, —P(═O)(OH)₂, —P(═O)(OH)(R^{d}), —P(═O)(OH)(H), P(═O)(OH)(OR^{d}), —B(OH)₂, —B(OR^{d})(OH), —NHCONHS(═O)₂(R^{d}), —N(R^{d})CONHS(═O)₂(R^{d}), —NHCON(R^{d})S(═O)₂(R^{d}), —C(═O)NHS(═O)₂(R^{d}), —S(═O)₂NHC(═O)R^{d}, —NHC(═NH)NH₂, —NHC(═NH)NHR^{d}, —NHC(═NH)N(R^{d})₂, —N(R^{d})C(═NH)NH₂, —N(R^{d})C(═NH)NH(R^{d}), —N(R^{d})C(═NH)N(R^{d})₂, —NHC(═N(R^{d}))NH₂, —NHC(═N(R^{d}))NHR^{d}, —NHC(═N(R^{d}))N(R^{d})₂, —N(R^{d})C(═N(R^{d}))NH₂, —N(R^{d})C(═N(R^{d}))NR^{d}, —N(R^{d})C(═N(R^{d}))N(R^{d})₂, —NHC(═NH)NHC(═NH)NH₂, —N(R^{d})C(═NH)NHC(═NH)NH₂, 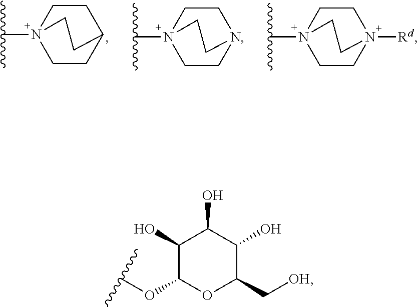
or a 4- to 6-membered heterocycle which is unsubstituted or substituted with 1, 2, 3, or 4 substituents selected from the group consisting of C₁₋₆ alkyl, —O—(C₁₋₆ alkyl), —OH, ═O and ═S;
each R^{d} is independently C₁₋₆ alkyl, C₁₋₆ fluoroalkyl, or C₃₋₆ cycloalkyl;
Ring A is phenyl, 5-membered monocyclic heteroaryl, or 6-membered monocyclic heteroaryl;
each R^{a} is independently halogen, —CN, C₁₋₆ alkyl, C₁₋₆ fluoroalkyl, or C₃₋₆ cycloalkyl;
X is —O—, —NR¹⁴—, #—CH₂O—, #—CH₂NR¹⁴—, #—C(═O)O—, #—C(═O)NR¹⁴—, #—CH₂C(═O)O—, #—CH₂C(═O)NR¹⁴—, #—OC(═O)—, #—NR¹⁴C(═O)—, #—CH₂OC(═O)—, or #—CH₂NR¹⁴C(═O)—, where # represents the attachment point to Ring A;
R¹⁴ is hydrogen or C₁₋₄ alkyl;
each R¹ is independently hydrogen, fluorine, —OH, C₁₋₆ alkyl, or C₁₋₆ alkoxy;
or two R¹ are taken together with the intervening atoms to which they are attached to form a C₃₋₆ cycloalkyl;
each R² is independently hydrogen, fluorine, or C₁₋₆ alkyl;
R³, R⁴, R⁵, R⁶, R⁷, R⁸, R⁹, and R¹⁰ are each independently hydrogen, C₁₋₆ alkyl, C₁₋₆ alkoxy, or C₁₋₆ fluoroalkyl;
or R³ and R⁷ or R³ and R⁹ or R⁵ and R⁹ are taken together with the intervening atoms to which they are attached to form a ring;
R¹¹ is hydrogen, C₁₋₆ alkyl, or C₁₋₆ fluoroalkyl;
or R¹¹ and one Rt are taken together with the intervening atoms to which they are attached to form a C₃₋₆ cycloalkyl;
W is phenyl, 5-membered monocyclic heteroaryl, or 6-membered monocyclic heteroaryl, wherein the phenyl or heteroaryl is unsubstituted or substituted with 1, 2, or 3 substituents selected from R^{e};
each R^{e} is independently halogen, —OH, —CN, —C(O)OH, —C(O)O(C₁₋₆ alkyl), C₁₋₆ alkyl, C₁₋₆ alkoxy, C₃₋₆ cycloalkyl, phenyl, or 5- to 6-membered heteroaryl; wherein each alkyl, alkoxy, and cycloalkyl is unsubstituted or substituted with 1, 2, or 3 substituents selected from the group consisting of halogen, —OH, C₁₋₆ alkyl, and C₁₋₆ alkoxy;
or W is —C(═O)O—R¹⁵;
R¹¹ is C₁₋₆ alkyl, C₃₋₈cycloalkyl, or 3- to 6-membered heterocycloalkyl, wherein the alkyl, cycloalkyl, and heterocycloalkyl are unsubstituted or substituted by 1-3 substituents independently selected from the group consisting of fluorine, —OH, C₁₋₆ alkyl, C₁₋₆ fluoroalkyl, or C₃₋₆ cycloalkyl;
m is 0, 1, 2, 3, or 4;
n is 0, 1, 2, 3, or 4; and
r is 1, 2, 3, 4, 5 or 6.

2. The compound of claim 1, or a pharmaceutically acceptable salt, solvate, stereoisomer, or prodrug thereof, wherein:
X is —O—, #—CH₂O—, #—C(═O)O—, or #—CH₂C(═O)O—, where # represents the attachment point to Ring A;
each R¹ is independently hydrogen, fluorine, —OH, C₁₋₆ alkyl, or C₁₋₆ alkoxy;
or two R¹ on adjacent carbon atoms are taken together with the intervening atoms to which they are attached to form a cyclopropyl;
each R² is independently hydrogen, fluorine, or C₁₋₆ alkyl;
R¹¹ is hydrogen or C₁₋₆ alkyl; and
R³, R⁴, R⁵, R⁶, R⁷, R⁸, R⁹, and R¹⁰ are each independently hydrogen or C₁₋₆ alkyl.

3. The compound of claim 1 or 2, or a pharmaceutically acceptable salt, solvate, stereoisomer, or prodrug thereof, wherein:
X is —O—;
each R¹ is hydrogen;
each R² is hydrogen;
R¹¹ is hydrogen; and
R³, R⁴, R¹, R⁶, R⁷, R⁸, R⁹, and R¹⁰ are each hydrogen.

4. The compound of any one of claims 1-3, or a pharmaceutically acceptable salt, solvate, stereoisomer, or prodrug thereof, wherein:
Ring A is phenyl or pyridinyl;
each R^{a} is independently halogen or C₁₋₆ alkyl; and
n is 1, 2, or 3.

5. The compound of any one of claims 1-4, or a pharmaceutically acceptable salt, solvate, stereoisomer, or prodrug thereof, wherein:
Ring A is phenyl;
each R^{a} is independently halogen; and
n is 1 or 2.

6. The compound of claim 1, wherein the compound has the structure of Formula (Ia), or a pharmaceutically acceptable salt, or solvate thereof:

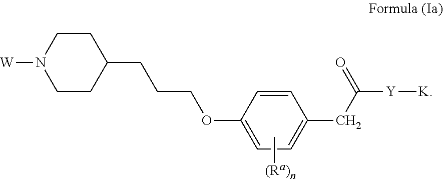

7. The compound of any one of claims 1-6, or a pharmaceutically acceptable salt, solvate, stereoisomer, or prodrug thereof, wherein:
W is phenyl, 5-membered monocyclic heteroaryl, or 6-membered monocyclic heteroaryl, wherein the phenyl or heteroaryl is unsubstituted or substituted with 1, 2, or 3 substituents selected from R^{e}.

8. The compound of claim 7, or a pharmaceutically acceptable salt, solvate, stereoisomer, or prodrug thereof, wherein:
W is 6-membered monocyclic heteroaryl, wherein the heteroaryl is unsubstituted or substituted with 1 or 2 substituents selected from R^{e}; and
each R^{e} is independently halogen, —C(O)OH, —C(O)O(C₁₋₆ alkyl), C₁₋₆ alkyl, C₁₋₆ alkoxy, or C₃₋₆ cycloalkyl; wherein each alkyl, alkoxy, and cycloalkyl is unsubstituted or substituted with 1, 2, or 3 substituents selected from the group consisting of halogen, —OH, C₁₋₆ alkyl, and C₁₋₆ alkoxy.

9. The compound of claim 1, wherein the compound has the structure of Formula (Ib), or a pharmaceutically acceptable salt, or solvate thereof:

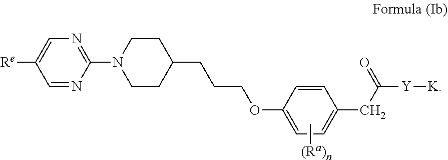

10. The compound of any one of claims 1-6, or a pharmaceutically acceptable salt, solvate, stereoisomer, or prodrug thereof, wherein:
W is —C(═O)O—R¹⁵; and
R¹⁵ is C₁₋₆ alkyl, C₃₋₆ cycloalkyl, or 3- to 6-membered heterocycloalkyl, wherein the alkyl, cycloalkyl, or heterocycloalkyl is unsubstituted or substituted by 1-3 substituents independently selected from the group consisting of C₁₋₆ alkyl and C₁₋₆ fluoroalkyl.

11. The compound of any one of claims 1-10, or a pharmaceutically acceptable salt, solvate, stereoisomer, or prodrug thereof, wherein:
Y is 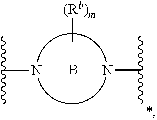
where Ring B is a heterocycloalkyl; wherein Ring B comprises 2 N atoms and 0 or 1 O or S atoms, and * represents the attachment point to K.

12. The compound of claim 11, or a pharmaceutically acceptable salt, solvate, stereoisomer, or prodrug thereof, wherein:
Ring B is a monocyclic heterocycloalkyl or a bicyclic heterocycloalkyl; wherein Ring B comprises 2 N atoms and 0 or 1 O or S atoms.

13. The compound of claim 11 or claim 12, or a pharmaceutically acceptable salt, solvate, stereoisomer, or prodrug thereof, wherein:
Ring B is a monocyclic heterocycloalkyl, fused bicyclic heterocycloalkyl, bridged bicyclic heterocycloalkyl, or spirocyclic bicyclic heterocycloalkyl; wherein Ring B comprises 2 N atoms and 0 or 1 O or S atoms.

14. The compound of any one of claims 11-13, or a pharmaceutically acceptable salt, solvate, stereoisomer, or prodrug thereof, wherein:
Ring B is a monocyclic 4- to 8-membered heterocycloalkyl; wherein Ring B comprises 2 N atoms and 0 or 1 O or S atoms.

15. The compound of claim 11, or a pharmaceutically acceptable salt, solvate, stereoisomer, or prodrug thereof, wherein:
Ring B is a 7- to 12-membered fused bicyclic heterocycloalkyl, 7- to 12-membered bridged bicyclic heterocycloalkyl, or 7- to 12-membered spirocyclic bicyclic heterocycloalkyl; wherein Ring B comprises 2 N atoms and 0 or 1 O or S atoms.

16. The compound of claim 15, or a pharmaceutically acceptable salt, solvate, stereoisomer, or prodrug thereof, wherein:
Ring B is a 7- to 12-membered spirocyclic bicyclic heterocycloalkyl; wherein Ring B comprises 2 N atoms and 0 or 1 O or S atoms.

17. The compound of claim 15 or claim 16, or a pharmaceutically acceptable salt, solvate, stereoisomer, or prodrug thereof, wherein:
Ring B is a 7- to 12-membered spirocyclic bicyclic heterocycloalkyl that is a 4,4-spiroheterocycloalkyl, a 4,5-spiroheterocycloalkyl, a 4,6-spiroheterocycloalkyl, a 5,5-spiroheterocycloalkyl, a 5,6-spiroheterocycloalkyl, or a 6,6-spiroheterocycloalkyl; wherein Ring B comprises 2 N atoms and 0 or 1 O or S atoms.

18. The compound of any one of claims 1-10, or a pharmaceutically acceptable salt, solvate, stereoisomer, or prodrug thereof, wherein:
Y is 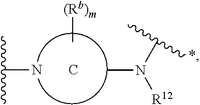
where Ring C is a bicyclic heterocycloalkyl; wherein Ring C comprises 1 or 2 N atoms and 0 or 1 O or S atoms, R¹² is hydrogen or C₁₋₄ alkyl, and * represents the attachment point to K.

19. The compound of claim 18, or a pharmaceutically acceptable salt, solvate, stereoisomer, or prodrug thereof, wherein:
Ring C is a fused bicyclic heterocycloalkyl, bridged bicyclic heterocycloalkyl, or spirocyclic bicyclic heterocycloalkyl; wherein Ring C comprises 1 or 2 N atoms and 0 or 1 O or S atoms; and
R¹² is hydrogen or C₁₋₄ alkyl.

20. The compound of claim 18 or claim 19, or a pharmaceutically acceptable salt, solvate, stereoisomer, or prodrug thereof, wherein:
Ring C is a 7- to 12-membered fused bicyclic heterocycloalkyl, 7- to 12-membered bridged bicyclic heterocycloalkyl, or 7- to 12-membered spirocyclic bicyclic heterocycloalkyl; wherein Ring C comprises 1 or 2 N atoms and 0 or 1 O or S atoms.

21. The compound of any one of claims 18-20, or a pharmaceutically acceptable salt, solvate, stereoisomer, or prodrug thereof, wherein:
Ring C is a 7- to 12-membered spirocyclic bicyclic heterocycloalkyl; wherein Ring C comprises 1 N atom and 0 or 1 O or S atoms; and
R¹² is hydrogen or C₁₂ alkyl.

22. The compound of claim 19, or a pharmaceutically acceptable salt, solvate, stereoisomer, or prodrug thereof, wherein:
Ring C is a 7- to 12-membered spirocyclic bicyclic heterocycloalkyl that is a 3,4-spiroheterocycloalkyl, a 3,5-spiroheterocycloalkyl, a 3,6-spiroheterocycloalkyl, 4,4-spiroheterocycloalkyl, a 4,5-spiroheterocycloalkyl, a 4,6-spiroheterocycloalkyl, a 5,5-spiroheterocycloalkyl, a 5,6-spiroheterocycloalkyl, or a 6,6-spiroheterocycloalkyl; wherein Ring C comprises 1 or 2 N atoms and 0 or 1 O or S atoms; and
R¹² is hydrogen or methyl.

23. The compound of any one of claims 1-22, or a pharmaceutically acceptable salt, solvate, stereoisomer, or prodrug thereof, wherein:
each R^{b} is independently fluoro or C₁₋₄ alkyl; and
m is 0, 1, or 2.

24. The compound of any one of claims 1-10, or a pharmaceutically acceptable salt, solvate, stereoisomer, or prodrug thereof, wherein:
—Y—K is

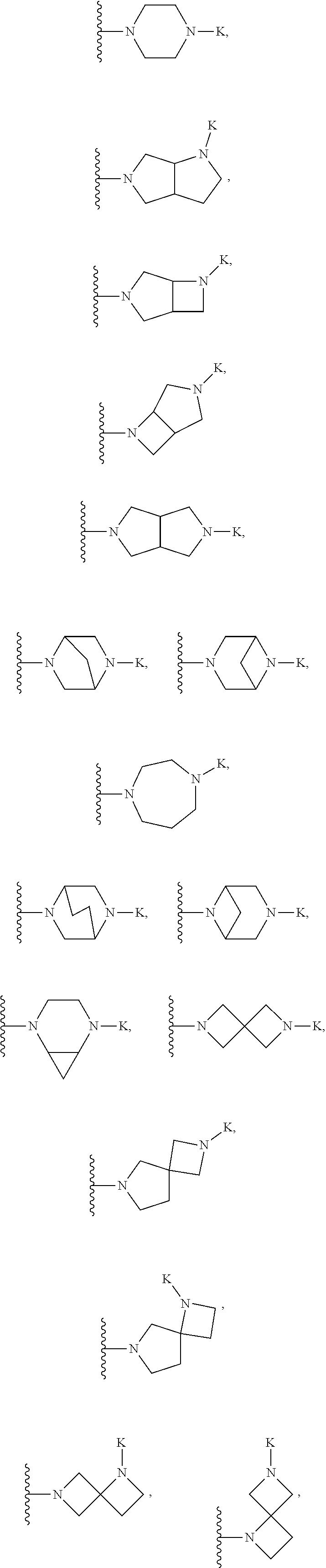

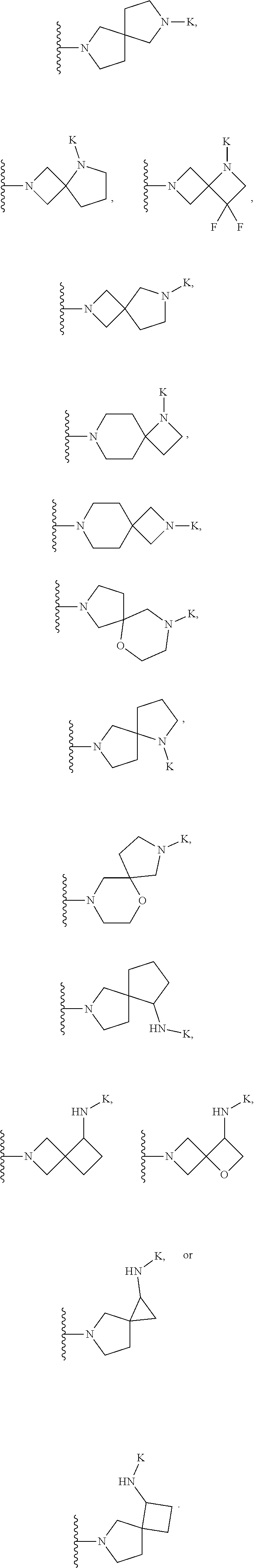

25. The compound of claim 1, wherein the compound has the structure of Formula (II), or a pharmaceutically acceptable salt, or solvate thereof:

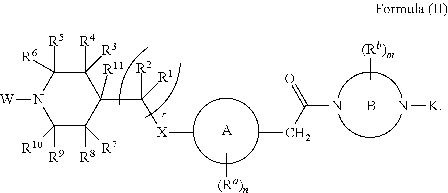

26. The compound of claim 25, wherein the compound has the structure of Formula (Ia), or a pharmaceutically acceptable salt, or solvate thereof:

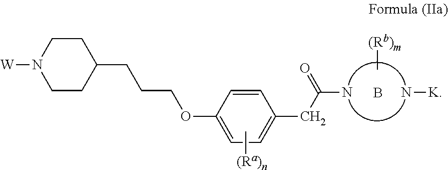

27. The compound of claim 25 or claim 26, or a pharmaceutically acceptable salt, solvate, stereoisomer, or prodrug thereof, wherein:
W is 6-membered monocyclic heteroaryl, wherein the heteroaryl is unsubstituted or substituted with 1 or 2 substituents selected from R^{e}; and
each R^{e} is independently halogen, —C(O)OH, —C(O)O(C₁₋₆ alkyl), C₁₋₆ alkyl, C₁₋₆ alkoxy, or C₃₋₆ cycloalkyl; wherein each alkyl, alkoxy, and cycloalkyl is unsubstituted or substituted with 1, 2, or 3 substituents selected from the group consisting of halogen, —OH, C₁₋₆ alkyl, and C₁₋₆ alkoxy.

28. The compound of any one of claims 25-27, wherein the compound has the structure of Formula (IIb), or a pharmaceutically acceptable salt, or solvate thereof:

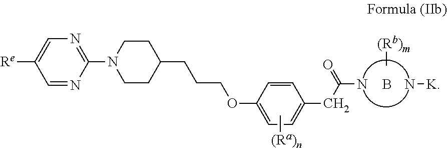

29. The compound of any one of claims 25-28, or a pharmaceutically acceptable salt, solvate, stereoisomer, or prodrug thereof, wherein:
Ring B is a monocyclic heterocycloalkyl, fused bicyclic heterocycloalkyl, bridged bicyclic heterocycloalkyl, or spirocyclic bicyclic heterocycloalkyl; wherein Ring B comprises 2 N atoms and 0 or 1 O or S atoms.

30. The compound of any one of claims 25-29, or a pharmaceutically acceptable salt, solvate, stereoisomer, or prodrug thereof, wherein:

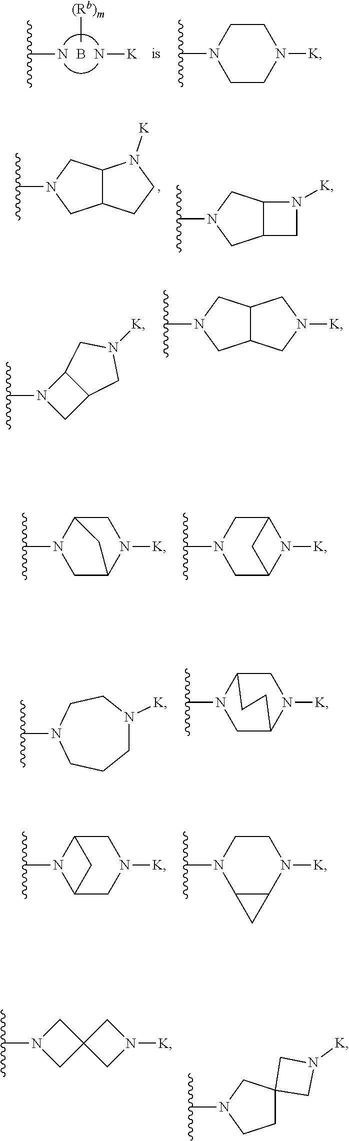

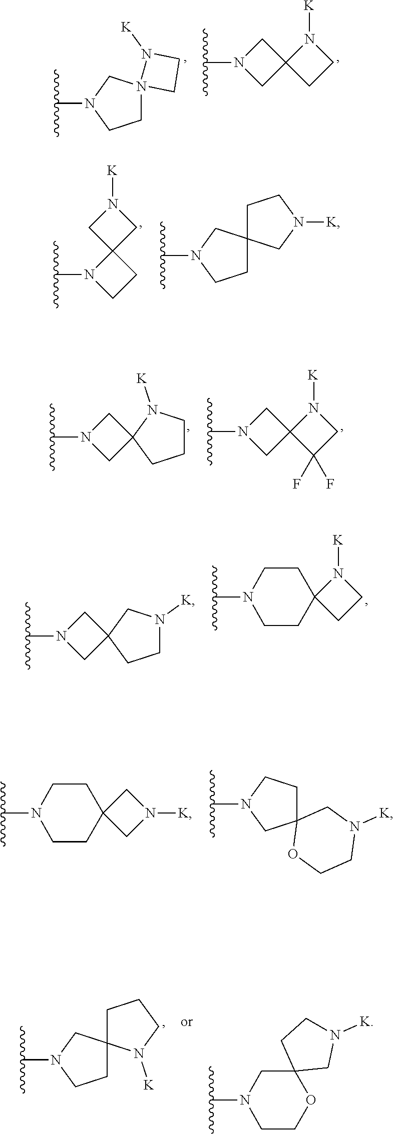

31. The compound of claim 1, wherein the compound has the structure of Formula (III), or a pharmaceutically acceptable salt, or solvate thereof:

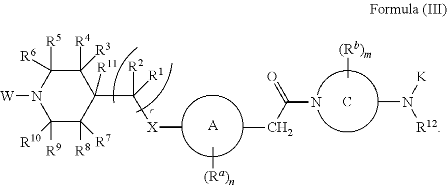

32. The compound of claim 31, wherein the compound has the structure of Formula (IIIa), or a pharmaceutically acceptable salt, or solvate thereof:

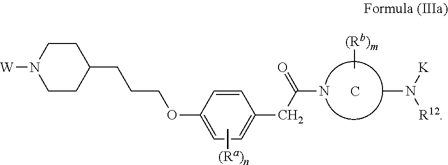

33. The compound of claim 31 or claim 32, or a pharmaceutically acceptable salt, solvate, stereoisomer, or prodrug thereof, wherein:
W is 6-membered monocyclic heteroaryl, wherein the heteroaryl is unsubstituted or substituted with 1 or 2 substituents selected from R^{e}; and
each R^{e} is independently halogen, —C(O)OH, —C(O)O(C₁₋₆ alkyl), C₁₋₆ alkyl, C₁₋₆ alkoxy, or C₃₋₆ cycloalkyl; wherein each alkyl, alkoxy, and cycloalkyl is unsubstituted or substituted with 1, 2, or 3 substituents selected from the group consisting of halogen, —OH, C₁₋₆ alkyl, and C₁₋₆ alkoxy.

34. The compound of any one of claims 31-33, wherein the compound has the structure of Formula (IIIb), or a pharmaceutically acceptable salt, or solvate thereof:

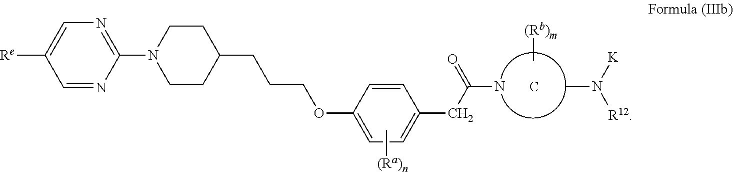

35. The compound of any one of claims 31-34, or a pharmaceutically acceptable salt, solvate, stereoisomer, or prodrug thereof, wherein:
Ring C is a 7- to 12-membered spirocyclic bicyclic heterocycloalkyl; wherein Ring C comprises 1 or 2 N atoms and 0 or 1 O or S atoms; and
R¹² is hydrogen or C₁₋₂ alkyl.

36. The compound of any one of claims 31-35, or a pharmaceutically acceptable salt, solvate, stereoisomer, or prodrug thereof, wherein:

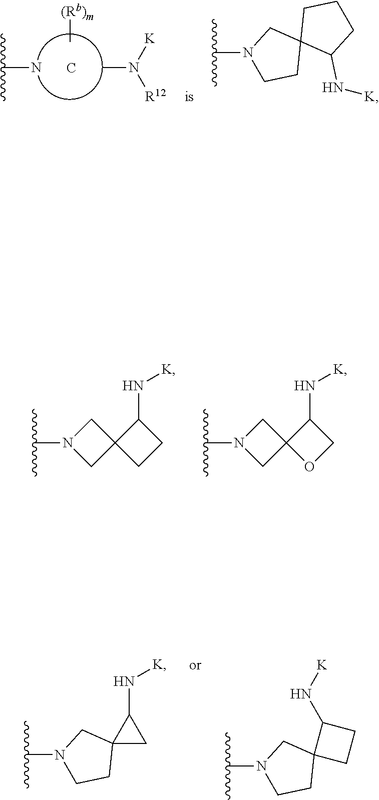

37. The compound of any one of claims 1-36, or a pharmaceutically acceptable salt, solvate, stereoisomer, or prodrug thereof, wherein:
K is C₁₋₈ alkyl, —C(═O)—C₁₋₈ alkyl, —[(CH₂)ₛ—Z]ₜ—R¹³, —[(CHR^{d})ₛ—Z]ₜ—R³, or —[(C(R^{d})₂)ₛ Z]ₜ—R¹³; wherein the alkyl is substituted by 1-6 R^{c} group;
each Z is independently CH₂O—, CH₂NH—, CH₂NR^{d}, or CH₂N⁺(R^{d})₂;
s is 1-3;
t is 1-3;
R¹³ is hydrogen or a C₁₋₈ alkyl that is unsubstituted or substituted by 1-6 R^{c} groups;
each R^{c} is independently —OH, —CH₂OH, —NH₂, —N(R^{d})₃⁺, —C(═O)OH, —S(═O)₂OH, —S(═O)₂NH₂, —P(═O)(OH)₂, —P(═O)(OH)(R^{d}), —P(═O)(OH)(OR^{d}), 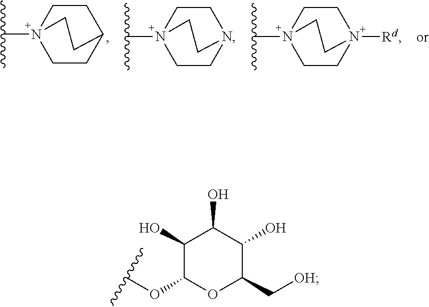
and
each R^{d} is independently C₁₋₆ alkyl.

38. The compound of any one of claims 1-37, or a pharmaceutically acceptable salt, solvate, stereoisomer, or prodrug thereof, wherein:
each R^{c} is independently —OH, —CH₂OH, —NH₂, —N(R^{d})₃⁺, —C(═O)OH, or —S(═O)₂OH.

39. The compound of any one of claims 1-38, or a pharmaceutically acceptable salt, solvate, stereoisomer, or prodrug thereof, wherein:
K is C₁₋₈ alkyl or —C(═O)—Cig alkyl; wherein the alkyl is substituted by 1-6 R^{c} groups; and
each R^{c} is independently —OH, —CH₂OH, —N(R^{d})₃⁺, —C(═O)OH, or —S(═O)₂OH.

40. The compound of any one of claims 1-39, or a pharmaceutically acceptable salt, solvate, stereoisomer, or prodrug thereof, wherein:
K is C₄₋₆ alkyl or —C(═O)—C₄₋₆ alkyl; wherein the alkyl is substituted by 1-6 —OH or —S(═O)₂OH groups.

41. The compound of any one of claims 1-36, or a pharmaceutically acceptable salt, solvate, stereoisomer, or prodrug thereof, wherein:
K is

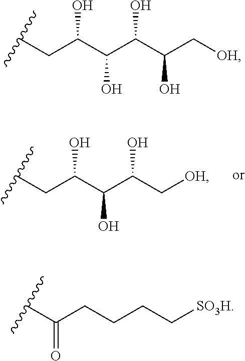 [SMILES: *C[C@H](O)[C@@H](O)[C@H](O)[C@H](O)CO ; *C[C@H](O)[C@H](O)[C@H](O)CO ; "or" ; *C(=O)CCCCS(=O)(=O)O]

42. The compound of claim 1, or a pharmaceutically acceptable salt, solvate, stereoisomer, or prodrug thereof, wherein:
X is —O—, #—CH₂O—, #—C(═O)O—, or #—CH₂C(═O)O—, where # represents the attachment point to Ring A;
each R¹ is independently hydrogen, fluorine, —OH, C₁₋₆ alkyl, or C₁₋₆ alkoxy;
or two R¹ on adjacent carbon atoms are taken together with the intervening atoms to which they are attached to form a cyclopropyl;
each R² is independently hydrogen, fluorine, or C₁₋₆ alkyl;
R¹¹ is hydrogen or C₁₋₆ alkyl;
R³, R⁴, R⁵, R⁶, R⁷, R⁸, R⁹, and R¹⁰ are each independently hydrogen or C₁₋₆ alkyl;
Ring A is phenyl or pyridinyl;
each R^{a} is independently halogen or C₁₋₆ alkyl;
n is 1, 2, or 3;
W is 6-membered monocyclic heteroaryl, wherein the heteroaryl is unsubstituted or substituted with 1 or 2 substituents selected from R^{e};
each R^{e} is independently halogen, —C(O)OH, —C(O)O(C₁₋₆ alkyl), C₁₋₆ alkyl, C₁₋₆ alkoxy, or C₃₋₆ cycloalkyl; wherein each alkyl, alkoxy, and cycloalkyl is unsubstituted or substituted with 1, 2, or 3 substituents selected from the group consisting of halogen, —OH, C₁₋₆ alkyl, and C₁₋₆ alkoxy;
or W is —C(═O)O—R¹⁵;
R¹⁵ is C₁₋₆ alkyl, C₃₋₆ cycloalkyl, or 3- to 6-membered heterocycloalkyl, wherein the alkyl, cycloalkyl, or heterocycloalkyl is unsubstituted or substituted by 1-3 substituents independently selected from the group consisting of C₁₋₆ alkyl and C₁₋₆ fluoroalkyl;
Y is 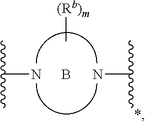
where Ring B is a monocyclic heterocycloalkyl or a bicyclic heterocycloalkyl; wherein Ring B comprises 2 N atoms and 0 or 1 O or S atoms, and * represents the attachment point to K;
or Y is 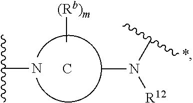
where Ring C is a fused bicyclic heterocycloalkyl, bridged bicyclic heterocycloalkyl, or spirocyclic bicyclic heterocycloalkyl; wherein Ring C comprises 1 or 2 N atoms and 0 or 1 O or S atoms; R¹² is hydrogen or C₁₋₄ alkyl; and * represents the attachment point to K;
each R^{b} is independently fluoro or C₁₋₄ alkyl;
m is 0, 1, or 2;
K is C₁₋₈ alkyl, —C(═O)—C₁₋₈ alkyl, —[(CH₂)ₛ—Z]ₜ—R¹³, —[(CHR^{d})ₛ—Z]ₜ—R³, or —[(C(R^{d})₂)ₛ Z]ₜ—R¹³; wherein the alkyl is substituted by 1-6 R^{c} group;
each Z is independently —CH₂O—, —CH₂NH—, —CH₂NR^{d}—, or —CH₂N⁺(R^{d})₂—;
s is 1-3;
t is 1-3;
R¹³ is hydrogen or a C₁₋₈ alkyl that is unsubstituted or substituted by 1-6 R^{c} groups;
each R^{c} is independently —OH, —CH₂OH, —NH₂, —N(R^{d})₃⁺, —C(═O)OH, —S(═O)₂OH, —S(═O)₂NH₂, —P(═O)(OH)₂, —P(═O)(OH)(R^{d}), —P(═O)(OH)(OR^{d}), 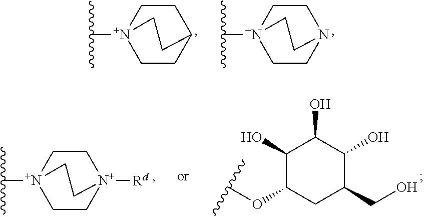
and
each R^{d} is independently C₁₋₆ alkyl.

43. The compound of claim 42, or a pharmaceutically acceptable salt, solvate, stereoisomer, or prodrug thereof, wherein:
X is —O—;
each R¹ is hydrogen;
each R² is hydrogen;
R¹¹ is hydrogen; and
R³, R⁴, R⁵, R⁶, R⁷, R⁸, R⁹, and R¹⁰ are each hydrogen;
Ring A is phenyl;
each R^{a} is independently halogen;
n is 1 or 2;
Y is 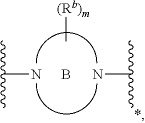
where Ring B is a monocyclic 4- to 8-membered heterocycloalkyl, 7- to 12-membered fused bicyclic heterocycloalkyl, 7- to 12-membered bridged bicyclic heterocycloalkyl, or 7- to 12-membered spirocyclic bicyclic heterocycloalkyl; wherein Ring B comprises 2 N atoms and 0 or 1 O or S atoms, and * represents the attachment point to K;
or Y is 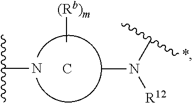
where Ring C is a 7- to 12-membered spirocyclic bicyclic heterocycloalkyl; wherein Ring C comprises 1 or 2 N atoms and 0 or 1 O or S atoms;
R¹² is hydrogen or C₁₋₂ alkyl; and * represents the attachment point to K;
K is C₁₋₈ alkyl or —C(═O)—C₁₋₈ alkyl; wherein the alkyl is substituted by 1-6 R^{c} groups;
each R^{c} is independently —OH, —CH₂OH, —N(R^{d})₃⁺, —C(═O)OH, or —S(═O)₂OH; and
each R^{d} is independently C₁₋₆ alkyl.

44. The compound of claim 42 or claim 43, or a pharmaceutically acceptable salt, solvate, stereoisomer, or prodrug thereof, wherein:
—Y—K is 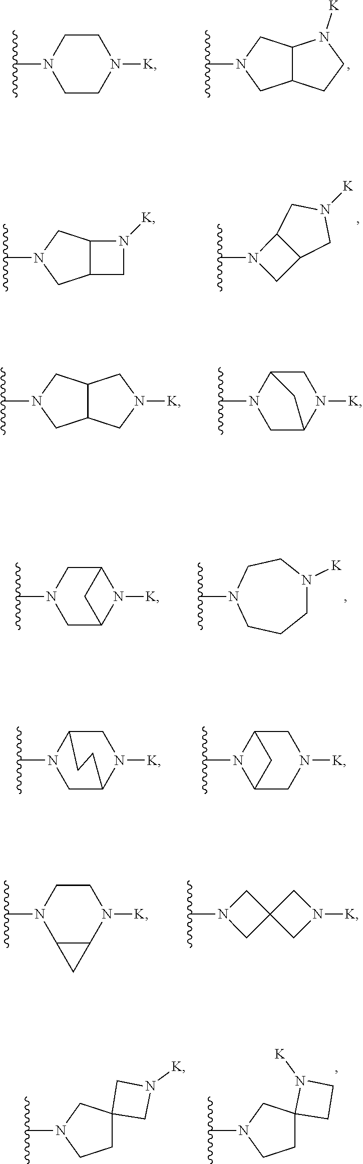 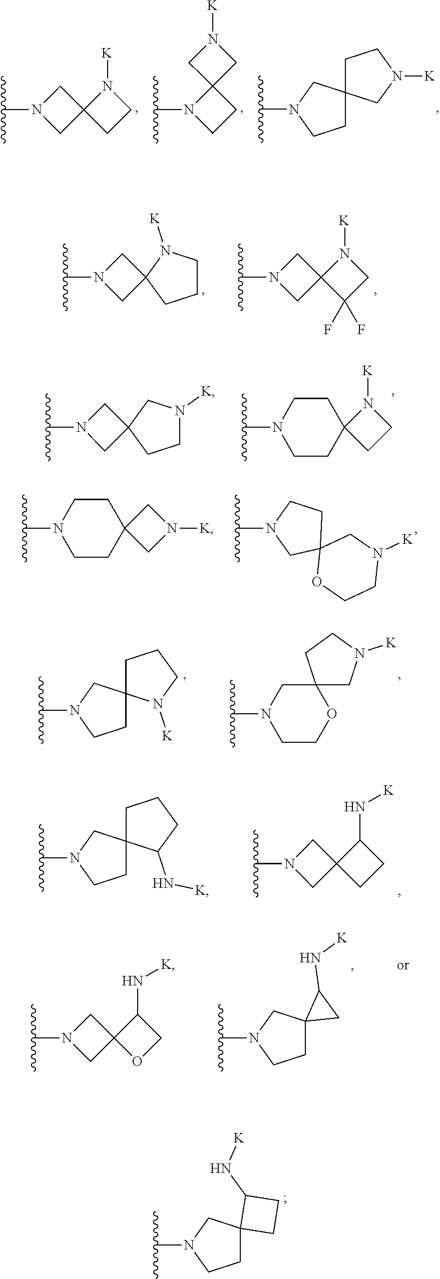
and
K is C₄₋₆ alkyl or —C(═O)—C₄₋₆ alkyl; wherein the alkyl is substituted by 1-6 —OH or —S(═O)₂OH groups.

45. The compound of claim 1, wherein the compound has the structure of Formula (IV): 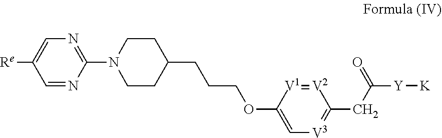
or a pharmaceutically acceptable salt, solvate, stereoisomer, or prodrug thereof, wherein:
V¹ is CH, CF, or N;
V² is CH, CF, or N; and
V³ is CH, CF, or N.

46. The compound of claim 45, wherein the compound has the structure of Formula (V): 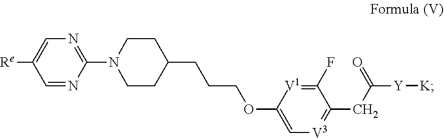
or a pharmaceutically acceptable salt, solvate, stereoisomer, or prodrug thereof, wherein:
V¹ is CH or CF; and
V³ is CH, CF, or N.

47. The compound of claim 45 or claim 46, or a pharmaceutically acceptable salt, solvate, stereoisomer, or prodrug thereof, wherein:
Y is 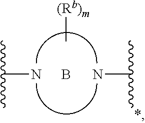
where Ring B is a monocyclic heterocycloalkyl or a bicyclic heterocycloalkyl; wherein Ring B comprises 2 N atoms and 0 or 1 O or S atoms, and * represents the attachment point to K;
or Y is 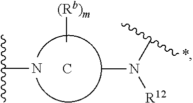
where Ring C is a fused bicyclic heterocycloalkyl, bridged bicyclic heterocycloalkyl, or spirocyclic bicyclic heterocycloalkyl; wherein Ring C comprises 1 or 2 N atoms and 0 or 1 O or S atoms; R¹² is hydrogen or C₁₋₄ alkyl; and * represents the attachment point to K;
each R^{b} is independently fluoro or C₁₋₄ alkyl;
m is 0, 1, or 2;
K is C₁₋₈ alkyl, —C(═O)—C₁₋₈ alkyl, —[(CH₂)ₛ—Z]ₜ—R¹³, —[(CHR^{d})ₛ—Z]ₜ—R¹³, or —[(C(R^{d})₂)ₛ—Z]ₜ—R¹³; wherein the alkyl is substituted by 1-6 R^{c} group;
each Z is independently —CH₂O—, —CH₂NH—, —CH₂NR^{d}—, or —CH₂N⁺(R^{d})₂—;
s is 1-3;
t is 1-3;
R¹³ is hydrogen or a C₁₋₈ alkyl that is unsubstituted or substituted by 1-6 R^{c} groups;
each R^{c} is independently —OH, —CH₂OH, —NH₂, —N(R^{d})₃⁺, —C(═O)OH, —S(═O)₂OH, —S(═O)₂NH₂, —P(═O)(OH)₂, —P(═O)(OH)(R^{d}), —P(═O)(OH)(OR^{d}), 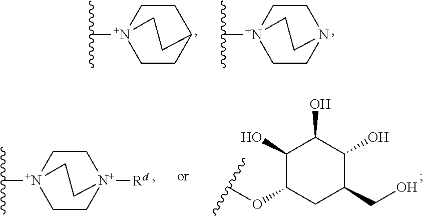
and
each R^{d} is independently C₁₋₆ alkyl.

48. The compound of any one of claims 45-47, or a pharmaceutically acceptable salt, solvate, stereoisomer, or prodrug thereof, wherein:
Y is 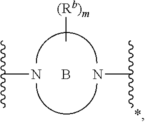
where Ring B is a monocyclic 4- to 8-membered heterocycloalkyl, 7- to 12-membered fused bicyclic heterocycloalkyl, 7- to 12-membered bridged bicyclic heterocycloalkyl, or 7- to 12-membered spirocyclic bicyclic heterocycloalkyl; wherein Ring B comprises 2 N atoms and 0 or 1 O or S atoms, and * represents the attachment point to K;
or Y is 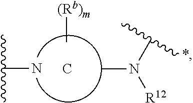
where Ring C is a 7- to 12-membered spirocyclic bicyclic heterocycloalkyl; wherein Ring C comprises 1 or 2 N atoms and 0 or 1 O or S atoms; R¹² is hydrogen or C₁₋₂ alkyl; and * represents the attachment point to K;
K is C₁₋₈ alkyl or —C(═O)—C₁₋₈ alkyl; wherein the alkyl is substituted by 1-6 R^{c} groups;
each R^{c} is independently —OH, —CH₂OH, —N(R^{d})₃⁺, —C(═O)OH, or —S(═O)₂OH; and
each R^{d} is independently C₁₋₆ alkyl.

49. The compound of any one of claims 45-48, or a pharmaceutically acceptable salt, solvate, stereoisomer, or prodrug thereof, wherein:
—Y—K is 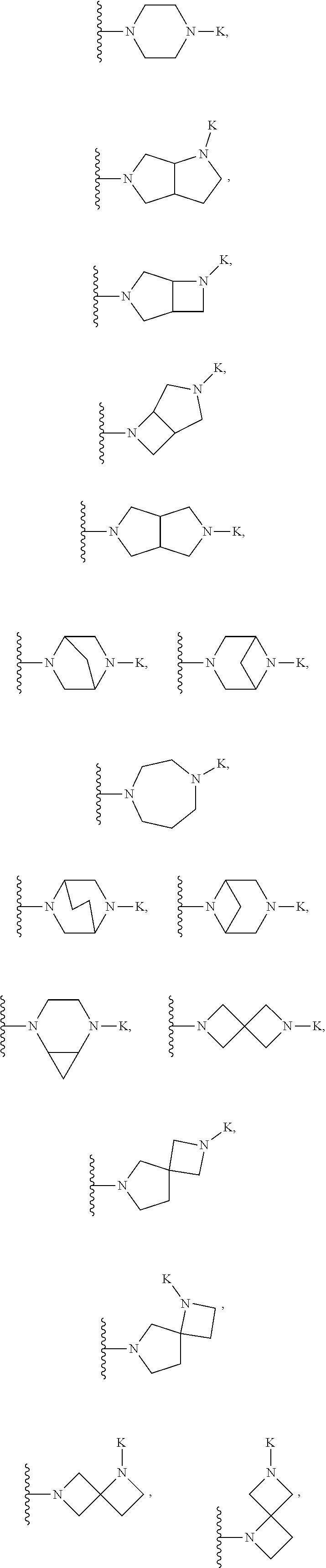 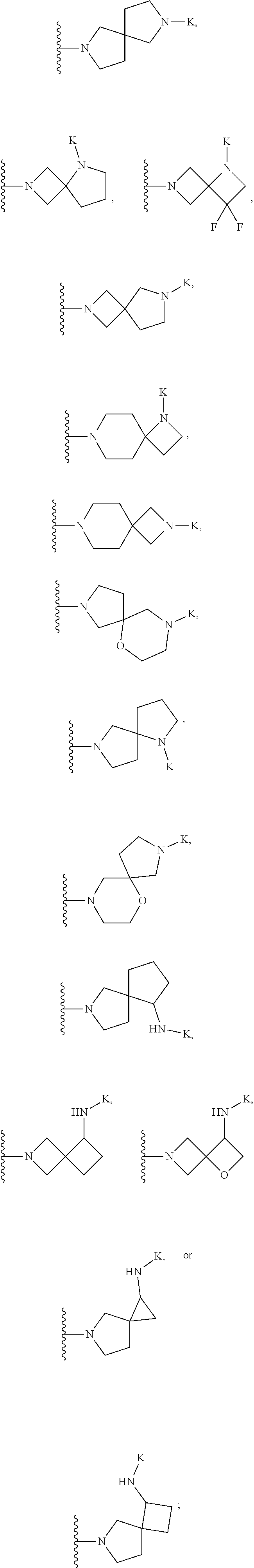
and
K is C₄₋₆ alkyl or —C(═O)—C₄₋₆ alkyl; wherein the alkyl is substituted by 1-6 —OH or —S(═O)₂OH groups.

50. The compound of any one of claims 42-49, or a pharmaceutically acceptable salt, solvate, stereoisomer, or prodrug thereof, wherein:
K is

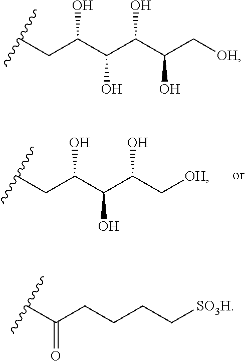 [SMILES: *C[C@H](O)[C@@H](O)[C@H](O)[C@H](O)CO ; *C[C@H](O)[C@H](O)[C@H](O)CO ; "or" ; *C(=O)CCCCS(=O)(=O)O]

51. The compound of claim 1, or a pharmaceutically acceptable salt, solvate, stereoisomer, or prodrug thereof, selected from the group consisting of:
1: 5-(7-(2-(4-(3-(1-(5-chloropyrimidin-2-yl)piperidin-4-yl)propoxy)-2-fluorophenyl)acetyl)-2,7-diazaspiro[4.4]nonan-2-yl)-5-oxopentane-1-sulfonic acid;
2: 5-(6-(2-(4-(3-(1-(5-chloropyrimidin-2-yl)piperidin-4-yl)propoxy)-2-fluorophenyl)acetyl)-2,6-diazaspiro[3.4]octan-2-yl)-5-oxopentane-1-sulfonic acid;
3: 5-(6-(2-(4-(3-(1-(5-chloropyrimidin-2-yl)piperidin-4-yl)propoxy)-2-fluorophenyl)acetyl)-2,6-diazaspiro[3.3]heptan-2-yl)-5-oxopentane-1-sulfonic acid;
4: 5-(6-(2-(4-(3-(1-(5-chloropyrimidin-2-yl)piperidin-4-yl)propoxy)-2-fluorophenyl)acetyl)-1,6-diazaspiro[3.4]octan-1-yl)-5-oxopentane-1-sulfonic acid;
5: 5-(6-(2-(4-(3-(1-(5-chloropyrimidin-2-yl)piperidin-4-yl)propoxy)-2-fluorophenyl)acetyl)-1,6-diazaspiro[3.3]heptan-1-yl)-5-oxopentane-1-sulfonic acid;
6: 5-((3aS,6aS)-5-(2-(4-(3-(1-(5-chloropyrimidin-2-yl)piperidin-4-yl)propoxy)-2-fluorophenyl)acetyl)hexahydropyrrolo[3,4-b]pyrrol-1(2H)-yl)-5-oxopentane-1-sulfonic acid;
7: 5-((1S,5R)-3-(2-(4-(3-(1-(5-chloropyrimidin-2-yl)piperidin-4-yl)propoxy)-2-fluorophenyl)acetyl)-3,6-diazabicyclo[3.2.0]heptan-6-yl)-5-oxopentane-1-sulfonic acid;
8: 5-((1S,5R)-6-(2-(4-(3-(1-(5-chloropyrimidin-2-yl)piperidin-4-yl)propoxy)-2-fluorophenyl)acetyl)-3,6-diazabicyclo[3.2.0]heptan-3-yl)-5-oxopentane-1-sulfonic acid;
9: 5-(1-(2-(4-(3-(1-(5-chloropyrimidin-2-yl)piperidin-4-yl)propoxy)-2-fluorophenyl)acetyl)-1,6-diazaspiro[3.3]heptan-6-yl)-5-oxopentane-1-sulfonic acid;
10: 5-((3aR,6aS)-5-(2-(4-(3-(1-(5-chloropyrimidin-2-yl)piperidin-4-yl)propoxy)-2-fluorophenyl)acetyl)hexahydropyrrolo[3,4-c]pyrrol-2(1H)-yl)-5-oxopentane-1-sulfonic acid;
11: 5-((1R,4R)-5-(2-(4-(3-(1-(5-chloropyrimidin-2-yl)piperidin-4-yl)propoxy)-2-fluorophenyl)acetyl)-2,5-diazabicyclo[2.2.1]heptan-2-yl)-5-oxopentane-1-sulfonic acid;
12: 5-(3-(2-(4-(3-(1-(5-chloropyrimidin-2-yl)piperidin-4-yl)propoxy)-2-fluorophenyl)acetyl)-3,6-diazabicyclo[3.1.1]heptan-6-yl)-5-oxopentane-1-sulfonic acid;
13: 5-(4-(2-(4-(3-(1-(5-chloropyrimidin-2-yl)piperidin-4-yl)propoxy)-2-fluorophenyl)acetyl)-1,4-diazepan-1-yl)-5-oxopentane-1-sulfonic acid;
14: 5-(5-(2-(4-(3-(1-(5-chloropyrimidin-2-yl)piperidin-4-yl)propoxy)-2-fluorophenyl)acetyl)-2,5-diazabicyclo[2.2.2]octan-2-yl)-5-oxopentane-1-sulfonic acid;
15: 5-((1S,4S)-5-(2-(4-(3-(1-(5-chloropyrimidin-2-yl)piperidin-4-yl)propoxy)-2-fluorophenyl)acetyl)-2,5-diazabicyclo[2.2.1]heptan-2-yl)-5-oxopentane-1-sulfonic acid;
16: 5-(6-(2-(4-(3-(1-(5-chloropyrimidin-2-yl)piperidin-4-yl)propoxy)-2-fluorophenyl)acetyl)-3,6-diazabicyclo[3.1.1]heptan-3-yl)-5-oxopentane-1-sulfonic acid;
17: 5-(5-(2-(4-(3-(1-(5-chloropyrimidin-2-yl)piperidin-4-yl)propoxy)-2-fluorophenyl)acetyl)-2,5-diazabicyclo[4.1.0]heptan-2-yl)-5-oxopentane-1-sulfonic acid;
18: 5-(2-(2-(4-(3-(1-(5-chloropyrimidin-2-yl)piperidin-4-yl)propoxy)-2-fluorophenyl)acetyl)-2,5-diazaspiro[3.4]octan-5-yl)-5-oxopentane-1-sulfonic acid;
19: 5-(6-(2-(4-(3-(1-(5-chloropyrimidin-2-yl)piperidin-4-yl)propoxy)-2-fluorophenyl)acetyl)-3,3-difluoro-1,6-diazaspiro[3.3]heptan-1-yl)-5-oxopentane-1-sulfonic acid;
20: 5-((3aR,6aR)-5-(2-(4-(3-(1-(5-chloropyrimidin-2-yl)piperidin-4-yl)propoxy)-2-fluorophenyl)acetyl)hexahydropyrrolo[3,4-b]pyrrol-1(2H)-yl)-5-oxopentane-1-sulfonic acid;
21: 5-(7-(2-(4-(3-(1-(5-chloropyrimidin-2-yl)piperidin-4-yl)propoxy)-2-fluorophenyl)acetyl)-1,7-diazaspiro[3.5]nonan-1-yl)-5-oxopentane-1-sulfonic acid;
22: 5-(7-(2-(4-(3-(1-(5-chloropyrimidin-2-yl)piperidin-4-yl)propoxy)-2-fluorophenyl)acetyl)-2,7-diazaspiro[3.5]nonan-2-yl)-5-oxopentane-1-sulfonic acid;
23: 5-(4-(2-(4-(3-(1-(5-chloropyrimidin-2-yl)piperidin-4-yl)propoxy)-2-fluorophenyl)acetyl)piperazin-1-yl)-5-oxopentane-1-sulfonic acid;
24: 5-(2-(2-(4-(3-(1-(5-chloropyrimidin-2-yl)piperidin-4-yl)propoxy)-2-fluorophenyl)acetyl)-6-oxa-2,9-diazaspiro[4.5]decan-9-yl)-5-oxopentane-1-sulfonic acid;
25: 2-(4-(3-(1-(5-chloropyrimidin-2-yl)piperidin-4-yl)propoxy)-2-fluorophenyl)-1-(6-((2S,3S,4R)-2,3,4,5-tetrahydroxypentyl)-2,6-diazaspiro[3.3]heptan-2-yl)ethan-1-one;
26: 2-(4-(3-(1-(5-chloropyrimidin-2-yl)piperidin-4-yl)propoxy)-2-fluorophenyl)-1-(6-((2S,3R,4R,5R)-2,3,4,5,6-pentahydroxyhexyl)-2,6-diazaspiro[3.3]heptan-2-yl)ethan-1-one;
27: 2-(4-(3-(1-(5-chloropyrimidin-2-yl)piperidin-4-yl)propoxy)-2-fluorophenyl)-1-(4-((2S,3R,4R,5R)-2,3,4,5,6-pentahydroxyhexyl)piperazin-1-yl)ethan-1-one;
28: 2-(4-(3-(1-(5-chloropyrimidin-2-yl)piperidin-4-yl)propoxy)-2-fluorophenyl)-1-(2-((2S,3 S,4R)-2,3,4,5-tetrahydroxypentyl)-2,6-diazaspiro[3.4]octan-6-yl)ethan-1-one;
29: 2-(4-(3-(1-(5-chloropyrimidin-2-yl)piperidin-4-yl)propoxy)-2-fluorophenyl)-1-(2-((2S,3R,4R,5R)-2,3,4,5,6-pentahydroxyhexyl)-2,6-diazaspiro[3.4]octan-6-yl)ethan-1-one;
30: 2-(4-(3-(1-(5-chloropyrimidin-2-yl)piperidin-4-yl)propoxy)-2-fluorophenyl)-1-(1-((2S,3 S,4R)-2,3,4,5-tetrahydroxypentyl)-1,6-diazaspiro[3.4]octan-6-yl)ethan-1-one;
31: 2-(4-(3-(1-(5-chloropyrimidin-2-yl)piperidin-4-yl)propoxy)-2-fluorophenyl)-1-(1-((2S,3R,4R,5R)-2,3,4,5,6-pentahydroxyhexyl)-1,6-diazaspiro[3.4]octan-6-yl)ethan-1-one;
32: 2-(4-(3-(1-(5-chloropyrimidin-2-yl)piperidin-4-yl)propoxy)-2-fluorophenyl)-1-(1-((2S,3 S,4R)-2,3,4,5-tetrahydroxypentyl)-1,6-diazaspiro[3.3]heptan-6-yl)ethan-1-one;
33: 2-(4-(3-(1-(5-chloropyrimidin-2-yl)piperidin-4-yl)propoxy)-2-fluorophenyl)-1-(1-((2S,3R,4R,5R)-2,3,4,5,6-pentahydroxyhexyl)-1,6-diazaspiro[3.3]heptan-6-yl)ethan-1-one;
34: 2-(4-(3-(1-(5-chloropyrimidin-2-yl)piperidin-4-yl)propoxy)-2-fluorophenyl)-1-((3aS,6aS)-1-((2S,3 S,4R)-2,3,4,5-tetrahydroxypentyl)hexahydropyrrolo[3,4-b]pyrrol-5(1H)-yl)ethan-1-one;
35: 2-(4-(3-(1-(5-chloropyrimidin-2-yl)piperidin-4-yl)propoxy)-2-fluorophenyl)-1-((3aS,6aS)-1-((2S,3R,4R,5R)-2,3,4,5,6-pentahydroxyhexyl)hexahydropyrrolo[3,4-b]pyrrol-5(1H)-yl)ethan-1-one;
36: 2-(4-(3-(1-(5-chloropyrimidin-2-yl)piperidin-4-yl)propoxy)-2-fluorophenyl)-1-((1R,5R)-6-((2S,3 S,4R)-2,3,4,5-tetrahydroxypentyl)-3,6-diazabicyclo[3.2.0]heptan-3-yl)ethan-1-one;
37: 2-(4-(3-(1-(5-chloropyrimidin-2-yl)piperidin-4-yl)propoxy)-2-fluorophenyl)-1-((1R,5R)-6-((2S,3R,4R,5R)-2,3,4,5,6-pentahydroxyhexyl)-3,6-diazabicyclo[3.2.0]heptan-3-yl)ethan-1-one;
38: 2-(4-(3-(1-(5-chloropyrimidin-2-yl)piperidin-4-yl)propoxy)-2-fluorophenyl)-1-((1S,5R)-3-((2S,3 S,4R)-2,3,4,5-tetrahydroxypentyl)-3,6-diazabicyclo[3.2.0]heptan-6-yl)ethan-1-one;
39: 2-(4-(3-(1-(5-chloropyrimidin-2-yl)piperidin-4-yl)propoxy)-2-fluorophenyl)-1-((1S,5R)-3-((2S,3R,4R,5R)-2,3,4,5,6-pentahydroxyhexyl)-3,6-diazabicyclo[3.2.0]heptan-6-yl)ethan-1-one;
40: 2-(4-(3-(1-(5-chloropyrimidin-2-yl)piperidin-4-yl)propoxy)-2-fluorophenyl)-1-(6-((2S,3 S,4R)-2,3,4,5-tetrahydroxypentyl)-1,6-diazaspiro[3.3]heptan-1-yl)ethan-1-one;
41: 2-(4-(3-(1-(5-chloropyrimidin-2-yl)piperidin-4-yl)propoxy)-2-fluorophenyl)-1-(6-((2S,3R,4R,5R)-2,3,4,5,6-pentahydroxyhexyl)-1,6-diazaspiro[3.3]heptan-1-yl)ethan-1-one;
42: 2-(4-(3-(1-(5-chloropyrimidin-2-yl)piperidin-4-yl)propoxy)-2-fluorophenyl)-1-(7-((2S,3 S,4R)-2,3,4,5-tetrahydroxypentyl)-2,7-diazaspiro[4.4]nonan-2-yl)ethan-1-one;
43: 2-(4-(3-(1-(5-chloropyrimidin-2-yl)piperidin-4-yl)propoxy)-2-fluorophenyl)-1-(7-((2S,3R,4R,5R)-2,3,4,5,6-pentahydroxyhexyl)-2,7-diazaspiro[4.4]nonan-2-yl)ethan-1-one;
44: 2-(4-(3-(1-(5-chloropyrimidin-2-yl)piperidin-4-yl)propoxy)-2-fluorophenyl)-1-((3aR,6aS)-5-((2S,3 S,4R)-2,3,4,5-tetrahydroxypentyl)hexahydropyrrolo[3,4-c]pyrrol-2(1H)-yl)ethan-1-one;
45: 2-(4-(3-(1-(5-chloropyrimidin-2-yl)piperidin-4-yl)propoxy)-2-fluorophenyl)-1-((3aR,6aS)-5-((2S,3R,4R,5R)-2,3,4,5,6-pentahydroxyhexyl)hexahydropyrrolo[3,4-c]pyrrol-2(1H)-yl)ethan-1-one;
46: 2-(4-(3-(1-(5-chloropyrimidin-2-yl)piperidin-4-yl)propoxy)-2-fluorophenyl)-1-((1R,4R)-5-((2S,3 S,4R)-2,3,4,5-tetrahydroxypentyl)-2,5-diazabicyclo[2.2.1]heptan-2-yl)ethan-1-one;
47: 2-(4-(3-(1-(5-chloropyrimidin-2-yl)piperidin-4-yl)propoxy)-2-fluorophenyl)-1-((1R,4R)-5-((2S,3R,4R,5R)-2,3,4,5,6-pentahydroxyhexyl)-2,5-diazabicyclo[2.2.1]heptan-2-yl)ethan-1-one;
48: 2-(4-(3-(1-(5-chloropyrimidin-2-yl)piperidin-4-yl)propoxy)-2-fluorophenyl)-1-(6-((2S,3 S,4R)-2,3,4,5-tetrahydroxypentyl)-3,6-diazabicyclo[3.1.1]heptan-3-yl)ethan-1-one;
49: 2-(4-(3-(1-(5-chloropyrimidin-2-yl)piperidin-4-yl)propoxy)-2-fluorophenyl)-1-(6-((2S,3R,4R,5R)-2,3,4,5,6-pentahydroxyhexyl)-3,6-diazabicyclo[3.1.1]heptan-3-yl)ethan-1-one;
50: 2-(4-(3-(1-(5-chloropyrimidin-2-yl)piperidin-4-yl)propoxy)-2-fluorophenyl)-1-(4-((2S,3 S,4R)-2,3,4,5-tetrahydroxypentyl)-1,4-diazepan-1-yl)ethan-1-one;
51: 2-(4-(3-(1-(5-chloropyrimidin-2-yl)piperidin-4-yl)propoxy)-2-fluorophenyl)-1-(4-((2S,3R,4R,5R)-2,3,4,5,6-pentahydroxyhexyl)-1,4-diazepan-1-yl)ethan-1-one;
52: 2-(4-(3-(1-(5-chloropyrimidin-2-yl)piperidin-4-yl)propoxy)-2-fluorophenyl)-1-(5-((2S,3 S,4R)-2,3,4,5-tetrahydroxypentyl)-2,5-diazabicyclo[2.2.2]octan-2-yl)ethan-1-one;
53: 2-(4-(3-(1-(5-chloropyrimidin-2-yl)piperidin-4-yl)propoxy)-2-fluorophenyl)-1-(5-((2S,3R,4R,5R)-2,3,4,5,6-pentahydroxyhexyl)-2,5-diazabicyclo[2.2.2]octan-2-yl)ethan-1-one;
54: 2-(4-(3-(1-(5-chloropyrimidin-2-yl)piperidin-4-yl)propoxy)-2-fluorophenyl)-1-((1S,4S)-5-((2S,3S,4R)-2,3,4,5-tetrahydroxypentyl)-2,5-diazabicyclo[2.2.1]heptan-2-yl)ethan-1-one;
55: 2-(4-(3-(1-(5-chloropyrimidin-2-yl)piperidin-4-yl)propoxy)-2-fluorophenyl)-1-((1S,4S)-5-((2S,3R,4R,5R)-2,3,4,5,6-pentahydroxyhexyl)-2,5-diazabicyclo[2.2.1]heptan-2-yl)ethan-1-one;
56: 2-(4-(3-(1-(5-chloropyrimidin-2-yl)piperidin-4-yl)propoxy)-2-fluorophenyl)-1-(3-((2S,3 S,4R)-2,3,4,5-tetrahydroxypentyl)-3,6-diazabicyclo[3.1.1]heptan-6-yl)ethan-1-one;
57: 2-(4-(3-(1-(5-chloropyrimidin-2-yl)piperidin-4-yl)propoxy)-2-fluorophenyl)-1-(3-((2S,3R,4R,5R)-2,3,4,5,6-pentahydroxyhexyl)-3,6-diazabicyclo[3.1.1]heptan-6-yl)ethan-1-one;
58: 2-(4-(3-(1-(5-chloropyrimidin-2-yl)piperidin-4-yl)propoxy)-2-fluorophenyl)-1-(5-((2S,3 S,4R)-2,3,4,5-tetrahydroxypentyl)-2,5-diazabicyclo[4.1.0]heptan-2-yl)ethan-1-one;
59: 2-(4-(3-(1-(5-chloropyrimidin-2-yl)piperidin-4-yl)propoxy)-2-fluorophenyl)-1-(5-((2S,3R,4R,5R)-2,3,4,5,6-pentahydroxyhexyl)-2,5-diazabicyclo[4.1.0]heptan-2-yl)ethan-1-one;
60: 2-(4-(3-(1-(5-chloropyrimidin-2-yl)piperidin-4-yl)propoxy)-2-fluorophenyl)-1-(5-((2S,3 S,4R)-2,3,4,5-tetrahydroxypentyl)-2,5-diazaspiro[3.4]octan-2-yl)ethan-1-one;
61: 2-(4-(3-(1-(5-chloropyrimidin-2-yl)piperidin-4-yl)propoxy)-2-fluorophenyl)-1-(5-((2S,3R,4R,5R)-2,3,4,5,6-pentahydroxyhexyl)-2,5-diazaspiro[3.4]octan-2-yl)ethan-1-one;
62: 2-(4-(3-(1-(5-chloropyrimidin-2-yl)piperidin-4-yl)propoxy)-2-fluorophenyl)-1-(3,3-difluoro-1-((2S,3 S,4R)-2,3,4,5-tetrahydroxypentyl)-1,6-diazaspiro[3.3]heptan-6-yl)ethan-1-one;
63: 2-(4-(3-(1-(5-chloropyrimidin-2-yl)piperidin-4-yl)propoxy)-2-fluorophenyl)-1-(3,3-difluoro-1-((2S,3R,4R,5R)-2,3,4,5,6-pentahydroxyhexyl)-1,6-diazaspiro[3.3]heptan-6-yl)ethan-1-one;
64: 2-(4-(3-(1-(5-chloropyrimidin-2-yl)piperidin-4-yl)propoxy)-2-fluorophenyl)-1-((3aR,6aR)-1-((2S,3S,4R)-2,3,4,5-tetrahydroxypentyl)hexahydropyrrolo[3,4-b]pyrrol-5(1H)-yl)ethan-1-one;
65: 2-(4-(3-(1-(5-chloropyrimidin-2-yl)piperidin-4-yl)propoxy)-2-fluorophenyl)-1-((3aR,6aR)-1-((2S,3R,4R,5R)-2,3,4,5,6-pentahydroxyhexyl)hexahydropyrrolo[3,4-b]pyrrol-5(1H)-yl)ethan-1-one;
66: 2-(4-(3-(1-(5-chloropyrimidin-2-yl)piperidin-4-yl)propoxy)-2-fluorophenyl)-1-(6-((2S,3 S,4R)-2,3,4,5-tetrahydroxypentyl)-2,6-diazaspiro[3.4]octan-2-yl)ethan-1-one;
67: 2-(4-(3-(1-(5-chloropyrimidin-2-yl)piperidin-4-yl)propoxy)-2-fluorophenyl)-1-(1-((2S,3 S,4R)-2,3,4,5-tetrahydroxypentyl)-1,7-diazaspiro[3.5]nonan-7-yl)ethan-1-one;
68: 2-(4-(3-(1-(5-chloropyrimidin-2-yl)piperidin-4-yl)propoxy)-2-fluorophenyl)-1-(1-((2S,3R,4R,5R)-2,3,4,5,6-pentahydroxyhexyl)-1,7-diazaspiro[3.5]nonan-7-yl)ethan-1-one;
69: 2-(4-(3-(1-(5-chloropyrimidin-2-yl)piperidin-4-yl)propoxy)-2-fluorophenyl)-1-(2-((2S,3 S,4R)-2,3,4,5-tetrahydroxypentyl)-2,7-diazaspiro[3.5]nonan-7-yl)ethan-1-one;
70: 2-(4-(3-(1-(5-chloropyrimidin-2-yl)piperidin-4-yl)propoxy)-2-fluorophenyl)-1-(2-((2S,3R,4R,5R)-2,3,4,5,6-pentahydroxyhexyl)-2,7-diazaspiro[3.5]nonan-7-yl)ethan-1-one;
71: 2-(4-(3-(1-(5-chloropyrimidin-2-yl)piperidin-4-yl)propoxy)-2-fluorophenyl)-1-(4-((2S,3 S,4R)-2,3,4,5-tetrahydroxypentyl)piperazin-1-yl)ethan-1-one;
72: 2-(4-(3-(1-(5-chloropyrimidin-2-yl)piperidin-4-yl)propoxy)-2-fluorophenyl)-1-(9-((2S,3 S,4R)-2,3,4,5-tetrahydroxypentyl)-6-oxa-2,9-diazaspiro[4.5]decan-2-yl)ethan-1-one;
73: 2-(4-(3-(1-(5-chloropyrimidin-2-yl)piperidin-4-yl)propoxy)-2-fluorophenyl)-1-(9-((2S,3R,4R,5R)-2,3,4,5,6-pentahydroxyhexyl)-6-oxa-2,9-diazaspiro[4.5]decan-2-yl)ethan-1-one;
74: 2-(4-(3-(1-(5-chloropyrimidin-2-yl)piperidin-4-yl)propoxy)-2-fluorophenyl)-1-(1-((2S,3 S,4R)-2,3,4,5-tetrahydroxypentyl)-1,7-diazaspiro[4.4]nonan-7-yl)ethan-1-one;
75: 2-(4-(3-(1-(5-chloropyrimidin-2-yl)piperidin-4-yl)propoxy)-2-fluorophenyl)-1-(1-((2S,3R,4R,5R)-2,3,4,5,6-pentahydroxyhexyl)-1,7-diazaspiro[4.4]nonan-7-yl)ethan-1-one;
76: 2-(4-(3-(1-(5-chloropyrimidin-2-yl)piperidin-4-yl)propoxy)-2-fluorophenyl)-1-(6-(((2S,3 S,4R)-2,3,4,5-tetrahydroxypentyl)amino)-2-azaspiro[4.4]nonan-2-yl)ethan-1-one;
77: 2-(4-(3-(1-(5-chloropyrimidin-2-yl)piperidin-4-yl)propoxy)-2-fluorophenyl)-1-(6-(((2S,3R,4R,5R)-2,3,4,5,6-pentahydroxyhexyl)amino)-2-azaspiro[4.4]nonan-2-yl)ethan-1-one;
78: 2-(4-(3-(1-(5-chloropyrimidin-2-yl)piperidin-4-yl)propoxy)-2-fluorophenyl)-1-(5-(((2S,3 S,4R)-2,3,4,5-tetrahydroxypentyl)amino)-2-azaspiro[3.3]heptan-2-yl)ethan-1-one;
79: 2-(4-(3-(1-(5-chloropyrimidin-2-yl)piperidin-4-yl)propoxy)-2-fluorophenyl)-1-(5-(((2S,3R,4R,5R)-2,3,4,5,6-pentahydroxyhexyl)amino)-2-azaspiro[3.3]heptan-2-yl)ethan-1-one;
80: 2-(4-(3-(1-(5-chloropyrimidin-2-yl)piperidin-4-yl)propoxy)-2-fluorophenyl)-1-(2-((2S,3 S,4R)-2,3,4,5-tetrahydroxypentyl)-6-oxa-2,9-diazaspiro[4.5]decan-9-yl)ethan-1-one;
81: 2-(4-(3-(1-(5-chloropyrimidin-2-yl)piperidin-4-yl)propoxy)-2-fluorophenyl)-1-(2-((2S,3R,4R,5R)-2,3,4,5,6-pentahydroxyhexyl)-6-oxa-2,9-diazaspiro[4.5]decan-9-yl)ethan-1-one;
82: 2-(4-(3-(1-(5-chloropyrimidin-2-yl)piperidin-4-yl)propoxy)-2-fluorophenyl)-1-(3-(((2S,3 S,4R)-2,3,4,5-tetrahydroxypentyl)amino)-1-oxa-6-azaspiro[3.3]heptan-6-yl)ethan-1-one;
83: 2-(4-(3-(1-(5-chloropyrimidin-2-yl)piperidin-4-yl)propoxy)-2-fluorophenyl)-1-(1-(((2S,3 S,4R)-2,3,4,5-tetrahydroxypentyl)amino)-5-azaspiro[2.4]heptan-5-yl)ethan-1-one;
84: 2-(4-(3-(1-(5-chloropyrimidin-2-yl)piperidin-4-yl)propoxy)-2-fluorophenyl)-1-(1-(((2S,3 S,4R)-2,3,4,5-tetrahydroxypentyl)amino)-6-azaspiro[3.4]octan-6-yl)ethan-1-one;
85: 2-(4-(3-(1-(5-chloropyrimidin-2-yl)piperidin-4-yl)propoxy)-2,6-difluorophenyl)-1-(4-((2S,3R,4R,5R)-2,3,4,5,6-pentahydroxyhexyl)piperazin-1-yl)ethan-1-one;
86: 2-(4-(4-(1-(5-chloropyrimidin-2-yl)piperidin-4-yl)butoxy)-2-fluorophenyl)-1-(4-((2S,3R,4R,5R)-2,3,4,5,6-pentahydroxyhexyl)piperazin-1-yl)ethan-1-one;
87: 2-(4-(4-(1-(5-chloropyrimidin-2-yl)piperidin-4-yl)butoxy)-2,6-difluorophenyl)-1-(4-((2S,3R,4R,5R)-2,3,4,5,6-pentahydroxyhexyl)piperazin-1-yl)ethan-1-one;
88: 2-(5-(3-(1-(5-chloropyrimidin-2-yl)piperidin-4-yl)propoxy)pyridin-2-yl)-1-(4-((2S,3R,4R,5R)-2,3,4,5,6-pentahydroxyhexyl)piperazin-1-yl)ethan-1-one;
89: 2-(5-(3-(1-(5-chloropyrimidin-2-yl)piperidin-4-yl)propoxy)-3-fluoropyridin-2-yl)-1-(4-((2S,3R,4R,5R)-2,3,4,5,6-pentahydroxyhexyl)piperazin-1-yl)ethan-1-one;
90: 2-(6-(3-(1-(5-chloropyrimidin-2-yl)piperidin-4-yl)propoxy)pyridin-3-yl)-1-(4-((2S,3R,4R,5R)-2,3,4,5,6-pentahydroxyhexyl)piperazin-1-yl)ethan-1-one;
91: 2-(4-(3-(1-(5-chloropyrimidin-2-yl)piperidin-4-yl)propoxy)-3-fluorophenyl)-1-(4-((2S,3R,4R,5R)-2,3,4,5,6-pentahydroxyhexyl)piperazin-1-yl)ethan-1-one;
92: 2-(4-(3-(1-(5-ethylpyrimidin-2-yl)piperidin-4-yl)propoxy)-2-fluorophenyl)-1-(4-((2S,3R,4R,5R)-2,3,4,5,6-pentahydroxyhexyl)piperazin-1-yl)ethan-1-one;
93: 2-(4-(3-(1-(5-ethylpyrimidin-2-yl)piperidin-4-yl)propoxy)-2,6-difluorophenyl)-1-(4-((2S,3R,4R,5R)-2,3,4,5,6-pentahydroxyhexyl)piperazin-1-yl)ethan-1-one;
94: 2-(2-fluoro-4-(3-(1-(5-propylpyrimidin-2-yl)piperidin-4-yl)propoxy)phenyl)-1-(4-((2S,3R,4R,5R)-2,3,4,5,6-pentahydroxyhexyl)piperazin-1-yl)ethan-1-one;
95: 2-(2,6-difluoro-4-(3-(1-(5-propylpyrimidin-2-yl)piperidin-4-yl)propoxy)phenyl)-1-(4-((2S,3R,4R,5R)-2,3,4,5,6-pentahydroxyhexyl)piperazin-1-yl)ethan-1-one;
96: 2-(4-(3-(1-(5-ethoxypyrimidin-2-yl)piperidin-4-yl)propoxy)-2-fluorophenyl)-1-(4-((2S,3R,4R,5R)-2,3,4,5,6-pentahydroxyhexyl)piperazin-1-yl)ethan-1-one;
97: 2-(4-(3-(1-(5-ethoxypyrimidin-2-yl)piperidin-4-yl)propoxy)-2,6-difluorophenyl)-1-(4-((2S,3R,4R,5R)-2,3,4,5,6-pentahydroxyhexyl)piperazin-1-yl)ethan-1-one;
98: 2-(2-fluoro-4-(3-(1-(5-(methoxymethyl)pyrimidin-2-yl)piperidin-4-yl)propoxy)phenyl)-1-(4-((2S,3R,4R,5R)-2,3,4,5,6-pentahydroxyhexyl)piperazin-1-yl)ethan-1-one;
99: 2-(2,6-difluoro-4-(3-(1-(5-(methoxymethyl)pyrimidin-2-yl)piperidin-4-yl)propoxy)phenyl)-1-(4-((2S,3R,4R,5R)-2,3,4,5,6-pentahydroxyhexyl)piperazin-1-yl)ethan-1-one;
100: 2-(2-fluoro-4-(4-(1-(5-(methoxymethyl)pyrimidin-2-yl)piperidin-4-yl)butoxy)phenyl)-1-(4-((2S,3R,4R,5R)-2,3,4,5,6-pentahydroxyhexyl)piperazin-1-yl)ethan-1-one;
101: 2-(2,6-difluoro-4-(4-(1-(5-(methoxymethyl)pyrimidin-2-yl)piperidin-4-yl)butoxy)phenyl)-1-(4-((2S,3R,4R,5R)-2,3,4,5,6-pentahydroxyhexyl)piperazin-1-yl)ethan-1-one;
102: 2-(2-fluoro-4-(2-((1S,2R)-2-(1-(5-(methoxymethyl)pyrimidin-2-yl)piperidin-4-yl)cyclopropyl)ethoxy)phenyl)-1-(4-((2S,3R,4R,5R)-2,3,4,5,6-pentahydroxyhexyl)piperazin-1-yl)ethan-1-one;
103: 2-(2-fluoro-4-((2-(1-(5-(methoxymethyl)pyrimidin-2-yl)piperidin-4-yl)ethoxy)methyl)phenyl)-1-(4-((2S,3R,4R,5R)-2,3,4,5,6-pentahydroxyhexyl)piperazin-1-yl)ethan-1-one;
104: isopropyl 4-(3-(3-fluoro-4-(2-oxo-2-(4-((2S,3R,4R,5R)-2,3,4,5,6-pentahydroxyhexyl)piperazin-1-yl)ethyl)phenoxy)propyl)piperidine-1-carboxylate;
105: 3-(trifluoromethyl)oxetan-3-yl 4-(3-(3-fluoro-4-(2-oxo-2-(4-((2S,3R,4R,5R)-2,3,4,5,6-pentahydroxyhexyl)piperazin-1-yl)ethyl)phenoxy)propyl)piperidine-1-carboxylate;
106: 3-(trifluoromethyl)oxetan-3-yl 4-(3-(3,5-difluoro-4-(2-oxo-2-(4-((2S,3R,4R,5R)-2,3,4,5,6-pentahydroxyhexyl)piperazin-1-yl)ethyl)phenoxy)propyl)piperidine-1-carboxylate;
107: 2-(4-(4-(1-(5-chloropyrimidin-2-yl)-4-methylpiperidin-4-yl)butoxy)-2-fluorophenyl)-1-(4-((2S,3R,4R,5R)-2,3,4,5,6-pentahydroxyhexyl)piperazin-1-yl)ethan-1-one;
108: 2-(4-(2-(6-(5-chloropyrimidin-2-yl)-6-azaspiro[2.5]octan-1-yl)ethoxy)-2-fluorophenyl)-1-(4-((2S,3R,4R,5R)-2,3,4,5,6-pentahydroxyhexyl)piperazin-1-yl)ethan-1-one;
109: 2-(4-(3-(6-(5-chloropyrimidin-2-yl)-6-azaspiro[2.5]octan-1-yl)propoxy)-2-fluorophenyl)-1-(4-((2S,3R,4R,5R)-2,3,4,5,6-pentahydroxyhexyl)piperazin-1-yl)ethan-1-one;
110: 2-(4-(2-(7-(5-chloropyrimidin-2-yl)-7-azaspiro[3.5]nonan-2-yl)ethoxy)-2-fluorophenyl)-1-(4-((2S,3R,4R,5R)-2,3,4,5,6-pentahydroxyhexyl)piperazin-1-yl)ethan-1-one;
111: 2-(4-(3-(7-(5-chloropyrimidin-2-yl)-7-azaspiro[3.5]nonan-2-yl)propoxy)-2-fluorophenyl)-1-(4-((2S,3R,4R,5R)-2,3,4,5,6-pentahydroxyhexyl)piperazin-1-yl)ethan-1-one;
112: 2-(4-(4-(7-(5-chloropyrimidin-2-yl)-7-azaspiro[3.5]nonan-2-yl)butoxy)-2-fluorophenyl)-1-(4-((2S,3R,4R,5R)-2,3,4,5,6-pentahydroxyhexyl)piperazin-1-yl)ethan-1-one;
113: 2-(4-(2-(7-(5-chloropyrimidin-2-yl)-7-azaspiro[3.5]nonan-1-yl)ethoxy)-2-fluorophenyl)-1-(4-((2S,3R,4R,5R)-2,3,4,5,6-pentahydroxyhexyl)piperazin-1-yl)ethan-1-one;
114: 2-(4-((3-(1-(5-chloropyrimidin-2-yl)piperidin-4-yl)cyclobutyl)methoxy)-2-fluorophenyl)-1-(4-((2S,3R,4R,5R)-2,3,4,5,6-pentahydroxyhexyl)piperazin-1-yl)ethan-1-one; and
115: 2-(4-(2-(3-(1-(5-chloropyrimidin-2-yl)piperidin-4-yl)cyclobutyl)ethoxy)-2-fluorophenyl)-1-(4-((2S,3R,4R,5R)-2,3,4,5,6-pentahydroxyhexyl)piperazin-1-yl)ethan-1-one;
or a pharmaceutically acceptable salt, solvate, stereoisomer, or prodrug thereof.

52. A pharmaceutical composition comprising a compound of any one of claims 1-51, or a pharmaceutically acceptable salt, solvate, stereoisomer, or prodrug thereof, and at least one pharmaceutically acceptable excipient.

53. A method of treating a condition or disorder involving the gut-brain axis in a subject in need thereof, the method comprising administering to the subject a therapeutically effective amount of a compound of any one of claims 1-51, or a pharmaceutically acceptable salt, solvate, stereoisomer, or prodrug thereof.

54. The method of claim 53, wherein the condition or disorder is associated with GPR119 activity.

55. The method of claim 53 or claim 54, wherein the condition or disorder is a metabolic disorder.

56. The method of claim 55, wherein the condition or disorder is type 2 diabetes, hyperglycemia, metabolic syndrome, obesity, hypercholesterolemia, nonalcoholic steatohepatitis, or hypertension.

57. The method of claim 53 or claim 54, wherein the condition or disorder is a nutritional disorder.

58. The method of claim 57, wherein the nutritional disorder is short bowel syndrome, intestinal failure, or intestinal insufficiency.

59. The method of claim 53 or claim 54, wherein the condition or disorder is chemotherapy-induced enteritis or radiation-induced enteritis.

60. The method of any one of claims 53-59, wherein the compound is gut-restricted.

61. The method of claim 60, wherein the compound has low systemic exposure.

62. The method of any one of claims 53-61, further comprising administering one or more additional therapeutic agents to the subject.

63. The method of claim 62, wherein the one or more additional therapeutic agents are selected from the group consisting of a TGR5 agonist, a GPR40 agonist, an SSTR5 antagonist, an SSTR5 inverse agonist, a CCK1 agonist, a PDE4 inhibitor, a DPP-4 inhibitor, a GLP-1 receptor agonist, and metformin, or a combination thereof.

64. The method of claim 63, wherein the TGR5 agonist, GPR40 agonist, SSTR5 antagonist, SSTR5 inverse agonist, or CCK1 agonist is gut-restricted.
